(19) United States
(12) Patent Application Publication
(10) Pub. No.: US 2026/0294890 A1
(43) Pub. Date: Oct. 1, 2026

(54) LONG-TERM PREVENTION AND/OR TREATMENT OF A DISEASE BY SLO-1 INHIBITORS

(71) Applicant: Elanco Animal Health GmbH, Monheim am Rhein (DE)

(72) Inventors: Nils GRIEBENOW, Dormagen (DE); Claudia SELBACH, Wermelskirchen (DE); Carolin LUDWIG ERDMANN, Meerbusch (DE); Iring HEISLER, Düsseldorf (DE); William Hunter WHITE, Düsseldorf (DE); Olaf WILL, Stolberg (DE); Dirk HEIMBACH, Düsseldorf (DE); Chouaib TAHTAOUI, Rixheim (FR); Heinz SAGER, Sugiez (CH); Brian MATHES, Indianapolis, IN (US); Sarah GEORGE, Oakdale, NSW (AU)

(21) Appl. No.: 19/474,373

(22) PCT Filed: Apr. 12, 2024
(86) PCT No.: PCT/EP2024/060054

§ 371 Date: Oct. 10, 2025

(30) Foreign Application Priority Data
Apr. 14, 2023 (EP) 23167900.2

(51) Int. Cl. A61K 31/4709; A61K 31/519; A61K 31/538; A61P 33/10

(52) U.S. Cl. CPC A61K 31/4709; A61K 31/519; A61K 31/538; A61P 33/10

(57) ABSTRACT

The present invention concerns modulators of the calcium-activated potassium channel Slo-1 of endoparasites and pharmaceutical compositions containing such modulators for use in the long-term prevention and/or treatment of a disease. Further, the invention concerns the use of a Slo-1 modulators or a pharmaceutical composition for long-term prevention and/or treatment of a disease as well as a method for long-term prevention and/or treatment of a disease comprising the administration of a Slo-1 modulator or a pharmaceutical composition comprising such a modulator at an effective dose to a subject in need thereof.

### FIELD OF THE INVENTION

The present invention concerns inhibitors of the calcium-activated potassium channel Slo-1 of endoparasites for use in the long-term prevention and/or treatment of a disease. Further, the invention concerns a pharmaceutical formulation for use in the long-term prevention and/or treatment of a disease comprising a Slo-1 inhibitor and at least one further ingredient. The invention also concerns the use of a Slo-1 inhibitor and a pharmaceutical composition comprising such an inhibitor for long-term prevention and/or treatment of a disease as well as a method for long-term prevention and/or treatment of a disease comprising the administration of a Slo-1 inhibitor or a pharmaceutical composition comprising such an inhibitor at an effective dose to a subject in need thereof.

### BACKGROUND OF THE INVENTION

Parasitic infections in animals and humans are responsible for significant suffering. Specifically, endoparasitic infections and in particular helminthiases caused by nematodes including roundworms (such as heartworms (Dirofilarai immitis) and hookworms particularly Ancylostoma caninum) can inflict diseases through infection of, and damage to various organ systems, for example, the gastrointestinal tract, the lungs and the heart so that metabolic dysfunction, nutritional deficiencies, delayed growth, loss of productivity and death are caused.

Numerous classes of drugs are used to treat endoparasitic infections and more specifically, anthelminthic drugs are used to treat nematode infections in animals.

WO 2017/178416 A1 concerns pyrazolopyrimidine compounds, methods of preparing said compounds, intermediate compounds useful for preparing said compounds, pharmaceutical compositions and combinations comprising said compounds and the use of said compounds for manufacturing pharmaceutical compositions for the treatment, control and/or prevention of diseases, in particular of helminth infections, as a sole agent or in combination with other active ingredients.

WO 2018/087036 A1, WO 2019/025341 A1, and WO 2019/215182 A1 relate to quinoline compounds, methods of preparing said compounds, intermediate compounds useful for preparing said compounds, pharmaceutical compositions and combinations comprising said compounds and the use of said compounds for manufacturing pharmaceutical compositions for the treatment, control and/or prevention of diseases, in particular of helminth infections, as a sole agent or in combination with other active ingredients.

WO 2018/197401 A1 covers bicyclic pyrazole compounds, methods of preparing said compounds, intermediate compounds useful for preparing said compounds, pharmaceutical compositions and combinations comprising said compounds and the use of said compounds for manufacturing pharmaceutical compositions for the treatment, control and/or prevention of diseases, in particular of helminth infections, as a sole agent or in combination with other active ingredients.

WO 2019/002132 A1 concerns azaquinoline compounds, methods of preparing said compounds, intermediate compounds useful for preparing said compounds, pharmaceutical compositions and combinations comprising said compounds and the use of said compounds for manufacturing pharmaceutical compositions for the treatment, control and/or prevention of diseases, in particular of helminth infections, as a sole agent or in combination with other active ingredients.

WO 2020/083971 A2 relates to compounds, methods of preparing said compounds, intermediate compounds useful for preparing said compounds, pharmaceutical compositions and combinations comprising said compounds and the use of said compounds for the treatment, control and/or prevention of diseases, in particular of helminth infections, as a sole agent or in combination with other active ingredients.

WO 2021/204930 A1 covers substituted condensed azines as anthelmintic compounds, methods of preparing said compounds, intermediate compounds useful for preparing said compounds, pharmaceutical compositions and combinations comprising said compounds and the use of said compounds for the treatment, control and/or prevention of diseases, in particular of helminth infections, as a sole agent or in combination with other active ingredients.

WO 2020/131629 A1, WO 2020/131631 A1, WO 2020/247747 A1, WO 2022/106469 A2 and WO 2022/117783 A1 concern compounds which are useful in the control of endoparasites, for example heartworms, in warm-blooded animals.

WO 2021/018839 A1 covers isoquinoline compounds, methods of preparing said compounds, intermediate compounds useful for preparing said compounds, pharmaceutical compositions and combinations comprising said compounds and the use of said compounds for manufacturing pharmaceutical compositions for the treatment, control and/or prevention of diseases, in particular of helminth infections, as a sole agent or in combination with other active ingredients.

WO 2021/122906 A1, WO 2021/122911 A1, WO 2022/122987 A1 and WO 2022/122988 A1 relate to anthelmintic compounds. These compounds can, for example, be used in the treatment of the kind of worm disease caused by helminths such as Dirofilaria, in particular Dirofilaria immitis.

WO 2020/014068 A1 concerns anthelminthic heterocyclic compounds and compositions comprising the same. A method of controlling helminths using these compounds is also described.

WO 2020/191091 A1 relates to anthelmintic aza-benzothiophene and aza-benzofuran compounds and a method for the treatment, control or prevention of a parasitic infestation or infection in an animal in need thereof by administering an effective amount of these compounds to said animal.

WO 2021/242581 A1 concerns anthelmintic heterocyclic compounds and a method for the treatment, control or prevention of a parasitic infestation or infection in an animal in need thereof by administering an effective amount of these compounds to said animal.

There is, however, still a pressing need for compounds with superior attributes in terms of activity and drug-ability profile to ensure effective prevention and/or treatment of parasitic infections, especially endoparasitic infections. In particular, there is a need for compounds with long lasting effects against (endo)parasitic infections.

### SUMMARY OF THE INVENTION

It is an object of the present invention to provide compounds with superior attributes, in particular in terms of activity, more particular in terms of long-lasting activity, to effectively prevent and/or treat a disease, in particular a disease caused by endoparasites such as helminths.

This problem is solved by the use of the compounds according to the claimed invention.

The present invention provides compounds of general formula I.1 or I.2

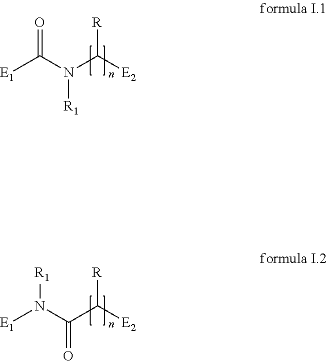

in which:
   E1 is an aromatic bicyclic heterocycle substituted with at least one substitutent and wherein the aromatic bicyclic heterocycle is a ring system selected from the group consisting of
      a ring system consisting of two 5-membered rings,
      a ring system consisting of a 5-membered ring and a 6-membered ring, or
      a ring system consisting of two 6-membered rings;
   E2 is selected from the group consisting of
      an optionally substituted aromatic benzyl,
      an optionally substituted 6-membered heterocycle comprising 1, 2, 3 or 4 heteroatoms in form of N, and
      an optionally substituted bicyclic residue wherein the bicyclic residue is selected from the group consisting of
         a ring system consisting of two 5-membered rings,
         a ring system consisting of a 5-membered ring and a 6-membered ring, or
         a ring system consisting of two 6-membered rings;
   n is 0 or 1;
   R1 is selected from the group consisting of hydrogen, cyano, —CHO, —OH, C₁-C₆-alkyl, C₁-C₉-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₂-C₄-alkenyl, C₂-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N—C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, —SO₂—C₁-C₄-halogenoalkyl having 1 to 5 halogen atoms, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl;
   phenyl-C₁-C₄-alkyl, optionally substituted with 1, 2, 3, 4 or 5 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
   heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
   R is hydrogen, C₁-C₃ alkyl; or
   when E2 is E2_5
   R and R14 together with the atoms to which they are attached form a 5 or 6-carbon atoms containing unsaturated ring, wherein the unsaturated ring is optionally substituted with one or more C₁-C₃-alkyl, and/or wherein one or more of the ring-forming carbon atoms are optionally replaced by —NH—, —N═, ═N—, —O— or —S— or
   R and R14 together with the atoms to which they are attached form a 5 or 6-carbon atoms containing unsaturated ring, wherein the unsaturated ring is optionally substituted with one or more C₁-C₃-alkyl, and/or wherein one or more of the ring-forming carbon atoms are optionally replaced by —NH—, —N═, ═N—, —O— or —S—;
   wherein E2_5 is

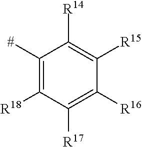

wherein
R15 is independently hydrogen, halogen, C₁₋₃ alkyl, C₁₋₃ alkoxy, or NR^{15′}R^{15″}, wherein R^{15′} and R^{15″} are independently C₁₋₃-alkyl;
R16 is independently hydrogen, halogen, C₁₋₃ alkyl, C₁₋₃ alkoxy, or NR^{16′}R^{16″}, wherein R^{16′} and R^{16″} are independently C₁₋₃-alkyl;
R17 is independently hydrogen, C₁₋₃ alkyl, C₁₋₃ alkoxy, or NR^{17′}R^{17″}, wherein R^{17′} and R^{17″} are independently C₁₋₃-alkyl;
R18 is independently hydrogen, C₁₋₃ alkyl, C₁₋₃ alkoxy, or NR^{18′}R^{18″}, wherein R^{18′} and R^{18″} are independently C₁₋₃-alkyl;
or when E2 is E2_6
R and R14 together with the atoms to which they are attached form a 5 or 6-carbon atoms containing non-aromatic ring, wherein the 5 or 6-carbon atoms containing ring is optionally substituted with one or more C1-3-alkyl or ═O, and/or wherein one or more of the ring forming carbon atoms are optionally replaced by —NH—, —N═, ═N—, —O—, —S(O)—, —S(O)2- or —S—, or
R and R14 together with the atoms to which they are attached form a 5 or 6-carbon atoms containing aromatic ring, wherein the 5 or 6-carbon atoms containing ring is optionally substituted with one or more C1-3-alkyl, and/or wherein one or more of the ring forming carbon atoms are optionally replaced by —NH—, —N═, ═N—, —O— or —S—:
   wherein E2_6 is
      E2_6 is

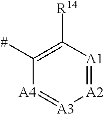

wherein
A1 is N or CR¹⁵, wherein R¹⁵ is independently hydrogen, halogen, NR^{15′}R^{15″}, wherein R^{15′} and R^{15″} are independently C₁₋₃-alkyl;
A2 is N or CR¹⁶, wherein R¹⁶ is independently hydrogen, halogen, NR^{16′}R^{16″}, wherein R^{16′} and R^{16″} are independently C₁₋₃-alkyl;
A3 is N or CR¹⁷, wherein R¹⁷ is independently hydrogen, halogen, NR^{17′}R^{17″}, wherein R^{17′} and R^{17″} are independently C₁₋₃-alkyl;
A4 is N or CR¹⁸, wherein R¹⁸ is independently hydrogen, halogen NR^{18′}R^{18″}, wherein R^{18′} and R^{18″} are independently C₁₋₃-alkyl;
or a stereoisomer, a tautomer, an N-oxide, a hydrate, a solvate, or a salt thereof,
or a mixture of the same,
for use in the long-term prevention and/or treatment of a disease.

The inventors of the present invention have found that the compounds of the claimed invention have surprising and advantageous long-lasting activity properties. It was particularly surprising and could not have been predicted by the skilled person that the pharmacokinetic properties of the compounds for use according to the invention, especially their distribution, metabolization and tolerance properties in the treated subjects, optionally further modified by formulation (e.g., by a sustained release), are so advantageous that the compounds can prevent and/or treat a disease in different body compartments for a long-term, i.e. for at least one month. Therefore, the compounds of the claimed invention can be effectively used for the long-term prevention and/or treatment of a disease, in particular a helminthic infection in an animal, preferably in a cat or a dog. The long-lasting activity properties also ensure that the compounds for use according to the invention can be applied in very convenient (i.e. less-frequent) and cost-efficient dosage regimens.

The compounds of the claimed invention are considered as modulators of the calcium-activated potassium channel Slo-1 of nematodes. Slo-1 can be regarded as the helminth's ortholog of the human KCa1.1 channel (potassium calcium-activated channel subfamily M alpha 1), which is encoded by the KCNMA1 gene (KCa1.1 and KCNMA1 are often used synonymously). Slo-1 exhibits calcium-activated potassium channel activity and voltage-gated potassium channel activity. Slo-1 channels play an important role in the neuromuscular system as well as in secretory cells among others. Thus, Slo-1 modulators are reported to be involved in several processes including behavioural response to ethanol, locomotion and pharyngeal pumping. More particularly, Slo-1 modulators disrupt neuromuscular transmission causing a flaccid paralysis and also affect feeding and egg-laying. Further, Slo-1 modulators also slow the development of the larvae and the adults of the corresponding helminth.

The claimed invention also provides a pharmaceutical composition for use in the long-term prevention and/or treatment of a disease, in particular a helminthic infection, comprising at least one compound of the claimed invention and at least one further ingredient. The pharmaceutical composition of the claimed invention is particularly suitable for the long-term prevention and/or treatment of a disease, in particular of a helminthic infection. The long-lasting activity properties of the pharmaceutical composition for use according to the invention have been surprising and could not have been predicted by the skilled person.

Further the invention concerns the use of the claimed compounds for long-term prevention and/or treatment of a disease, in particular of a helminthic infection. The claimed compounds are particularly suitable for this use due to their surprising long-lasting activity properties.

Lastly, the invention concerns a method for long-term prevention and/or treatment of a disease comprising the administration of a compound of the claimed invention or a pharmaceutical composition of the claimed invention at an effective dose to a subject in need thereof. The method of the claimed invention is particularly suitable for long-term prevention and/or treatment of a disease, in particular of a helminthic infection, due to the compounds of the invention which have surprising long-lasting activity properties.

### DETAILED DESCRIPTION OF ILLUSTRATIVE EMBODIMENTS

### General Definitions

The term “substituted” means that one or more hydrogen atoms on the designated atom or group are replaced with a different atom or group, preferably with a selection from an indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3.

The term “nil” as used herein with reference to a group, substituent, moiety, or the like, indicates that that group, substituent, or moiety is not present. Wherein a group, substituent, or moiety is ordinarily bonded to two or more other groups, substituents, or moieties, the others are bonded together in lieu of the group, substituent, or moiety which is nil. For example, with a compound having the structure A-B-C; wherein B is nil, then A is directly bonded to C and the compound is A-C. As another example, with a compound having the structure A-B-C; wherein C is nil, then the compound is A-B.

As used herein, the term “one or more” means “1, 2, 3, 4 or 5, particularly 1, 2, 3 or 4, more particularly 1, 2 or 3, even more particularly 1 or 2.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

Should a composite substituent be composed of more than one parts, for example. (C₁-C₄-alkoxy)-(C₁-C₄-alkyl)-, it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)-group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

As used herein, the position via which a respective substituent is connected to the rest of the molecule may in a drawn structure be depicted by a hash sign (#) or a dashed line in said substituent.

The term “comprising” when used in the specification includes “consisting of”. For example a composition “comprising” X may consist exclusively of X or may include something additional, for example, X+Y.

Preparation of the compounds according to the invention and the determination of properties of compounds according to the invention (for example half-life values and plasma clearance values) requires conventional techniques known to the skilled person unless otherwise indicated. These techniques are fully explained in the literature.

Unless otherwise defined herein, scientific and technical terms used in this application shall have the meanings that are commonly understood by those of ordinary skill in the art. In case of conflict, the present specification, including definitions, will control.

The terms as mentioned in the present text have the following meanings:

As used herein, the term “about” modifying the quantity of an ingredient, parameter, calculation, or measurement in the compositions employed in the methods of the disclosure refers to the variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within an order of magnitude, typically within 10%, more typically still within 5%, of a given value or range. The term “about” also encompasses amounts that differ due to different equilibrium conditions for a composition resulting from a particular initial mixture. Whether or not modified by the term “about”, the paragraphs include equivalents to the quantities. Reference to “about” a value or parameter herein includes (and describes) embodiments that are directed to that value or parameter per se. For example, description referring to “about X” includes description of “X”. Numeric ranges are inclusive of the numbers defining the range.

The word “substantially” does not exclude “completely”. For example a composition which is “substantially free” from Y may be completely free from Y.

If within the present text any item is referred to as “as mentioned herein”, it means that it may be mentioned anywhere in the present text.

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms.

The term “C₁-C₄-alkyl” means a linear or branched, saturated, monovalent hydrocarbon group having 1, 2, 3, or 4 carbon atoms, for example a methyl, ethyl, n-propyl, isopropyl, n-butyl, sec-butyl, isobutyl or a tert-butyl group, or a further constitutional isomer thereof. Particularly, said group has 1, 2 or 3 carbon atoms (“C₁-C₃-alkyl”), for example a methyl, ethyl, n-propyl or isopropyl group.

The term “C₁-C₄-hydroxyalkyl” means a linear or branched, saturated, monovalent hydrocarbon group in which the term “C₁-C₄-alkyl” is defined supra, and in which 1 hydrogen atoms are replaced with a hydroxy group, for example a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “—NH(C₁-C₄-alkyl)” or “—N(C₁-C₄-alkyl)₂” means a linear or branched, saturated, monovalent group in which the term “C₁-C₄-alkyl” is as defined supra, for example a methylamino, ethylamino, n-propylamino, isopropylamino, N,N-dimethylamino, N-methyl-N-ethylamino or N,N-diethylamino group.

The term “—S—C₁-C₄-alkyl”, “—S(O)—C₁-C₄-alkyl” or “—SO₂—C₁-C₄-alkyl” means a linear or branched, saturated group in which the term “C₁-C₄-alkyl” is as defined supra, for example a methylsulfanyl, ethylsulfanyl, n-propylsulfanyl, isopropylsulfanyl, n-butylsulfanyl, sec-butylsulfanyl, isobutylsulfanyl or tert-butylsulfanyl group, a methylsulfinyl, ethylsulfinyl, n-propylsulfinyl, isopropylsulfinyl, n-butylsulfinyl, sec-butylsulfinyl, isobutylsulfinyl or tert-butylsulfinyl group, or a methylsulfonyl, ethylsulfonyl, n-propylsulfonyl, isopropylsulfonyl, n-butylsulfonyl, sec-butylsulfonyl, isobutylsulfonyl or tert-butylsulfonyl group.

The term “C₁-C₄-halogenoalkyl” means a linear or branched, saturated, monovalent hydrocarbon group in which the term “C₁-C₄-alkyl” is as defined supra, and in which one or more of the hydrogen atoms are replaced, identically or differently, with a halogen atom. Particularly, said halogen atom is a fluorine atom. More particularly, all said halogen atoms are fluorine atoms (“C₁-C₄-fluoroalkyl”). Said C₁-C₄-halogenoalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₄-alkoxy” means a linear or branched, saturated, monovalent group of formula (C₁-C₄-alkyl)-O—, in which the term “C₁-C₄-alkyl” is as defined supra, for example a methoxy, ethoxy, n-propoxy, isopropoxy, n-butoxy, sec-butoxy, isobutoxy or tert-butoxy group, or a further constitutional isomer thereof.

The term “C₁-C₄-halogenoalkoxy” means a linear or branched, saturated, monovalent C₁-C₄-alkoxy group, as defined supra, in which one or more of the hydrogen atoms is replaced, identically or differently, with a halogen atom. Particularly, said halogen atom is a fluorine atom. Said C₁-C₄-halogenoalkoxy group is, for example, fluoromethoxy, difluoromethoxy, trifluoromethoxy, 2,2,2-trifluoroethoxy or pentafluoroethoxy.

The term “C₂-C₄-alkenyl” means a linear or branched, monovalent hydrocarbon group, which contains one double bond, and which has 2, 3 or 4 carbon atoms. Said C₂-C₄-alkenyl group is, for example, an ethenyl (or “vinyl”), a prop-2-en-1-yl (or “allyl”), prop-1-en-1-yl, but-3-enyl, but-2-enyl, but-1-enyl, prop-1-en-2-yl (or “isopropenyl”), 2-methylprop-2-enyl, 1-methylprop-2-enyl, 2-methylprop-1-enyl or a 1-methylprop-1-enyl, group. Particularly, said group is allyl.

The term “C₂-C₄-alkynyl” means a linear monovalent hydrocarbon group which contains one triple bond, and which contains 2, 3 or 4 carbon atoms. Said C₂-C₄-alkynyl group is, for example, an ethynyl, a prop-1-ynyl, prop-2-ynyl (or “propargyl”), but-1-ynyl, but-2-ynyl, but-3-ynyl or 1-methylprop-2-ynyl, group. Particularly, said alkynyl group is prop-1-ynyl or prop-2-ynyl.

The term “C₃-C₆-cycloalkyl” means a saturated, monovalent, monocyclic hydrocarbon ring which contains 3, 4, 5 or 6 carbon atoms (“C₃-C₆-cycloalkyl”). Said C₃-C₆-cycloalkyl group is for example, a monocyclic hydrocarbon ring, for example a cyclopropyl, cyclobutyl, cyclopentyl or cyclohexyl group.

The term “C₃-C₆-halogenocycloalkyl” means a saturated, monovalent, monocyclic hydrocarbon ring in which the term “C₃-C₆-cycloalkyl” is as defined supra, and in which one or more of the hydrogen atoms are replaced, identically or differently, with a halogen atom. Particularly, said halogen atom is a fluorine or chlorine atom. Said C₃-C₆-halogenocycloalkyl group is for example, a monocyclic hydrocarbon ring substituted with one or two fluorine or chlorine atoms, for example a 1-fluorocyclopropyl, 2-fluorocyclopropyl, 2,2-difluorocyclopropyl, 2,3-difluorocyclopropyl, 1-chlorocyclopropyl, 2-chlorocyclopropyl, 2,2-dichlorocyclopropyl, 2,3-dichlorocyclopropyl, 2-fluoro-2-chlorocyclopropyl and 2-fluoro-3-chlorocyclopropyl group.

The term “benzo-C₅-C₆-cycloalkyl” means a monovalent, bicyclic hydrocarbon ring wherein a saturated, monovalent, monocyclic hydrocarbon ring which contains 5 or 6 carbon atoms (“C₅-C₆-cycloalkyl”) is annelated to a phenyl ring. Said benzo-C₅-C₆-cycloalkyl group is for example, a bicyclic hydrocarbon ring, for example an indane (i.e. 2,3-dihydro-1H-indene) or tetraline (i.e. 1,2,3,4-tetrahydronaphthalene) group.

The term “spirocycloalkyl” means a saturated, monovalent bicyclic hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl, spiro[2.4]heptyl, spiro[2.5]octyl, spiro[2.6]nonyl, spiro[3.3]heptyl, spiro[3.4]octyl, spiro[3.5]nonyl, spiro[3.6]decyl, spiro[4.4]nonyl, spiro[4.5]decyl, spiro[4.6]undecyl or spiro[5.5]undecyl.

The term “heterocycloalkyl” means a monocyclic or bicyclic, saturated or partially saturated heterocycle with 4, 5, 6, 7, 8, 9 or 10 ring atoms in total (a “4- to 10-membered heterocycloalkyl” group), particularly 4, 5 or 6 ring atoms (a “4- to 6-membered heterocycloalkyl” group), which contains one or two identical or different ring heteroatoms from the series N, O and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as tetrahydrofuranyl, oxolanyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl, 1,3-thiazolidinyl or 1,2,4-triazolidinyl, for example; or a 6-membered ring, such as tetrahydropyranyl, tetrahydrothiopyranyl, piperidinyl, morpholinyl, dithianyl, thiomorpholinyl, piperazinyl, oxanyl, 1,3-dioxanyl, 1,4-dioxanyl or 1,2-oxazinanyl, for example; or a 7-membered ring, such as azepanyl, 1,4-diazepanyl or 1,4-oxazepanyl, for example; or a bicyclic 7-membered ring, such as 2-oxa-5-azabicyclo[4.1.0]heptan-5-yl or 6-oxa-3-azabicyclo[3.1.1]heptan, for example; or a bicyclic 8-membered ring, such as 5,6-dihydro-4H-furo[2,3-c]pyrrole or 8-oxa-3-azabicyclo[3.2.1]octan, for example; or a bicyclic 9-membered ring, such as octahydro-1H-pyrrolo[3,4-b]pyridine, 1,3-dihydro-isoindol, 2,3-dihydro-indol, 3,7-dioxa-9-azabicyclo[3.3.1]nonan or 3,9-dioxa-7-azabicyclo[3.3.1]nonan, for example; or a bicyclic 10-membered ring, such as decahydroquinoline or 3,4-dihydroisoquinolin, for example.

The term “heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one or two identical or different ring heteroatoms from the series: N, O, S; it being possible for said heterospirocycloalkyl group to be attached to the rest of the molecule via any one of the carbon atoms, except the spiro carbon atom, or, if present, a nitrogen atom. Said heterospirocycloalkyl group is, for example, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, oxaazaspiro[2.5]octyl, azaspiro[4.5]decyl, oxazaspiro[5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “6- or 10-membered aryl” means a monovalent, monocyclic or bicyclic aromatic ring having 6 or 10 carbon ring atoms, for example a phenyl or naphthyl group.

The term “heteroaryl” means a monovalent, monocyclic, bicyclic or tricyclic aromatic ring having 5, 6, 9 or 10 ring atoms (a “5- to 10-membered heteroaryl” group), particularly 5 or 6 ring atoms (a “5- to 6-membered heteroaryl” group), which contains at least one ring heteroatom and optionally one, two or three further ring heteroatoms from the series: N, O and/or S, and which is bound via a ring carbon atom or optionally via a ring nitrogen atom (if allowed by valency). Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, dihydropyridinyl, pyridazinyl, pyrimidinyl, tetrahydropyrimidinyl, pyrazinyl or triazinyl.

The term “heterocyclyl” means a heterocycle selected from the group consisting of heterocycloalkyl and heteroaryl. Particularly, the term “4- to 6-membered heterocyclyl” means a heterocycle selected from the group consisting of 4- to 6-membered heterocycloalkyl and 5- to 6-membered heteroaryl.

In general, and unless otherwise mentioned, the heteroaryl or heteroarylene groups include all possible constitutional isomeric forms thereof, for example: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₄”, as used in the present text, for example in the context of the definition of “C₁-C₄-alkyl”, “C₁-C₄-halogenoalkyl”, “C₁-C₄-hydroxyalkyl”, “C₁-C₄-alkoxy” or “C₁-C₄-halogenoalkoxy” means an alkyl group having a finite number of carbon atoms of 1 to 4, i.e. 1, 2, 3 or 4 carbon atoms.

Further, as used herein, the term “C₃-C₆”, as used in the present text, for example in the context of the definition of “C₃-C₆-cycloalkyl” or C₃-C₆-halogenocycloalkyl, means a cycloalkyl group having a finite number of carbon atoms of 3 to 6, i.e. 3, 4, 5 or 6 carbon atoms.

When a range of values is given, said range encompasses each value and sub-range within said range.

For example:
“C₁-C₄” encompasses C₁, C₂, C₃, C₄, C₁-C₄, C₁-C₃, C₁-C₂, C₂-C₄, C₂-C₃, and C₃-C₄;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₄” encompasses C₃, C₄, and C₃-C₄;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄-C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀1, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

It is possible for the compounds of general formula (I) to exist as isotopic variants. The invention therefore includes one or more isotopic variant(s) of the compounds of general formula (I), particularly deuterium-containing compounds of general formula (I).

The term “isotopic variant” of a compound or a reagent is defined as a compound exhibiting an unnatural proportion of one or more of the isotopes that constitute such a compound. The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), 3H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ²⁹I and ¹³¹I, respectively.

With respect to the treatment and/or prevention of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful, for example, in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (for example Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (for example Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (for example lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as for example cytochrome P₄₅₀.

Where the plural form of the word compounds, salts, polymorph, stereoisomer, hydrates, solvates and the like, is used herein, this is taken to mean also a single compound, salt, polymorph, stereoisomer, hydrate, solvate or the like. Also, when referring to a ‘noun’ in singular, the plural is meant to be included, unless specified otherwise.

As used in the present disclosure and claims the singular forms “a”, “an” and “the” include plural forms unless the context clearly dictates otherwise.

By “stable compound’ or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds. In certain instances, it is possible that asymmetry may also be present due to restricted rotation around a double bond or due to a ring structure, wherein the rotation of bonds is restricted or prevented. These geometric isomers may be indicated as cis- or trans-isomers or as (E)- and (Z)-isomers.

Preferred compounds are those which produce the more desirable biological activity. Separated, pure or partially purified constitutional isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (for example, HPLC columns using a chiral phase), with or without conventional derivatisation, optimally chosen to maximise the separation of the enantiomers. Suitable HPLC columns using a chiral phase are commercially available, such as those manufactured by Daicel, for example, Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of stereoisomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, for example, (R)- or (S)-stereoisomers, (E)- or (Z)-isomers, cis- or trans-isomers, in any ratio. Isolation of a single stereoisomer, for example, a single enantiomer or a single diastereomer, of a compound of the present invention is achieved by any suitable state of the art method, such as chromatography, especially chiral chromatography.

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, for example a hydrate, hemi-, (semi-), mono-, sesqui-, di-, tri-, tetra-, penta-etc. solvates or hydrates, respectively, are possible. The present invention includes all such hydrates or solvates.

Further, it is possible for the compounds of the present invention to exist in free form, for example as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric,
D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

Unless specified otherwise, suffixes to chemical names or structural formulae relating to salts, such as “hydrochloride”, “trifluoroacetate”, “sodium salt”, or “x HCl”, “x CF₃COOH”, “x Na”, for example, mean a salt form, the stoichiometry of which salt form not being specified.

Furthermore, the present invention includes all possible crystalline forms, or polymorphs, of the compounds of the present invention, either as single polymorph, or as a mixture of more than one polymorph, in any ratio.

It belongs to the skilled person's common general knowledge and thus includes standard techniques to produce any stereoisomer, tautomer, N-oxide, hydrate, solvate, salt, or mixture of the same of a compound for use according to the claimed invention.

### Compounds of the Present Invention

In one embodiment, the compound for use in the long-term prevention and/or treatment of a disease is a compound
wherein E1 and E2 are selected from the following pairs when the compound of formula I is a compound of formula I.1:
E1_1 and E2_1 wherein n is 0;
E1_2 and E2_1 wherein n is 0;
E1_3 and E2_1 wherein n is 0;
E1_6 and E2_1 wherein n is 0;
E1_7 and E2_2 wherein n is 0;
or
wherein E1 and E2 form the following pair when the compound of formula I is a compound of formula I.2:
E1_7 and E2_2 wherein n is 0;
in which
E1_is

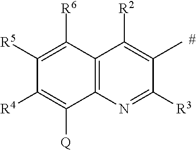

wherein
R2 is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —NH(C(O)—C₁-C₄-alkyl), —N(C₁-C₄-alkyl)(C(O)—C₁-C₄-alkyl), —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl;
2-oxocyclobutyl, 3-oxocyclobutyl, 2-thiooxocyclobutyl, 3-thiooxocyclobutyl, 3-thietanyl, 2-thietanyl, oxetan-3-yl, oxetan-2-yl, 1-oxidothietan-3-yl, 1-oxidothietan-2-yl, 1-imino-1-oxido-1-thietan-3-yl, 1-imino-1-oxido-1-thietan-2-yl, 1,1-dioxidothietan-3-yl, 1,1-dioxidothietan-2-yl, 1,1-dioxido-1,2-thiazetidin-3-yl, 1,1-dioxido-1,2-thiazetidin-4-yl, 1-oxido-1,2-thiazetidin-3-yl, 1-oxido-1,2-thiazetidin-4-yl, 2-oxido-1,2-oxathietan-3-yl, 2-oxido-1,2-oxathietan-4-yl, 2,2-dioxido-1,2-oxathietan-3-yl, 2,2-dioxido-1,2-oxathietan-4-yl, 4-oxoazetidin-2-yl, 2-oxoazetidin-3-yl, 4-thioxoazetidin-2-yl, 2-thioxoazetidin-3-yl, 2-hydroxycyclobutyl, 3-hydroxycyclobutyl, 2-mercaptocyclobutyl, 3-mercaptocyclobutyl, 2-fluorocyclobutyl, 3-fluorocyclobutyl, 2,2-difluorocyclobutyl, 3,3-difluorocyclobutyl, 2-chlorocyclobutyl, 3-chlorocyclobutyl, 2,2-dichlorocyclobutyl, 3,3-dichlorocyclobutyl, 2-bromocyclobutyl, 3-bromocyclobutyl, 2,2-dibromocyclobutyl, 3,3-dibromocyclobutyl, 2-iodocyclobutyl, 3-iodocyclobutyl, 2,2-diiodocyclobutyl, 3,3-diiodocyclobutyl, 3-methoxyiminocyclobutyl, 2-fluoro-3-(methoxyimino)cyclobutyl, 2,2-difluoro-3-(methoxyimino)cyclobutyl, 2-chloro-3-(methoxyimino)cyclobutyl, 2,2-dichloro-3-(methoxyimino)cyclobutyl, 2-bromo-3-(methoxyimino)cyclobutyl, 2,2-dibromo-3-(methoxyimino)cyclobutyl, 2-iodo-3-(methoxyimino)cyclobutyl, 2,2-diiodo-3-(methoxyimino)cyclobutyl, 3-(hydroxyimino)cyclobutyl, 2-fluoro-3-(hydroxyimino)cyclobutyl, 2,2-difluoro-3-(hydroxyimino)cyclobutyl, 2-chloro-3-(hydroxyimino)cyclobutyl, 2,2-dichloro-3-(hydroxyimino)cyclobutyl, 2-bromo-3-(hydroxyimino)cyclobutyl, 2,2-dibromo-3-(hydroxyimino)cyclobutyl, 2-iodo-3-(hydroxyimino)cyclobutyl, 2,2-diiodo-3-(hydroxyimino)cyclobutyl, tetrahydro-2H-pyran-4-yl, 3,6 dihydro-2H-pyran-4-yl, ethyl and 3-fluoroazetidin-1-yl; and
5- to 10-membered heterocycloalkyl, 5-membered heteroaryl and 6-membered heteroaryl, each of which is optionally substituted by 1, 2 or 3 substituents independently selected from the group consisting of halogen, —OH, -oxo, —NO₂, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R3 is selected from the group consisting of
hydrogen, halogen, —OH, C₁-C₄-alkyl, and C₁-C₄-alkoxy;
R4 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R5 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R6 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R12 and R13 are independently selected from the group consisting of
hydrogen, OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy, C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, OH, cyano, COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, OH, cyano, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl,
—S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
hydrogen;
C₁-C₄-alkyl, C₃-C₆-cycloalkyl, phenyl-C₁-C₄-alkyl, each of which is optionally substituted by 1, 2 or 3 substituents independently selected from the group consisting of halogen, OH, cyano, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
Q is selected from the group consisting of
(i) 6- to 10-membered aryl and 5- to 10-membered heteroaryl, each of which is optionally substituted by 1, 2, 3, 4 or 5 substituents selected from the group consisting of halogen, SF₅, cyano, —CHO, nitro, oxo, C₁-C₄-alkyl, C₁-C₄-hydroxyalkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms;
(ii) —NH₂, —NH(C₁-C₄-alkyl), —NH(C₃-C₆-cycloalkyl), —NH(phenyl-C₁-C₄-alkyl), —NH(C₁-C₄-alkoxy), —NH(C₁-C₄-alkyl-C(O)—), (—NH(C₁-C₄-alkoxy-C(O)—), —N(C₁-C₄-alkyl)₂, C₁-C₆-alkyl, C₃-C₁₀-cycloalkyl, C₂-C₆-alkenyl, C₃-C₁₀-cycloalkenyl, C₂-C₆-alkynyl, each of which is optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, benzyloxy-C(O)—, C₁-C₄-alkoxy-C₁-C₄-alkyl-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl-C₁-C₄-alkyl-, optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl,
—S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl-, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano,
C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
a C- or N-bound monocyclic or bicyclic heterocycle selected from the group consisting of 4- to 10-membered heterocycloalkyl and heterospirocycloalkyl, each of which is optionally substituted by 1, 2, 3 or 4 substituents independently selected from the group consisting of halogen, cyano, nitro, —OH, oxo, thiono, —COOH, C₁-C₄-alkoxy-C(O)—,
—C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl;
E1_2 is

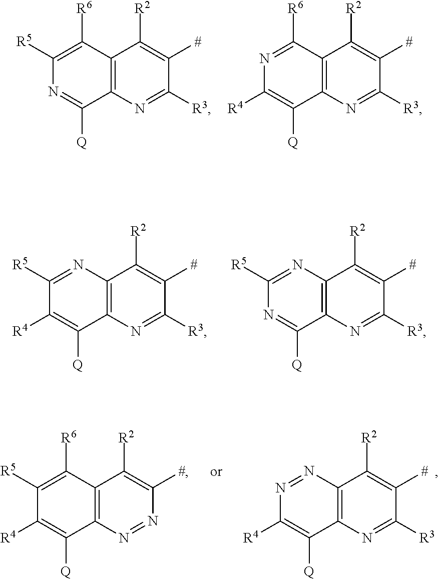

wherein
R2 is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl;
R3 is selected from the group consisting of
hydrogen, halogen and C₁-C₄-alkyl;
R4 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R5 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R6 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R12 and R13 are independently selected from the group consisting of
hydrogen, —OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy, C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
Q is selected from the group consisting of
6- to 10-membered aryl and 5- to 10-membered heteroaryl, each of which is optionally substituted by 1, 2, 3, 4 or 5 substituents selected from the group consisting of halogen, SF₅, cyano, —CHO, nitro, oxo, C₁-C₄-alkyl, C₁-C₄-hydroxyalkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms;
E1_3 is

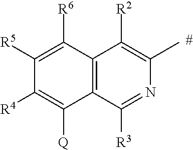

wherein
R2 is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —NH(C(O)—C₁-C₄-alkyl), —N(C₁-C₄-alkyl)(C(O)—C₁-C₄-alkyl), —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl;
R3 is hydrogen or C₁-C₄-alkyl;
R4 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms,
C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
preferably R4 is selected from the group consisting of hydrogen and halogen; more preferably R4 is selected from the group consisting of fluorine and chlorine;
R5 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R6 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms,
C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R12 and R13 are independently selected from the group consisting of
hydrogen, —OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy,
C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂,
—NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
hydrogen;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂,
—C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl,
—SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
Q is selected from the group consisting of 6- or 10-membered aryl and 5- to 10-membered heteroaryl, each of which is optionally substituted by 1, 2, 3, 4 or 5 substituents selected from the group consisting of halogen, SF₅, cyano, —CHO, nitro, oxo, C₁-C₄-alkyl, C₁-C₄-hydroxyalkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms,
wherein when Y is O, S or N—R9, none of R⁷, R⁸, R¹⁰ and R¹¹ is —OH or C₁-C₄-alkoxy, and wherein when X is O, S or N—R⁹, none of R⁷ and R⁸ is —OH or C₁-C₄-alkoxy;
E1_6 is

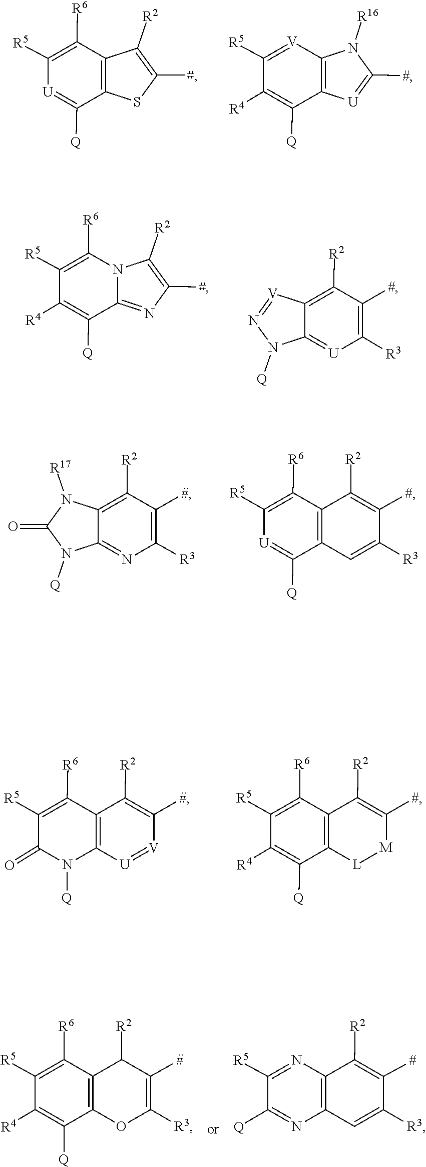

wherein
R2 is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl),
—C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms,
C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms,
—SO₂—C₁-C₄-halogenoalkyl having 1 to 5 halogen atoms, and 4- to 10-membered heterocycloalkyl;
R3 is selected from the group consisting of
hydrogen, halogen and C₁-C₄-alkyl;
R4 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms,
C₁-C₄-alkyl-C(O)—, —NH₂, —NH(C₁-C₄-alkyl), —N(C₁-C₄-alkyl)₂, —S—C₁-C₄-alkyl, —S—C₁-C₄-halogenoalkyl having 1 to 5 halogen atoms —S(O)—C₁-C₄-alkyl, —SO₂—C₁-C₄-alkyl;
R5 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R6 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms,
C₁-C₄-alkyl-C(O)—, —NH₂, —NH(C₁-C₄-alkyl), —N(C₁-C₄-alkyl)₂, —S—C₁-C₄-alkyl, —S—C₁-C₄-halogenoalkyl having 1 to 5 halogen atoms, —S(O)—C₁-C₄-alkyl, and —SO₂—C₁-C₄-alkyl;
R7 is selected from the group consisting of
hydrogen, —OH, halogen, C₁-C₄-alkyl and C₁-C₄-alkoxy;
R8 is selected from the group consisting of
hydrogen, —OH, halogen, C₁-C₄-alkyl and C₁-C₄-alkoxy;
or R7 and R8 form, together with the carbon atom to which they are attached, a 3- to 6-membered ring selected from the group consisting of C₃-C₆-cycloalkyl and 3- to 6-membered heterocycloalkyl;
R12 and R13 are independently selected from the group consisting of
hydrogen, —OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy, and
C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms,
—S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl,
—SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R16 is selected from the group consisting of
hydrogen, C₁-C₄-alkyl, C₁-C₄-halogenoalkyl having 1 to 5 halogen atoms and C₁-C₄-alkoxy;
R17 is selected from the group consisting of
hydrogen, C₁-C₄-alkyl, C₁-C₄-halogenoalkyl having 1 to 5 halogen atoms and C₁-C₄-alkoxy;
U is selected from the group consisting of CR⁷ and N;
V is selected from the group consisting of CR⁷ and N;
M is selected from the group consisting of C═O and CR⁷R⁸;
L is selected from the group consisting of 0 and NR⁹;
Q is selected from the group consisting of 6- to 10-membered aryl and 5- to 10-membered heteroaryl, each of which is optionally substituted by 1, 2, 3, 4 or 5 substituents selected from the group consisting of halogen, SF₅, cyano, —CHO, nitro, oxo, C₁-C₄-alkyl, C₁-C₄-hydroxyalkyl, C₁-C₄-halogenoalkyl having 1 to 5 halogen atoms, —OH, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl),
—NHCO(C₁-C₄-halogenoalkyl) having 1 to 5 halogen atoms;
E1_7 is

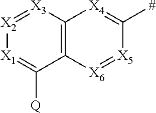

wherein
X1 is selected from the group consisting of N and CR₁;
X2 is selected from the group consisting of N and CR₂;
X3 is selected from the group consisting of N and CR₃;
X4 is selected from the group consisting of N and CR₄;
X5 is selected from the group consisting of N and CR₅;
X6 is selected from the group consisting of N and CR₆;
wherein at least one of X₁, X₂, X₃, and X₅ is N or wherein none of X₁, X₂, X₃, X₄, X₅, and X₆ are N;
R1 is selected from the group consisting of
hydrogen, halogen, hydroxyl, —SH, —SC₁-C₄ alkyl, —S(O)(C₁-C₄ alkyl), —S(O)₂(C₁-C₄ alkyl), cyano, C₁-C₉ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R2 is selected from the group consisting of
hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R3 is selected from the group consisting of
hydrogen, halogen, hydroxyl, —SH, —SC₁-C₄ alkyl, —S(O)(C₁-C₄ alkyl), —S(o)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R4 is selected from the group consisting of
halogen, cyano, —CHO, hydroxyl, C₁-C₄ alkyl, C₂-C₄ alkenyl, C₂-C₄ alkynyl, C₃-C₆ cycloalkyl, C₁-C₄ halogenoalkyl, C₁-C₄-alkoxy substituted-C₁-C₄ alkyl, benzyl optionally substituted with 1 to 5 halogen atoms, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₁-C₄ alkoxy substituted C₁-C₄ alkyl), —N(C₁-C₄ alkoxy substituted C₁-C₄ alkyl)₂, —N(C(O)C₁-C₄ alkyl)(C₁-C₄ alkyl), —N(C₁-C₄ alkyl)(C₃-C₆-cycloalkyl), —N(C₁-C₄ alkyl)(4- to 7-membered heterocycloalkyl), —NH(4- to 7-membered heterocycloalkyl), —N(C₁-C₄ alkyl)(C₁-C₄ alkoxy), —C(O)NH(C₁-C₄ alkyl), —C(O)N(C₁-C₄ alkyl)₂, —C(O)N(C₁-C₄ alkyl)(4- to 7-membered heterocycloalkyl), —NHSO₂(C₁-C₄ alkyl), —SC₁-C₄ alkyl, —S(O)C₁-C₄ alkyl, —SO₂C₁-C₄ alkyl, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; 6- or 10 membered aryl; a monocyclic heterocycle selected from the group of 4- to 7-membered heterocycloalkyl, 5-membered heteroaryl having at least one nitrogen atom via which the 5-membered heteroaryl ring is connected to the rest of the molecule, and 6-membered heteroaryl having at least one nitrogen atom; each of the aryl, heterocycloalkyl, and heteroaryl rings in R₄ is optionally substituted with 1, 2 or 3 substituents independently selected from the group consisting of halogen, cyano, nitro, hydroxy, oxo, imino, 1-imino-1-oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; wherein the C₃-C₆ cycloalkyl and the heterocycloalkyl rings in R₄ are optionally substituted with a spiro group, wherein said spiro group is a 3- to 6-membered cycloalkyl or 4- to 6-membered heterocycloalkyl containing 1, 2, or 3 heteroatoms independently selected from N, S or O, wherein said spiro group is optionally substituted with 1, 2 or 3 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and wherein each C₁-C₄ alkyl, C₃-C₆ cycloalkyl and C₁-C₄ alkoxy in R₄ may be optionally substituted with 1, 2 or 3 substituents independently selected from the group consisting of halogen, hydroxy, oxo, —NH₂, —NH(C₁-C₄ alkyl), —N(C₁-C₄ alkyl)₂, cyano, carboxy, carbamoyl, C₁-C₄ alkoxycarbonyl, —C(O)NH(C₁-C₄ alkyl), —C(O)N(C₁-C₄ alkyl)₂, C₁-C₄ halogenoalkyl, and C₁-C₄ alkoxy;
R5 is selected from the group consisting of
hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R6 is selected from the group consisting of
hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
Q is selected from the group consisting of
(i) 6- or 10 membered aryl optionally substituted with 1, 2, 3, 4, or 5 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl, —C(O)NH₂, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl, —SO₂C₁-C₄ halogenoalkyl, and pentafluoro-sulfanyl, wherein the 6- or 10 membered aryl is optionally fused with a 4-to 7-membered heterocycloalkyl having 1 or 2 heteroatoms selected from the group O, S, and N and wherein the carbons of the heterocycloalkyl are optionally substituted with 1, 2 or 3 substituents independently selected from the group halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is, valency permitting, substituted with a substituent selected from the group consisting of hydrogen, C₁-C₄ alkyl, and C₃-C₆ cycloalkyl;
(ii) 5- to 10-membered heteroaryl having 1, 2, or 3 heteroatoms independently selected from the group O, S, and N and wherein the carbons of the 5- to 10-membered heteroaryl are optionally substituted with 1, 2, 3, 4, or 5 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, benzyloxy, —C(O)R₁₇, —NH₂, —NH(C₁-C₄ alkyl), —N(C₁-C₄ alkyl)₂, —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, and any N in the heteroaryl, valency permitting, is optionally substituted with a substituent selected from the group consisting of hydrogen, C₁-C₄ alkyl, and C₃-C₆ cycloalkyl;
(iii) 4- to 7-membered heterocycloalkyl having 1, 2, or 3 heteroatoms independently selected from the group O, S, N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1, 2, 3, or 4 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), and —N(C₁-C₄ alkyl)₂ and any N in the heterocycloalkyl is optionally substituted with a substituent selected from the group consisting of hydrogen, C₁-C₄ alkyl, and C₃-C₆ cycloalkyl;
(iv) 6- or 10 membered aryloxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
(v) 6- or 10 membered arylthio-oxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and
(vi) 5- to 10-membered heteroaryloxy optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
E2_1 is

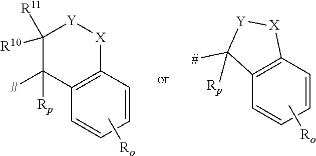

wherein
R10 is selected from the group consisting of hydrogen, —OH, C₁-C₄-alkyl and C₁-C₄-alkoxy;
R11 is selected from the group consisting of hydrogen, C₁-C₄-alkyl and C₁-C₄-alkoxy;
or R10 and R11 form, together with the carbon atom to which they are attached, a 3-to 6-membered ring selected from the group consisting of C₃-C₆-cycloalkyl and 3- to 6-membered heterocycloalkyl;
Rp is hydrogen or C₁-C₄-alkyl;
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms;
o is 0, 1, 2, 3 or 4;
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—;
R7 is selected from the group consisting of hydrogen, —OH, fluorine, C₁-C₄-alkyl and C₁-C₄-alkoxy;
R8 is selected from the group consisting of hydrogen, —OH, halogen, C₁-C₄-alkyl and C₁-C₄-alkoxy, preferably from the group consisting of hydrogen, —OH, fluorine, C₁-C₄-alkyl and C₁-C₄-alkoxy;
or R7 and R8 together form an oxo group (═O);
or R7 and R8 form, together with the carbon atom to which they are attached, a 3- to 6-membered ring selected from the group consisting of C₃-C₆-cycloalkyl and 3- to 6-membered heterocycloalkyl;
R9 is selected from the group consisting of hydrogen, C₁-C₄-alkyl, C₁-C₄-halogenoalkyl having 1 to 5 halogen atoms and C₁-C₄-alkoxy;
wherein when Y is O, S or N—R⁹, none of R⁷, R⁸, R¹⁰ and R¹¹ is —OH, and wherein when X is O, S or N—R⁹, none of R⁷ and R⁸ is —OH;
E2_2 is

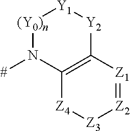

wherein
n is 0 or 1; when n is 1, Y₀ is CH₂ or C═O;
Y₁ is selected from the group consisting of CR₈R₉, O, S, and NR₁₀;
Y₂ is selected from the group consisting of CR₈R₉, O, S, and NR₁₀;
wherein at least one of the groups Y₁ or Y₂ is CR₈R₉;
Z₁ is selected from the group consisting of N, O, S, and CR₁₁;
Z₂ is selected from the group consisting of Nil, N, and CR₁₁;
Z₃ is selected from the group consisting of Nil, N and CR₁₁;
Z₄ is selected from the group consisting of N, O, S, and CR₁₁;
R8 is each time selected, independently selected from the group consisting of hydrogen, fluoro, and C₁-C₄ alkyl;
R9 is, each time selected, independently selected from the group consisting of hydrogen, fluoro, and C₁-C₄ alkyl;
R10 is selected from the group consisting of hydrogen and C₁-C₄ alkyl;
R11 is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and —N(C₁-C₄ alkyl)₂;
E2_3 is

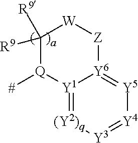

wherein
R9 and R9′ are independently hydrogen, halo, C₁-C₄-alkyl, C₁-C₄-haloalkyl, C₁-C₄-alkoxy, C₁-C₄-haloalkoxy or cycloalkoxy, or R9 together with R9′ form a 2-6-membered chain optionally containing one or two heteroatoms selected from the group consisting of N, O, Si and S to
form carbocyclic or heterocyclic ring together with the carbon atom to which they are attached;
W is CR⁵R⁶, O, SOp, or N—R;
Z is CR⁵R⁶, O, SOp, or N—R;
Q is N or C—R⁸;
Y1 and Y6 are each independently N, C or —CR⁴—;
Y2, Y3, Y4 and Y5 are each independently N, NR′, S, O, —CR⁴— or CR⁴R^{4′};
R4 and R4′ are independently in each occurrence, hydrogen, halogen, cyano, nitro, hydroxyl, optionally substituted alkyl, optionally substituted alkoxy, optionally substituted cycloalkyl, optionally substituted cycloalkoxy, optionally substituted alkylcarbonyl, optionally substituted alkoxycarbonyl, optionally substituted aminocarbonyl, alkylaminocarbonyl, or di(alkyl)aminocarbonyl, optionally substituted alkylcarbonyloxy, optionally substituted alkyl carbonylamino, optionally substituted aryl, optionally substituted heteroaryl, —SF5, —SOp (optionally substituted alkyl or haloalkyl); or R4 together with R4′ together form a 2-6-membered chain optionally containing one or two heteroatoms
selected from the group consisting of N, O, Si and S to form carbocyclic or heterocyclic ring together with the carbon atom to which they are attached; or —NR^{c}R^{d}, wherein R^{C} and R^{d} are independently H or optionally substituted alkyl; or R^{c} and R^{d} may form, with the nitrogen to which they are attached, a 3-, 4-, 5-, 6-, 7-, or 8-membered-heterocyclyl group, which may include one to three additional heteroatoms selected from the group consisting of N, O, Si and S and may be optionally substituted;
R5 and R6 are independently in each occurrence hydrogen, halo, C₁-C₄-alkyl, C₁-C₄-haloalkyl, C₁-C₄-alkoxy, C₁-C₄-haloalkoxy or cycloalkoxy; or R5 together with R6 form a 2-6-membered chain optionally containing one or two heteroatoms selected from the group consisting of N, O, Si and S to form carbocyclic or heterocyclic ring together with the carbon atom to which they are attached;
R7 is hydrogen or C₁-C₄-alkyl;
wherein at most three of Y1, Y2, Y3, Y4, Y5 and Y6 are heteroatoms;
a is 0 or 1;
q is 0 or 1;
p is independently in each occurrence is 0, 1, or 2;

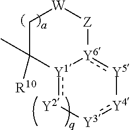

or E2_3 is
wherein
W is CR⁶R⁷, O, S, or N—R⁸;
Z is CR⁶R⁷, O, S, or N—R⁸;
Y1′ and Y6′ are each independently N, C, or —CR⁵—;
Y2′, Y3′, Y4′, Y5′ are each independently N, NR², S, O, —CR⁵— or CR⁵R^{5′};
a is 0 or 1;
q is 0 or 1;
R10 hydrogen, halogen, alkyl, haloalkyl, cycloalkyl, alkenyl or alkynyl;
R5 and R5′ are independently in each occurrence, hydrogen, halogen, cyano, nitro, hydroxyl, optionally substituted alkyl, optionally substituted alkoxy, optionally substituted cycloalkyl, optionally substituted cycloalkyloxy, optionally substituted aryl, optionally
substituted heteroaryl, —SF₅, —SOₚ (optionally substituted alkyl or haloalkyl), or —NR^{c}R^{d} wherein R^{C} and R^{d} are independently H or optionally substituted alkyl; or R^{C} and R^{d} may form, with the nitrogen to which they are attached, a 3-, 4-, 5-, 6-, 7-, or 8-membered-heterocyclyl group, which may include one to three additional heteroatoms selected from the group consisting of N, O and S and may be optionally substituted;
R6 and R7 are independently in each occurrence hydrogen, halo, C₁-C₄-alkyl, C₁-C₄-haloalkyl, C₁-C₄-alkoxy, C₁-C₄-haloalkoxy or C₃-C₈-cycloalkoxy;
R8 is hydrogen or C₁-C₄-alkyl; and
wherein at most three of Y1, Y2′, Y3′, Y4′, Y5′ and Y6′ are heteroatoms;
E2_4 is

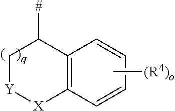

wherein
X and Y are independently CR⁵R⁶, O, S, or N—R⁷, wherein at least one of X and Y is CR⁵R⁶;
R5 and R6 are independently hydrogen, fluorine or C₁-C₄-alkyl;
R7 is hydrogen or C₁-C₄-alkyl;
q is 0 or 1;
o is 0, 1, 2, 3, or 4;
each R4 is independently hydrogen, halogen, cyano, nitro, —OH, optionally substituted alkyl, optionally substituted alkoxy, optionally substituted cycloalkyl, -amino, NH-optionally substituted alkyl, SF₅, or NR^{c}R^{d} wherein R^{c} and R^{d} are independently optionally substituted alkyl;
or R^{c} and R^{d} may form, with the nitrogen to which they are attached, a 3, 4, 5, 6, 7, or 8 membered-heterocyclyl group, which may be optionally substituted, SOₚ (optionally substituted C₁-C₄-alkyl);
p is 0, 1, or 2;
E2_5 is

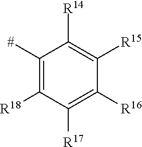

wherein
R and R14 together with the atoms to which they are attached form a 5 or 6-carbon atoms containing non-aromatic ring, wherein the 5 or 6-carbon atoms containing ring is optionally substituted with one or more C₁₋₃-alkyl or ═O, and/or wherein one or more of the ring forming
carbon atoms are optionally replaced by —NH—, —N═, ═N—, —O—, —S(O)—, —S(O)₂— or —S—, or
R and R14 together with the atoms to which they are attached form a 5 or 6-carbon atoms containing aromatic ring, wherein the 5 or 6-carbon atoms containing ring is optionally substituted with one or more C₁₋₃-alkyl, and/or wherein one or more of the ring forming carbon atoms are
optionally replaced by —NH—, —N═, ═N—, —O— or —S—;
R15 is independently hydrogen, halogen, C₁₋₃ alkyl, C₁₋₃ alkoxy, or NR^{15′}R^{15″}, wherein R^{15′} and R^{15″} are independently C₁₋₃-alkyl;
R16 is independently hydrogen, halogen, C₁₋₃ alkyl, C₁₋₃ alkoxy, or NR^{16′}R^{16″}, wherein R^{16′} and R^{16″} are independently C₁₋₃-alkyl;
R17 is independently hydrogen, C₁₋₃ alkyl, C₁₋₃ alkoxy, or NR^{17′}R^{17″}, wherein R^{17′} and R^{7″} are independently C₁₋₃-alkyl;
R18 is independently hydrogen, C₁₋₃ alkyl, C₁₋₃ alkoxy, or NR^{18′}R^{18″}, wherein R^{18′} and R^{18″} are independently C₁₋₃-alkyl;
E2_6 is

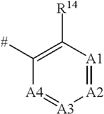

wherein
A1 is N or CR¹⁵, wherein R¹⁵ is independently hydrogen, halogen,
NR^{15′}R^{15″}, wherein R^{15′} and R^{15″} are independently C₁₋₃-alkyl;
A2 is N or CR¹⁶, wherein R¹⁶ is independently hydrogen, halogen, NR^{16′}R^{16″}, wherein R^{16′} and R^{16″} are independently C₁₋₃-alkyl;
A3 is N or CR¹⁷, wherein R¹⁷ is independently hydrogen, halogen, NR^{17′}R^{17″}, wherein R^{17′} and R^{17″} are independently C₁₋₃-alkyl;
A4 is N or CR¹⁸, wherein R¹⁸ is independently hydrogen, halogen NR^{18′}R^{18″}, wherein R^{18′} and R^{18″} are independently C₁₋₃-alkyl;
   R14 is hydrogen, C₁₋₃ alkyl, C₁₋₃ alkoxy, NR^{14′}R^{14″}, wherein R^{14′} and R^{14″} are independently C₁₋₃-alkyl;
   R and R14 together with the atoms to which they are attached form a 5 or 6-carbon atoms containing non-aromatic ring, wherein the 5 or 6-carbon atoms containing ring is optionally substituted with one or more C1-3-alkyl or ═O, and/or wherein one or more of the ring forming carbon atoms are optionally replaced by —NH—, —N═, ═N—, —O—, —S(O)—, —S(O)2- or —S—, or
   R and R14 together with the atoms to which they are attached form a 5 or 6-carbon atoms containing aromatic ring, wherein the 5 or 6-carbon atoms containing ring is optionally substituted with one or more C1-3-alkyl, and/or wherein one or more of the ring forming carbon atoms are optionally replaced by —NH—, —N═, ═N—, —O— or —S—.

The compounds of this embodiment are particularly suitable for the long-term prevention and/or treatment of a disease, in particular for the long-term prevention and/or treatment of a helminthic infection in an animal.

In one embodiment, the compound for use in the long-term prevention and/or treatment of a disease is a compound wherein E1 and E2 are selected from the following pairs when the compound of formula I is a compound of formula I.1:
E1_2 and E2_1 wherein n is 0;
E1_4 and E2_1 wherein n is 0;
E1_7 and E2_2 wherein n is 0.

The compounds of this embodiment are particularly suitable for the long-term prevention and/or treatment of a disease, in particular for the long-term prevention and/or treatment of a helminthic infection in an animal.

In one embodiment, the compound for use in the long-term prevention and/or treatment of a disease is a compound, wherein
E1 and E2 are selected from the following pairs when the compound of formula I is a compound of formula I.1:
E1_2 and E2_1′ wherein n is 0;
E1_4 and E2_1″ wherein n is 0;
E1_7 and E2_2 wherein n is 0;
   in which
   E1_2 is

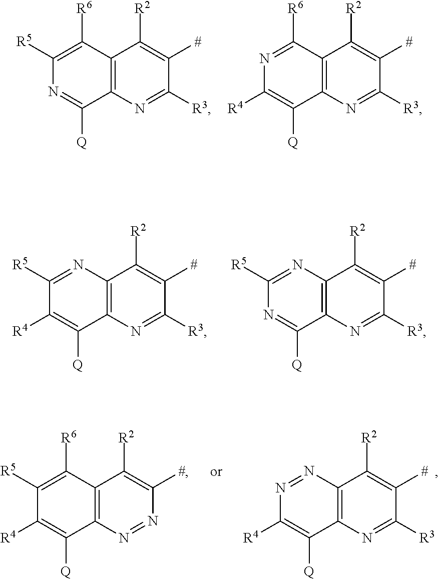

wherein
R2 is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂,
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and 4- to 10-membered heterocycloalkyl;
R3 is selected from the group consisting of hydrogen, halogen or C₁-C₄-alkyl;
R4 is selected from the group consisting of hydrogen, halogen, cyano, C₁-C₄-alkyl, C₁-C₄-halogenoalkyl having 1 to 5 halogen atoms, C₁-C₄-alkoxy;
R5 is selected from the group consisting of hydrogen, halogen, cyano, C₁-C₄-alkyl, C₁-C₄-halogenoalkyl having 1 to 5 halogen atoms, C₁-C₄-alkoxy;
R6 is selected from the group consisting of hydrogen, halogen, cyano, C₁-C₄-alkyl, C₁-C₄-halogenoalkyl having 1 to 5 halogen atoms, C₁-C₄-alkoxy;
R12 and R13 are independently selected from the group consisting of hydrogen, —OH, —NH₂, —NH(C₁-C₄-alkyl), —N(C₁-C₄-alkyl)₂, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)-(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
Q is a substituted phenyl ring of the formula (Q1)

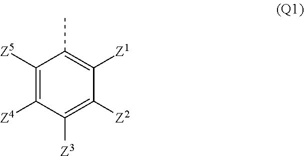

in which:
Z¹, Z², Z³, Z⁴, and Z⁵ are independently selected from the group consisting of hydrogen, halogen, SF₅, cyano, —CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z¹ and Z² form, together with the carbon atoms that they are connected to, a 5- or 6-membered saturated or partially saturated heterocyclic ring, a 5-membered heteroaryl, or a 6-membered heteroaryl, each of which may be optionally substituted with one or two substitutents selected from the group consisting of methyl, fluorine and oxo, and
Z³, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-alkoxy-C(O)—, C₁-C₄-halogenoalkoxy having 1 to 5 halogen atoms, —NH(C₁-C₄-alkyl), —N(C₁-C₄-alkyl), —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4-to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z² and Z³ form, together with the carbon atoms that they are connected to, a 5- or 6-membered saturated or partially saturated heterocyclic ring, a 5-membered heteroaryl, or a 6-membered heteroaryl, each of which may be optionally substituted with one or two substitutents selected from the group consisting of methyl, fluorine and oxo, and
Z¹, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Q is a pyridine ring of the formula (Q2)

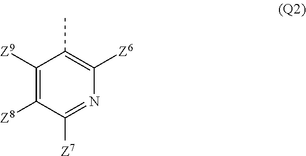

in which:
Z⁶, Z⁷, Z⁸ and Z⁹ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyrimidine ring of the formula (Q3)

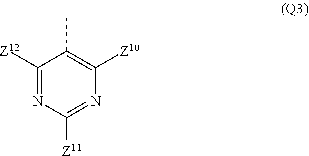

in which:
Z¹⁰, Z¹¹ and Z¹² are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyridine ring of the formula (Q4)

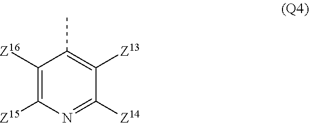

in which:
Z¹³, Z¹⁴, Z¹⁵ and Z¹⁶ are independently selected from the group consisting of hydrogen halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₁-C₄-hydroxyalkyl, NH₂, —NH(C₁-C₄-alkyl), —N(C₁-C₄-alkyl)₂, —NH—CO—C₁-C₄-alkyl, and monocyclic heterocycles selected from the group of 4- to 7-membered heterocycloalkyl or 5-membered heteroaryls having at least one nitrogen atom via which the heteroaryl ring is connected to the pyridine ring, each of which is optionally substituted with 1, 2 or 3 substituents independently selected from the group consisting of halogen, cyano, nitro, —OH, oxo, thiono, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, or
Q is a pyridine ring of the formula (Q5)

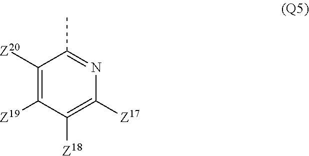

in which:
Z¹⁷, Z¹⁸, Z¹⁹ and Z²⁰ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a 5-membered aromatic heterocycle of the formula (Q6)

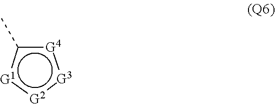

in which:
G¹-G⁴ are independently selected from the group consisting of N, O, S, C—Z²¹ and N—Z²² wherein not more than one of G¹-G⁴ is 0, not more than one of G¹-G⁴ is S, not more than one of G¹-G⁴ is N—Z²², and wherein
each Z²¹ is independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, and each Z²² is independently selected from the group consisting of hydrogen, C₁-C₄-alkyl, C₁-C₄-halogenoalkyl having 1 to 5 halogen atoms, C₁-C₄-alkyl-C₃-C₆-cycloalkyl, C₁-C₄-alkoxy-C₁-C₄-alkyl, or
Q is a 5-membered aromatic heterocycle of the formula (Q7)

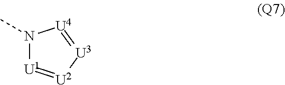

in which:
U¹-U⁴ are independently selected from the group consisting of N and C—Z²³, wherein not more than three of U¹—U⁴ are N, and wherein
each Z²³ is independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
wherein when Y is O, S or N—R⁹, none of R⁷, R⁸, R¹⁰ and R¹¹ is —OH, and wherein when X is 0, S or N—R⁹, none of R⁷ and R⁸ is —OH;
E2_1′ is

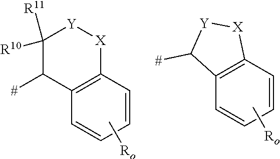

wherein
o is 0, 1, 2, 3 or 4;
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃—C-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms;
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—;
R7 is selected from the group consisting of hydrogen, —OH, fluorine, C₁-C₄-alkyl and C₁-C₄-alkoxy;
R8 is selected from the group consisting of hydrogen, —OH, fluorine, C₁-C₄-alkyl and C₁-C₄-alkoxy;
R9 is selected from the group consisting of hydrogen, C₁-C₄-alkyl, C₁-C₄-halogenoalkyl having 1 to 5 halogen atoms and C₁-C₄-alkoxy;
R10 is selected from the group consisting of hydrogen, —OH, C₁-C₄-alkyl and C₁-C₄-alkoxy;
R11 is selected from the group consisting of hydrogen, C₁-C₄-alkyl and C₁-C₄-alkoxy;
E1_4 is

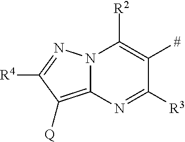

wherein
R2 is selected from the group consisting of hydrogen, halogen, cyano, C₁-C₄-alkyl, C₂-C₄-alkenyl, C₂-C₄-alkinyl, C₃-C₆-cycloalkyl, C₁-C₄-halogenoalkyl having 1 to 5 halogen atoms, C₁-C₄-alkoxy-C₁-C₄-alkyl, benzyl having 1 to 5 halogen atoms, C₁-C₄-alkoxy, —NH₂, —NH(C₁-C₄-alkyl), —N(C₁-C₄-alkyl)₂, —NH(C₃-C₆-cycloalkyl), —N(C₁-C₄-alkyl)(C₃-C₆-cycloalkyl), —NH(4- to 7-membered heterocycloalkyl), —N(C₁-C₄-alkyl)(4- to 7-membered heterocycloalkyl), —NH(C₁-C₄-alkoxy), —N(C₁-C₄-alkyl)(C₁-C₄-alkoxy), —NH—SO₂—(C₁-C₄-alkyl), —N(SO₂—[C₁-C₄-alkyl])(C₁-C₄-alkyl), (C₁-C₄-alkyl)-NH—C₁-C₄-alkyl-, (C₁-C₄-alkyl)₂-N—C₁-C₄-alkyl-, —S—C₁-C₄-alkyl, —S(O)—C₁-C₄-alkyl, —SO₂—C₁-C₄-alkyl, (C₁-C₄-alkoxyimino)-C₁-C₄-alkyl, and a monocyclic heterocycle selected from the group of 4- to 7-membered heterocycloalkyl, 5-membered heteroaryl having at least one nitrogen atom via which the heteroaryl ring is connected to the rest of the molecule, and 6-membered heteroaryl having at least one nitrogen atom, each of which in R² is optionally substituted with 1, 2 or 3 substituents independently selected from the group consisting of hydrogen, halogen, cyano, nitro, —OH, oxo, thiono, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms, and wherein each C₁-C₄-alkyl, C₃-C₆-cycloalkyl and C₁-C₄-alkoxy in R² may be optionally substituted with halogen, OH, NH₂, —NH(C₁-C₄-alkyl), —N(C₁-C₄-alkyl)₂, cyano, carboxy, carbamoyl, alkoxycarbonyl, —C(O)—NH(C₁-C₄-alkyl), —C(O)—N(C₁-C₄-alkyl)₂, C₁-C₄-alkoxy, —S—C₁-C₄-alkyl, —S(O)—C₁-C₄-alkyl, —SO₂—C₁-C₄-alkyl, or optionally substituted by a monocyclic heterocycle selected from the group of azetidines, pyrrolidines, morpholines, piperidines, piperazines, pyrrolidinones, morpholinones, piperidinones, piperazinones, pyrazoles, triazoles, imidazoles and pyrroles, wherein a heteroaryl ring is connected to the C₁-C₄-alkyl or C₃-C₆-cycloalkyl via one of its nitrogen atoms, each of which as a substituent of C₁-C₄-alkyl, C₃-C₆-cycloalkyl and C₁-C₄-alkoxy in R2 is optionally substituted with 1, 2 or 3 substituents independently selected from the group consisting of hydrogen, halogen, cyano, oxo, C₁-C₄-alkyl, C₁-C₄-halogenoalkyl having 1 to 5 halogen atoms;
R3 is selected from the group consisting of hydrogen, halogen, C₁-C₄-alkyl, C₁-C₄-halogenoalkyl having 1 to 5 halogen atoms, C₃-C₆-cycloalkyl;
R4 is selected from the group consisting of hydrogen, halogen, C₁-C₄-alkyl, C₁-C₄-halogenoalkyl having 1 to 5 halogen atoms, C₃-C₆-cycloalkyl, —S—C₁-C₄-alkyl, —S(O)—C₁-C₄-alkyl, —SO₂—C₁-C₄-alkyl;
Q is selected from the group consisting of 6- or 10-membered aryl and 5- to 10-membered heteroaryl, each of which may be optionally substituted with 1, 2, 3, 4 or 5 substituents;
E2_1″ is

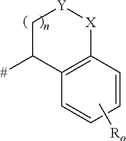

wherein
o is 0, 1, 2, 3 or 4;
R is selected from the group consisting of hydrogen, halogen, C₁-C₄-alkyl and C₁-C₄-halogenoalkyl having 1 to 5 halogen atoms;
n is 0 or 1;
X, Y are independently selected from the group consisting of CR⁵R⁶, O, S, and N—R⁷, wherein at least one of X and Y is CR⁵R⁶;
R5 is selected from the group consisting of hydrogen, fluorine or C₁-C₄-alkyl;
R6 is selected from the group consisting of hydrogen, fluorine or C₁-C₄-alkyl;
R7 is selected from the group consisting of hydrogen or C₁-C₄-alkyl;
E1_7 is

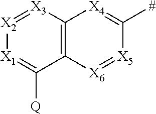

X₁ is CR₁; X₂ is CR₂; X₃ is CR₃; X₄ is CR₄; X₅ is CR₅; and X₆ is CR₆;
E2_2 is

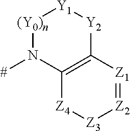

wherein
n is 0 or 1; when n is 1, Y₀ is CH₂ or C═O;
Y₁ is selected from the group consisting of CR₈R₉, O, S, and NR₁₀;
Y₂ is selected from the group consisting of CR₈R₉, O, S, and NR₁₀;
wherein at least one of the groups Y₁ or Y₂ is CR₈R₉;
Z₁ is selected from the group consisting of N, O, S, and CR₁₁;
Z₂ is selected from the group consisting of Nil, N, and CR₁₁;
Z₃ is selected from the group consisting of Nil, N and CR₁₁;
Z₄ is selected from the group consisting of N, O, S, and CR₁₁;
R8 is each time selected, independently selected from the group consisting of hydrogen, fluoro, and C₁-C₄ alkyl;
R9 is, each time selected, independently selected from the group consisting of hydrogen, fluoro, and C₁-C₄ alkyl;
R10 is selected from the group consisting of hydrogen and C₁-C₄ alkyl;
R11 is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and
—N(C₁-C₄ alkyl)₂.

The compounds of this embodiment are particularly suitable for the long-term prevention and/or treatment of a disease, in particular for the long-term prevention and/or treatment of a helminthic infection in an animal.

In one embodiment, the compound for use in the long-term prevention and/or treatment of a disease is a compound, wherein E1 and E2 are selected from the following pairs when the compound of formula I is a compound of formula I.1:
E1_2 and E2_1′ wherein n is 0;
E1_4 and E2_1″ wherein n is 0;
E1_7 and E2_2 wherein n is 0;
in which
   E1_2 is

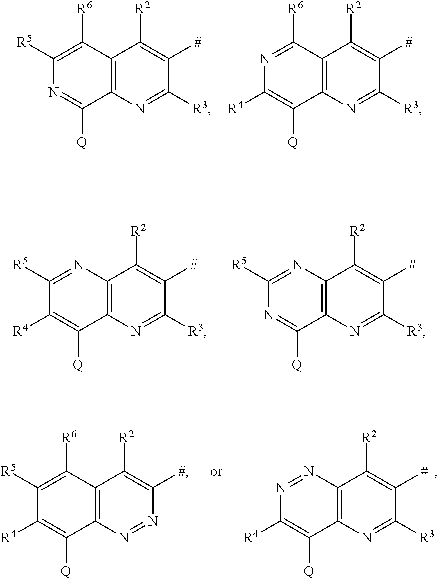

wherein
R2 is selected from the group consisting of hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂,
—NR¹²R¹³;
—OR¹⁴;
—SR¹⁵, —S(O)R¹⁵, —SO₂R¹⁵;
C₁-C₄-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, cyano, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, —OH, oxo, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, and 4- to 10-membered heterocycloalkyl;
R3 is selected from the group consisting of hydrogen, halogen or C₁-C₄-alkyl;
R4 is selected from the group consisting of hydrogen, halogen, cyano, C₁-C₄-alkyl, C₁-C₄-halogenoalkyl having 1 to 5 halogen atoms, C₁-C₄-alkoxy;
R5 is selected from the group consisting of hydrogen, halogen, cyano, C₁-C₄-alkyl, C₁-C₄-halogenoalkyl having 1 to 5 halogen atoms, C₁-C₄-alkoxy;
R6 is selected from the group consisting of hydrogen, halogen, cyano, C₁-C₄-alkyl, C₁-C₄-halogenoalkyl having 1 to 5 halogen atoms, C₁-C₄-alkoxy;
R12 and R13 are independently selected from the group consisting of hydrogen, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)-(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
phenyl, benzo-C₅-C₆-cycloalkyl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
R14 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl; and
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
R15 is selected from the group consisting of
C₁-C₄-alkyl, phenyl-C₁-C₄-alkyl, each of which is optionally substituted by 1, 2 or 3 substituents independently selected from the group consisting of halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
Q is a substituted phenyl ring of the formula (Q1)

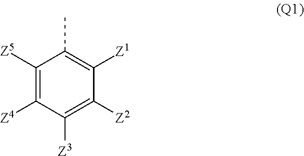

in which:
Z¹, Z², Z³, Z⁴, and Z⁵ are independently selected from the group consisting of hydrogen, halogen, cyano, C₁-C₄-alkyl, C₁-C₄-halogenoalkyl having 1 to 5 halogen atoms, hydroxy, C₁-C₄-alkoxy, C₁-C₄-halogenoalkoxy having 1 to 5 halogen atoms, —NH(C₁-C₄-alkyl), —N(C₁-C₄-alkyl)₂, 4- to 6-membered heterocyclyl, which is optionally substituted with 1 or 2 substituents selected from the group consisting of fluorine, chlorine, bromine, methyl and cyano, —S—(C₁-C₄-alkyl), —S(O)—(C₁-C₄-alkyl), —SO₂—(C₁-C₄-alkyl), or
Z¹ and Z² form, together with the carbon atoms that they are connected to, a 5- or 6-membered heterocycloalkyl, a 5-membered heteroaryl, or a 6-membered heteroaryl, each of which may be optionally substituted with one or two substitutents selected from the group consisting of methyl, fluorine and oxo, and
Z³, Z⁴, and Z⁵ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-alkoxy-C(O)—, C₁-C₄-halogenoalkoxy having 1 to 5 halogen atoms, or
Z² and Z³ form, together with the carbon atoms that they are connected to, a 5- or 6-membered saturated or partially saturated heterocyclic ring, a 5-membered heteroaryl, or a 6-membered heteroaryl, each of which may be optionally substituted with one or two substitutents selected from the group consisting of methyl, fluorine and oxo, and
Z¹, Z⁴, and Z⁵ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
E2_1′ is

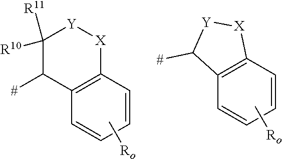

o is 0, 1 or 2;
R is selected from the group consisting of hydrogen, halogen, C₁-C₄-alkyl and C₁-C₄-alkoxy, cyano, C₁-C₄-halogenoalkyl having 1 to 5 halogen atoms;
X, Y are independently selected from the group consisting of CR⁷R⁸, O, S, and N—R⁹, wherein at least one of X and Y is CR⁷R⁸;
R7 is selected from the group consisting of hydrogen and C₁-C₄-alkyl;
R8 is selected from the group consisting of hydrogen and C₁-C₄-alkyl;
R9 is C₁-C₄-alkyl;
R10 is selected from the group consisting of hydrogen, —OH, C₁-C₄-alkyl and C₁-C₄-alkoxy;
R11 is hydrogen;
E1_4 is

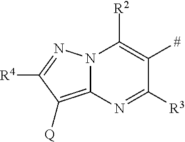

wherein
R2 is selected from the group consisting of hydrogen, C₁-C₄-alkyl, C₃-C₆-cycloalkyl, C₁-C₄-halogenoalkyl having 1 to 5 halogen atoms, C₁-C₄-alkoxy-C₁-C₄-alkyl, benzyl having 1 to 5 halogen atoms, C₁-C₄-alkoxy, —NH(C₁-C₄-alkyl), —N(C₁-C₄-alkyl)₂, —NH(C₃-C₆-cycloalkyl), —N(C₁-C₄-alkyl)(C₃-C₆-cycloalkyl), —N(C₁-C₄-alkyl)(6-membered heterocycloalkyl), —N(C₁-C₄-alkyl)(C₁-C₄-alkoxy), (C₁-C₄-alkyl)₂-N—C₁-C₄-alkyl-, and 4- to 6-membered heterocycloalkyl having at least one nitrogen atom via which the heterocycloalkyl ring is connected to the rest of the molecule, wherein a heterocycloalkyl group in R² may be optionally substituted with 1 to 4 substituents selected from the group consisting of fluorine, chlorine, cyano, oxo, C₁-C₄-alkyl, C₁-C₄-alkoxy, —N(C₁-C₄-alkyl)₂, and wherein each C₁-C₄-alkyl, C₃-C₆-cycloalkyl and C₁-C₄-alkoxy in R² may be optionally substituted with halogen, OH, NH₂, —NH(C₁-C₄-alkyl), —N(C₁-C₄-alkyl)₂, cyano, carboxy, carbamoyl, alkoxycarbonyl, —C(O)—NH(C₁-C₄-alkyl), —C(O)—N(C₁-C₄-alkyl)₂, C₁-C₄-alkoxy, —S—C₁-C₄-alkyl, —S(O)—C₁-C₄-alkyl, —SO₂—C₁-C₄-alkyl, or optionally substituted by a monocyclic heterocycle selected from the group of azetidines, pyrrolidines, morpholines, piperidines, and piperazines, each of which as a substituent of C₁-C₄-alkyl, C₃-C₆-cycloalkyl and C₁-C₄-alkoxy in R² is optionally substituted with 1, 2 or 3 substituents independently selected from the group consisting of hydrogen, halogen, cyano, oxo, C₁-C₄-alkyl, C₁-C₄-halogenoalkyl having 1 to 5 halogen atoms;
R3 is selected from the group consisting of hydrogen and C₁-C₄-alkyl;
R4 is selected from the group consisting of hydrogen, halogen, C₁-C₄-alkyl, C₁-C₄-halogenoalkyl having 1 to 5 halogen atoms, C₃-C₆-cycloalkyl, —S—C₁-C₄-alkyl, —S(O)—C₁-C₄-alkyl, —SO₂—C₁-C₄-alkyl;
Q is a substituted phenyl ring of the formula (Q1)

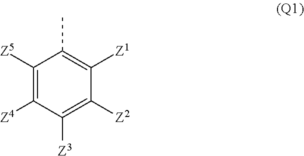

in which:
Z¹, Z², Z³, Z⁴, and Z⁵ are independently selected from the group consisting of hydrogen, halogen, SF₅, cyano, —CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, —O—(C₃-C₆-cycloalkyl), cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl and cyano, or 5-membered heteroaryl having at least one nitrogen atom via which the heteroaryl ring is connected to the rest of the molecule, —CH₂—O—(C₁-C₄-alkyl), —CH₂—NH(C₁-C₄-alkyl), —CH₂—N(C₁-C₄-alkyl)₂, methyl substituted with C₁-C₄-halogenoalkoxy having 1 to 5 halogen atoms, methyl substituted with C₃-C₆-cycloalkyl-C₁-C₄-alkoxy or methyl substituted with a 4- to 6-membered heterocycloalkyl which itself is optionally substituted with 1 or 2 substituents selected from the group consisting of fluorine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl), —S(O)—(C₁-C₄-halogenoalkyl), —SO₂—(C₁-C₄-halogenoalkyl), —S—(C₁-C₄-cycloalkyl), —S(O)—(C₁-C₄-cycloalkyl), —SO₂—(C₁-C₄-cycloalkyl), —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z¹ and Z² form, together with the carbon atoms that they are connected to, a 5- or 6-membered heterocycloalkyl, a 5-membered heteroaryl, or a 6-membered heteroaryl, each of which may be optionally substituted with one or two substituents selected from the group consisting of methyl, fluoro and oxo, and
Z³, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl), —S(O)—(C₁-C₄-halogenoalkyl), —SO₂—(C₁-C₄-halogenoalkyl), —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z² and Z³ form, together with the carbon atoms that they are connected to, a 5- or 6-membered cycloalkyl or heterocycloalkyl, a 5-membered heteroaryl, or a 6-membered heteroaryl, each of which may be optionally substituted with one or two substituents selected from the group consisting of methyl, fluoro and oxo, and
Z¹, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl), —S(O)—(C₁-C₄-halogenoalkyl), —SO₂—(C₁-C₄-halogenoalkyl), —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Q is a pyridine ring of the formula (Q2)

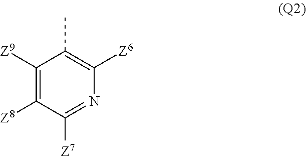

in which:
Z⁶, Z⁷, Z⁸ and Z⁹ are independently selected from the group consisting of hydrogen fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyrimidine ring of the formula (Q3)

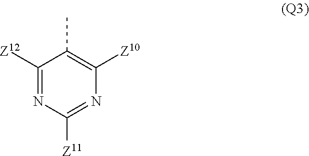

in which:
Z¹⁰, Z¹¹ and Z¹² are independently selected from the group consisting of hydrogen, fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyridine ring of the formula (Q4)

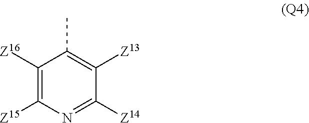

in which:
Z¹³, Z¹⁴, Z¹⁵ and Z¹⁶ are independently selected from the group consisting of hydrogen, fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —NH—CO—C₁-C₄-alkyl, and monocyclic heterocycles selected from the group of 4- to 7-membered heterocycloalkyl or 5-membered heteroaryls having at least one nitrogen atom via which the heteroaryl ring is connected to the pyridine ring, each of which is optionally substituted with 1, 2 or 3 substituents independently selected from the group consisting of hydrogen, halogen, cyano, nitro, —OH, oxo, thiono, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms, or
Q is a pyridine ring of the formula (Q5)

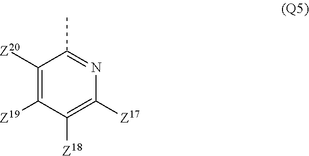

in which:
Z¹⁷, Z¹⁸, Z¹⁹ and Z²⁰ are independently selected from the group consisting of hydrogen, fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q a pyrazole ring of the formula (Q6)

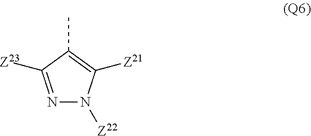

in which:
Z²¹ and Z²³ are independently selected from the group consisting of hydrogen, C₁-C₄-alkyl, C₁-C₄-halogenoalkyl having 1 to 5 halogen atoms, and
Z²² is selected from the group consisting of hydrogen, C₁-C₄-alkyl, C₁-C₄-halogenoalkyl having 1 to 5 halogen atoms, C₁-C₄-alkyl-C₃-C₆-cycloalkyl, C₁-C₄-alkoxy-C₁-C₄-alkyl, (C₁-C₄-alkyl)₂-N—C₁-C₄-alkyl-, morpholino-C₁-C₄-alkyl, (C₁-C₄-alkyl)-NH—C₁-C₄-alkyl-, or
Q is a pyrazole ring of the formula (Q7)

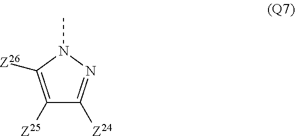

in which:
Z²⁴, Z²⁵ and Z²⁶ are independently selected from the group consisting of hydrogen, fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
E2_1″ is

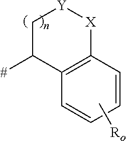

wherein
o is 0, 1, 2, 3 or 4;
R is selected from the group consisting of hydrogen, fluorine, chlorine, C₁-C₄-alkyl;
n is 0 or 1;
X, Y are independently selected from the group consisting of CR⁵R⁶, O, S, and N—R⁷, wherein at least one of X and Y is CR⁵R⁶;
R5 is hydrogen or methyl;
R6 is hydrogen or methyl;
R7 is hydrogen or methyl;
E1_7 is

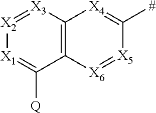

X₁ is N; X₂ is CR₂; X₃ is CR₃; X₄ is CR₄; X₅ is CR₅; and X₆ is N;
E2_2 is

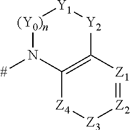

wherein
n is 0 or 1; when n is 1, Y₀ is CH₂ or C═O;
Y₁ is selected from the group consisting of CR₈R₉, O, S, and NR₁₀;
Y₂ is selected from the group consisting of CR₈R₉, O, S, and NR₁₀;
wherein at least one of the groups Y₁ or Y₂ is CR₈R₉;
Z₁ is selected from the group consisting of N, O, S, and CR₁₁;
Z₂ is selected from the group consisting of Nil, N, and CR₁₁;
Z₃ is selected from the group consisting of Nil, N and CR₁₁;
Z₄ is selected from the group consisting of N, O, S, and CR₁₁;
R8 is each time selected, independently selected from the group consisting of hydrogen, fluoro, and C₁-C₄ alkyl;
R9 is, each time selected, independently selected from the group consisting of hydrogen, fluoro, and C₁-C₄ alkyl;
R10 is selected from the group consisting of hydrogen and C₁-C₄ alkyl;
R11 is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and
—N(C₁-C₄ alkyl)₂.

The compounds of this embodiment are particularly suitable for the long-term prevention and/or treatment of a disease, in particular for the long-term prevention and/or treatment of a helminthic infection in an animal.

In one embodiment, the compound for use in the long-term prevention and/or treatment of a disease is a compound, wherein E1 and E2 are selected from the following pairs when the compound of formula I is a compound of formula I.1:
E1_2 and E2_1′ wherein n is 0;
E1_4 and E2_1″ wherein n is 0;
E1_7 and E2_2 wherein n is 0;
   in which
   E1_2 is

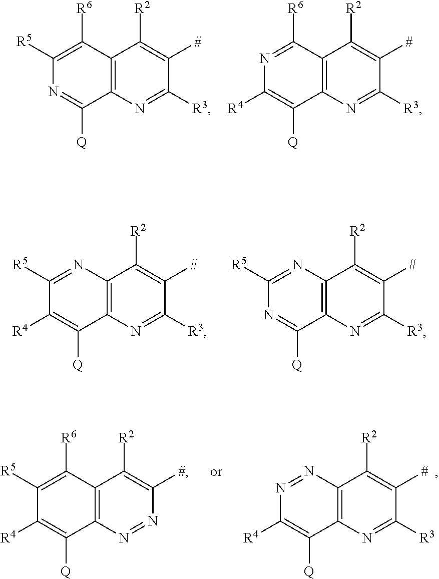

wherein
R2 is selected from the group consisting of
hydrogen, halogen,
—NR¹²R¹³;
—OR¹⁴;
—SR¹⁵, —S(O)R¹⁵, —SO₂R¹⁵;
C₁-C₄-alkyl, C₃-C₆-cycloalkyl, C₂-C₄-alkenyl or C₃-C₆-cycloalkenyl, each of which is optionally substituted by 1, 2, 3, 4 or 5 substituents independently selected from the group consisting of halogen, cyano, C₁-C₄-alkoxy-C(O)— and —C(O)—NH₂; and
a monocyclic or a bicyclic heterocycle selected from the group consisting of 4- to 10-membered heterocycloalkyl, heterospirocycloalkyl, 5-membered heteroaryl, and 6-membered heteroaryl, each of which is optionally substituted by 1, 2, 3 or 4 substituents independently selected from the group consisting of halogen, —OH, oxo, —COOH, C₁-C₄-alkoxy-C(O)—, —C(O)—NH₂, C₁-C₄-alkyl, C₁-C₄-alkyl-C(O)—, C₁-C₄-halogenoalkyl having 1 to 5 halogen atoms, hydroxy-C₁-C₄-alkyl-, C₁-C₄-alkoxy-C₁-C₄-alkyl-, —NH₂, —N(C₁-C₄-alkyl)₂, and 4- to 10-membered heterocycloalkyl,
R3 is selected from the group consisting of hydrogen, halogen or C₁-C₄-alkyl;
R4 is selected from the group consisting of hydrogen, halogen, cyano, C₁-C₄-alkyl, C₁-C₄-halogenoalkyl having 1 to 5 halogen atoms, C₁-C₄-alkoxy;
R5 is selected from the group consisting of hydrogen, halogen, cyano, C₁-C₄-alkyl, C₁-C₄-halogenoalkyl having 1 to 5 halogen atoms, C₁-C₄-alkoxy;
R6 is selected from the group consisting of hydrogen, halogen, cyano, C₁-C₄-alkyl, C₁-C₄-halogenoalkyl having 1 to 5 halogen atoms, C₁-C₄-alkoxy;
R12 and R13 are independently selected from the group consisting of hydrogen, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, —COOH, C₁-C₄-alkoxy-C(O)—, —C(O)—NH₂, —C(O)—N(C₁-C₄-alkyl)₂, —NH—C(O)—C₁-C₄-alkyl, C₁-C₄-alkyl, C₁-C₄-alkoxy, C₃-C₆-cycloalkyl, —NH₂, —N(C₁-C₄-alkyl)₂, —S—C₁-C₄-alkyl, —S(O)—C₁-C₄-alkyl, —SO₂—C₁-C₄-alkyl, and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms and C₁-C₄-alkoxy;
phenyl and benzo-C₅-C₆-cycloalkyl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms; and
a monocyclic or a bicyclic heterocycle selected from the group of 4- to 10-membered heterocycloalkyl, 5-membered heteroaryl and 6-membered heteroaryl each of which is optionally substituted by 1, 2 or 3 substituents independently selected from the group consisting of halogen, —OH, oxo, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
R14 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, C₁-C₄-alkyl, C₁-C₄-alkoxy and C₃-C₆-cycloalkyl; and 4- to 10-membered heterocycloalkyl,
R15 is selected from the group consisting of
C₁-C₄-alkyl, which is optionally substituted by 1, 2 or 3 substituents independently selected from the group consisting of —OH and —COOH; and a 6-membered heteroaryl;
Q is a substituted phenyl ring of the formula (Q1)

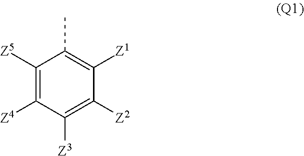

in which:
Z¹ is selected from the group consisting of hydrogen, halogen, C₁-C₄-alkyl and C₁-C₄-alkoxy,
Z² is selected from the group consisting of hydrogen, halogen, —OH, C₁-C₄-alkyl, C₁-C₄-alkoxy, —NH(C₁-C₄-alkyl), —N(C₁-C₄-alkyl)₂, —NH(C₃-C₆-cycloalkyl), —N(C₁-C₄-alkyl)(C₃-C₆-cycloalkyl), C₁-C₄-halogenoalkyl having 1 to 5 halogen atoms, C₁-C₄-halogenoalkoxy having 1 to 5 halogen atoms, —S—(C₁-C₄-alkyl) and a 4- to 6-membered heterocycloalkyl, and
Z³ is selected from the group consisting of hydrogen, halogen, C₁-C₄-alkyl, C₁-C₄-alkoxy, —NH(C₁-C₄-alkyl) and —N(C₁-C₄-alkyl)₂,
Z⁴ is selected from the group consisting of hydrogen, halogen, —OH, C₁-C₄-alkyl, C₁-C₄-alkoxy, —NH(C₁-C₄-alkyl), —N(C₁-C₄-alkyl)₂, —NH(C₃-C₆-cycloalkyl), —N(C₁-C₄-alkyl)(C₃-C₆-cycloalkyl), C₁-C₄-halogenoalkyl having 1 to 5 halogen atoms, C₁-C₄-halogenoalkoxy having 1 to 5 halogen atoms, —S—(C₁-C₄-alkyl) and a 4- to 6-membered heterocycloalkyl,
Z⁵ is selected from the group consisting of hydrogen, halogen, C₁-C₄-alkyl, and C₁-C₄-alkoxy;
E2_1′ is

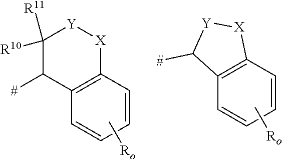

o is 0 or 1;
R is selected from the group consisting of hydrogen, halogen, C₁-C₄-alkyl and C₁-C₄-alkoxy;
X is selected from the group consisting of CR⁷R⁸, O, S, and N—R⁹;
Y is CR⁷R⁸;
R⁷ is selected from the group consisting of hydrogen and C₁-C₄-alkyl;
R⁸ is selected from the group consisting of hydrogen and C₁-C₄-alkyl;
R⁹ is C₁-C₄-alkyl;
R¹⁰ is selected from the group consisting of hydrogen, —OH and C₁-C₄-alkyl;
R¹¹ is hydrogen;
E1_4 is

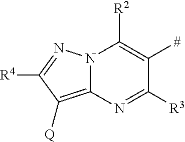

wherein
R2 is selected from the group consisting of hydrogen, C₁-C₄-alkyl, C₃-C₆-cycloalkyl, C₁-C₄-fluoroalkyl having 1 to 5 fluorine atoms, C₁-C₄-alkoxy-C₁-C₄-alkyl, benzyl having 1 to 5 halogen atoms, C₁-C₄-alkoxy, NH(C₁-C₄-alkyl), N(C₁-C₄-alkyl)₂, —NH(C₃-C₆-cycloalkyl), —N(C₁-C₄-alkyl)(C₃-C₆-cycloalkyl), —N(C₁-C₄-alkyl)(6-membered heterocycloalkyl), —N(C₁-C₄-alkyl)(C₁-C₄-alkoxy), morpholino optionally substituted with 1 to 2 C₁-C₄-alkyl groups, C₁-C₄-alkyl-N(C₁-C₄-alkyl)₂, wherein each C₁-C₄-alkyl in R2 may be optionally substituted with halogen, —N(C₁-C₄-alkyl)₂, C₁-C₄-alkoxy which itself may be substituted with C₁-C₂-alkoxy-substituted C₁-C₂-alkoxy, —S—C₁-C₄-alkyl, —S(O)—C₁-C₄-alkyl, —SO₂—C₁-C₄-alkyl, or optionally substituted by a monocyclic heterocycle selected from the group of 4- to 7-membered heterocycloalkyl, which itself may be substituted with methyl or oxo;
R3 is selected from the group consisting of hydrogen and C₁-C₄-alkyl;
R4 is selected from the group consisting of hydrogen, halogen, C₁-C₄-alkyl, C₁-C₄-halogenoalkyl having 1 to 5 halogen atoms, C₃-C₆-cycloalkyl, —S—C₁-C₄-alkyl, —S(O)—C₁-C₄-alkyl, —SO₂—C₁-C₄-alkyl; and
Q is a substituted phenyl ring of the formula (Q1)

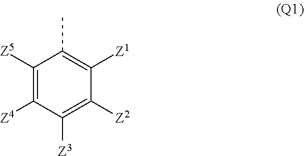

in which:
Z¹, Z², Z³, Z⁴, and Z⁵ are independently selected from the group consisting of hydrogen, fluorine, chlorine, bromine, cyano, methyl, propyl, difluoromethyl, trifluoromethyl, hydroxy, methoxy, ethoxy, isopropoxy, —O-cyclopropyl, —OCH₂-cyclopropyl, —OCH₂CN, trifluoromethoxy, difluoromethoxy, trifluoroethoxy, methylamino, dimethylamino, methylethylamino, diethylamino, acetylamino, methylsulfonamide, trifluoroacetylamino, —SO₂Me, —SO₂-cyclopropyl, —CH₂—O-methyl, —CH₂—O-ethyl, —CH₂—O—CH₂-cyclopropyl, —CH₂—O— isopropyl, —CH₂—N(CH₃)₂, —CH₂—N(CH₂CH₃)₂, —CH₂—N(CH₃)(CH₂CH₃), —CH₂—SCH₃, —CH₂—S(O)CH₃, —CH₂—SO₂—CH₃, —C(O)NH-cyclopropyl, and

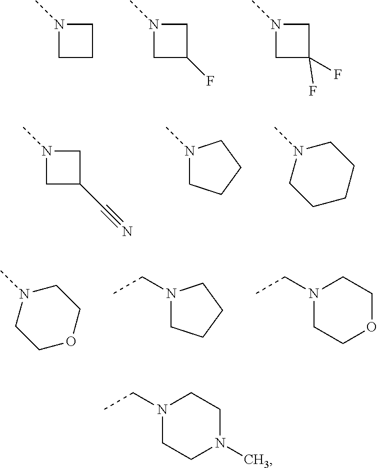

or
Z¹ and Z² form, together with the carbon atoms that they are connected to, a 5-membered heterocycloalkyl or a 5-membered heteroaryl, each of which may be optionally substituted with one or two substituents selected from the group consisting of methyl, fluorine and oxo, and
Z³, Z⁴, and Z⁵ are independently selected from the group consisting of hydrogen, fluorine, chlorine, bromine, cyano, methyl, propyl, difluoromethyl, trifluoromethyl, hydroxy, methoxy, ethoxy, isopropoxy, —O-cyclopropyl, —OCH₂-cyclopropyl, —OCH₂CN, trifluoromethoxy, difluoromethoxy, trifluoroethoxy, methylamino, dimethylamino, methylethylamino, diethylamino, acetylamino, methylsulfonamide, trifluoroacetylamino, —SO₂Me, —SO₂— cyclopropyl, —CH₂—O-methyl, —CH₂—O-ethyl, —CH₂—O—CH₂-cyclopropyl, —CH₂—O-isopropyl, —CH₂—N(CH₃)₂, —CH₂—N(CH₂CH₃)₂, —CH₂—N(CH₃)(CH₂CH₃), —CH₂—SCH₃, —CH₂—S(O)CH₃, —CH₂—SO₂—CH₃, —C(O)NH-cyclopropyl, and

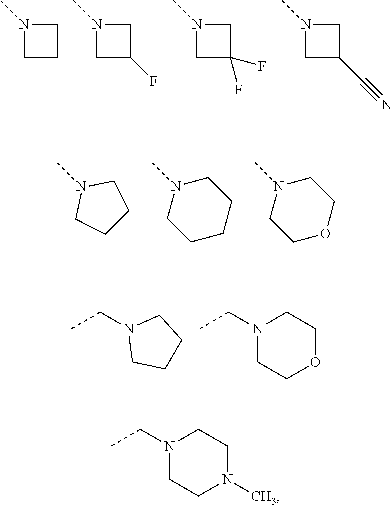

or
Z² and Z³ form, together with the carbon atoms that they are connected to, a 5-membered cycloalkyl or heterocycloalkyl or a 5-membered heteroaryl, each of which may be optionally substituted with one or two substituents selected from the group consisting of methyl, fluorine and oxo, and
Z¹, Z⁴, and Z⁵ are independently selected from the group consisting of hydrogen, fluorine, chlorine, bromine, cyano, methyl, propyl, difluoromethyl, trifluoromethyl, hydroxy, methoxy, ethoxy, isopropoxy, —O-cyclopropyl, —OCH₂-cyclopropyl, —OCH₂CN, trifluoromethoxy, difluoromethoxy, trifluoroethoxy, methylamino, dimethylamino, methylethylamino, diethylamino, acetylamino, methylsulfonamide, trifluoroacetylamino, —SO₂Me, —SO₂— cyclopropyl, —CH₂—O-methyl, —CH₂—O-ethyl, —CH₂—O—CH₂-cyclopropyl, —CH₂—O-isopropyl, —CH₂—N(CH₃)₂, —CH₂—N(CH₂CH₃)₂, —CH₂—N(CH₃)(CH₂CH₃), —CH₂—SCH₃, —CH₂—S(O)CH₃, —CH₂—SO₂—CH₃, —C(O)NH-cyclopropyl, and

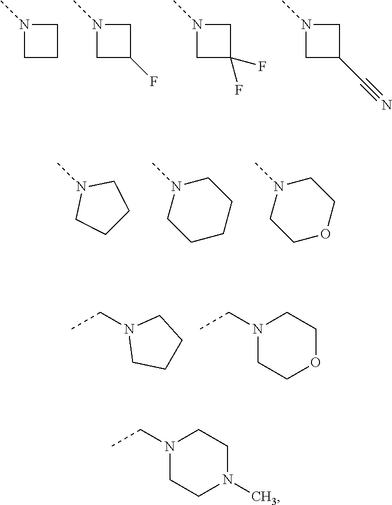

or
Q is a pyridine ring of the formula (Q2)

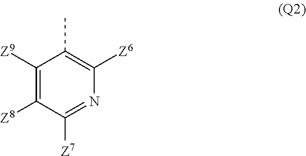

in which:
Z⁶, Z⁷, Z⁸ and Z⁹ are independently selected from the group consisting of hydrogen, fluorine or chlorine, or
Q is a pyrimidine ring of the formula (Q3)

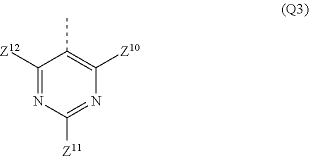

in which:
Z¹⁰, Z¹¹ and Z¹² are independently selected from the group consisting of hydrogen, fluorine, chlorine, C₁-C₄-alkyl, or
Q is a pyridine ring of the formula (Q4)

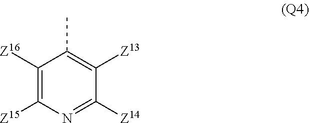

in which:
Z¹³, Z¹⁴, Z¹⁵ and Z¹⁶ are independently selected from the group consisting of hydrogen, fluorine, chlorine, C₁-C₄-alkyl, NH₂, —NH(C₁-C₄-alkyl), —N(C₁-C₄-alkyl)₂, —NH—CO—C₁-C₄-alkyl, and morpholino, pyrazoles, triazoles, imidazoles and pyrroles, wherein a heteroaryl ring is connected to the pyridine ring via one of its nitrogen atoms, each of which is optionally substituted with 1, 2 or 3 substituents independently selected from the group consisting of hydrogen, halogen, cyano, C₁-C₄-alkyl, C₁-C₄-halogenoalkyl having 1 to 5 halogen atoms, or
Q is a pyridine ring of the formula (Q5)

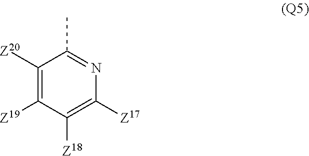

in which:
Z¹⁷, Z¹⁸, Z¹⁹ and Z²⁰ are independently selected from the group consisting of hydrogen, fluorine, chlorine, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, or
Q a pyrazole ring of the formula (Q6)

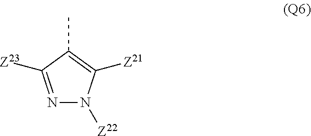

in which:
Z²¹ and Z²³ are hydrogen, and
Z²² is selected from the group consisting of hydrogen, C₁-C₄-alkyl, C₁-C₄-halogenoalkyl having 1 to 5 halogen atoms, C₁-C₄-alkyl-C₃-C₆-cycloalkyl, C₁-C₄-alkoxy-C₁-C₄-alkyl, C₁-C₄-alkyl-N(C₁-C₄-alkyl)₂, morpholino-C₁-C₄-alkyl, or
Q is a pyrazole ring of the formula (Q7)

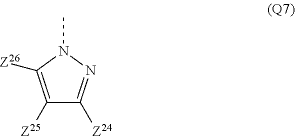

in which:
Z²⁴, Z²⁵ and Z²⁶ are independently selected from the group consisting of hydrogen, fluorine, chlorine, cyano, methyl, trifluoromethyl;
E2_1″ is

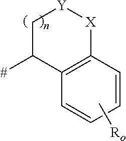

wherein
o is 0 or 1;
R is selected from the group consisting of hydrogen, fluorine, chlorine, C₁-C₄-alkyl;
n is 0 or 1;
X, Y are independently selected from the group consisting of CH₂ and O, wherein at least one of X and Y is CH₂;
E1_7 is

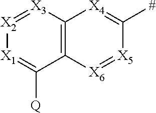

wherein
X₁ is CR₁; X₂ is CR₂; X₃ is CR₃; X₄ is CR₄; X₅ is N; and X₆ is N;
E2_2 is

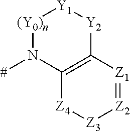

wherein
n is 0 or 1; when n is 1, Y₀ is CH₂ or C═O;
Y₁ is selected from the group consisting of CR₈R₉, O, S, and NR₁₀;
Y₂ is selected from the group consisting of CR₈R₉, O, S, and NR₁₀;
wherein at least one of the groups Y₁ or Y₂ is CR₈R₉;
Z₁ is selected from the group consisting of N, O, S, and CR₁₁;
Z₂ is selected from the group consisting of Nil, N, and CR₁₁;
Z₃ is selected from the group consisting of Nil, N and CR₁₁;
Z₄ is selected from the group consisting of N, O, S, and CR₁₁;
R8 is each time selected, independently selected from the group consisting of hydrogen, fluoro, and C₁-C₄ alkyl;
R9 is, each time selected, independently selected from the group consisting of hydrogen, fluoro, and C₁-C₄ alkyl;
R10 is selected from the group consisting of hydrogen and C₁-C₄ alkyl;
R11 is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and
—N(C₁-C₄ alkyl)₂;

The compounds of this embodiment are particularly suitable for the long-term prevention and/or treatment of a disease, in particular for the long-term prevention and/or treatment of a helminthic infection in an animal.

In one embodiment, the compound for use in the long-term prevention and/or treatment of a disease is a compound, wherein
E1 and E2 are selected from the following pairs when the compound of formula I is a compound of formula I.1:
E1_2 and E2_1′ wherein n is 0;
E1_4 and E2_1″ wherein n is 0;
E1_7 and E2_2 wherein n is 0;
   in which
   E1_2 is

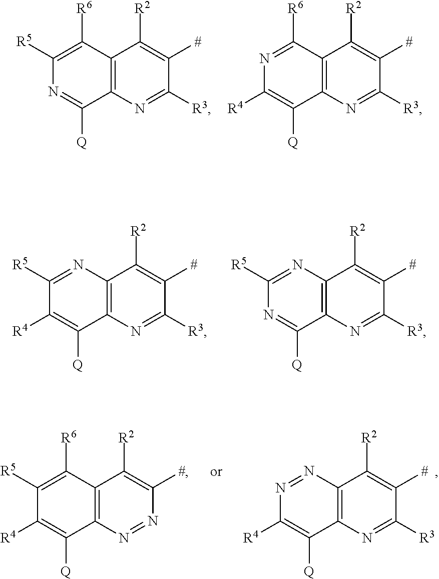

wherein
R2 is selected from the group consisting of hydrogen, chlorine, fluorine, bromine;
—NR¹²R¹³;
—OR¹⁴;
—SR¹⁵, —S(O)R¹⁵, —SO₂R¹⁵;
methyl, ethyl, propyl, isopropyl, cyclopropyl, cyclohexyl, propenyl, cyclopentenyl, cyclohexenyl, each of which is optionally substituted by 1 or 2 substituents independently selected from the group consisting of cyano, ethoxy-C(O)—, and —C(O)—NH₂; and
a monocyclic or a bicyclic heterocycle selected from the group consisting of azetidine, pyrrolidine, pyrazolidine, imidazolidine, 1,2,4-triazolidine, piperidine, piperazine, tetrahydropyridine, dihydro-2H-pyrane, tetrahydropyrane, 1,2-oxazolidine, 1,2-oxazine, morpholine, thiomorpholine, 3,4-dihydroisoquinoline, 2,3-dihydro-indole, 1,3-dihydro-isoindole, 3,9-dioxa-7-azabicyclo[3.3.1]nonane, 6-oxa-3-azabicyclo[3.1.1]heptane, 8-oxa-3-azabicyclo[3.2.1]octane, imidazole, pyrazole, 1,2,4-triazole, 1,2,3-triazole, 4-oxa-7-azaspiro[2.5]octane, each of which is optionally substituted by 1, 2, 3 or 4 substituents independently selected from the group consisting of fluorine, chlorine, cyano, —OH, oxo, —COOH, methoxy-C(O)—, ethoxy-C(O)—, tert-butoxy-C(O)—, —C(O)—NH₂, methyl, methyl-C(O)—, trifluoromethyl, hydroxymethyl-, methoxymethyl-, —NH₂, —NMe₂, pyrrolidine;
R3 is hydrogen, chlorine or methyl;
R4 is selected from the group consisting of hydrogen, halogen, cyano, C₁-C₄-alkyl, C₁-C₄-halogenoalkyl having 1 to 5 halogen atoms, C₁-C₄-alkoxy;
R5 is selected from the group consisting of hydrogen, fluorine, chlorine and methyl;
R6 is selected from the group consisting of hydrogen, fluorine, chlorine, methyl and methoxy;
R12 and R13 are independently selected from the group consisting of hydrogen, —NH(—C(O)-methyl), methoxy;
methyl, ethyl, propyl, isopropyl, butyl, isobutyl, cyclopropyl, cyclobutyl, benzyl, 1-phenylethyl, each of which is optionally substituted by 1, 2 or 3 substituents independently selected from the group consisting of fluorine, —OH, —COOH, methoxy-C(O)—, ethoxy-C(O)—, tert-butoxy-C(O)—, —C(O)—NH₂, —C(O)—NMe₂, —NH—C(O)-methyl, methyl, methoxy, cyclopropyl, —NH₂, NMe₂, S-methyl, S(O)-methyl, SO₂-methyl, and (EtO)₂P(═O)—;
heterocyclyl-methyl, heterocyclyl-ethyl, wherein the heterocyclyl substituent is selected from the group consisting of pyrrolidine, morpholine, pyrazole, 1, 2, 4-oxadiazole, pyridine, each of which is optionally substituted by 1 substituent independently selected from the group consisting of fluorine, chlorine, —OH, oxo and methyl;
phenyl; and
a monocyclic or a bicyclic heterocycle selected from the group of oxetane, thietane, pyrrolidine, morpholine, tetrahydropyrane, pyridine and pyrazole, each of which is optionally substituted by 1 or 2 substituents independently selected from the group consisting of fluorine, chlorine, —OH, oxo, methyl;
R14 is selected from the group consisting of methyl, ethyl, isopropyl, butyl, cyclopentyl, benzyl, each of which is optionally substituted by 1 or 2 substituents independently selected from the group consisting of fluorine, —OH, methyl, methoxy and cyclopentyl; and
a monocyclic or a bicyclic heterocycle selected from the group consisting of pyrrolidine and tetrahydropyrane,
R15 is selected from the group consisting of
methyl and ethyl, each of which is optionally substituted by 1 substituent independently selected from the group consisting of —OH and —COOH; and
pyridine,
Q is a substituted phenyl ring of the formula (Q1)

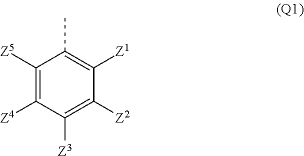

in which:
Z¹ and Z⁵ are independently selected from the group consisting of hydrogen, fluorine, chlorine, methyl and methoxy,
Z² and Z⁴ are independently selected from the group consisting of hydrogen, fluorine, chlorine, —OH, methyl, ethyl, —NHMe, —NMe₂, trifluoromethyl, methoxy, trifluoromethoxy, —SMe and morpholinyl, and
Z³ is independently selected from the group consisting of hydrogen, fluorine, chlorine, methyl, methoxy and —NMe₂;
wherein E2_1′ is

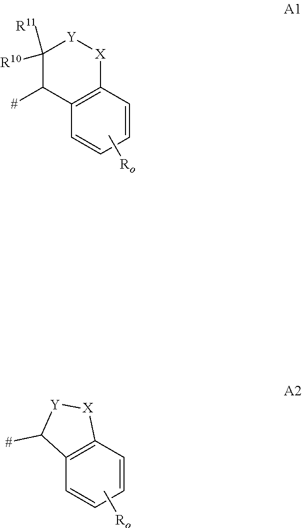

wherein
o is 0 or 1;
R is selected from the group consisting of hydrogen, halogen, C₁-C₄-alkyl and C₁-C₄-alkoxy;
X is selected from the group consisting of CR⁷R⁸, O, S, and N—R9;
Y is CR⁷R⁸;
R7 is selected from the group consisting of hydrogen and C₁-C₄-alkyl;
R8 is selected from the group consisting of hydrogen and C₁-C₄-alkyl;
R9 is C₁-C₄-alkyl;
R10 is selected from the group consisting of hydrogen, —OH and C₁-C₄-alkyl;
R11 is hydrogen;
E1_4 is

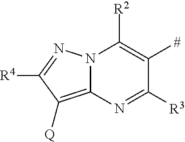

wherein
R2 is selected from the group consisting of hydrogen, methyl, ethyl, isopropyl, isobutyl, sec-butyl, cyclopropyl, methoxymethyl, difluoromethyl, trifluoromethyl, 4-fluorobenzyl, methoxy, methylamino, dimethylamino, cyclopropylamino,
—N(CH₃)(cyclopropyl), —N(CH₃)(CH₂—N(CH₃)₂), —N(CH₃)(CH₂—CHF₂), —N(CH₃)((CH₂)₂O(CH₂)₂)O(CH₂)₂)OCH₃), —N(CH₃)((CH₂)₂—S—CH₃), —N(CH₃)((CH₂)₂—S(O)—CH₃), —N(CH₃)((CH₂)₂—SO₂—CH₃), —N(CH₃)(1-methyl-piperidin-4-yl), —N(CH₃)((CH₂)₂-(oxopyrrolidin-1-yl)), morpholino, CH₂—N(CH₃)₂;
R3 is selected from the group consisting of hydrogen and methyl;
R4 is selected from the group consisting of hydrogen, chlorine, methyl, cyclopropyl, difluoromethyl, trifluoromethyl, —S-methyl, —S-ethyl, —S-isopropyl, —S(O)₂-methyl, —S(O)₂-ethyl, —S(O)₂-isopropyl; and
Q is a substituted phenyl ring of the formula (Q1)

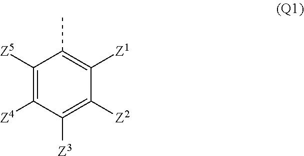

in which:
Z¹, Z², Z³, Z⁴, and Z⁵ are independently selected from the group consisting of hydrogen, fluorine, chlorine, bromine, cyano, methyl, propyl, difluoromethyl, trifluoromethyl, hydroxy, methoxy, ethoxy, isopropoxy, —O-cyclopropyl, —OCH₂-cyclopropyl, —OCH₂CN, trifluoromethoxy, difluoromethoxy, trifluoroethoxy, methylamino, dimethylamino, methylethylamino, diethylamino, acetylamino, methylsulfonamide, trifluoroacetylamino, —SO₂Me, —SO₂-cyclopropyl, —CH₂—O-methyl, —CH₂—O-ethyl, —CH₂—O—CH₂-cyclopropyl, —CH₂—O— isopropyl, —CH₂—N(CH₃)₂, —CH₂—N(CH₂CH₃)₂, —CH₂—N(CH₃)(CH₂CH₃), —CH₂—SCH₃, —CH₂—S(O)CH₃, —CH₂—SO₂—CH₃, —C(O)NH-cyclopropyl, and

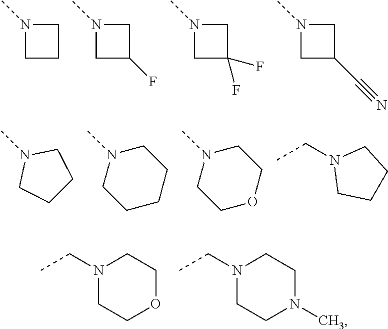

wherein at least two of Z¹, Z², Z³, Z⁴, and Z⁵ are hydrogen, or
Q is a pyridine ring of the formula (Q2)

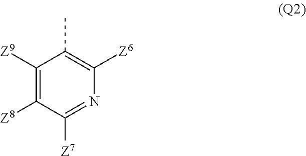

in which:
Z⁶ is hydrogen,
Z⁷, Z⁸ are independently selected from the group consisting of hydrogen, fluorine, chlorine, and
Z⁹ is selected from the group consisting of hydrogen and chlorine, or
Q is a pyrimidine ring of the formula (Q3)

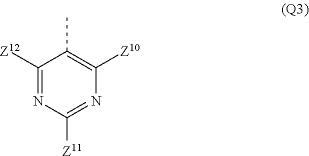

in which:
Z¹⁰ and Z¹² are hydrogen, and
Z¹¹ is selected from the group consisting of hydrogen and chlorine, or
Q is a pyridine ring of the formula (Q4)

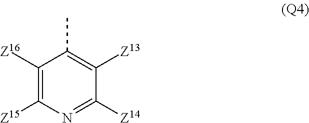

in which:
Z¹³, Z¹⁵ and Z¹⁶ are hydrogen, and
Z¹⁴ is selected from the group consisting of hydrogen and chlorine, NH₂, —NH—CO—C₁-C₄-alkyl, —NH(C₁-C₄-alkyl), —N(C₁-C₄-alkyl)₂, morpholino, or
Q is a pyridine ring of the formula (Q5)

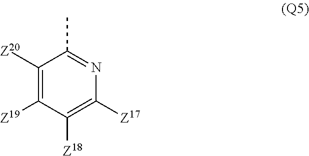

in which:
Z¹⁷ is selected from the group consisting of fluorine, chlorine, methoxy, trifluoromethyl,
Z¹⁸ and Z²⁰ are selected from the group consisting of hydrogen and chlorine,
Z¹⁹ is hydrogen, or
Q a pyrazole ring of the formula (Q6)

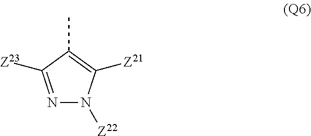

in which:
Z²¹ and Z²³ are hydrogen, and
Z²² is selected from the group consisting of hydrogen, methyl, ethyl, isopropyl, methoxyethyl, —CH₂-cyclopropyl, —CH₂CF₃, —CH₂CHF₂, —CH₂-morpholino, —CH₂—CH₂—N(CH₃)₂, and/or —CH₂—CH₂-morpholino, or
Q is a pyrazole ring of the formula (Q7)

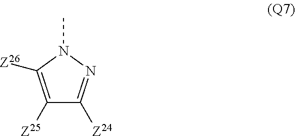

in which:
Z²⁴ and Z²⁶ are hydrogen, and
Z²⁵ is selected from the group consisting of hydrogen and chlorine, or
Q is selected from the group consisting of

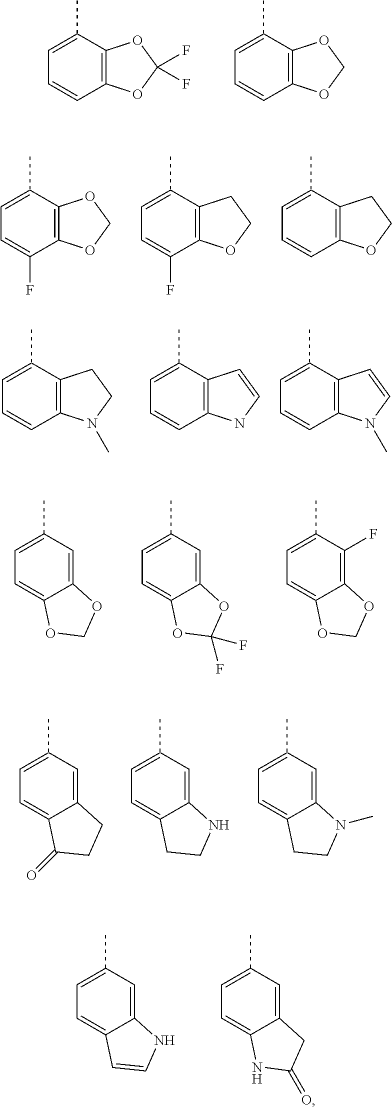

E2_1″ is

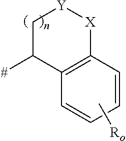

wherein
o is 0 or 1;
R is selected from the group consisting of hydrogen, fluorine, chlorine, methyl;
n is 0 or 1;
X is selected from the group consisting of CH₂ and O; and
Y is CH₂;
E1_7 is

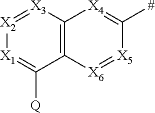

wherein
X₁ is CR₁; X₂ is CR₂; X₃ is CR₃; X₄ is CR₄; X₅ is N; and X₆ is CR₆;
E2_2 is

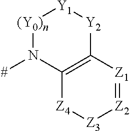

wherein
n is 0 or 1; when n is 1, Y₀ is CH₂ or C═O;
Y₁ is selected from the group consisting of CR₈R₉, O, S, and NR₁₀;
Y₂ is selected from the group consisting of CR₈R₉, O, S, and NR₁₀;
wherein at least one of the groups Y₁ or Y₂ is CR₈R₉;
Z₁ is selected from the group consisting of N, O, S, and CR₁₁;
Z₂ is selected from the group consisting of Nil, N, and CR₁₁;
Z₃ is selected from the group consisting of Nil, N and CR₁₁;
Z₄ is selected from the group consisting of N, O, S, and CR₁₁;
R8 is each time selected, independently selected from the group consisting of hydrogen, fluoro, and C₁-C₄ alkyl;
R9 is, each time selected, independently selected from the group consisting of hydrogen, fluoro, and C₁-C₄ alkyl;
R10 is selected from the group consisting of hydrogen and C₁-C₄ alkyl;
R11 is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and
—N(C₁-C₄ alkyl)₂.

The compounds of this embodiment are particularly suitable for the long-term prevention and/or treatment of a disease, in particular for the long-term prevention and/or treatment of a helminthic infection in an animal.

In one embodiment, the compound for use in the long-term prevention and/or treatment of a disease is a compound wherein
E1 and E2 are selected from the following pairs when the compound of formula I is a compound of formula I.1:
E1_7 and E2_1″ wherein n is 0;
E1_4 and E2_1″ wherein n is 0;
   in which
   E1_4 is

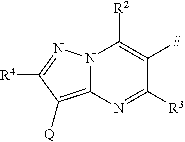

wherein
R4 is methyl;
Q is optionally substituted phenyl, wherein the optional substituent of phenyll is selected from the group consisting of halogen, alkyl optionally substituted with halogen, alkoxy optionally substituted with halogen, and —SF5;
R2 is selected from the group consisting of alkyl, cycloalkyl optionally substituted with halogen, CN, SO₂, 5- or 6-membered lactone, and 5- or 6-membered lactame;
R3 is H;
E1_7 is

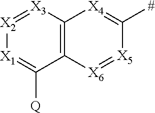

wherein
X1 is N or CR₁;
X2 is N or CR₂;
X3 is N or CR₃;
R1, R2 and R3 are selected from the group consisting of halogen, alkyl optionally substituted with halogen, alkoxy optionally substituted with halogen, —SF₅;
X4 is CR₄;
R4 is selected from the group consisting of alkyl, cycloalkyl optionally substituted with halogen, CN, SO₂, 5- or 6-membered lactone, and 5- or 6-membered lactame optionally substituted with halogen, 4- to 8-membered heterocycloalkyl optionally substituted with halogen;
X5 is CH;
X6 is N;
wherein at least one of X₁, X₂, X₃, is N;
E2_1″ is

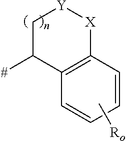

wherein
X is —CH₂— or O;
Y is —CH₂—;
n is 0 or 1;
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃—C-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms;
o is 0, 1, 2, 3 or 4.

The compounds of this embodiment are particularly suitable for the long-term prevention and/or treatment of a disease, in particular for the long-term prevention and/or treatment of a helminthic infection in an animal.

### PCT/EP2022/074866

In one embodiment, the compound for use in the long-term prevention and/or treatment of a disease is a compound according to the disclosure of PCT/EP2022/074866. The disclosure of PCT/EP2022/074866 is incorporated herein by reference in its entirety. In one embodiment, the compound for use in the long-term prevention and/or treatment of a disease according to the invention is a compound according to the following aspects of PCT/EP2022/074866:

### Aspect I of PCT/EP2022/074866:

A compound of general formula (I):

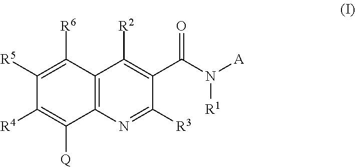

in which:
A is A1 or A2;

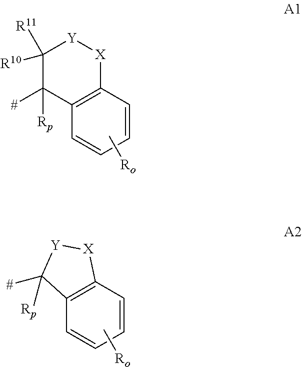

o is 0, 1, 2, 3 or 4;
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms;
Rₚ is selected from the group consisting of hydrogen, C₁-C₄-alkyl;
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—;
R¹ is selected from the group consisting of hydrogen, cyano, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N-C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, and —SO₂—C₁-C₄-halogenoalkyl having 1 to 5 halogen atoms;
phenyl-C₁-C₄-alkyl, optionally substituted by 1, 2, 3, 4 or 5 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R² is selected from the group consisting of 2-oxocyclobutyl, 3-oxocyclobutyl, 2-thiooxocyclobutyl, 3-thiooxocyclobutyl, 3-thietanyl, 2-thietanyl, oxetan-3-yl, oxetan-2-yl, 1-oxidothietan-3-yl, 1-oxidothietan-2-yl, 1-imino-1-oxido-1-thietan-3-yl, 1-imino-1-oxido-1-thietan-2-yl, 1,1-dioxidothietan-3-yl, 1,1-dioxidothietan-2-yl, 1,1-dioxido-1,2-thiazetidin-3-yl, 1,1-dioxido-1,2-thiazetidin-4-yl, 1-oxido-1,2-thiazetidin-3-yl, 1-oxido-1,2-thiazetidin-4-yl, 2-oxido-1,2-oxathietan-3-yl, 2-oxido-1,2-oxathietan-4-yl, 2,2-dioxido-1,2-oxathietan-3-yl, 2,2-dioxido-1,2-oxathietan-4-yl, 4-oxoazetidin-2-yl, 2-oxoazetidin-3-yl, 4-thioxoazetidin-2-yl, 2-thioxoazetidin-3-yl, 2-hydroxycyclobutyl, 3-hydroxycyclobutyl, 2-mercaptocyclobutyl, 3-mercaptocyclobutyl, 2-fluorocyclobutyl, 3-fluorocyclobutyl, 2,2-difluorocyclobutyl, 3,3-difluorocyclobutyl, 2-chlorocyclobutyl, 3-chlorocyclobutyl, 2,2-dichlorocyclobutyl, 3,3-dichlorocyclobutyl, 2-bromocyclobutyl, 3-bromocyclobutyl, 2,2-dibromocyclobutyl, 3,3-dibromocyclobutyl, 2-iodocyclobutyl, 3-iodocyclobutyl, 2,2-diiodocyclobutyl, 3,3-diiodocyclobutyl, 3-methoxyiminocyclobutyl, 2-fluoro-3-(methoxyimino)cyclobutyl, 2,2-difluoro-3-(methoxyimino)cyclobutyl, 2-chloro-3-(methoxyimino)cyclobutyl, 2,2-dichloro-3-(methoxyimino)cyclobutyl, 2-bromo-3-(methoxyimino)cyclobutyl, 2,2-dibromo-3-(methoxyimino)cyclobutyl, 2-iodo-3-(methoxyimino)cyclobutyl, 2,2-diiodo-3-(methoxyimino)cyclobutyl, 3-(hydroxyimino)cyclobutyl, 2-fluoro-3-(hydroxyimino)cyclobutyl, 2,2-difluoro-3-(hydroxyimino)cyclobutyl, 2-chloro-3-(hydroxyimino)cyclobutyl, 2,2-dichloro-3-(hydroxyimino)cyclobutyl, 2-bromo-3-(hydroxyimino)cyclobutyl, 2,2-dibromo-3-(hydroxyimino)cyclobutyl, 2-iodo-3-(hydroxyimino)cyclobutyl, 2,2-diiodo-3-(hydroxyimino)cyclobutyl, 5- to 10-membered heterocycloalkyl, 5-membered heteroaryl and 6-membered heteroaryl, each of which is optionally substituted by 1, 2 or 3 substituents independently selected from the group consisting of halogen, —OH, -oxo, —NO₂, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R³ is hydrogen or C₁-C₄-alkyl;
R⁴ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —S—C₁-C₄-alkyl, —S(O)—C₁-C₄-alkyl, —SO₂—C₁-C₄-alkyl, preferably hydrogen, halogen and C₁-C₄-alkoxy, more preferably fluorine, chlorine, methoxy and isopropoxy;
R⁵ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl;
R⁶ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl;
R⁷ is selected from the group consisting of hydrogen, —OH, fluorine, C₁-C₄-alkyl and C₁-C₄-alkoxy;
R⁸ is selected from the group consisting of hydrogen, —OH, fluorine, C₁-C₄-alkyl and C₁-C₄-alkoxy;
or R⁷ and R⁸ together form an oxo group (═O);
or R⁷ and R⁸ form, together with the carbon atom to which they are attached, a 3- to 6-membered ring selected from the group consisting of C₃-C₆-cycloalkyl and 3- to 6-membered heterocycloalkyl;
R⁹ is selected from the group consisting of hydrogen, C₁-C₄-alkyl, C₁-C₄-halogenoalkyl having 1 to 5 halogen atoms and C₁-C₄-alkoxy;
R¹⁰ is selected from the group consisting of hydrogen, —OH, C₁-C₄-alkyl and C₁-C₄-alkoxy;
R¹¹ is selected from the group consisting of hydrogen, C₁-C₄-alkyl and C₁-C₄-alkoxy;
or R¹⁰ and R¹¹ form, together with the carbon atom to which they are attached, a 3- to 6-membered ring selected from the group consisting of C₃-C₆-cycloalkyl and 3- to 6-membered heterocycloalkyl;
Q represents phenyl having 1 to 5 halogen atoms,
wherein when Y is O, S or N—R⁹, none of R⁷, R⁸, R¹⁰ and R¹¹ is —OH or C₁-C₄-alkoxy, and
wherein when X is O, S or N—R⁹, none of R⁷ and R⁸ is —OH or C₁-C₄-alkoxy,
or a stereoisomer, a tautomer, an N-oxide, a hydrate, a solvate, or a salt thereof, or a mixture of same.

### Aspect II PCT/EP2022/074866:

The compound according to Aspect I of PCT/EP2022/074866, wherein:
A is A1 or A2;

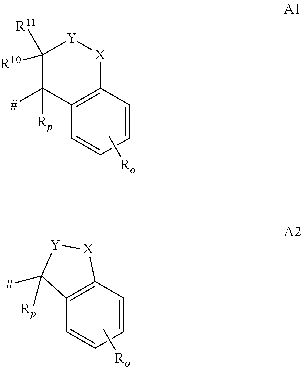

o is 0, 1, 2, 3 or 4;
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms;
Rₚ is selected from the group consisting of hydrogen, C₁-C₄-alkyl;
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—;
R¹ is selected from the group consisting of hydrogen, cyano, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N—C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, and —SO₂—C₁-C₄-halogenoalkyl having 1 to 5 halogen atoms;
phenyl-C₁-C₄-alkyl, optionally substituted by 1, 2, 3, 4 or 5 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R² is selected from the group consisting of 2-oxocyclobutyl, 3-oxocyclobutyl, 2-thiooxocyclobutyl, 3-thiooxocyclobutyl, 3-thietanyl, 2-thietanyl, oxetan-3-yl, oxetan-2-yl, 1-oxidothietan-3-yl, 1-oxidothietan-2-yl, 1-imino-1-oxido-1-thietan-3-yl, 1-imino-1-oxido-1-thietan-2-yl, 1,1-dioxidothietan-3-yl, 1,1-dioxidothietan-2-yl, 1,1-dioxido-1,2-thiazetidin-3-yl, 1,1-dioxido-1,2-thiazetidin-4-yl, 1-oxido-1,2-thiazetidin-3-yl, 1-oxido-1,2-thiazetidin-4-yl, 2-oxido-1,2-oxathietan-3-yl, 2-oxido-1,2-oxathietan-4-yl, 2,2-dioxido-1,2-oxathietan-3-yl, 2,2-dioxido-1,2-oxathietan-4-yl, 4-oxoazetidin-2-yl, 2-oxoazetidin-3-yl, 4-thioxoazetidin-2-yl, 2-thioxoazetidin-3-yl, 2-hydroxycyclobutyl, 3-hydroxycyclobutyl, 2-mercaptocyclobutyl, 3-mercaptocyclobutyl, 2-fluorocyclobutyl, 3-fluorocyclobutyl, 2,2-difluorocyclobutyl, 3,3-difluorocyclobutyl, 2-chlorocyclobutyl, 3-chlorocyclobutyl, 2,2-dichlorocyclobutyl, 3,3-dichlorocyclobutyl, 2-bromocyclobutyl, 3-bromocyclobutyl, 2,2-dibromocyclobutyl, 3,3-dibromocyclobutyl, 2-iodocyclobutyl, 3-iodocyclobutyl, 2,2-diiodocyclobutyl, 3,3-diiodocyclobutyl, 3-methoxyiminocyclobutyl, 2-fluoro-3-(methoxyimino)cyclobutyl, 2,2-difluoro-3-(methoxyimino)cyclobutyl, 2-chloro-3-(methoxyimino)cyclobutyl, 2,2-dichloro-3-(methoxyimino)cyclobutyl, 2-bromo-3-(methoxyimino)cyclobutyl, 2,2-dibromo-3-(methoxyimino)cyclobutyl, 2-iodo-3-(methoxyimino)cyclobutyl, 2,2-diiodo-3-(methoxyimino)cyclobutyl, 3-(hydroxyimino)cyclobutyl, 2-fluoro-3-(hydroxyimino)cyclobutyl, 2,2-difluoro-3-(hydroxyimino)cyclobutyl, 2-chloro-3-(hydroxyimino)cyclobutyl, 2,2-dichloro-3-(hydroxyimino)cyclobutyl, 2-bromo-3-(hydroxyimino)cyclobutyl, 2,2-dibromo-3-(hydroxyimino)cyclobutyl, 2-iodo-3-(hydroxyimino)cyclobutyl, 2,2-diiodo-3-(hydroxyimino)cyclobutyl, tetrahydrofuran-2-yl, tetrahydrofuran-3-yl, 2,5-dihydrofuran-3-yl, 2,3-dihydrofuran-3-yl, 4,5-dihydrofuran-3-yl, 4,5-dihydrofuran-2-yl, 2,5-dihydrofuran-2-yl, 2,3-dihydrofuran-2-yl, furan-3-yl, furan-2-yl, tetrahydrothiophen-3-yl, tetrahydrothiophen-2-yl, 2,5-dihydrothiophen-3-yl, 2,3-dihydrothiophen-3-yl, 4,5-dihydrothiophen-3-yl, 4,5-dihydrothiophen-2-yl, 2,5-dihydrothiophen-2-yl, 2,3-dihydrothiophen-2-yl, thiophen-3-yl, thiophen-2-yl, pyrrolidine-2-yl, pyrrolidine-3-yl, 1-methylpyrrolidine-2-yl, 1-methylpyrrolidine-3-yl, 4,5-dihydro-1H-pyrrol-2-yl, 2,5-dihydro-1H-pyrrol-2-yl, 2,3-dihydro-1H-pyrrol-2-yl, 3,4-dihydro-2H-pyrrol-2-yl, 3,4-dihydro-2H-pyrrol-5-yl, 4,5-dihydro-1H-pyrrol-3-yl, 3,4-dihydro-2H-pyrrol-4-yl, 3,4-dihydro-2H-pyrrol-3-yl, 2,3-dihydro-1H-pyrrol-3-yl, 2,5-dihydro-1H-pyrrol-3-yl, 2H-pyrrol-5-yl, 3H-pyrrol-2-yl, 2H-pyrrol-4-yl, 1H-pyrrol-3-yl, 5-oxopyrrolidine-3-yl, 2-oxopyrrolidine-3-yl, 5-oxopyrrolidine-2-yl, tetrahydropyran-4-yl, 3-oxopiperazin-1-yl, 2-oxopiperazin-1-yl, 4-alkyl-3-oxopiperazin-1-yl, 4-alkyl-2-oxopiperazin-1-yl, wherein the alkyl is C₁-C₆-alkyl, 2-oxa-5-azabicyclo[4.1.0]heptan-5-yl, 3,7-dioxa-9-azabicyclo[3.3.1]nonan-9-yl;
R³ is hydrogen, or C₁-C₄-alkyl;
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, preferably hydrogen, halogen and C₁-C₄-alkoxy, more preferably fluorine, chlorine, methoxy and isopropoxy;
R⁵ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂;
R⁶ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂;
R⁷ is selected from the group consisting of hydrogen, —OH, fluorine, C₁-C₄-alkyl and C₁-C₄-alkoxy;
R⁸ is selected from the group consisting of hydrogen, —OH, fluorine, C₁-C₄-alkyl and C₁-C₄-alkoxy;
or R⁷ and R⁸ together form an oxo group (═O);
R⁹ is selected from the group consisting of hydrogen, C₁-C₄-alkyl, C₁-C₄-halogenoalkyl having 1 to 5 halogen atoms and C₁-C₄-alkoxy;
R¹⁰ is selected from the group consisting of hydrogen, —OH, C₁-C₄-alkyl and C₁-C₄-alkoxy;
R¹¹ is selected from the group consisting of hydrogen, C₁-C₄-alkyl and C₁-C₄-alkoxy;
Q represents phenyl having 1 to 5 halogen atoms,
wherein when Y is O, S or N—R⁹, none of R⁷, R⁸, R¹⁰ and R¹¹ is —OH or C₁-C₄-alkoxy, and
wherein when X is O, S or N—R⁹, none of R⁷ and R⁸ is —OH or C₁-C₄-alkoxy,
or a stereoisomer, a tautomer, an N-oxide, a hydrate, a solvate, or a salt thereof, or a mixture of same.

### Aspect III of PCT/EP2022/074866:

The compound according to Aspect 1 or 2 of PCT/EP2022/074866, wherein:
A is A1 or A2,

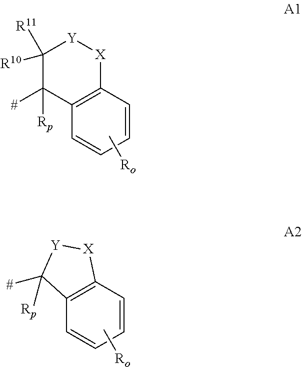

o is 0, 1 or 2,
R is selected from the group consisting of hydrogen, halogen, C₁-C₄-alkyl and C₁-C₄-alkoxy, cyano, C₁-C₄-halogenoalkyl having 1 to 5 halogen atoms,
Rₚ is selected from the group consisting of hydrogen, C₁-C₄-alkyl,
X, Y are independently selected from the group consisting of CR⁷R⁸, O, S, and N—R⁹, wherein at least one of X and Y is CR⁷R⁸,
R¹ is selected from the group consisting of hydrogen, C₁-C₄-alkyl, C₃-C₆-cycloalkyl, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl,
R² is selected from the group consisting of 2-oxocyclobutyl, 3-oxocyclobutyl, 3-thietanyl, 2-thietanyl, 1-oxidothietan-3-yl, 1-oxidothietan-2-yl, 1-imino-1-oxido-1-thietan-3-yl, 1-imino-1-oxido-1-thietan-2-yl, 1,1-dioxidothietan-3-yl, 1,1-dioxidothietan-2-yl, 4-oxoazetidin-2-yl, 2-oxoazetidin-3-yl, 2-hydroxycyclobutyl, 3-hydroxycyclobutyl, 2-fluorocyclobutyl, 3-fluorocyclobutyl, 2,2-difluorocyclobutyl, 3,3-difluorocyclobutyl, tetrahydrofuran-2-yl, tetrahydrofuran-3-yl, 1-methylpyrrolidine-2-yl, 1-methylpyrrolidine-3-yl, 5-oxopyrrolidine-3-yl, 2-oxopyrrolidine-3-yl, 5-oxopyrrolidine-2-yl, tetrahydropyran-4-yl, 3-oxopiperazin-1-yl, 2-oxopiperazin-1-yl, 2-oxa-5-azabicyclo[4.1.0]heptan-5-yl, 3,7-dioxa-9-azabicyclo[3.3.1]nonan-9-yl,
R³ is hydrogen or C₁-C₄-alkyl,
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, preferably hydrogen, halogen and C₁-C₄-alkoxy, more preferably fluorine, chlorine, methoxy and isopropoxy,
R⁵ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁶ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁷ is selected from the group consisting of hydrogen and C₁-C₄-alkyl,
R⁸ is selected from the group consisting of hydrogen and C₁-C₄-alkyl,
or R⁷ and R⁸ together form an oxo group (═O),
R⁹ is hydrogen or C₁-C₄-alkyl,
R¹⁰ is selected from the group consisting of hydrogen, —OH, C₁-C₄-alkyl and C₁-C₄-alkoxy,
R¹¹ is hydrogen,
Q represents phenyl having 1 to 5 substituents independently selected from fluorine, chlorine, or bromine
wherein when Y is O, S or N—R⁹, R¹⁰ is not —OH or C₁-C₄-alkoxy, or a stereoisomer, a tautomer, an N-oxide, a hydrate, a solvate, or a salt thereof, or a mixture of same.

### Aspect IV of PCT/EP2022/074866:

The compound according to any one of Aspects 1, 2 or 3 of PCT/EP2022/074866, wherein:

A is A1 or A2,

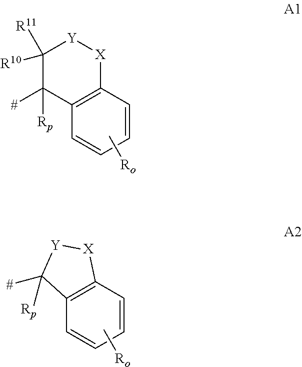

o is 0, 1 or 2,
R is selected from the group consisting of hydrogen, halogen, C₁-C₄-alkyl, C₁-C₄-alkoxy, and cyano,
Rₚ is selected from the group consisting of hydrogen, C₁-C₄-alkyl,
X is selected from the group consisting of CR⁷R⁸, O, S, and N—R⁹,
Y is CR⁷R⁸ or O,
R¹ is hydrogen or C₁-C₄-alkyl,
R² is selected from the group consisting of 2-oxocyclobutyl, 3-oxocyclobutyl, 3-thietanyl, 2-thietanyl, 1-oxidothietan-3-yl, 1-oxidothietan-2-yl, 1-imino-1-oxido-1-thietan-3-yl, 1-imino-1-oxido-1-thietan-2-yl, 1,1-dioxidothietan-3-yl, 1,1-dioxidothietan-2-yl, 4-oxoazetidin-2-yl, 2-oxoazetidin-3-yl, 2-hydroxycyclobutyl, 3-hydroxycyclobutyl, 2-fluorocyclobutyl, 3-fluorocyclobutyl, tetrahydrofuran-2-yl, tetrahydrofuran-3-yl, 1-methylpyrrolidine-2-yl, 1-methylpyrrolidine-3-yl, 5-oxopyrrolidine-3-yl, 2-oxopyrrolidine-3-yl, 5-oxopyrrolidine-2-yl, tetrahydropyran-4-yl, 3-oxopiperazin-1-yl, 2-oxopiperazin-1-yl, 2-oxa-5-azabicyclo[4.1.0]heptan-5-yl, 3,7-dioxa-9-azabicyclo[3.3.1]nonan-9-yl,
R³ is hydrogen or C₁-C₄-alkyl,
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, preferably hydrogen, halogen and C₁-C₄-alkoxy, more preferably fluorine, chlorine, methoxy and isopropoxy,
R⁵ is selected from the group consisting of hydrogen, halogen, —OH, —NH₂, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
R⁶ is selected from the group consisting of hydrogen, halogen, —OH, cyano, C₁-C₄-alkyl, C₁-C₄-halogenoalkyl having 1 to 5 halogen atoms, C₁-C₄-alkoxy,
R⁷ is selected from the group consisting of hydrogen and C₁-C₄-alkyl,
R⁸ is selected from the group consisting of hydrogen and C₁-C₄-alkyl,
or R⁷ and R⁸ together form an oxo group (═O),
R⁹ is hydrogen or C₁-C₄-alkyl,
R¹⁰ is selected from the group consisting of hydrogen, —OH and C₁-C₄-alkyl,
R¹¹ is hydrogen,
Q represents phenyl having 2 or 3 substituents independently selected from fluorine, chlorine, or bromine
wherein when Y is O, R¹⁰ is not —OH,
or a stereoisomer, a tautomer, an N-oxide, a hydrate, a solvate, or a salt thereof, or a mixture of same.

### Aspect V of PCT/EP2022/074866:

The compound according to Aspect 1, 2, 3 or 4 of PCT/EP2022/074866, wherein:
A is selected from the group consisting of

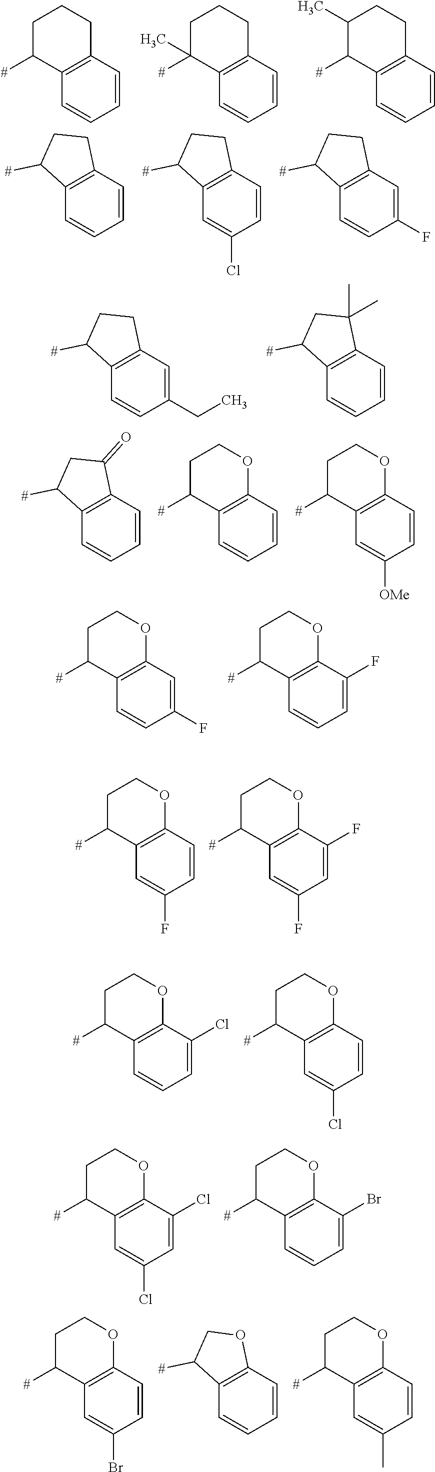

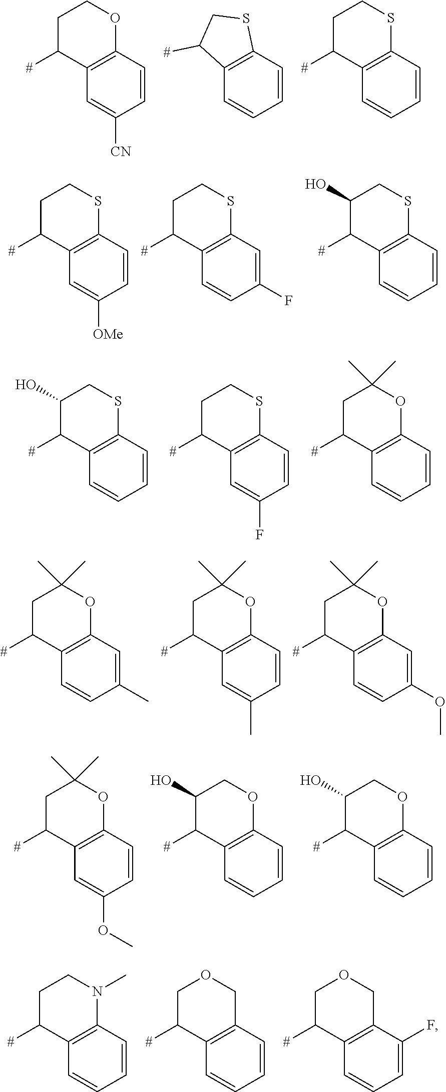

preferably

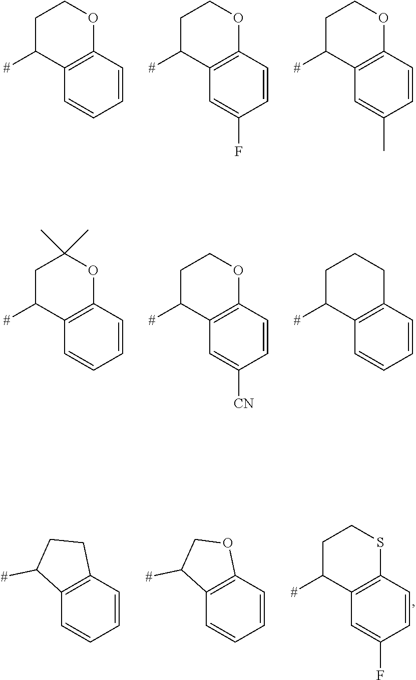

R¹ is hydrogen or methyl,
R² is selected from the group consisting of 2-oxocyclobutyl, 3-oxocyclobutyl, 3-thietanyl, 2-thietanyl, 1-oxidothietan-3-yl, 1-oxidothietan-2-yl, 1-imino-1-oxido-1-thietan-3-yl, 1-imino-1-oxido-1-thietan-2-yl, 1,1-dioxidothietan-3-yl, 1,1-dioxidothietan-2-yl, 4-oxoazetidin-2-yl, 2-oxoazetidin-3-yl, 2-hydroxycyclobutyl, 3-hydroxycyclobutyl, 2-fluorocyclobutyl, 3-fluorocyclobutyl, tetrahydrofuran-2-yl, tetrahydrofuran-3-yl, 1-methylpyrrolidine-2-yl, 1-methylpyrrolidine-3-yl, 5-oxopyrrolidine-3-yl, 2-oxopyrrolidine-3-yl, 5-oxopyrrolidine-2-yl, tetrahydropyran-4-yl, 3-oxopiperazin-1-yl, 2-oxopiperazin-1-yl, 2-oxa-5-azabicyclo[4.1.0]heptan-5-yl, 3,7-dioxa-9-azabicyclo[3.3.1]nonan-9-yl,
R³ is hydrogen or methyl,
R⁴ is selected from the group consisting of hydrogen, fluorine, chlorine, —OH, cyano, methyl, methoxy, trifluoromethyl, isopropoxy, and trifluoromethoxy, preferably hydrogen, fluorine, chlorine, methoxy and isopropoxy,
R⁵ is selected from the group consisting of hydrogen, fluorine, chlorine, —OH, cyano, methyl, methoxy, trifluoromethyl, trifluoromethoxy and NH₂,
R⁶ is selected from the group consisting of hydrogen, fluorine, chlorine, —OH, cyano, methyl and methoxy,
Q is selected from the group consisting of 2,3-dichlorophenyl, 3,5-dichlorophenyl, and 2,3,5-trifluorophenyl,
or a stereoisomer, a tautomer, an N-oxide, a hydrate, a solvate, or a salt thereof, or a mixture of same.

### Aspect VI of PCT/EP2022/074866:

The compound according to Aspect 1, 2, 3, 4 or 5 of PCT/EP2022/074866, wherein:
A is selected from the group consisting of

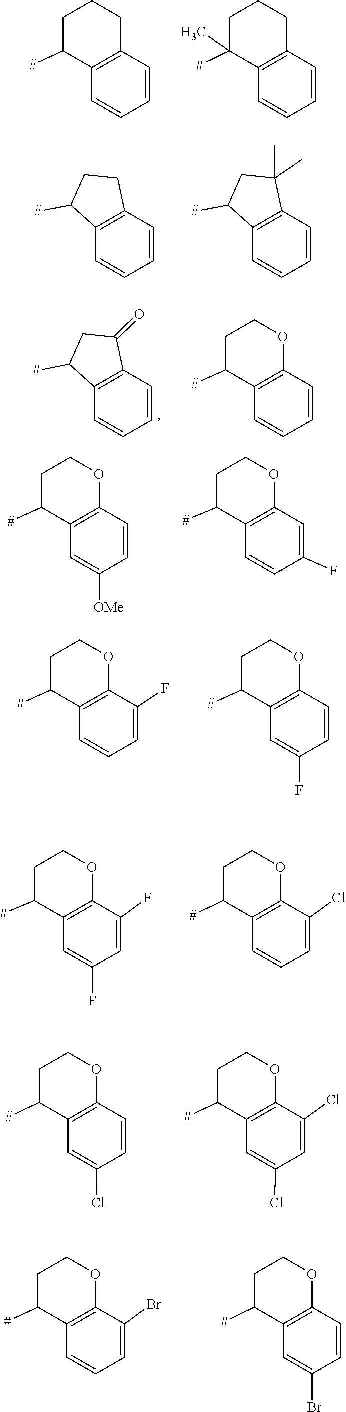

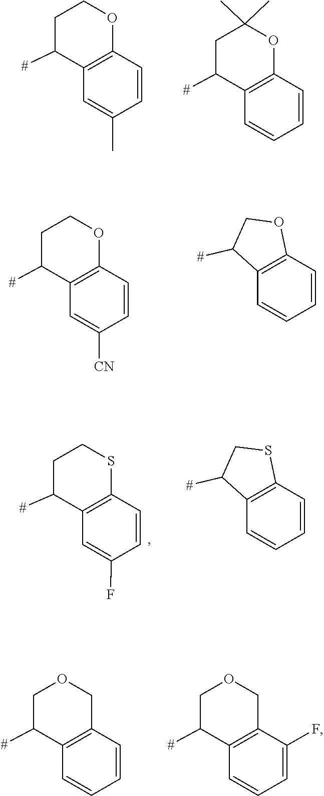

preferably

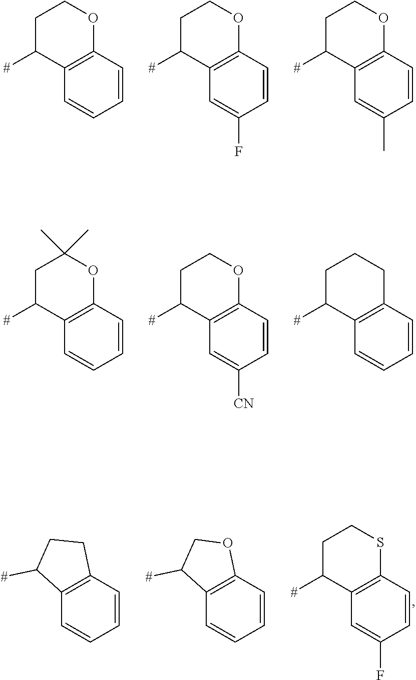

R¹ is hydrogen or methyl,
R² is selected from the group consisting of 2-oxocyclobutyl, 3-oxocyclobutyl, 3-thietanyl, 2-thietanyl, 1-oxidothietan-3-yl, 1-oxidothietan-2-yl, 1-imino-1-oxido-1-thietan-3-yl, 1-imino-1-oxido-1-thietan-2-yl, 1,1-dioxidothietan-3-yl, 1,1-dioxidothietan-2-yl, 4-oxoazetidin-2-yl, 2-oxoazetidin-3-yl, 2-hydroxycyclobutyl, 3-hydroxycyclobutyl, 2-fluorocyclobutyl, 3-fluorocyclobutyl, 3,3-difluorocyclobutyl, tetrahydrofuran-2-yl, tetrahydrofuran-3-yl, 1-methylpyrrolidine-2-yl, 1-methylpyrrolidine-3-yl, 5-oxopyrrolidine-3-yl, 2-oxopyrrolidine-3-yl, 5-oxopyrrolidine-2-yl, tetrahydropyran-4-yl, 3-oxopiperazin-1-yl, 2-oxopiperazin-1-yl, 2-oxa-5-azabicyclo[4.1.0]heptan-5-yl, 3,7-dioxa-9-azabicyclo[3.3.1]nonan-9-yl,
R³ is hydrogen or methyl,
R⁴ is selected from the group consisting of hydrogen, chlorine, fluorine, methyl, methoxy, isopropoxy and trifluoromethyl, preferably hydrogen, chlorine, fluorine, methoxy and isopropoxy,
R⁵ is selected from the group consisting of hydrogen, chlorine, fluorine, —OH, cyano, methyl, trifluoromethoxy and NH₂,
R⁶ is selected from the group consisting of hydrogen, fluorine, chlorine, —OH, cyano, methyl and methoxy,
Q is selected from the group of 2,3-dichlorophenyl, 3,5-dichlorophenyl, and 2,3,5-trifluorophenyl,
or a stereoisomer, a tautomer, an N-oxide, a hydrate, a solvate, or a salt thereof, or a mixture of same.

### Aspect VII of PCT/EP2022/074866:

The compound according to Aspect 1, 2, 3, 4, 5, or 6 of PCT/EP2022/074866, wherein:
R² is selected from the group consisting of 3-oxocyclobutyl, 3-thietanyl, 1-oxidothietan-3-yl, 1-imino-1-oxido-1-thietan-3-yl, 1,1-dioxidothietan-3-yl, 2-oxoazetidin-3-yl, 3-hydroxycyclobutyl, 3-fluorocyclobutyl, 3,3-difluorocyclobutyl, tetrahydrofuran-3-yl, 1-methylpyrrolidine-2-yl, 1-methylpyrrolidine-3-yl, 5-oxopyrrolidine-3-yl, 2-oxopyrrolidine-3-yl, 5-oxopyrrolidine-2-yl, tetrahydropyran-4-yl, 3-oxopiperazin-1-yl, 2-oxa-5-azabicyclo[4.1.0]heptan-5-yl, 3,7-dioxa-9-azabicyclo[3.3.1]nonan-9-yl,
Q is 2,3,5-trifluorophenyl or 3,5-dichlorophenyl,
or a stereoisomer, a tautomer, an N-oxide, a hydrate, a solvate, or a salt thereof, or a mixture of same.

### Aspect VIII of PCT/EP2022/074866:

The compound according to Aspect 1, 2, 3, 4, 5, 6, or 7 of PCT/EP2022/074866, wherein:
R² is selected from the group consisting of 3-oxocyclobutyl, 3-thietanyl, 1-oxidothietan-3-yl, 1-imino-1-oxido-1-thietan-3-yl, 1,1-dioxidothietan-3-yl, 2-oxoazetidin-3-yl, 3-hydroxycyclobutyl, 3-fluorocyclobutyl, tetrahydrofuran-3-yl, 1-methylpyrrolidine-2-yl, 1-methylpyrrolidine-3-yl, 5-oxopyrrolidine-3-yl, 2-oxopyrrolidine-3-yl, 5-oxopyrrolidine-2-yl, tetrahydropyran-4-yl, 2-oxa-5-azabicyclo[4.1.0]heptan-5-yl, 3,7-dioxa-9-azabicyclo[3.3.1]nonan-9-yl,
R⁴ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —S—C₁-C₄-alkyl, —S(O)—C₁-C₄-alkyl, —SO₂—C₁-C₄-alkyl, preferably hydrogen, halogen and C₁-C₄-alkoxy, more preferably fluorine, chlorine, methoxy and isopropoxy,
Q is 2,3,5-trifluorophenyl or 3,5-dichlorophenyl,
or a stereoisomer, a tautomer, an N-oxide, a hydrate, a solvate, or a salt thereof, or a mixture of same.

### Aspect IX of PCT/EP2022/074866:

The compound according to Aspect 1, 2, 3, 4, 5, 6, 7, or 8 of PCT/EP2022/074866, wherein:
R² is 3-hydroxycyclobutyl, 3-fluorocyclobutyl, tetrahydrofuran-3-yl or 3-oxocyclobutyl,
Q is 2,3,5-trifluorophenyl or 3,5-dichlorophenyl

In one embodiment, E1_1, E2_1, n and R1 are as defined in PCT/EP2022/074866. In one embodiment, E1_1, E2_1, n and R1 are as defined in the above-described aspects of PCT/EP2022/074866. In one embodiment, the compound of general formula (I) is a compound of formula I.1 wherein E1_1, E2_1, and R1, are as defined in the above-described aspects of PCT/EP2022/074866 and n is 0.

WO 2018/087036A1 In one embodiment, the compound for use in the long-term prevention and/or treatment of a disease is a compound according to the disclosure of WO 2018/087036 A1. The disclosure of WO 2018/087036 A1 is incorporated herein by reference in its entirety. In one embodiment, the compound for use in the long-term prevention and/or treatment of a disease according to the invention is a compound according to the following aspects of WO 2018/087036 A1:

### Aspect I of WO 2018/087036 A1:

A compound of general formula (I):

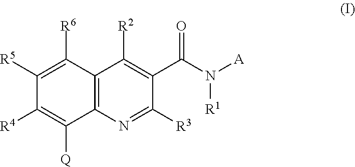

in which:
A is A1 or A2,

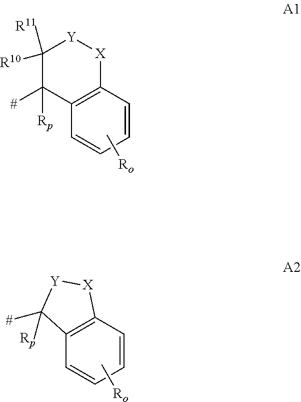

o is 0, 1, 2, 3 or 4,
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms,
Rₚ is selected from the group consisting of hydrogen, C₁-C₄-alkyl,
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—,
R¹ is selected from the group consisting of hydrogen, cyano, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N—C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, and —SO₂—C₁-C₄-halogenoalkyl having 1 to 5 halogen atoms;
phenyl-C₁-C₄-alkyl, optionally substituted by 1, 2, 3, 4 or 5 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R² is selected from the group consisting of hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —NH(C(O)—C₁-C₄-alkyl), —N(C₁-C₄-alkyl)(C(O)—C₁-C₄-alkyl), —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl,
R³ is hydrogen or C₁-C₄-alkyl,
R⁴ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl,
R⁵ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl,
R⁶ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl,
R⁷ is selected from the group consisting of hydrogen, —OH, fluorine, C₁-C₄-alkyl and C₁-C₄-alkoxy,
R⁸ is selected from the group consisting of hydrogen, —OH, fluorine, C₁-C₄-alkyl and C₁-C₄-alkoxy,
or R⁷ and R⁸ together form an oxo group (═O),
or R⁷ and R⁸ form, together with the carbon atom to which they are attached, a 3- to 6-membered ring selected from the group consisting of C₃-C₆-cycloalkyl and 3- to 6-membered heterocycloalkyl,
R⁹ is selected from the group consisting of hydrogen, C₁-C₄-alkyl, C₁-C₄-halogenoalkyl having 1 to 5 halogen atoms and C₁-C₄-alkoxy,
R¹⁰ is selected from the group consisting of hydrogen, —OH, C₁-C₄-alkyl and C₁-C₄-alkoxy,
R¹¹ is selected from the group consisting of hydrogen, C₁-C₄-alkyl and C₁-C₄-alkoxy,
or R¹⁰ and R¹¹ form, together with the carbon atom to which they are attached, a 3- to 6-membered ring selected from the group consisting of C₃-C₆-cycloalkyl and 3- to 6-membered heterocycloalkyl,
R¹² and R¹³ are independently selected from the group consisting of hydrogen, —OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy, C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)P(═O)—;
   heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R¹⁴ is selected from the group consisting of —NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R¹⁵ is selected from the group consisting of hydrogen;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
Q is selected from the group consisting of 6- or 10-membered aryl and 5- to 10-membered heteroaryl, each of which is optionally substituted by 1, 2, 3, 4 or 5 substituents selected from the group consisting of halogen, SF₅, cyano, —CHO, nitro, oxo, C₁-C₄-alkyl, C₁-C₄-hydroxyalkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms,
wherein when Y is O, S or N—R⁹, none of R⁷, R⁸, R¹⁰ and R¹¹ is —OH, and wherein when X is O, S or N—R9, none of R⁷ and R⁸ is —OH,
or a stereoisomer, a tautomer, an N-oxide, a hydrate, a solvate, or a salt thereof, or a mixture of same.

### Aspect II of WO 2018/087036 A1:

The compound according to Aspect 1 of WO 2018/087036 A1, wherein:
A is A1 or A2,

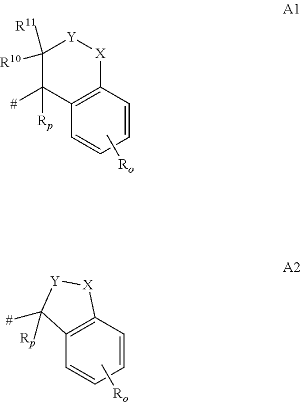

o is 0, 1, 2, 3 or 4,
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms,
Rₚ is selected from the group consisting of hydrogen, C₁-C₄-alkyl,
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—,
R¹ is selected from the group consisting of hydrogen, cyano, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N—C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, and —SO₂—C₁-C₄-halogenoalkyl having 1 to 5 halogen atoms;
phenyl-C₁-C₄-alkyl, optionally substituted by 1, 2, 3, 4 or 5 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R² is selected from the group consisting of hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂,
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —NH(C(O)—C₁-C₄-alkyl), —N(C₁-C₄-alkyl)(C(O)—C₁-C₄-alkyl), —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and 4- to 10-membered heterocycloalkyl,
R³ is hydrogen, or C₁-C₄-alkyl,
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁵ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁶ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁷ is selected from the group consisting of hydrogen, —OH, fluorine, C₁-C₄-alkyl and C₁-C₄-alkoxy,
R⁸ is selected from the group consisting of hydrogen, —OH, fluorine, C₁-C₄-alkyl and C₁-C₄-alkoxy,
or R⁷ and R⁸ together form an oxo group (═O),
R⁹ is selected from the group consisting of hydrogen, C₁-C₄-alkyl, C₁-C₄-halogenoalkyl having 1 to 5 halogen atoms and C₁-C₄-alkoxy,
R¹⁰ is selected from the group consisting of hydrogen, —OH, C₁-C₄-alkyl and C₁-C₄-alkoxy,
R¹¹ is selected from the group consisting of hydrogen, C₁-C₄-alkyl and C₁-C₄-alkoxy,
R¹² and R¹³ are independently selected from the group consisting of hydrogen, —OH, —NH₂, —NH(C₁-C₄-alkyl), —N(C₁-C₄-alkyl)₂, —NH(—C(O)—C₁-C₄-alkyl), C₁-C₄-alkoxy; C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)-(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R¹⁴ is selected from the group consisting of —NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R¹⁵ is selected from the group consisting of hydrogen;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
Q is a substituted phenyl ring of the formula (Q1)

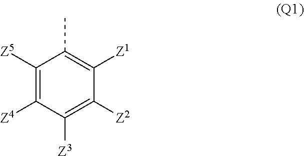

in which:
Z¹, Z², Z³, Z⁴, and Z⁵ are independently selected from the group consisting of hydrogen, halogen, SF₅, cyano, —CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z¹ and Z² form, together with the carbon atoms that they are connected to, a 5- or 6-membered saturated or partially saturated heterocyclic ring, a 5-membered heteroaryl, or a 6-membered heteroaryl, each of which may be optionally substituted with one or two substituents selected from the group consisting of methyl, fluorine and oxo, and
Z³, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-alkoxy-C(O)—, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z² and Z³ form, together with the carbon atoms that they are connected to, a 5- or 6-membered saturated or partially saturated heterocyclic ring, a 5-membered heteroaryl, or a 6-membered heteroaryl, each of which may be optionally substituted with one or two substituents selected from the group consisting of methyl, fluorine and oxo, and
Z¹, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Q is a pyridine ring of the formula (Q2)

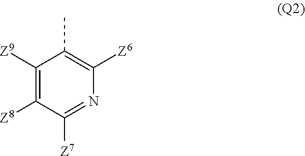

in which:
Z⁶, Z⁷, Z⁸ and Z⁹ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyrimidine ring of the formula (Q3)

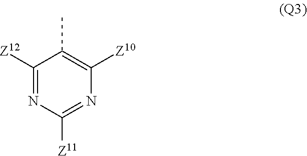

in which:
Z¹⁰, Z¹¹ and Z¹² are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyridine ring of the formula (Q4)

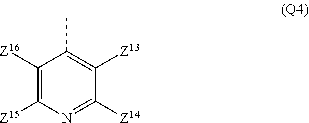

in which:
Z¹³, Z¹⁴, Z¹⁵ and Z¹⁶ are independently selected from the group consisting of hydrogen halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₁-C₄-hydroxyalkyl, NH₂, —NH(C₁-C₄-alkyl), —N(C₁-C₄-alkyl)₂, —NH—CO—C₁-C₄-alkyl, and monocyclic heterocycles selected from the group of 4- to 7-membered heterocycloalkyl or 5-membered heteroaryls having at least one nitrogen atom via which the heteroaryl ring is connected to the pyridine ring, each of which is optionally substituted with 1, 2 or 3 substituents independently selected from the group consisting of halogen, cyano, nitro, —OH, oxo, thiono, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, or
Q is a pyridine ring of the formula (Q5)

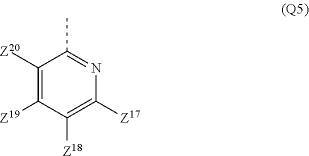

in which:
Z¹⁷, Z¹⁸, Z¹⁹ and Z²⁰ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a 5-membered aromatic heterocycle of the formula (Q6)

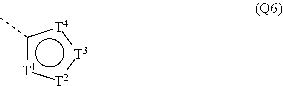

in which:
T¹-T⁴ are independently selected from the group consisting of N, O, S, C—Z²¹ and N—Z²², wherein not more than one of T¹-T⁴ is 0, not more than one of T¹-T⁴ is S, not more than one of T¹-T⁴ is N—Z²² and wherein
each Z²¹ is independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, and
each Z²² is independently selected from the group consisting of hydrogen, C₁-C₄-alkyl, C₁-C₄-halogenoalkyl having 1 to 5 halogen atoms, C₁-C₄-alkyl-C₃—C-cycloalkyl, C₁-C₄-alkoxy-C₁-C₄-alkyl, or
Q is a 5-membered aromatic heterocycle of the formula (Q7)

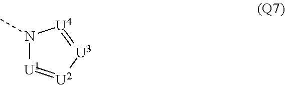

in which:
U¹-U⁴ are independently selected from the group consisting of N and C—Z²³, wherein not more than three of U¹-U⁴ are N, and wherein
each Z²³ is independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
wherein when Y is O, S or N—R⁹, none of R⁷, R⁸, R¹⁰ and R¹¹ is —OH, and wherein when X is O, S or N—R9, none of R⁷ and R⁸ is —OH,
or a stereoisomer, a tautomer, an N-oxide, a hydrate, a solvate, or a salt thereof, or a mixture of same.

### Aspect III of WO 2018/087036 A1:

The compound according to Aspect 1 or 2 of WO 2018/087036 A1, wherein:
A is A1 or A2,

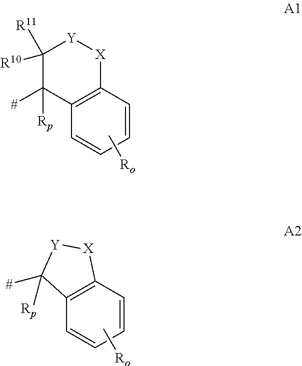

o is 0, 1 or 2,
R is selected from the group consisting of halogen, C₁-C₄-alkyl and C₁-C₄-alkoxy, cyano, C₁-C₄-halogenoalkyl having 1 to 5 halogen atoms,
Rₚ is selected from the group consisting of hydrogen, C₁-C₄-alkyl,
X, Y are independently selected from the group consisting of CR⁷R⁸, O, S, and N—R⁹, wherein at least one of X and Y is CR⁷R⁸,
R¹ is selected from the group consisting of hydrogen, C₁-C₄-alkyl, C₃-C₆-cycloalkyl, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl,
R² is selected from the group consisting of hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂,
—NR¹²R¹³;
—OR¹⁴;
—SR¹⁵, —S(O)R¹⁵, —SO₂R¹⁵;
C₁-C₄-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, cyano, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —NH(C(O)—C₁-C₄-alkyl), —N(C₁-C₄-alkyl)(C(O)—C₁-C₄-alkyl), —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, —OH, oxo, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, and 4- to 10-membered heterocycloalkyl,
R³ is hydrogen or C₁-C₄-alkyl,
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁵ is selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁶ is selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁷ is selected from the group consisting of hydrogen and C₁-C₄-alkyl,
R⁸ is selected from the group consisting of hydrogen and C₁-C₄-alkyl,
or R⁷ and R⁸ together form an oxo group (═O),
R⁹ is C₁-C₄-alkyl,
R¹⁰ is selected from the group consisting of hydrogen, —OH, C₁-C₄-alkyl and C₁-C₄-alkoxy,
R¹¹ is hydrogen,
R¹² and R¹³ are independently selected from the group consisting of hydrogen, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)-(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
phenyl, benzo-C₅-C₆-cycloalkyl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
R¹⁴ is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl; and
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
R¹⁵ is selected from the group consisting of hydrogen;
C₁-C₄-alkyl, phenyl-C₁-C₄-alkyl, each of which is optionally substituted by 1, 2 or 3 substituents independently selected from the group consisting of halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
Q is a substituted phenyl ring of the formula (Q1)

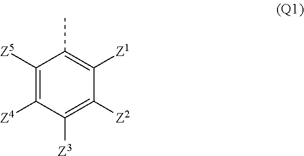

in which:
Z¹, Z², Z³, Z⁴, and Z⁵ are independently selected from the group consisting of hydrogen, halogen, cyano, C₁-C₄-alkyl, C₁-C₄-halogenoalkyl having 1 to 5 halogen atoms, hydroxy, C₁-C₄-alkoxy, C₁-C₄-halogenoalkoxy having 1 to 5 halogen atoms, —NH(C₁-C₄-alkyl), —N(C₁-C₄-alkyl)₂, 4- to 6-membered heterocyclyl, which is optionally substituted with 1 or 2 substituents selected from the group consisting of fluorine, chlorine, bromine, methyl and cyano, —S—(C₁-C₄-alkyl), —S(O)—(C₁-C₄-alkyl), —SO₂—(C₁-C₄-alkyl), or
Z¹ and Z² form, together with the carbon atoms that they are connected to, a 5- or 6-membered heterocycloalkyl, a 5-membered heteroaryl, or a 6-membered heteroaryl, each of which may be optionally substituted with one or two substituents selected from the group consisting of methyl, fluorine and oxo, and
Z³, Z⁴, and Z⁵ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-alkoxy-C(O)—, C₁-C₄-halogenoalkoxy having 1 to 5 halogen atoms, or
Z² and Z³ form, together with the carbon atoms that they are connected to, a 5- or 6-membered saturated or partially saturated heterocyclic ring, a 5-membered heteroaryl, or a 6-membered heteroaryl, each of which may be optionally substituted with one or two substituents selected from the group consisting of methyl, fluorine and oxo, and
Z¹, Z⁴, and Z⁵ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, or
Q is a pyridine ring of the formula (Q2)

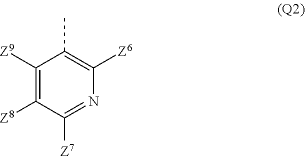

in which:
Z⁶, Z⁷, Z⁸ and Z⁹ are independently selected from the group consisting of hydrogen halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyrimidine ring of the formula (Q3)

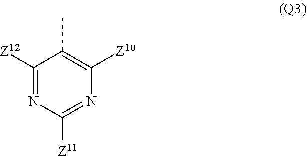

in which:
Z¹⁰, Z¹¹ and Z¹² are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyridine ring of the formula (Q4)

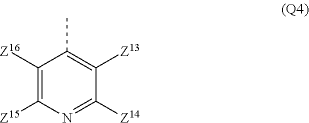

in which:
Z¹³, Z¹⁴, Z¹⁵ and Z¹⁶ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₁-C₄-hydroxyalkyl, NH₂, —NH(C₁-C₄-alkyl), —N(C₁-C₄-alkyl)₂, —NH—CO—C₁-C₄-alkyl, and monocyclic heterocycles selected from the group of 4- to 7-membered heterocycloalkyl or 5-membered heteroaryls having at least one nitrogen atom via which the heteroaryl ring is connected to the pyridine ring, each of which is optionally substituted with 1, 2 or 3 substituents independently selected from the group consisting of halogen, cyano, nitro, —OH, oxo, thiono, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, or
Q is a pyridine ring of the formula (Q5)

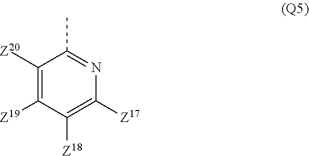

in which:
Z¹⁷, Z¹⁸, Z¹⁹ and Z²⁰ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a 5-membered aromatic heterocycle of the formula (Q6)

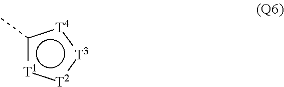

in which:
T¹-T⁴ are independently selected from the group consisting of N, O, S, C—Z²¹ and N—Z²², wherein not more than one of T¹-T⁴ is O, not more than one of T¹-T⁴ is S, not more than one of T¹-T⁴ is N—Z²² and wherein
each Z²¹ is independently selected from the group consisting of hydrogen, halogen, cyano, C₁-C₄-alkyl, C₁-C₄-halogenoalkyl having 1 to 5 halogen atoms, C₁-C₄-alkoxy, and
each Z²² is independently selected from the group consisting of hydrogen, C₁-C₄-alkyl, C₁-C₄-halogenoalkyl having 1 to 5 halogen atoms, C₁-C₄-alkyl-C₃—C-cycloalkyl, C₁-C₄-alkoxy-C₁-C₄-alkyl, or
Q is a 5-membered aromatic heterocycle of the formula (Q7)

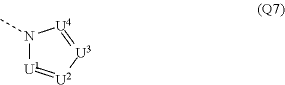

in which:
U¹-U⁴ are independently selected from the group consisting of N and C—Z²³, wherein not more than three of U¹-U⁴ are N, and wherein each Z²³ is independently selected from the group consisting of hydrogen, halogen, cyano, C₁-C₄-alkyl, C₁-C₄-halogenoalkyl having 1 to 5 halogen atoms, C₁-C₄-alkoxy,
wherein when Y is O, S or N—R⁹, R¹⁰ is not —OH,
or a stereoisomer, a tautomer, an N-oxide, a hydrate, a solvate, or a salt thereof, or a mixture of same.

### Aspect IV of WO 2018/087036 A1:

The compound according to Aspect 1, 2 or 3 of WO 2018/087036 A1, wherein:
A is A1 or A2,

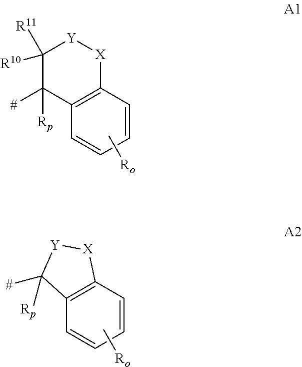

o is 0, 1 or 2,
R is selected from the group consisting of halogen, C₁-C₄-alkyl and C₁-C₄-alkoxy,
Rₚ is selected from the group consisting of hydrogen, C₁-C₄-alkyl,
X is selected from the group consisting of CR⁷R⁸, O, S, and N—R⁹,
Y is CR⁷R⁸ or O,
R¹ is hydrogen or C₁-C₄-alkyl,
R² is selected from the group consisting of hydrogen, halogen, —C(O)—N(C₁-C₄-alkyl)₂;
—NR¹²R¹³.
—OR¹⁴;
—SR⁵, —S(O)R¹⁵, —SO₂R¹⁵;
C₁-C₄-alkyl, C₃-C₆-cycloalkyl, C₂-C₄-alkenyl or C₃-C₆-cycloalkenyl, each of which is optionally substituted by 1, 2, 3, 4 or 5 substituents independently selected from the group consisting of halogen, —OH, cyano, C₁-C₄-alkoxy-C(O)— and —C(O)—NH₂, C₁-C₄-alkoxy, —NH₂, —N(C₁-C₄-alkyl)₂, —N(C₁-C₄-alkyl)(C(O)—C₁-C₄-alkyl); and
a monocyclic or a bicyclic heterocycle selected from the group consisting of 4- to 10-membered heterocycloalkyl, heterospirocycloalkyl, 5-membered heteroaryl, and 6-membered heteroaryl, each of which is optionally substituted by 1, 2, 3 or 4 substituents independently selected from the group consisting of halogen, —OH, oxo, —COOH, C₁-C₄-alkoxy-C(O)—, —C(O)—NH₂, C₁-C₄-alkyl, C₁-C₄-alkyl-C(O)—, C₁-C₄-halogenoalkyl having 1 to 5 halogen atoms, hydroxy-C₁-C₄-alkyl-, C₁-C₄-alkoxy-C₁-C₄-alkyl-, —NH₂, —N(C₁-C₄-alkyl)₂, and 4- to 10-membered heterocycloalkyl,
R³ is hydrogen or C₁-C₄-alkyl,
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
R⁵ is selected from the group consisting of hydrogen, halogen, —OH, cyano, C₁-C₄-alkyl, C₁-C₄-halogenoalkyl having 1 to 5 halogen atoms, C₁-C₄-alkoxy,
R⁶ is selected from the group consisting of hydrogen, halogen, —OH, cyano, C₁-C₄-alkyl, C₁-C₄-halogenoalkyl having 1 to 5 halogen atoms, C₁-C₄-alkoxy,
R⁷ is selected from the group consisting of hydrogen and C₁-C₄-alkyl,
R⁸ is selected from the group consisting of hydrogen and C₁-C₄-alkyl,
or R⁷ and R⁸ together form an oxo group (═O),
R⁹ is C₁-C₄-alkyl,
R¹⁰ is selected from the group consisting of hydrogen, —OH and C₁-C₄-alkyl,
R¹¹ is hydrogen,
R¹² and R¹³ are independently selected from the group consisting of hydrogen, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, —COOH, C₁-C₄-alkoxy-C(O)—, —C(O)—NH₂, —C(O)—N(C₁-C₄-alkyl)₂, —NH—C(O)—C₁-C₄-alkyl, C₁-C₄-alkyl, C₁-C₄-alkoxy, C₃-C₆-cycloalkyl, —NH₂, —N(C₁-C₄-alkyl)₂, —S—C₁-C₄-alkyl, —S(O)—C₁-C₄-alkyl, —SO₂—C₁-C₄-alkyl, and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms and C₁-C₄-alkoxy;
phenyl and benzo-C₅-C₆-cycloalkyl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms; and
a monocyclic or a bicyclic heterocycle selected from the group of 4- to 10-membered heterocycloalkyl, 5-membered heteroaryl and 6-membered heteroaryl each of which is optionally substituted by 1, 2 or 3 substituents independently selected from the group consisting of halogen, —OH, oxo, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
R¹⁴ is selected from the group consisting of C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, C₁-C₄-alkyl, C₁-C₄-alkoxy and C₃-C₆-cycloalkyl; and
4- to 10-membered heterocycloalkyl,
R¹⁵ is selected from the group consisting of hydrogen;
C₁-C₄-alkyl, which is optionally substituted by 1, 2 or 3 substituents independently selected from the group consisting of —OH and —COOH; and
a 6-membered heteroaryl,
Q is a substituted phenyl ring of the formula (Q1)

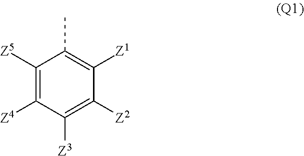

in which:
Z¹ and Z⁵ are independently selected from the group consisting of hydrogen, halogen, C₁-C₄-alkyl, C₁-C₄-alkoxy, C₁-C₄-halogenoalkoxy having 1 to 5 halogen atoms,
Z² and Z⁴ are independently selected from the group consisting of hydrogen, halogen, cyano, —OH, C₁-C₄-alkyl, C₁-C₄-alkoxy, —NH(C₁-C₄-alkyl), —N(C₁-C₄-alkyl)₂, C₁-C₄-halogenoalkyl having 1 to 5 halogen atoms, C₁-C₄-halogenoalkoxy having 1 to 5 halogen atoms, —S—(C₁-C₄-alkyl) and a 4- to 6-membered heterocycloalkyl, and
Z³ is selected from the group consisting of hydrogen, halogen, C₁-C₄-alkyl, C₁-C₄-alkoxy, C₁-C₄-halogenoalkoxy having 1 to 5 halogen atoms, and —N(C₁-C₄-alkyl)₂, or
Z¹ and Z² form, together with the carbon atoms that they are connected to, a 5-membered heterocycloalkyl or a 5-membered heteroaryl, each of which may be optionally substituted with one or two substituents selected from the group consisting of methyl, fluorine and oxo,
Z³ and Z⁵ are hydrogen, and
Z⁴ is selected from the group consisting of hydrogen and C₁-C₄-alkoxy-C(O)—, or
Q is a pyridine ring of the formula (Q4)

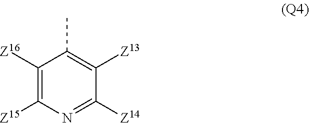

in which:
Z¹³, Z¹⁴, Z¹⁵ and Z¹⁶ are independently selected from the group consisting of hydrogen, halogen, cyano, C₁-C₄-alkyl, C₁-C₄-alkoxy, C₁-C₄-hydroxyalkyl, NH₂, —NH(C₁-C₄-alkyl), —N(C₁-C₄-alkyl)₂, —NH—CO—C₁-C₄-alkyl, and monocyclic heterocycles selected from the group of 4- to 7-membered heterocycloalkyl or 5-membered heteroaryls having at least one nitrogen atom via which the heteroaryl ring is connected to the pyridine ring, each of which is optionally substituted with 1, 2 or 3 substituents independently selected from the group consisting of halogen, cyano, nitro, —OH, oxo, thiono, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, or
Q is a pyridine ring of the formula (Q5)

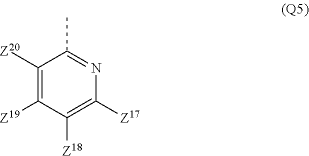

in which:
Z¹⁷, Z¹⁸, and Z¹⁹ are hydrogen, and
Z²⁰ is halogen, or
Q is a 5-membered aromatic heterocycle of the formula (Q6)

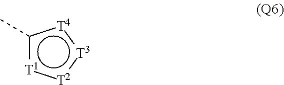

in which:
T¹-T⁴ are independently selected from the group consisting of N, O, S, C—Z²¹ and N—Z²², wherein not more than one of T¹-T⁴ is 0, not more than one of T¹-T⁴ is S, not more than one of T¹-T⁴ is N—Z²² and wherein
each Z²¹ is independently selected from the group consisting of hydrogen, halogen, cyano, C₁-C₄-alkyl, C₁-C₄-halogenoalkyl having 1 to 5 halogen atoms, C₁-C₄-alkoxy, and
each Z²² is independently selected from the group consisting of hydrogen, C₁-C₄-alkyl, C₁-C₄-halogenoalkyl having 1 to 5 halogen atoms, C₁-C₄-alkyl-C₃-C₆-cycloalkyl, C₁-C₄-alkoxy-C₁-C₄-alkyl, or
Q is a 5-membered aromatic heterocycle of the formula (Q7)

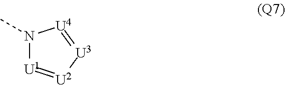

in which:
U¹-U⁴ are independently selected from the group consisting of N and C—Z²³, wherein not more than three of U¹-U⁴ are N, and wherein
each Z²³ is independently selected from the group consisting of hydrogen, halogen, cyano, C₁-C₄-alkyl, C₁-C₄-halogenoalkyl having 1 to 5 halogen atoms, C₁-C₄-alkoxy,
or a stereoisomer, a tautomer, an N-oxide, a hydrate, a solvate, or a salt thereof, or a mixture of same.

### Aspect V of WO 2018/087036 A1:

The compound according to Aspect 1, 2, 3 or 4 of WO 2018/087036 A1, wherein:
A is selected from the group consisting of

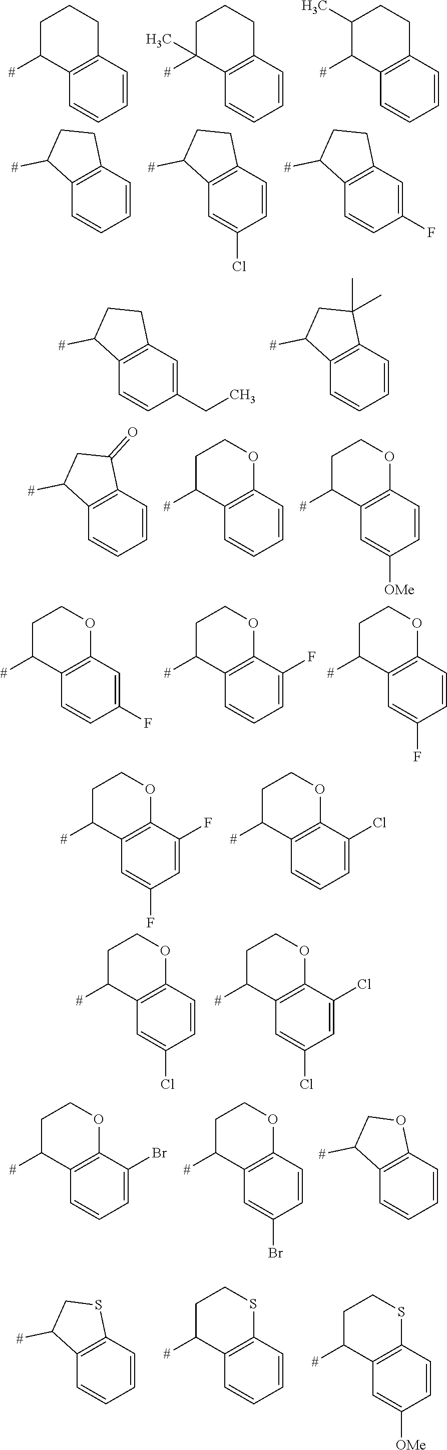

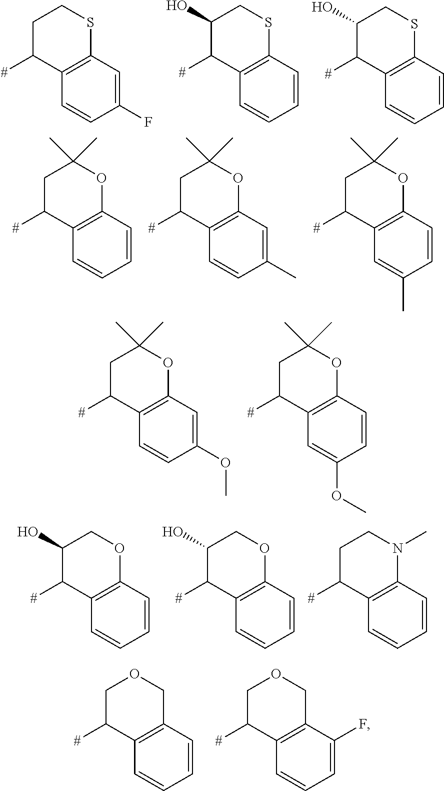

R¹ is hydrogen or methyl,
R² is selected from the group consisting of hydrogen, chlorine, iodine, —C(O)—N(CH₃)₂,
—OR¹⁴;
—SR¹⁵, —S(O)R¹⁵, —SO₂R¹⁵;
methyl, ethyl, propyl, isopropyl, cyclopropyl, cyclobutyl, cyclopentyl, cyclohexyl, ethenyl, propenyl, cyclopentenyl, cyclohexenyl, each of which is optionally substituted by 1 or 2 substituents independently selected from the group consisting of —OH, cyano, ethoxy-C(O)—, —C(O)—NH₂, methoxy, NH₂, N(CH₃)₂, N(CH₃)(C(O)CH₃); and
a monocyclic or a bicyclic heterocycle selected from the group consisting of azetidine, oxetane, pyrrolidine, tetrahydrofurane, pyrazolidine, imidazolidine, 1,2,4-triazolidine, piperidine, piperazine, tetrahydropyrane, tetrahydropyridine, dihydro-2H-pyrane, 1,2-oxazolidine, 1,2-oxazine, morpholine, thiomorpholine, 3,4-dihydroisoquinoline, 2,3-dihydro-indole, 1,3-dihydro-isoindoel, 3,9-dioxa-7-azabicyclo[3.3.1]nonane, 6-oxa-3-azabicyclo[3.1.1]heptane, 8-oxa-3-azabicyclo[3.2.1]octane, thiophene, imidazole, pyrazole, 1,2,4-triazole, 1,2,3-triazole, 1,2,3,4-tetrazole, pyridine, dihydropyridine, pyrimidine, tetrahydropyrimidine, 4-oxa-7-azaspiro[2.5]octane, each of which is optionally substituted by 1, 2, 3 or 4 substituents independently selected from the group consisting of fluorine, chlorine, cyano, —OH, oxo, —COOH, methoxy-C(O)—, ethoxy-C(O)—, tert-butoxy-C(O)—, —C(O)—NH₂, methyl, methyl-C(O)—, difluoromethyl, trifluoromethyl, hydroxymethyl-, methoxymethyl-, —NH₂, —NMe₂, pyrrolidine,
R³ is hydrogen or methyl,
R⁴ is selected from the group consisting of hydrogen, fluorine, chlorine, —OH, cyano, methyl, methoxy, trifluoromethyl, trifluoromethoxy and NH₂,
R⁵ is selected from the group consisting of hydrogen, fluorine, chlorine, —OH, cyano, methyl, methoxy and trifluoromethyl,
R⁶ is selected from the group consisting of hydrogen, fluorine, chlorine, —OH, cyano, methyl and methoxy,
R¹² and R¹³ are independently selected from the group consisting of hydrogen, —NH(—C(O)-methyl), methoxy;
methyl, ethyl, propyl, isopropyl, butyl, isobutyl, cyclopropyl, cyclobutyl, benzyl, 1-phenylethyl, each of which is optionally substituted by 1, 2 or 3 substituents independently selected from the group consisting of fluorine, —OH, —COOH, methoxy-C(O)—, ethoxy-C(O)—, tert-butoxy-C(O)—, —C(O)—NH₂, —C(O)—NMe₂, —NH—C(O)-methyl, methyl, methoxy, cyclopropyl, —NH₂, NMe₂, S-methyl, S(O)-methyl, SO₂-methyl, and (EtO)₂P(═O)—;
heterocyclyl-methyl, heterocyclyl-ethyl, wherein the heterocyclyl substituent is selected from the group consisting of oxetane, tetrahydrofurane, tetrahydropyrane pyrrolidine, morpholine, pyrazole, imidazole, 1, 2, 4-oxadiazole, pyridine, each of which is optionally substituted by 1 substituent independently selected from the group consisting of fluorine, chlorine, —OH, oxo and methyl;
phenyl;
2,3-dihydro-1H-indene, and
a monocyclic or a bicyclic heterocycle selected from the group of oxetane, thietane, pyrrolidine, morpholine, tetrahydropyrane, pyridine and pyrazole, each of which is optionally substituted by 1 or 2 substituents independently selected from the group consisting of fluorine, chlorine, —OH, oxo, methyl;
R¹⁴ is selected from the group consisting of methyl, ethyl, isopropyl, butyl, cyclopentyl, benzyl, each of which is optionally substituted by 1 or 2 substituents independently selected from the group consisting of fluorine, —OH, methyl, methoxy and cyclopentyl; and
a monocyclic or a bicyclic heterocycle selected from the group consisting of pyrrolidin and tetrahydropyran,
R¹⁵ is selected from the group consisting of methyl and ethyl, each of which is optionally substituted by 1 substituent independently selected from the group consisting of —OH and —COOH; and
pyridine,
Q is a substituted phenyl ring of the formula (Q1)

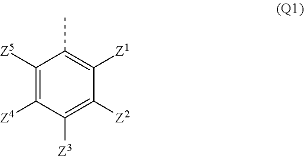

in which:
Z¹ and Z⁵ are independently selected from the group consisting of hydrogen, fluorine, chlorine, methyl, trifluoromethyl and methoxy,
Z² and Z⁴ are independently selected from the group consisting of hydrogen, fluorine, chlorine, —OH, cyano, methyl, ethyl, tert-butyl, —NHMe, —NMe₂, trifluoromethyl, methoxy, trifluoromethoxy, —SMe and morpholinyl, and
Z³ is independently selected from the group consisting of hydrogen, fluorine, chlorine, methyl, methoxy, difluoromethoxy and —NMe₂, or
Q is a pyridine ring of the formula (Q4)

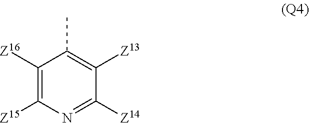

in which:
Z¹³, Z¹⁴, Z¹⁵ and Z¹⁶ are independently selected from the group consisting of hydrogen, fluorine, chlorine, cyano, methyl, methoxy, ethoxy, isopropoxy, hydroxymethyl, NH₂, —NHMe —NMe₂, —NH—C(O)-Me, morpholinyl, or
Q is a pyridine ring of the formula (Q5)

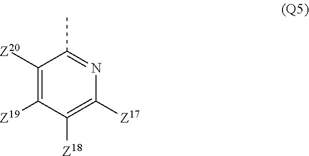

in which:
Z¹⁷, Z¹⁸, and Z¹⁹ are hydrogen, and
Z²⁰ is fluorine, chlorine, or
Q is selected from the group consisting of

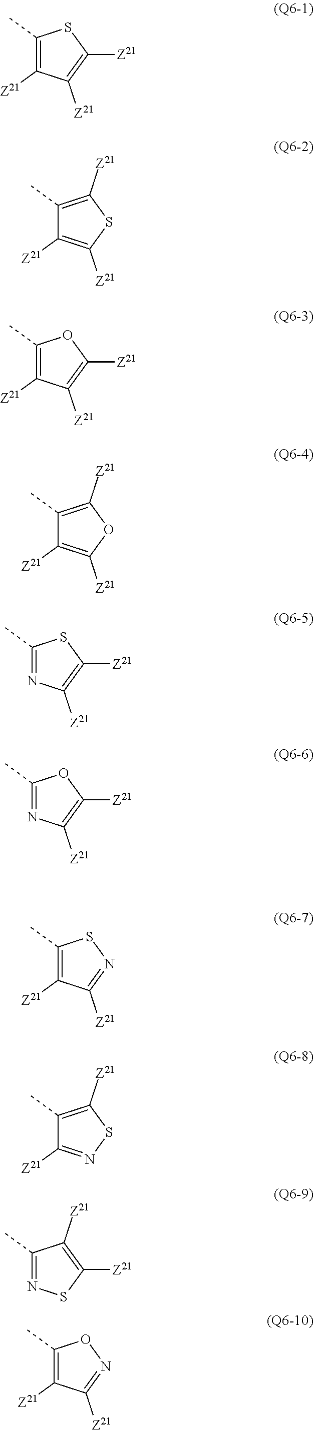

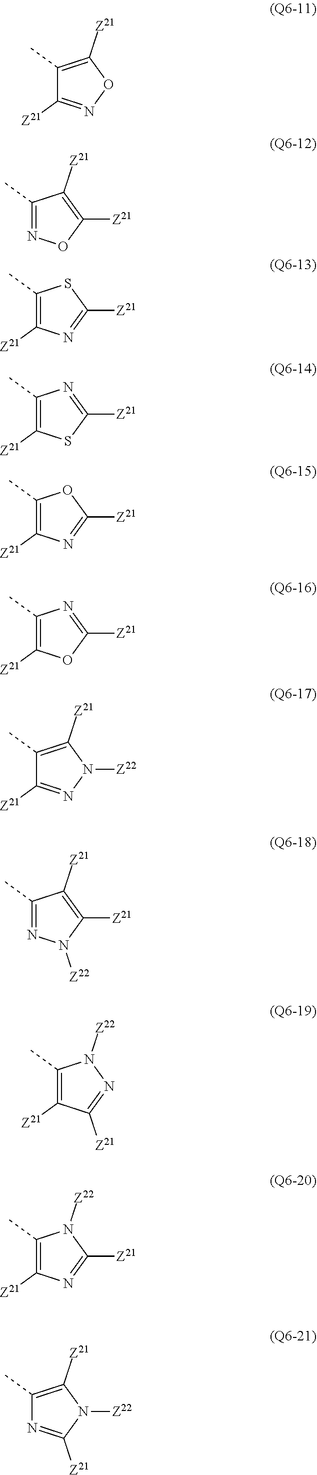

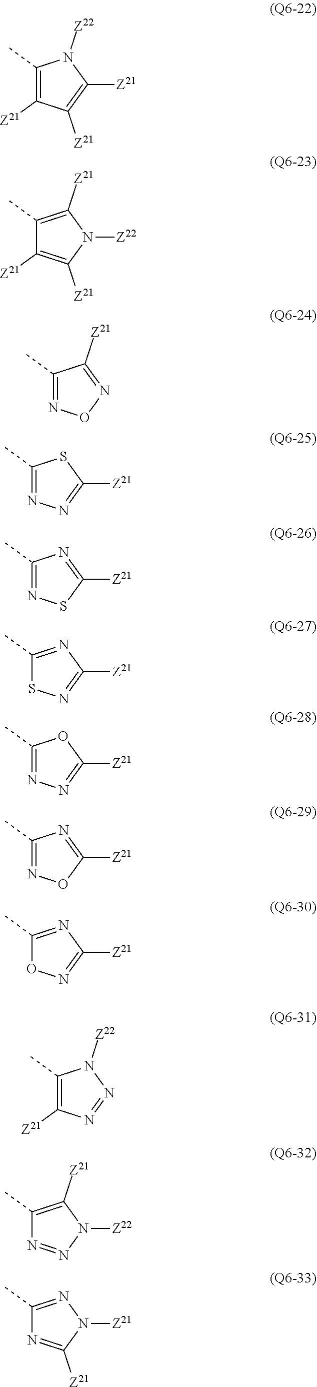

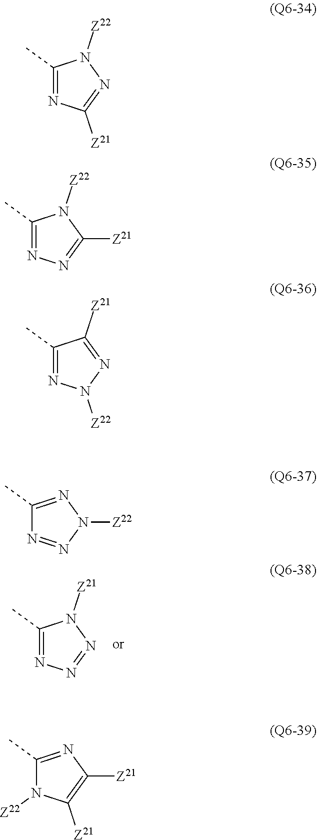

in which:
each Z²¹ is independently selected from the group consisting of hydrogen, fluorine, chlorine, cyano, methyl, trifluoromethyl, methoxy and
Z²² is hydrogen, methyl, or
Q is selected from the group consisting of

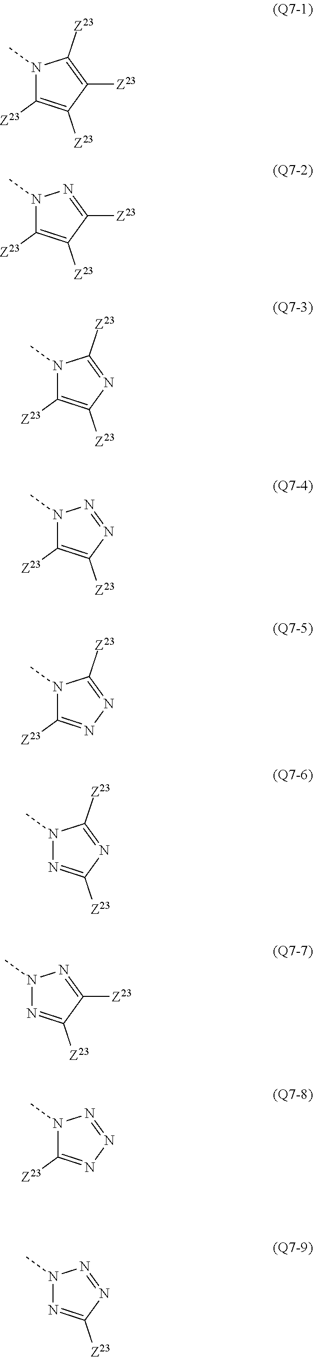

in which:
each Z²³ is independently selected from the group consisting of hydrogen, fluorine, chlorine, cyano, methyl, trifluoromethyl, methoxy, or
Q is selected from the group consisting of

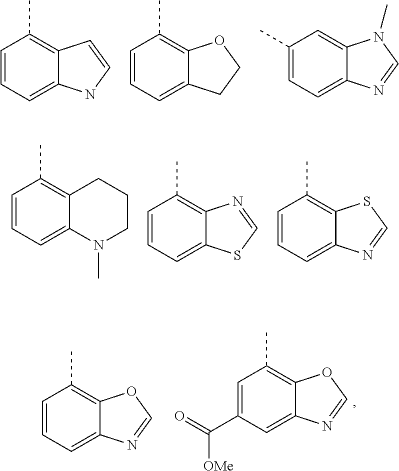

or a stereoisomer, a tautomer, an N-oxide, a hydrate, a solvate, or a salt thereof, or a mixture of same.

### Aspect VI of WO 2018/087036 A1:

The compound according to Aspect 1, 2, 3, 4 or 5 of WO 2018/087036 A1, wherein:
A is selected from the group consisting of

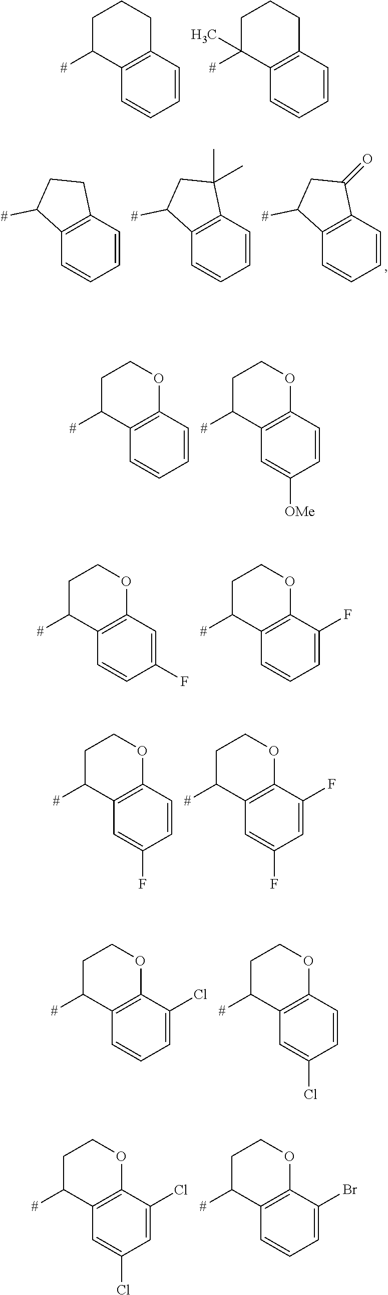

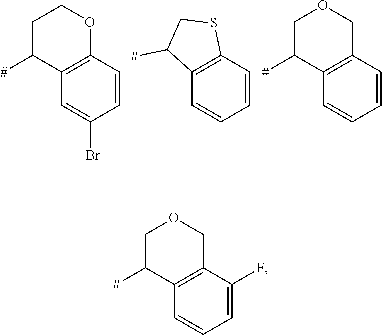

R¹ is hydrogen or methyl,
R² is selected from the group consisting of chlorine, iodine, —C(O)—N(CH₃)₂,
—NR¹²R¹³;
—OR¹⁴;
—SR¹⁵, —S(O)R¹⁵, —SO₂R¹⁵;
methyl, ethyl, propyl, isopropyl, cyclopropyl, cyclobutyl, cyclopentyl, ethenyl, propenyl, each of which is optionally substituted by 1 or 2 substituents independently selected from the group consisting of —OH, cyano, ethoxy-C(O)—, —C(O)—NH₂, methoxy, NH₂, N(CH₃)₂, N(CH₃)(C(O)CH₃); and
a monocyclic or a bicyclic heterocycle selected from the group consisting of azetidine, oxetane, pyrrolidine, tetrahydrofurane, pyrazolidine, imidazolidine, 1,2,4-triazolidine, piperidine, piperazine, tetrahydropyrane, dihydro-2H-pyrane, 1,2-oxazolidine, morpholine, thiomorpholine, 3,4-dihydroisoquinoline, 2,3-dihydro-indoel, 1,3-dihydro-isoindole, 3,9-dioxa-7-azabicyclo[3.3.1]nonane, 6-oxa-3-azabicyclo[3.1.1]heptane, 8-oxa-3-azabicyclo[3.2.1]octane, thiophene, imidazole, pyrazole, 1,2,3-triazole, 1,2,3,4-tetrazole, pyridine, dihydropyridine, pyrimidine, tetrahydropyrimidine, each of which is optionally substituted by 1, 2, 3 or 4 substituents independently selected from the group consisting of fluorine, —OH, oxo, —COOH, methoxy-C(O)—, ethoxy-C(O)—, tert-butoxy-C(O)—, —C(O)—NH₂, methyl, methyl-C(O)—, difluoromethyl, trifluoromethyl, hydroxymethyl-, methoxymethyl-, —NH₂, —NMe₂, pyrrolidine,
R³ is hydrogen or methyl,
R⁴ is selected from the group consisting of hydrogen, chlorine, fluorine, methyl, methoxy and trifluoromethyl,
R⁵ is selected from the group consisting of hydrogen, chlorine, fluorine, —OH, cyano, methyl, trifluoromethoxy and NH₂,
R⁶ is selected from the group consisting of hydrogen, fluorine, chlorine, —OH, cyano, methyl and methoxy,
R¹² and R¹³ are independently selected from the group consisting of hydrogen, —NH(—C(O)-methyl), methoxy;
methyl, ethyl, propyl, isopropyl, butyl, isobutyl, cyclopropyl, cyclobutyl, benzyl, 1-phenylethyl, each of which is optionally substituted by 1, 2 or 3 substituents independently selected from the group consisting of fluorine, —OH, —COOH, methoxy-C(O)—, ethoxy-C(O)—, tert-butoxy-C(O)—, —C(O)—NH₂, —C(O)—NMe₂, —NH—C(O)-methyl, methyl, methoxy, cyclopropyl, —NH₂, —NMe₂, SO₂-methyl and (EtO)₂P(═O)—;
heterocyclyl-methyl, heterocyclyl-ethyl, wherein the heterocycyl substituent is selected from the group consisting of oxetane, tetrahydrofurane, tetrahydropyrane, pyrrolidine, pyrazole, imidazole 1, 2, 4-oxadiazole, morpholine, pyridine, each of which is optionally substituted by 1 substituent independently selected from the group consisting of oxo and methyl;
phenyl;
2,3-dihydro-1H-indene, and
a monocyclic or a bicyclic heterocycle selected from the group of oxetane, morpholine, tetrahydropyrane, pyridine and pyrazole;
R¹⁴ is selected from the group consisting of
methyl, ethyl, isopropyl, butyl, cyclopentyl, benzyl, each of which is optionally substituted by 1 or 2 substituents independently selected from the group consisting of fluorine, —OH, methyl, methoxy and cyclopentyl; and
a monocyclic or a bicyclic heterocycle selected from the group consisting of pyrrolidin and tetrahydropyran,
R¹⁵ is selected from the group consisting of
methyl and ethyl, each of which is optionally substituted by 1 substituent independently selected from the group consisting of —OH and —COOH; and
pyridine,
Q is a substituted phenyl ring of the formula (Q1)

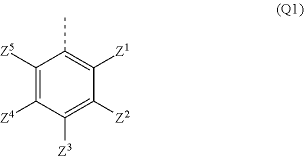

in which:
Z¹ and Z⁵ are independently selected from the group consisting of hydrogen, fluorine, chlorine, methyl, methoxy and trifluoromethyl,
Z² and Z⁴ are independently selected from the group consisting of hydrogen, fluorine, chlorine, —OH, cyano, methyl, ethyl, tert-butyl, —NHMe, —NMe₂, trifluoromethyl, methoxy, trifluoromethoxy, —SMe and morpholinyl, and
Z³ is independently selected from the group consisting of hydrogen, fluorine, chlorine, methyl, methoxy, difluoromethoxy and —NMe₂, or
Q is a pyridine ring of the formula (Q4)

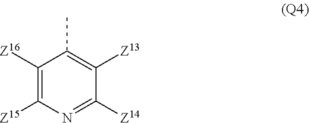

in which:
Z⁴ and Z⁵ are independently selected from the group consisting of hydrogen, fluorine, chlorine, cyano, methyl, methoxy, ethoxy, isopropoxy, hydroxymethyl, NH₂, morpholinyl and
Z¹³ and Z¹⁶ are independently selected from the group consisting of hydrogen, fluorine, chlorine, methyl, methoxy, or
Q is a pyridine ring of the formula (Q5)

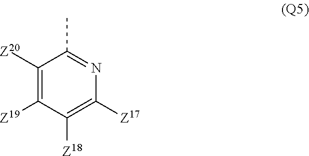

in which:
Z¹⁷, Z¹⁸, and Z¹⁹ are hydrogen, and
Z²⁰ is fluorine, or
Q is selected from the group consisting of

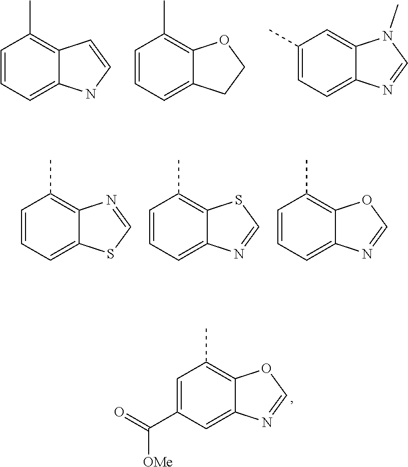

or a stereoisomer, a tautomer, an N-oxide, a hydrate, a solvate, or a salt thereof, or a mixture of same.

In one embodiment, E1_1, E2_1, n and R1 are as defined in WO 2018/087036 A1. In one embodiment, E1_1, E2_1, n and R1 are as defined in the above-described aspects of WO 2018/087036 A1. In one embodiment, the compound of general formula (I) is a compound of formula I.1 wherein E1_1, E2_1 and R1 are as defined in the above-described aspects of WO 2018/087036 A1 and n is 0.

### WO 2019/025341 A1

In one embodiment, the compound for use in the long-term prevention and/or treatment of a disease is a compound according to the disclosure of WO 2019/025341 A1. The disclosure of WO 2019/025341 A1 is incorporated herein by reference in its entirety. In one embodiment, the compound for use in the long-term prevention and/or treatment of a disease according to the invention is a compound according to the following aspects of WO 2019/025341 A1:

### Aspect I of WO 2019/025341 A1:

A compound of general formula (I):

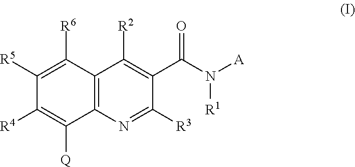

in which:
A is A1 or A2,

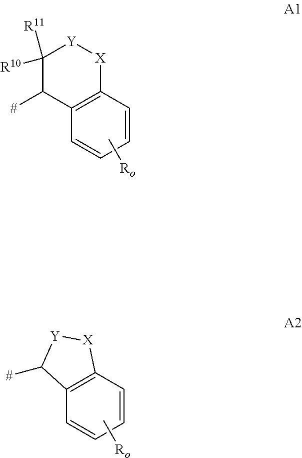

o is 0, 1, 2, 3 or 4,
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms,
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—,
R¹ is selected from the group consisting of hydrogen, cyano, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N—C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, and —SO₂—C₁-C₄-halogenoalkyl having 1 to 5 halogen atoms;
phenyl-C₁-C₄-alkyl, optionally substituted by 1, 2, 3, 4 or 5 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R² is selected from the group consisting of hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl,
R³ is hydrogen, halogen, —OH, C₁-C₄-alkyl or C₁-C₄-alkoxy,
R⁴ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl,
R⁵ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl,
R⁶ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl,
R⁷ is selected from the group consisting of hydrogen, —OH, fluorine, C₁-C₄-alkyl and C₁-C₄-alkoxy,
R⁸ is selected from the group consisting of hydrogen, —OH, fluorine, C₁-C₄-alkyl and C₁-C₄-alkoxy,
or R⁷ and R⁸ form, together with the carbon atom to which they are attached, a 3- to 6-membered ring selected from the group consisting of C₃-C₆-cycloalkyl and 3- to 6-membered heterocycloalkyl,
R⁹ is selected from the group consisting of hydrogen, C₁-C₄-alkyl, C₁-C₄-halogenoalkyl having 1 to 5 halogen atoms and C₁-C₄-alkoxy,
R¹⁰ is selected from the group consisting of hydrogen, —OH, C₁-C₄-alkyl and C₁-C₄-alkoxy,
R¹¹ is selected from the group consisting of hydrogen, C₁-C₄-alkyl and C₁-C₄-alkoxy,
or R¹⁰ and R¹¹ form, together with the carbon atom to which they are attached, a 3- to 6-membered ring selected from the group consisting of C₃-C₆-cycloalkyl and 3- to 6-membered heterocycloalkyl,
R¹² and R¹³ are independently selected from the group consisting of hydrogen, —OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy, C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R¹⁴ is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R¹⁵ is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
Q is selected from the group consisting of
—NH₂, —NH(C₁-C₄-alkyl), —NH(C₃-C₆-cycloalkyl), —NH(phenyl-C₁-C₄-alkyl), —NH(C₁-C₄-alkoxy), —NH(C₁-C₄-alkyl-C(O)—), (—NH(C₁-C₄-alkoxy-C(O)—), —N(C₁-C₄-alkyl)₂,
C₁-C₆-alkyl, C₃-C₁₀-cycloalkyl, C₂-C₆-alkenyl, C₃-C₁₀-cycloalkenyl, C₂-C₆-alkynyl,
each of which is optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, benzyloxy-C(O)—, C₁-C₄-alkoxy-C₁-C₄-alkyl-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl-C₁-C₄-alkyl-, optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl-, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano,
C₁-C₄-alkyl-C(O)—, C₁-C₄-halogenoalkyl-C(O)— having 1 to 5 halogen atoms,
C₁-C₄-alkoxy-C(O)—, C₁-C₄-halogenoalkoxy-C(O)— having 1 to 5 halogen atoms,
C₁-C₄-alkyl, C₁-C₄-halogenoalkyl having 1 to 5 halogen atoms, C₁-C₄-alkoxy,
C₁-C₄-halogenoalkoxy having 1 to 5 halogen atoms, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl,
—S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
a C- or N-bound monocyclic or bicyclic heterocycle selected from the group consisting of 4- to 10-membered heterocycloalkyl and heterospirocycloalkyl, each of which is optionally substituted by 1, 2, 3 or 4 substituents independently selected from the group consisting of halogen, cyano, nitro, —OH, oxo, thiono, —COOH, C₁-C₄-alkoxy-C(O)—,
—C(O)—NH₂, —C(O)—NH(C₁-C₄-alkyl), —C(O)—N(C₁-C₄-alkyl)₂, C₁-C₄-alkyl,
C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂,
—S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms,
—SO₂—C₁-C₄-halogenoalkyl having 1 to 5 halogen atoms, and 4- to 10-membered heterocycloalkyl wherein when Y is O, S or N—R⁹, none of R⁷, R⁸, R¹⁰ and R¹¹ is —OH, and wherein when X is O, S or N—R⁹, none of R⁷ and R⁸ is —OH,
or a stereoisomer, a tautomer, an N-oxide, a hydrate, a solvate, or a salt thereof, or a mixture of same.

### Aspect II of WO 2019/025341 A1:

The compound according to Aspect 1 of WO 2019/025341 A1, wherein:
A is A1 or A2,

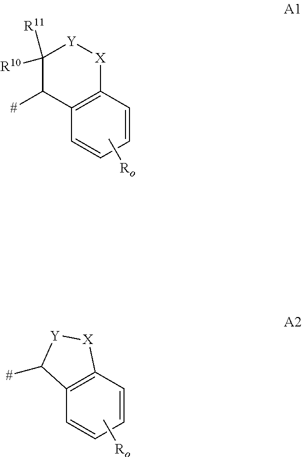

o is 0, 1, 2, 3 or 4,
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms,
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—,
R⁸ is selected from the group consisting of hydrogen, cyano, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N—C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, and —SO₂—C₁-C₄-halogenoalkyl having 1 to 5 halogen atoms;
phenyl-C₁-C₄-alkyl, optionally substituted by 1, 2, 3, 4 or 5 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R² is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂,
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and 4- to 10-membered heterocycloalkyl,
R³ is hydrogen, halogen, —OH, C₁-C₄-alkyl or C₁-C₄-alkoxy,
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, and C₁-C₄-halogenoalkoxy having 1 to 5 halogen atoms,
R⁵ is selected from the group consisting of hydrogen, halogen, —OH, cyano, C₁-C₄-alkyl, C₁-C₄-halogenoalkyl having 1 to 5 halogen atoms, C₁-C₄-alkoxy, and C₁-C₄-halogenalkoxy having 1 to 5 halogen atoms,
R⁶ is selected from the group consisting of hydrogen, halogen, —OH, cyano, C₁-C₄-alkyl, C₁-C₄-halogenoalkyl having 1 to 5 halogen atoms, C₁-C₄-alkoxy, and C₁-C₄-halogenalkoxy having 1 to 5 halogen atoms,
R⁷ is selected from the group consisting of hydrogen, —OH, fluorine, C₁-C₄-alkyl and C₁-C₄-alkoxy,
R⁸ is selected from the group consisting of hydrogen, —OH, fluorine, C₁-C₄-alkyl and C₁-C₄-alkoxy,
R⁹ is selected from the group consisting of hydrogen, C₁-C₄-alkyl, C₁-C₄-halogenoalkyl having 1 to 5 halogen atoms and C₁-C₄-alkoxy,
R¹⁰ is selected from the group consisting of hydrogen, —OH, C₁-C₄-alkyl and C₁-C₄-alkoxy,
R¹¹ is selected from the group consisting of hydrogen, C₁-C₄-alkyl and C₁-C₄-alkoxy,
R¹² and R¹³ are independently selected from the group consisting of
hydrogen, —OH, —NH₂, —NH(C₁-C₄-alkyl), —N(C₁-C₄-alkyl)₂, —NH(—C(O)—C₁-C₄-alkyl), C₁-C₄-alkoxy; C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)-(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R¹⁴ is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R¹⁵ is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
Q is selected from the group consisting of
—NH₂, —NH(C₁-C₄-alkyl), —NH(C₃-C₆-cycloalkyl), —NH(phenyl-C₁-C₄-alkyl), —NH(C₁-C₄-alkoxy), —NH(C₁-C₄-alkyl-C(O)—), (—NH(C₁-C₄-alkoxy-C(O)—), —N(C₁-C₄-alkyl)₂, C₁-C₆-alkyl, C₃-C₁₀-cycloalkyl, C₂-C₆-alkenyl, C₃-C₁₀-cycloalkenyl, C₂-C₆-alkynyl,
each of which is optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, benzyloxy-C(O)—, C₁-C₄-alkoxy-C₁-C₄-alkyl-C(O)—, —C(O)—NH₂, —C(O)—NH(C₁-C₄-alkyl),
—C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl-C₁-C₄-alkyl-, optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl-, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
a C- or N-bound monocyclic or bicyclic heterocycle selected from the group consisting of 4- to 10-membered heterocycloalkyl and heterospirocycloalkyl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl
wherein when Y is O, S or N—R⁹, none of R⁷, R⁸, R¹⁰ and R¹¹ is —OH, and wherein when X is O, S or N—R⁹, none of R⁷ and R⁸ is —OH,
or a stereoisomer, a tautomer, an N-oxide, a hydrate, a solvate, or a salt thereof, or a mixture of same.

### Aspect III of WO 2019/025341 A1:

The compound according to Aspect 1 or 2 of WO 2019/025341 A1, wherein:
A is A1 or A2,

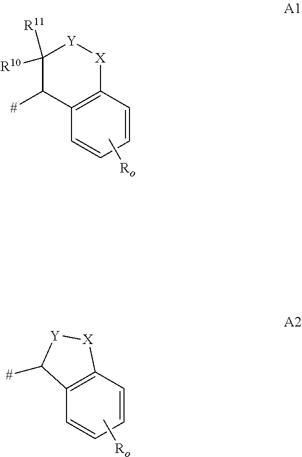

o is 0, 1 or 2,
R is selected from the group consisting of hydrogen, halogen, C₁-C₄-alkyl and C₁-C₄-alkoxy, cyano, C₁-C₄-halogenoalkyl having 1 to 5 halogen atoms,
X, Y are independently selected from the group consisting of CR⁷R⁸, O, S, and N—R⁹, wherein at least one of X and Y is CR⁷R⁸,
R⁸ is selected from the group consisting of hydrogen, C₁-C₄-alkyl, C₃-C₆-cycloalkyl, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl,
R² is selected from the group consisting of hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂,
—NR¹²R¹³;
—OR¹⁴;
—SR¹⁵, —S(O)R¹⁵, —SO₂R¹⁵;
C₁-C₄-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, cyano, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, —OH, oxo, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, and 4- to 10-membered heterocycloalkyl,
R³ is hydrogen, halogen, —OH, C₁-C₄-alkyl or C₁-C₄-alkoxy,
R⁴ is selected from the group consisting of hydrogen, halogen, cyano, C₁-C₄-alkyl, C₁-C₄-halogenoalkyl having 1 to 5 halogen atoms, C₁-C₄-alkoxy, and C₁-C₄-halogenalkoxy having 1 to 5 halogen atoms,
R⁵ is selected from the group consisting of hydrogen, halogen, cyano, C₁-C₄-alkyl, C₁-C₄-halogenoalkyl having 1 to 5 halogen atoms, C₁-C₄-alkoxy, and C₁-C₄-halogenalkoxy having 1 to 5 halogen atoms,
R⁶ is selected from the group consisting of hydrogen, halogen, cyano, C₁-C₄-alkyl, C₁-C₄-halogenoalkyl having 1 to 5 halogen atoms, C₁-C₄-alkoxy, and C₁-C₄-halogenalkoxy having 1 to 5 halogen atoms,
R⁷ is selected from the group consisting of hydrogen and C₁-C₄-alkyl,
R⁸ is selected from the group consisting of hydrogen and C₁-C₄-alkyl,
R⁹ is C₁-C₄-alkyl,
R¹⁰ is selected from the group consisting of hydrogen, —OH, C₁-C₄-alkyl and C₁-C₄-alkoxy,
R¹¹ is hydrogen,
R¹² and R¹³ are independently selected from the group consisting of hydrogen, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)-(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
phenyl, benzo-C₅-C₆-cycloalkyl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
R¹⁴ is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl; and
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
R¹⁵ is selected from the group consisting of
C₁-C₄-alkyl, phenyl-C₁-C₄-alkyl, each of which is optionally substituted by 1, 2 or 3 substituents independently selected from the group consisting of halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
Q is selected from the group consisting of
—NH₂, —NH(C₁-C₄-alkyl), —NH(C₃-C₆-cycloalkyl), —NH(phenyl-C₁-C₄-alkyl), —NH(C₁-C₄-alkoxy), —NH(C₁-C₄-alkyl-C(O)—), (—NH(C₁-C₄-alkoxy-C(O)—), —N(C₁-C₄-alkyl)₂, C₁-C₆-alkyl, C₃-C₁₀-cycloalkyl, C₂-C₆-alkenyl, C₃-C₁₀-cycloalkenyl, C₂-C₆-alkynyl,
each of which is optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, benzyloxy-C(O)—, C₁-C₄-alkoxy-C₁-C₄-alkyl-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl-C₁-C₄-alkyl-, optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl-, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
a C- or N-bound monocyclic or bicyclic heterocycle selected from the group consisting of 4- to 10-membered heterocycloalkyl and heterospirocycloalkyl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl
wherein when Y is O, S or N—R⁹, R¹⁰ is not —OH,
or a stereoisomer, a tautomer, an N-oxide, a hydrate, a solvate, or a salt thereof, or a mixture of same.

### Aspect IV of WO 2019/025341 A1:

The compound according to Aspect 1, 2 or 3 of WO 2019/025341 A1, wherein:
A is A1 or A2,

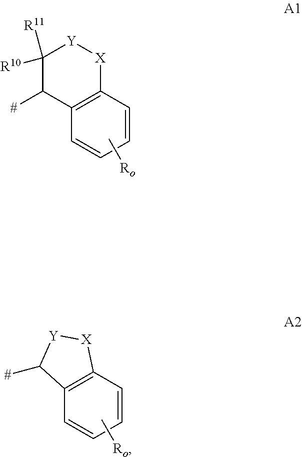

o is 0 or 1,
R is selected from the group consisting of halogen, C₁-C₄-alkyl and C₁-C₄-alkoxy,
X is selected from the group consisting of CR⁷R⁸, O, S, and N—R⁹,
Y is CR⁷R⁸,
R¹ is hydrogen or C₁-C₄-alkyl,
R² is selected from the group consisting of hydrogen, halogen,
—NR¹²R¹³;
—OR¹⁴;
—SR¹⁵, —S(O)R¹⁵, —SO₂R¹⁵;
C₁-C₄-alkyl, C₃-C₆-cycloalkyl, C₂-C₄-alkenyl or C₃-C₆-cycloalkenyl, each of which is optionally substituted by 1, 2, 3, 4 or 5 substituents independently selected from the group consisting of halogen, cyano, C₁-C₄-alkoxy-C(O)— and —C(O)—NH₂; and
a monocyclic or a bicyclic heterocycle selected from the group consisting of 4- to 10-membered heterocycloalkyl, heterospirocycloalkyl, 5-membered heteroaryl, and 6-membered heteroaryl, each of which is optionally substituted by 1, 2, 3 or 4 substituents independently selected from the group consisting of halogen, —OH, oxo, —COOH, C₁-C₄-alkoxy-C(O)—, —C(O)—NH₂, C₁-C₄-alkyl, C₁-C₄-alkyl-C(O)—, C₁-C₄-halogenoalkyl having 1 to 5 halogen atoms, hydroxy-C₁-C₄-alkyl-, C₁-C₄-alkoxy-C₁-C₄-alkyl-, —NH₂, —N(C₁-C₄-alkyl)₂, and 4- to 10-membered heterocycloalkyl,
R³ is hydrogen, halogen, —OH, C₁-C₄-alkyl or C₁-C₄-alkoxy,
R⁴ is selected from the group consisting of hydrogen, halogen, cyano, C₁-C₄-alkyl, C₁-C₄-halogenoalkyl having 1 to 5 halogen atoms, C₁-C₄-alkoxy, and C₁-C₄-halogenalkoxy having 1 to 5 halogen atoms,
R⁵ is selected from the group consisting of hydrogen, halogen, cyano, C₁-C₄-alkyl, C₁-C₄-halogenoalkyl having 1 to 5 halogen atoms, C₁-C₄-alkoxy, and C₁-C₄-halogenalkoxy having 1 to 5 halogen atoms,
R⁶ is selected from the group consisting of hydrogen, halogen, cyano, C₁-C₄-alkyl, C₁-C₄-halogenoalkyl having 1 to 5 halogen atoms, C₁-C₄-alkoxy, and C₁-C₄-halogenalkoxy having 1 to 5 halogen atoms,
R⁷ is selected from the group consisting of hydrogen and C₁-C₄-alkyl,
R⁸ is selected from the group consisting of hydrogen and C₁-C₄-alkyl,
R⁹ is C₁-C₄-alkyl,
R¹⁰ is selected from the group consisting of hydrogen, —OH and C₁-C₄-alkyl,
R¹¹ is hydrogen,
R¹² and R¹³ are independently selected from the group consisting of hydrogen, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, —COOH, C₁-C₄-alkoxy-C(O)—, —C(O)—NH₂, —C(O)—N(C₁-C₄-alkyl)₂, —NH—C(O)—C₁-C₄-alkyl, C₁-C₄-alkyl, C₁-C₄-alkoxy, C₃-C₆-cycloalkyl, —NH₂, —N(C₁-C₄-alkyl)₂, —S—C₁-C₄-alkyl, —S(O)—C₁-C₄-alkyl, —SO₂—C₁-C₄-alkyl, and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms and C₁-C₄-alkoxy;
phenyl and benzo-C₅-C₆-cycloalkyl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms; and
a monocyclic or a bicyclic heterocycle selected from the group of 4- to 10-membered heterocycloalkyl, 5-membered heteroaryl and 6-membered heteroaryl each of which is optionally substituted by 1, 2 or 3 substituents independently selected from the group consisting of halogen, —OH, oxo, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
R¹⁴ is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, C₁-C₄-alkyl, C₁-C₄-alkoxy and C₃-C₆-cycloalkyl; and 4- to 10-membered heterocycloalkyl,
R¹⁵ is selected from the group consisting of C₁-C₄-alkyl, which is optionally substituted by 1, 2 or 3 substituents independently selected from the group consisting of —OH and —COOH; and
a 6-membered heteroaryl,
Q is selected from the group consisting of —NH₂, —NH(C₁-C₄-alkyl), —NH(C₃-C₆-cycloalkyl), —NH(phenyl-C₁-C₄-alkyl), —NH(C₁-C₄-alkoxy), —NH(C₁-C₄-alkyl-C(O)—), (—NH(C₁-C₄-alkoxy-C(O)—), —N(C₁-C₄-alkyl)₂, C₁-C₆-alkyl, C₃-C₁₀-cycloalkyl, C₂-C₆-alkenyl, C₃-C₁₀-cycloalkenyl, C₂-C₆-alkynyl,
each of which is optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, benzyloxy-C(O)—, C₁-C₄-alkoxy-C₁-C₄-alkyl-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl-C₁-C₄-alkyl-, optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl-, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
a C- or N-bound monocyclic or bicyclic heterocycle selected from the group consisting of 4- to 10-membered heterocycloalkyl and heterospirocycloalkyl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl
or a stereoisomer, a tautomer, an N-oxide, a hydrate, a solvate, or a salt thereof, or a mixture of same.

### Aspect V of WO 2019/025341 A1:

The compound according to Aspect 1, 2, 3 or 4 of WO 2019/025341 A1, wherein:
A is selected from the group consisting of

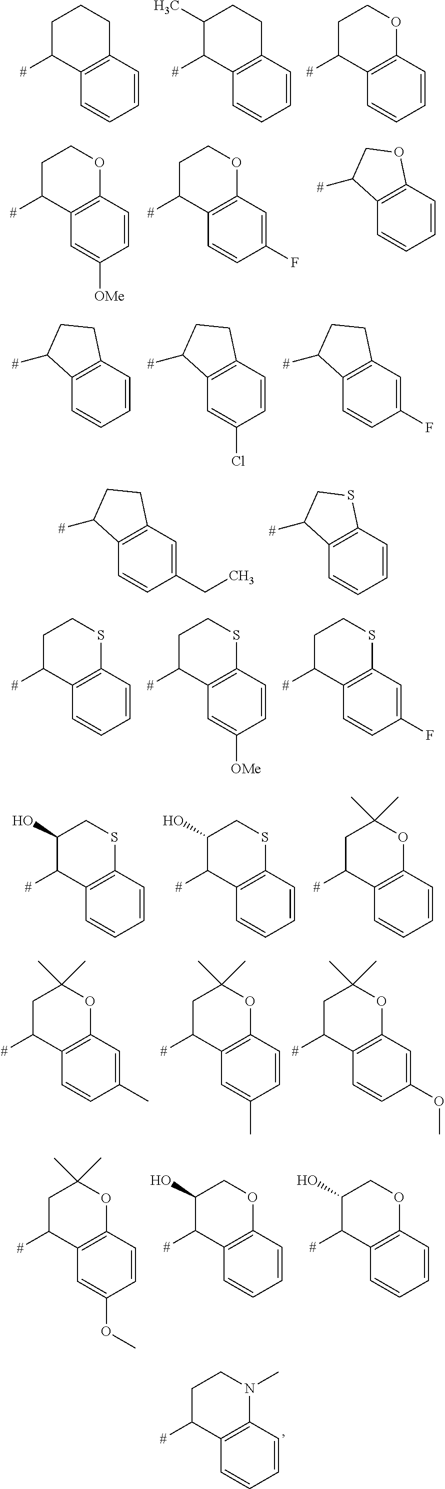

R¹ is hydrogen or methyl,
R² is selected from the group consisting of hydrogen, chlorine,
—NR¹²R¹³;
—OR¹⁴;
—SR¹⁵, —S(O)R¹⁵, —SO₂R¹⁵;
methyl, ethyl, propyl, isopropyl, cyclopropyl, cyclohexyl, propenyl, cyclopentenyl, cyclohexenyl, each of which is optionally substituted by 1 or 2 substituents independently selected from the group consisting of cyano, ethoxy-C(O)—, and —C(O)—NH₂; and
a monocyclic or a bicyclic heterocycle selected from the group consisting of azetidine, pyrrolidine, pyrazolidine, imidazolidine, 1,2,4-triazolidine, piperidine, piperazine, tetrahydropyridine, dihydro-2H-pyrane, tetrahydropyrane, 1,2-oxazolidine, 1,2-oxazine, morpholine, thiomorpholine, 3,4-dihydroisoquinoline, 2,3-dihydro-indole, 1,3-dihydro-isoindole, 3,9-dioxa-7-azabicyclo[3.3.1]nonane, 6-oxa-3-azabicyclo[3.1.1]heptane, 8-oxa-3-azabicyclo[3.2.1]octane, imidazole, pyrazole, 1,2,4-triazole, 1,2,3-triazole, 4-oxa-7-azaspiro[2.5]octane, each of which is optionally substituted by 1, 2, 3 or 4 substituents independently selected from the group consisting of fluorine, chlorine, cyano, —OH, oxo, —COOH, methoxy-C(O)—, ethoxy-C(O)—, tert-butoxy-C(O)—, —C(O)—NH₂, methyl, methyl-C(O)—, trifluoromethyl, hydroxymethyl-, methoxymethyl-, —NH₂, —NMe₂, pyrrolidine,
R³ is hydrogen, chlorine, —OH, methyl or methoxy,
R⁴ is selected from the group consisting of hydrogen, fluorine, chlorine, —OH, cyano, methyl, methoxy, trifluoromethyl, and trifluoromethoxy,
R⁵ is selected from the group consisting of hydrogen, fluorine, chlorine, —OH, cyano, methyl, methoxy, trifluoromethyl and trifluoromethoxy,
R⁶ is selected from the group consisting of hydrogen, fluorine, chlorine, —OH, cyano, methyl, methoxy, trifluoromethyl and trifluoromethoxy,
R¹² and R¹³ are independently selected from the group consisting of hydrogen, —NH(—C(O)-methyl), methoxy;
methyl, ethyl, propyl, isopropyl, butyl, isobutyl, cyclopropyl, cyclobutyl, benzyl, 1-phenylethyl, each of which is optionally substituted by 1, 2 or 3 substituents independently selected from the group consisting of fluorine, —OH, —COOH, methoxy-C(O)—, ethoxy-C(O)—, tert-butoxy-C(O)—, —C(O)—NH₂, —C(O)—NMe₂, —NH—C(O)-methyl, methyl, methoxy, cyclopropyl, —NH₂, NMe₂, S-methyl, S(O)-methyl, SO₂-methyl, and (EtO)₂P(═O)—;
heterocyclyl-methyl, heterocyclyl-ethyl, wherein the heterocyclyl substituent is selected from the group consisting of pyrrolidine, morpholine, pyrazole, 1, 2, 4-oxadiazole, pyridine, each of which is optionally substituted by 1 substituent independently selected from the group consisting of fluorine, chlorine, —OH, oxo and methyl;
phenyl; and
a monocyclic or a bicyclic heterocycle selected from the group of oxetane, thietane, pyrrolidine, morpholine, tetrahydropyrane, pyridine and pyrazole, each of which is optionally substituted by 1 or 2 substituents independently selected from the group consisting of fluorine, chlorine, —OH, oxo, methyl;
R¹⁴ is selected from the group consisting of methyl, ethyl, isopropyl, butyl, cyclopentyl, benzyl, each of which is optionally substituted by 1 or 2 substituents independently selected from the group consisting of fluorine, —OH, methyl, methoxy and cyclopentyl; and
a monocyclic or a bicyclic heterocycle selected from the group consisting of pyrrolidine and tetrahydropyrane,
R¹⁵ is selected from the group consisting of methyl and ethyl, each of which is optionally substituted by 1 substituent independently selected from the group consisting of —OH and —COOH; and
pyridine,
Q is selected from the group consisting of —NH₂, —NH(CH₃), —NH-cyclohexyl, CH₃—C(O)—NH—, (CH₃)₃C—O—C(O)—NH—, —N(CH₃)₂, methyl, ethyl, isopropyl, isobutyl, isopentyl, cyclopentyl, cyclohexyl, cycloheptyl, allyl, prop-1-en-2-yl, cyclopentenyl, cyclohexenyl, cycloheptenyl, prop-1-ynyl, prop-2-ynyl, 3-methyl-but-1-ynyl,
each of which is optionally substituted by 1, 2 or 3 substituents independently selected from the group consisting of halogen, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
a C- or N-bound monocyclic or bicyclic heterocycle selected from the group of oxetane, azetidine, thietane, pyrrolidine, morpholine, thiomorpholine, piperidine tetrahydropyrane, and tetrahydropyridine, each of which is optionally substituted by 1 or 2 substituents independently selected from the group consisting of fluorine, chlorine, —OH, oxo, —COOH, C₁-C₄-alkyl-C(O)—, C₁-C₄-alkoxy-C(O)—, methyl;
or a stereoisomer, a tautomer, an N-oxide, a hydrate, a solvate, or a salt thereof, or a mixture of same.

### Aspect VI of WO 2019/025341 A1:

The compound according to Aspect 1, 2, 3 or 4 of WO 2019/025341 A1, wherein:
A is A1

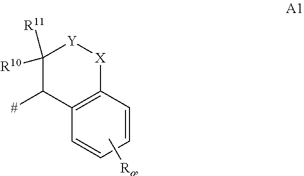

o is 0 or 1,
R is selected from the group consisting of halogen, C₁-C₄-alkyl and C₁-C₄-alkoxy,
X is selected from the group consisting of CR⁷R⁸, O, S, and N—R⁹,
Y is CR⁷R⁸,
R¹ is hydrogen or methyl,
R² is selected from the group consisting of hydrogen, amino, methylamino, ethylamino, dimethylamino, diethylamino, methyl, ethyl, and morpholin-4-yl;
R³ is hydrogen,
R⁴ is selected from the group consisting of hydrogen, chlorine, fluorine, methyl, methoxy and trifluoromethyl,
R⁵ is selected from the group consisting of hydrogen, chlorine, fluorine and methyl,
R⁶ is selected from the group consisting of hydrogen, fluorine and methyl,
Q is selected from the group consisting
cyclohexylamino, acetylamino and tert-butylcarboxylamino, isopropyl, isopentyl, 4-methylpentan-2-yl, 3-methoxypropyl, cyclopentyl, cyclohexyl, cycloheptyl, 4-(trifluoromethyl)cyclohexyl, 4,4-dimethylcyclohexyl, prop-1-en-2-yl, cyclopent-1-en-1-yl, cyclohex-1-en-1-yl, cyclohept-1-en-1-yl, 4-(trifluoromethyl)cyclohex-1-en-1-yl, 4,4-dimethylcyclohex-1-en-1-yl,
3-methylbut-1-yn-1-yl, 3-methoxyprop-1-yn-1-yl, oxetan-3-yl, tetrahydro-2H-pyran-4-yl, 3,6-dihydro-2H-pyran-4-yl, 1,2,3,6-tetrahydropyridin-4-yl,
tert-butyl-azetidine-1-carboxylate, morpholin-4-yl, 3-methylmorpholin-4-yl, 2,6-dimethylmorpholin-4-yl, piperidin-1-yl, and 3,5-dimethylpiperidin-1-yl,
and stereoisomers, tautomers, N-oxides, hydrates, solvates, and salts thereof, and mixtures of same.

### Aspect VII of WO 2019/025341 A1:

The compound according to Aspect 1, 2, 3 or 4 of WO 2019/025341 A1, wherein:
A is A2

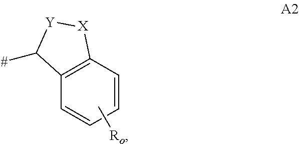

is 0 or 1,
R is selected from the group consisting of halogen, C₁-C₄-alkyl and C₁-C₄-alkoxy,
X is selected from the group consisting of CR⁷R⁸, O, S, and N—R⁹,
Y is CR⁷R⁸,
R¹ is hydrogen or methyl,
R² is selected from the group consisting of hydrogen, amino, methylamino, ethylamino, dimethylamino, diethylamino, methyl, ethyl, isopropyl, and morpholin-4-yl;
R³ is hydrogen,
R⁴ is selected from the group consisting of hydrogen, chlorine, fluorine, methyl, methoxy and trifluoromethyl,
R⁵ is selected from the group consisting of hydrogen, chlorine, fluorine and methyl,
R⁶ is selected from the group consisting of hydrogen, fluorine and methyl,
Q is selected from the group consisting of
pyrrolidin-1-yl, morpholin-4-yl, 1,1-dioxidothiomorpholin-4-yl, piperidin-1-yl, 3,5-dimethylpiperidin-1-yl, and 4,4-difluoropiperidin-1-yl;
and stereoisomers, tautomers, N-oxides, hydrates, solvates, and salts thereof, and mixtures of same.

### Aspect VIII of WO 2019/025341 A1:

The compound according to any one of Aspects 1, 2, 3, 4, 5, 6 or 7 of WO 2019/025341 A1, wherein in the definition of the substituent R² the meaning of hydrogen is excluded.

In one embodiment, E1_1, E2_1′, n and R1 are as defined in WO 2019/025341 A1. In one embodiment, E1_1, E2_1′, n and R1 are as defined in the above-described aspects of WO 2019/025341 A1. In one embodiment, the compound of general formula (I) is a compound of formula I.1 wherein E1_1, E2_1′ and R1 are as defined in the above-described aspects of WO 2019/025341 A1 and n is 0.

### WO 2019/215182 A1

In one embodiment, the compound for use in the long-term prevention and/or treatment of a disease is a compound according to the disclosure of WO 2019/215182 A1. The disclosure of WO 2019/215182 A1 is incorporated herein by reference in its entirety. In one embodiment, the compound for use in the long-term prevention and/or treatment of a disease according to the invention is a compound according to the following aspects of WO 2019/215182 A1:

### Aspect I of WO 2019/215182 A1:

A compound of general formula (I):

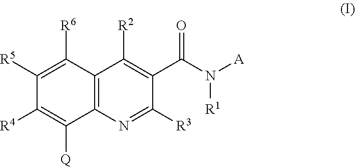

in which:
A is A1 or A2,

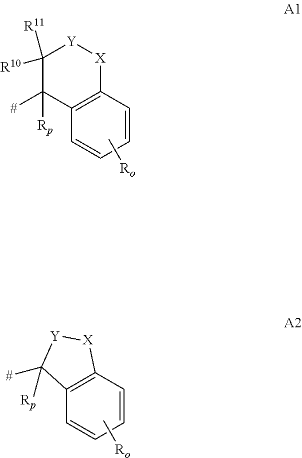

is 0, 1, 2, 3 or 4,
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms,
Rₚ is selected from the group consisting of hydrogen, C₁-C₄-alkyl,
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—,
R¹ is selected from the group consisting of hydrogen, cyano, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N—C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, and —SO₂—C₁-C₄-halogenoalkyl having 1 to 5 halogen atoms;
phenyl-C₁-C₄-alkyl, optionally substituted by 1, 2, 3, 4 or 5 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R² is selected from the group consisting of tetrahydro-2H-pyran-4-yl, 3,6 dihydro-2H-pyran-4-yl, ethyl and 3-fluoroazetidin-1-yl,
R³ is hydrogen or C₁-C₄-alkyl,
R⁴ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, preferably hydrogen, halogen and C₁-C₄-alkoxy, more preferably fluorine, chlorine, methoxy and isopropoxy,
R⁵ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl,
R⁶ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl,
R⁷ is selected from the group consisting of hydrogen, —OH, fluorine, C₁-C₄-alkyl and C₁-C₄-alkoxy,
R⁸ is selected from the group consisting of hydrogen, —OH, fluorine, C₁-C₄-alkyl and C₁-C₄-alkoxy,
or R⁷ and R⁸ together form an oxo group (═O),
or R⁷ and R⁸ form, together with the carbon atom to which they are attached, a 3- to 6-membered ring selected from the group consisting of C₃-C₆-cycloalkyl and 3- to 6-membered heterocycloalkyl,
R⁹ is selected from the group consisting of hydrogen, C₁-C₄-alkyl, C₁-C₄-halogenoalkyl having 1 to 5 halogen atoms and C₁-C₄-alkoxy,
R¹⁰ is selected from the group consisting of hydrogen, —OH, C₁-C₄-alkyl and C₁-C₄-alkoxy,
R¹¹ is selected from the group consisting of hydrogen, C₁-C₄-alkyl and C₁-C₄-alkoxy,
or R¹⁰ and R¹¹ form, together with the carbon atom to which they are attached, a 3- to 6-membered ring selected from the group consisting of C₃-C₆-cycloalkyl and 3- to 6-membered heterocycloalkyl,
Q is 2,3,5-trifluorophenyl,
wherein when Y is O, S or N—R⁹, none of R7, R⁸, R¹⁰ and R¹¹ is —OH or C₁-C₄-alkoxy, and
wherein when X is O, S or N—R⁹, none of R⁷ and R⁸ is —OH or C₁-C₄-alkoxy,
and wherein a compound according to the formula

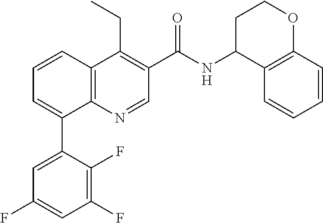 [SMILES: CCc1c(C(=O)NC2CCOc3ccccc32)cnc2c(-c3cc(F)cc(F)c3F)cccc12]

is excluded;
or a stereoisomer, a tautomer, an N-oxide, a hydrate, a solvate, or a salt thereof, or a mixture of same.

### Aspect II of WO 2019/215182 A1:

The compound according to Aspect 1 WO 2019/215182 A1, wherein:
A is A1 or A2,

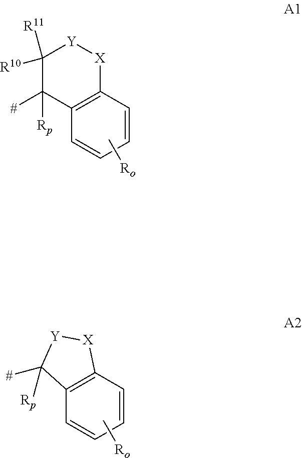

is 0, 1, 2, 3 or 4,
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms,
Rₚ is selected from the group consisting of hydrogen, C₁-C₄-alkyl,
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—,
R¹ is selected from the group consisting of hydrogen, cyano, —CHO, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N—C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, and —SO₂—C₁-C₄-halogenoalkyl having 1 to 5 halogen atoms;
phenyl-C₁-C₄-alkyl, optionally substituted by 1, 2, 3, 4 or 5 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms,
R² is selected from the group consisting of tetrahydro-2H-pyran-4-yl, 3,6 dihydro-2H-pyran-4-yl, ethyl and 3-fluoroazetidin-1-yl,
R³ is hydrogen, or C₁-C₄-alkyl,
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, preferably hydrogen, halogen and C₁-C₄-alkoxy, more preferably fluorine, chlorine, methoxy and isopropoxy,
R⁵ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁶ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁷ is selected from the group consisting of hydrogen, —OH, fluorine, C₁-C₄-alkyl and C₁-C₄-alkoxy,
R⁸ is selected from the group consisting of hydrogen, —OH, fluorine, C₁-C₄-alkyl and C₁-C₄-alkoxy,
or R⁷ and R⁸ together form an oxo group (═O),
R⁹ is selected from the group consisting of hydrogen, C₁-C₄-alkyl, C₁-C₄-halogenoalkyl having 1 to 5 halogen atoms and C₁-C₄-alkoxy,
R¹⁰ is selected from the group consisting of hydrogen, —OH, C₁-C₄-alkyl and C₁-C₄-alkoxy,
R¹¹ is selected from the group consisting of hydrogen, C₁-C₄-alkyl and C₁-C₄-alkoxy,
Q is 2,3,5-trifluorophenyl,
wherein when Y is O, S or N—R⁹, none of R⁷, R⁸, R¹⁰ and R¹¹ is —OH or C₁-C₄-alkoxy, and wherein when X is O, S or N—R⁹, none of R⁷ and R⁸ is —OH or C₁-C₄-alkoxy,
and wherein a compound according to the formula

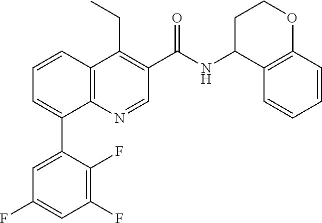 [SMILES: CCc1c(C(=O)NC2CCOc3ccccc32)cnc2c(-c3cc(F)cc(F)c3F)cccc12]

is excluded;
or a stereoisomer, a tautomer, an N-oxide, a hydrate, a solvate, or a salt thereof, or a mixture of same.

### Aspect III of WO 2019/215182 A1:

The compound according to Aspect 1 or 2 of WO 2019/215182 A1, wherein:
A is A1 or A2,

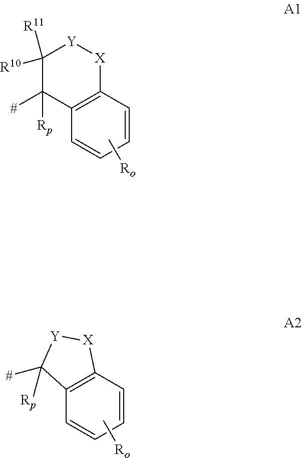

is 0, 1 or 2,
R is selected from the group consisting of halogen, C₁-C₄-alkyl and C₁-C₄-alkoxy, cyano, C₁-C₄-halogenoalkyl having 1 to 5 halogen atoms,
Rₚ is selected from the group consisting of hydrogen, C₁-C₄-alkyl,
X, Y are independently selected from the group consisting of CR⁷R⁸, O, S, and N—R⁹, wherein at least one of X and Y is CR⁷R⁸,
R¹ is selected from the group consisting of hydrogen, C₁-C₄-alkyl, C₃-C₆-cycloalkyl, C₃-C₄-alkenyl, C₃-C₄-alkynyl, C₁-C₄-alkoxy-C₁-C₄-alkyl, C₃-C₆-cycloalkyl-C₁-C₃-alkyl, cyano-C₁-C₄-alkyl,
R² is selected from the group consisting of tetrahydro-2H-pyran-4-yl, 3,6 dihydro-2H-pyran-4-yl, ethyl and 3-fluoroazetidin-1-yl,
R³ is hydrogen or C₁-C₄-alkyl,
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, preferably hydrogen, halogen and C₁-C₄-alkoxy, more preferably fluorine, chlorine, methoxy and isopropoxy,
R⁵ is selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁶ is selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂,
R⁷ is selected from the group consisting of hydrogen and C₁-C₄-alkyl,
R⁸ is selected from the group consisting of hydrogen and C₁-C₄-alkyl,
or R⁷ and R⁸ together form an oxo group (═O),
R⁹ is C₁-C₄-alkyl,
R¹⁰ is selected from the group consisting of hydrogen, —OH, C₁-C₄-alkyl and C₁-C₄-alkoxy,
R¹¹ is hydrogen,
Q is 2,3,5-trifluorophenyl,
wherein when Y is O, S or N—R⁹, R¹⁰ is not —OH or C₁-C₄-alkoxy,
and wherein a compound according to the formula

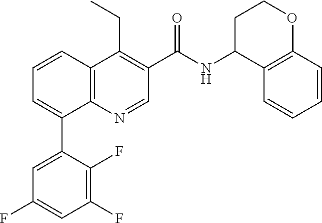 [SMILES: CCc1c(C(=O)NC2CCOc3ccccc32)cnc2c(-c3cc(F)cc(F)c3F)cccc12]

is excluded;
or a stereoisomer, a tautomer, an N-oxide, a hydrate, a solvate, or a salt thereof, or a mixture of same.

### Aspect IV of WO 2019/215182 A1:

The compound according to Aspect 1, 2 or 3 of WO 2019/215182 A1, wherein:
A is A1 or A2,

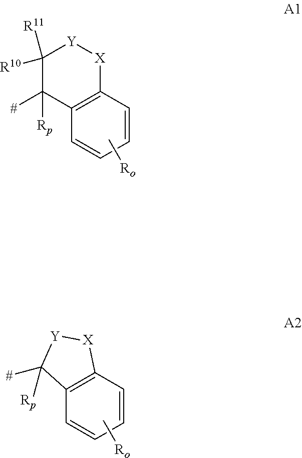

is 0, 1 or 2,
R is selected from the group consisting of halogen, C₁-C₄-alkyl and C₁-C₄-alkoxy,
Rₚ is selected from the group consisting of hydrogen, C₁-C₄-alkyl,
X is selected from the group consisting of CR⁷R⁸, O, S, and N—R⁹,
Y is CR⁷R⁸ or O,
R¹ is hydrogen or C₁-C₄-alkyl,
R² is selected from the group consisting of tetrahydro-2H-pyran-4-yl, 3,6 dihydro-2H-pyran-4-yl, ethyl and 3-fluoroazetidin-1-yl,
R³ is hydrogen or C₁-C₄-alkyl,
R⁴ is selected from the group consisting of hydrogen, halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, preferably hydrogen, halogen and C₁-C₄-alkoxy, more preferably fluorine, chlorine, methoxy and isopropoxy,
R⁵ is selected from the group consisting of hydrogen, halogen, —OH, cyano, C₁-C₄-alkyl, C₁-C₄-halogenoalkyl having 1 to 5 halogen atoms, C₁-C₄-alkoxy,
R⁶ is selected from the group consisting of hydrogen, halogen, —OH, cyano, C₁-C₄-alkyl, C₁-C₄-halogenoalkyl having 1 to 5 halogen atoms, C₁-C₄-alkoxy,
R⁷ is selected from the group consisting of hydrogen and C₁-C₄-alkyl,
R⁸ is selected from the group consisting of hydrogen and C₁-C₄-alkyl,
or R⁷ and R⁸ together form an oxo group (═O),
R⁹ is C₁-C₄-alkyl,
R¹⁰ is selected from the group consisting of hydrogen, —OH and C₁-C₄-alkyl,
R¹¹ is hydrogen,
Q is 2,3,5-trifluorophenyl,
wherein a compound according to the formula

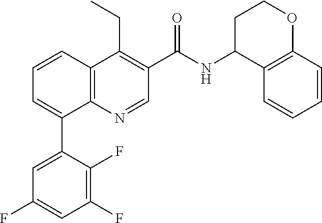 [SMILES: CCc1c(C(=O)NC2CCOc3ccccc32)cnc2c(-c3cc(F)cc(F)c3F)cccc12]

is excluded;
or a stereoisomer, a tautomer, an N-oxide, a hydrate, a solvate, or a salt thereof, or a mixture of same.

### Aspect V of WO 2019/215182 A1:

The compound according to Aspect 1, 2, 3 or 4, wherein:
A is selected from the group consisting of

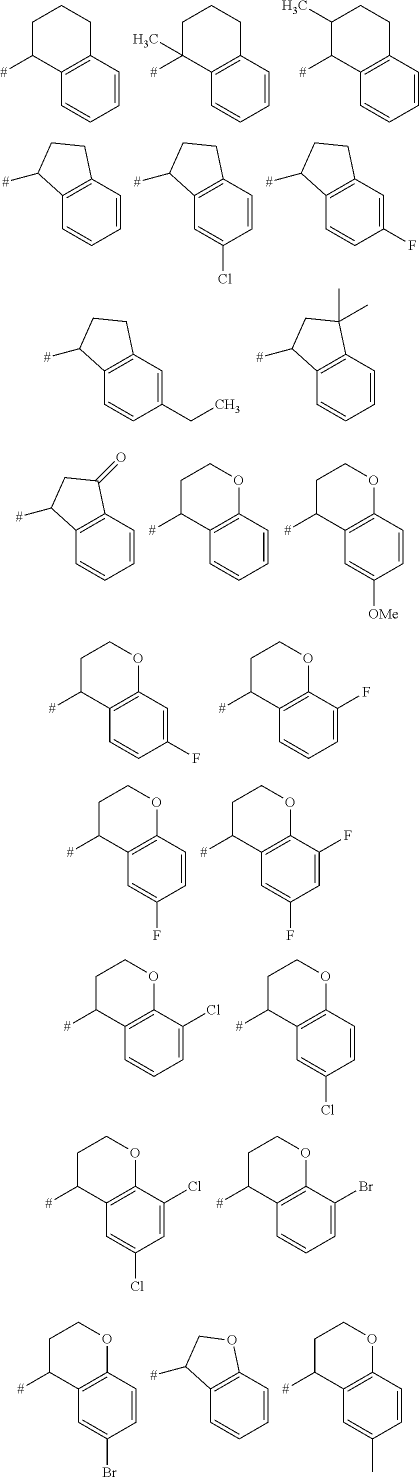

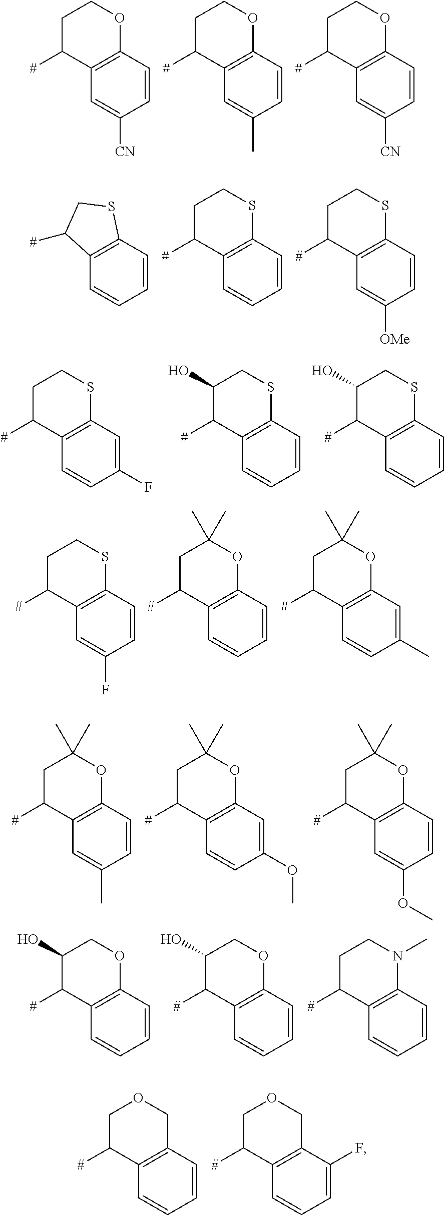

preferably

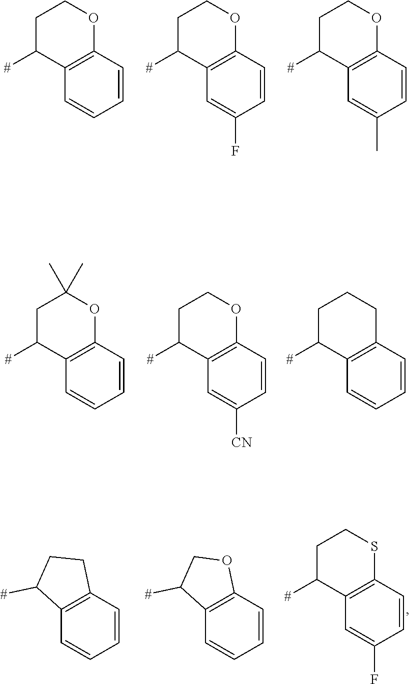

R¹ is hydrogen or methyl,
R² is selected from the group consisting of tetrahydro-2H-pyran-4-yl, 3,6 dihydro-2H-pyran-4-yl, ethyl and 3-fluoroazetidin-1-yl,
R³ is hydrogen or methyl,
R⁴ is selected from the group consisting of hydrogen, fluorine, chlorine, —OH, cyano, methyl, methoxy, trifluoromethyl, trifluoromethoxy and NH₂, preferably hydrogen, fluorine, chlorine, methoxy and isopropoxy,
R⁵ is selected from the group consisting of hydrogen, fluorine, chlorine, —OH, cyano, methyl, methoxy and trifluoromethyl,
R⁶ is selected from the group consisting of hydrogen, fluorine, chlorine, —OH, cyano, methyl and methoxy,
Q is 2,3,5-trifluorophenyl,
wherein a compound according to the formula

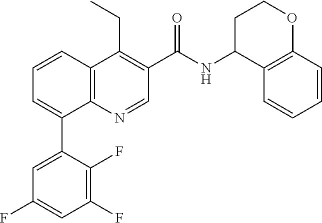 [SMILES: CCc1c(C(=O)NC2CCOc3ccccc32)cnc2c(-c3cc(F)cc(F)c3F)cccc12]

is excluded;
or a stereoisomer, a tautomer, an N-oxide, a hydrate, a solvate, or a salt thereof, or a mixture of same.

### Aspect VI of WO 2019/215182 A1:

The compound according to Aspect 1, 2, 3, 4 or 5 of WO 2019/215182 A1, wherein:
A is selected from the group consisting of

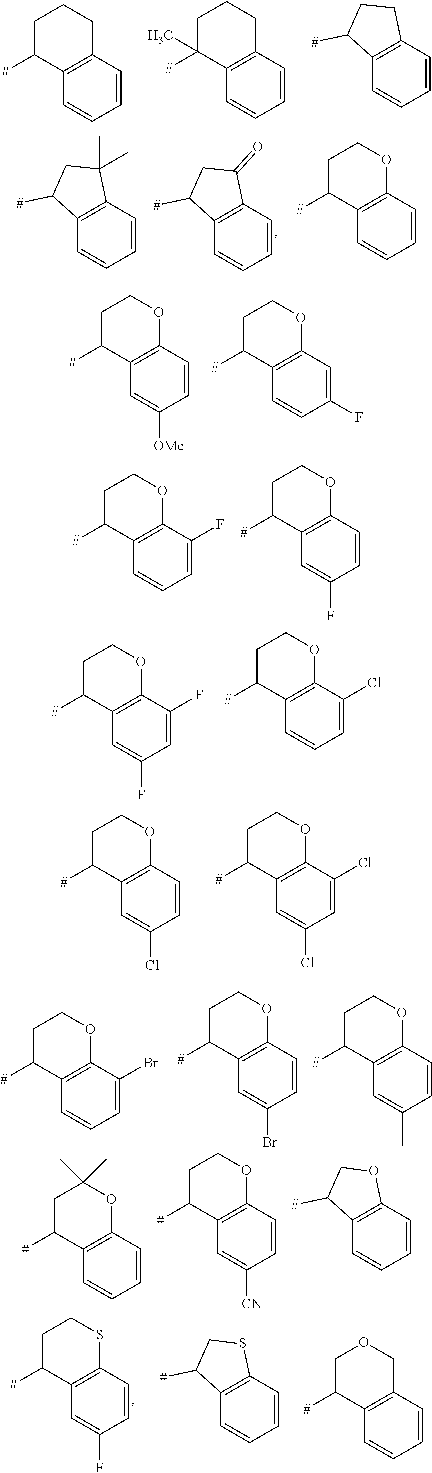

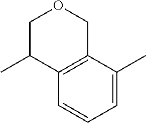

preferably

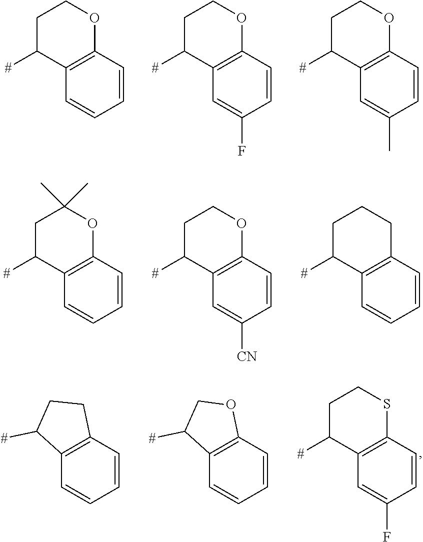

R¹ is hydrogen or methyl,
R² is selected from the group consisting of tetrahydro-2H-pyran-4-yl, 3,6 dihydro-2H-pyran-4-yl, ethyl and 3-fluoroazetidin-1-yl,
R³ is hydrogen or methyl,
R⁴ is selected from the group consisting of hydrogen, chlorine, fluorine, methyl, methoxy, isopropoxy and trifluoromethyl, preferably chlorine, fluorine, methoxy and isopropoxy,
R⁵ is selected from the group consisting of hydrogen, chlorine, fluorine, —OH, cyano, methyl, trifluoromethoxy and NH₂,
R⁶ is selected from the group consisting of hydrogen, fluorine, chlorine, —OH, cyano, methyl and methoxy,
Q is 2,3,5-trifluorophenyl,
wherein a compound according to the formula

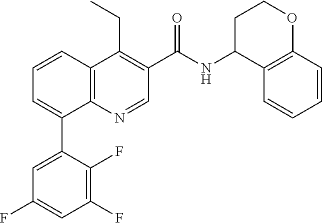 [SMILES: CCc1c(C(=O)NC2CCOc3ccccc32)cnc2c(-c3cc(F)cc(F)c3F)cccc12]

is excluded;
or a stereoisomer, a tautomer, an N-oxide, a hydrate, a solvate, or a salt thereof, or a mixture of same.

### Aspect VII of WO 2019/215182 A1:

The compound according to any one of Aspects 1-6 of WO 2019/215182 A1, wherein:
R² is tetrahydro-2H-pyran-4-yl,
Q is 2,3,5-trifluorophenyl,
and stereoisomers, tautomers, N-oxides, hydrates, solvates, and salts thereof, and mixtures of same.

### Aspect VIII of WO 2019/215182 A1:

The compound according to any one of Aspects 1-6, wherein:
R² is 3,6-dihydro-2H-pyran-4-yl,
Q is 2,3,5-trifluorophenyl,
or a stereoisomers, tautomers, N-oxides, hydrates, solvates, and salts thereof, and mixtures of same.

### Aspect IX of WO 2019/215182 A1:

The compound according to any one of Aspects 1-6, wherein:
R² is ethyl,
R⁴ is selected from the group consisting of hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, preferably hydrogen, halogen and C₁-C₄-alkoxy, more preferably fluorine, chlorine, methoxy and isopropoxy,
Q is 2,3,5-trifluorophenyl,
with the proviso that R⁴ is not hydrogen, when A is

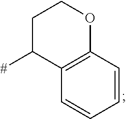

and stereoisomers, tautomers, N-oxides, hydrates, solvates, and salts thereof, and mixtures of same.

### Aspect X of WO 2019/215182 A1:

The compound according to any one of Aspects 1-6, wherein:
R² is 3-fluoroazetidin-1-yl,
Q is 2,3,5-trifluorophenyl,
and stereoisomers, tautomers, N-oxides, hydrates, solvates, and salts thereof, and mixtures of same.

In one embodiment, E1_1, E2_1, n and R1 are as defined in WO 2019/215182 A1. In one embodiment, E1_1, E2_1, n and R1 are as defined in the above-described aspects of WO 2019/215182 A1. In one embodiment, the compound of general formula (I) is a compound of formula I.1 wherein E1_1, E2_1 and R1 are as defined in the above-described aspects of WO 2019/215182 A1 and n is 0.

### WO 2020/131631 A1

In one embodiment, the compound for use in the long-term prevention and/or treatment of a disease is a compound according to the disclosure of WO 2020/131631 A1. The disclosure of WO 2020/131631 A1 is incorporated herein by reference in its entirety. In one embodiment, the compound for use in the long-term prevention and/or treatment of a disease according to the invention is a compound according to the following aspects of WO 2020/131631 A1:

### Aspect I of WO 2020/131631 A1:

A compound of compounds of formula (I):

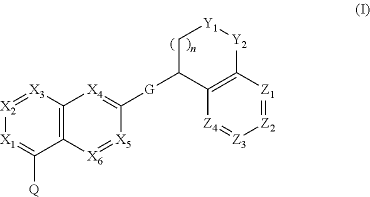

wherein
n is 0 or 1;
X₁ is selected from the group consisting of N and CR₁;
X₂ is selected from the group consisting of N and CR₂;
X₃ is selected from the group consisting of N and CR₃;
X₄ is selected from the group consisting of N and CR₄;
X₅ is selected from the group consisting of N and CR₅;
X₆ is selected from the group consisting of N and CR₆;
G is the group

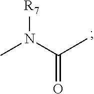

Y₁ is selected from the group consisting of CR₈R₉, O, S, and NR₁₀;
Y₂ is selected from the group consisting of CR₈R₉, O, S, and NR₁₀;
wherein at least one of the groups Y₁ or Y₂ is CR₈R₉;
Z₁ is selected from the group consisting of N, O, S, and CR₁₁;
Z₂ is selected from the group consisting of nil, N, and CR₁₁;
Z₃ is selected from the group consisting of nil, N and CR₁₁;
Z₄ is selected from the group consisting of N, O, S, and CR₁₁;
wherein no more than 2 of Z₁, Z₂, Z₃, and Z₄ are N and wherein only one of Z₁ and Z₄ is O or S, Z₂ is nil only when Z₁ is O or S, and Z₃ is nil only when Z₄ is O or S;
R₁ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₆)(OR₁₇) wherein R₁₆ is, each time taken, selected from the group consisting or hydrogen, C₁-C₄ alkyl, and C₃-C₆ cycloalkyl, R₁₇ is, each time taken, selected from the group consisting or hydrogen, C₁-C₄ alkyl, and C₃-C₆ cycloalkyl, or R₁₆ and R₁₇ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₂ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₆)(OR₁₇) wherein R₁₆ is, each time taken, selected from the group consisting or hydrogen, C₁-C₄ alkyl, and C₃-C₆ cycloalkyl, R₁₇ is, each time taken, selected from the group consisting or hydrogen, C₁-C₄ alkyl, and C₃-C₆ cycloalkyl, or R₁₆ and R₁₇ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₃ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₆)(OR₁₇) wherein R₁₆ is, each time taken, selected from the group consisting or hydrogen, C₁-C₄ alkyl, and C₃-C₆ cycloalkyl, R₁₇ is, each time taken, selected from the group consisting or hydrogen, C₁-C₄ alkyl, and C₃-C₆ cycloalkyl, or R₁₆ and R₁₇ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₄ is selected from the group consisting of halogen, cyano, —CHO, hydroxyl, C₁-C₄ alkyl, C₂-C₄ alkenyl, C₂-C₄ alkynyl, C₃-C₆ cycloalkyl, C₁-C₄ halogenoalkyl, C₁-C₄-alkoxy substituted-C₁-C₄ alkyl, benzyl optionally substituted with 1 to 5 halogen atoms, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NH(4- to 7-membered heterocycloalkyl), —N(C₁-C₄ alkyl)(4- to 7-membered heterocycloalkyl), —N(C₁-C₄ alkyl)(C₃-C₆-cycloalkyl), —N(C₁-C₄ alkyl)(C₁-C₄ alkoxy), —C(O)NH(C₁-C₄ alkyl), —C(O)N(C₁-C₄ alkyl)₂, —NHSO₂(C₁-C₄ alkyl), —SC₁-C₄ alkyl, —S(O)C₁-C₄ alkyl, —SO₂C₁-C₄ alkyl, —B(OR₁₆)(OR₁₇) wherein R₁₆ is, each time taken, selected from the group consisting or hydrogen, C₁-C₄ alkyl, and C₃-C₆ cycloalkyl, R₁₇ is, each time taken, selected from the group consisting or hydrogen, C₁-C₄ alkyl, and C₃-C₆ cycloalkyl, or R₁₆ and R₁₇ together with the oxygen atoms to which they are attached form a 5- to 7-membered ring which is optionally substituted with 1 to 4 C₁-C₄ alkyl; 6- or 10 membered aryl; and a monocyclic heterocycle selected from the group of 4-to 7-membered heterocycloalkyl, 5-membered heteroaryl having at least one nitrogen atom via which the 5-membered heteroaryl ring is connected to the rest of the molecule; 6-membered heteroaryl having at least one nitrogen atom; each of the aryl, heterocycloalkyl, and heteroaryl ring in R₄ is optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂,
—NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl,
—S(O)C₁-C₄-halogenoalkyl and —SO₂C₁-C₄ halogenoalkyl; and wherein each C₁-C₄ alkyl, C₃-C₆ cycloalkyl and C₁-C₄ alkoxy in R₄ may be optionally substituted with 1, 2 or 3 substituents independently selected from the group consisting of halogen, hydroxyl,
—NH₂, —NH(C₁-C₄ alkyl), —N(C₁-C₄ alkyl)₂, cyano, carboxyl, carbamoyl, C₁-C₄ alkoxycarbonyl, —C(O)NH(C₁-C₄ alkyl), —C(O)N(C₁-C₄ alkyl)₂, and C₁-C₄ alkoxy;
R₅ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₆)(OR₁₇) wherein R₁₆ is, each time taken, selected from the group consisting or hydrogen, C₁-C₄ alkyl, and C₃-C₆ cycloalkyl, R₁₇ is, each time taken, selected from the group consisting or hydrogen, C₁-C₄ alkyl, and C₃-C₆ cycloalkyl, or R₁₆ and R₁₇ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₆ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₆)(OR₁₇) wherein R₁₆ is, each time taken, selected from the group consisting or hydrogen, C₁-C₄ alkyl, and C₃-C₆ cycloalkyl, R₁₇ is, each time taken, selected from the group consisting or hydrogen, C₁-C₄ alkyl, and C₃-C₆ cycloalkyl, or R₁₆ and R₁₇ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₇ is selected from the group consisting of hydrogen, C₁-C₄ alkyl, and C₃-C₆ cycloalkyl optionally substituted with 1 to 5 halogen atoms, —C(H)O, C₂-C₄ alkenyl, C₂-C₄ alkynyl, C₁-C₄ halogenoalkyl, and C₁-C₄-alkoxy;
R₈ is, each time selected, independently selected from the group consisting of hydrogen, fluoro, and C₁-C₄ alkyl;
R₉ is, each time selected, independently selected from the group consisting of hydrogen, fluoro, and C₁-C₄ alkyl;
R₁₀ is selected from the group consisting of hydrogen and C₁-C₄ alkyl;
R₁₁ is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and —N(C₁-C₄ alkyl)₂; and
Q is selected from the group consisting of 6- or 10 membered aryl optionally substituted with 1, 2 or 3 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl,
—NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl,
—S(O)C₁-C₄-halogenoalkyl and —SO₂C₁-C₄ halogenoalkyl, wherein the 6- or 10 membered aryl is optionally fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is, valency permitting, substituted with a substituent selected from the group consisting of hydrogen, C₁-C₄ alkyl, and C₃-C₆ cycloalkyl; 5- to 10-membered heteroaryl having 1 or 2 heteroatoms selected from the group O, S, and N and wherein the carbons of the 5- to 10-membered heteroaryl are optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heteroaryl, valency permitting, is optionally substituted with a substituent selected from the group consisting of hydrogen, C₁-C₄ alkyl, and C₃-C₆ cycloalkyl; a 4- to 7-membered heterocycloalkyl having 1 or 2 heteroatoms selected from the group O, S, N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1, 2, 3, or 4 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is optionally substituted with a substituent selected from the group consisting of hydrogen, C₁-C₄ alkyl, and C₃-C₆ cycloalkyl; 6- or 10 membered aryloxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl),
—N(C₁-C₄ alkyl)₂, —NH(C₃-C₆ cycloalkyl), —N(C₁-C₄ alkyl)(C₃-C₆-cycloalkyl),
—NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; 6- or 10 membered arylthio-oxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and 5- to 10-membered heteroaryloxy optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and
—SO₂C₁-C₄ halogenoalkyl;
or a salt thereof.

### Aspect VI of WO 2020/131631 A1:

A compound according to any one of Aspects 1 to 5 of WO 2020/131631 A1_wherein X₁ is CR₁; X₂ is CR₂; X₃ is CR₃; X₄ is CR₄; X₅ is CR₅; and X₆ is CR₆; or a salt thereof.

### Aspect VII of WO 2020/131631 A1:

A compound according to any one of Aspects 1 to 5 of WO 2020/131631 A1, wherein X₁ is CR₁; X₂ is CR₂; X₃ is CR₃; X₄ is CR₄; X₅ is CR₅; and X₆ is N; or a salt thereof.

### Aspect VIII of WO 2020/131631 A1:

A compound according to any one of Aspects 1 to 5 of WO 2020/131631 A1, wherein X₁ is N; X₂ is CR₂; X₃ is CR₃; X₄ is CR₄; X₅ is CR₅; and X₆ is N; or a salt thereof.

### Aspect IX of WO 2020/131631 A1:

A compound according to any one of Aspects 1 to 5 of WO 2020/131631 A1, wherein X₁ is N; X₂ is CR₂; X₃ is CR₃; X₄ is CR₄; X₅ is CR₅; and X₆ is N; or a salt thereof.

### Aspect X of WO 2020/131631 A1:

A compound according to any one of Aspects 1 to 9 of WO 2020/131631 A1, wherein
Q is a 6- or 10 membered aryl optionally substituted with 1, 2 or 3 substituents independently selected from the group consisting of halogen, cyano, nitro, hydroxy, C₁-C₄ alkyl, C₁-C₄ halogenoalkyl, C₁-C₄ alkoxy, C₃-C₆ cycloalkyl, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
or a salt thereof.

### Aspect XI of WO 2020/131631 A1:

A compound according to any one of Aspects 1 to 9 of WO 2020/131631 A1, wherein
Q is 6-membered aryl optionally substituted with 1, 2 or 3 substituents independently selected from the group consisting of halogen, cyano, nitro, hydroxy, C₁-C₄ alkyl, C₁-C₄ halogenoalkyl, C₁-C₄ alkoxy, C₃-C₆ cycloalkyl, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, wherein the 6-membered aryl is fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂,
—NH(C₁-C₄ alkyl), and —N(C₁-C₄ alkyl)₂ and any N in the heterocyclalkyl is substituted with a substituent selected from the group consisting of hydrogen, C₁-C₄ alkyl, and C₃-C₆ cycloalkyl; or a salt thereof.

### Aspect XII of WO 2020/131631 A1:

A compound according to any one of Aspects 1 to 9 of WO 2020/131631 A1, wherein
Q is a 5- to 10-membered heteroaryl having 1 or 2 heteroatoms selected from the group O, S, and N and wherein the carbons of the heteroaryl are optionally substituted with 1, 2 or 3 substituents independently selected from the group consisting of halogen, cyano, nitro, —OH, C₁-C₄ alkyl, C₃-C₆ cycloalkyl, C₁-C₄ halogenoalkyl, C₁-C₄ alkoxy, —NH₂,
—NH(C₁-C₄ alkyl), and —N(C₁-C₄ alkyl)₂ and any N in the heteroaryl is optionally substituted with a substituent selected from the group consisting of hydrogen, C₁-C₄ alkyl, and C₃-C₆ cycloalkyl;
or a salt thereof.

### Aspect XIII of WO 2020/131631 A1:

A compound according to any one of Aspects 1 to 9 of WO 2020/131631 A1, wherein
Q is a 4- to 7-membered heterocycloalkyl having 1 or 2 heteroatoms selected from the group O, S, N, wherein the heterocycloalkyl is optionally benzo-fused, wherein the carbons of the heterocycloalkyl or optionally benzo-fused heterocycloalkyl are optionally substituted with 1, 2, 3, or 4 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocyclalkyl is optionally substituted with a substituent selected from the group consisting of hydrogen, C₁-C₄ alkyl, and C₃-C₆ cycloalkyl;
or a salt thereof.

### Aspect XIV of WO 2020/131631 A1:

The compound according to any one of Aspects 1 to 13 of WO 2020/131631 A1, wherein n is 1; or a salt thereof.

### Aspect XV of WO 2020/131631 A1:

The compound according to any one of Aspects 1 to 14 of WO 2020/131631 A1, wherein Y₁ is CR₈R₉ and Y₂ is O; or a salt thereof.

### Aspect XVI of WO 2020/131631 A1:

The compound according to any one of Aspects 1 to 15 of WO 2020/131631 A1, wherein R₄ is selected from the group consisting of C₁-C₄ alkyl, C₃-C₆ cycloalkyl, —N(C₁-C₄ alkyl)₂, and 4- to 7-membered heterocycloalkyl; or a salt thereof.

### Aspect XVII of WO 2020/131631 A1:

A compound of Aspect 1 wherein the compound is selected from the group consisting of
(4R)—N-[8-(3,5-dichlorophenyl)-4-(dimethylamino)-3-quinolyl]chromane-4-carboxamide;
(4S)—N-[8-(3,5-dichlorophenyl)-4-(dimethylamino)-3-quinolyl]chromane-4-carboxamide;
(1R)—N-[8-(3,5-dichlorophenyl)-4-(dimethylamino)-3-quinolyl]tetralin-1-carboxamide
(1S)—N-[8-(3,5-dichlorophenyl)-4-(dimethylamino)-3-quinolyl]tetralin-1-carboxamide;
(4R)—N-[4-cyclopropyl-8-(3,5-dichlorophenyl)-3-quinolyl]chromane-4-carboxamide;
(4S)—N-[4-cyclopropyl-8-(3,5-dichlorophenyl)-3-quinolyl]chromane-4-carboxamide;
(4R)—N-[8-(3,5-dichlorophenyl)-4-[methoxy(methyl)amino]-3-quinolyl]chromane-4-carboxamide;
(4S)—N-[8-(3,5-dichlorophenyl)-4-[methoxy(methyl)amino]-3-quinolyl]chromane-4-carboxamide;
(4S)—N-[8-(2,3-dichlorophenyl)-4-(dimethylamino)-3-quinolyl]chromane-4-carboxamide;
(4R)—N-[8-[3-chloro-5-(trifluoromethyl)phenyl]-4-(dimethylamino)-3-quinolyl]chromane-4-carboxamide;
(4S)—N-[8-[3-chloro-5-(trifluoromethyl)phenyl]-4-(dimethylamino)-3-quinolyl]chromane-4-carboxamide;
(4S)—N-[8-(3,5-dichlorophenyl)-4-morpholino-3-quinolyl]chromane-4-carboxamide;
(4R)—N-[8-(3,5-dichlorophenyl)-4-morpholino-3-quinolyl]chromane-4-carboxamide;
(4S)—N-[4-morpholino-8-(2,3,5-trifluorophenyl)-3-quinolyl]chromane-4-carboxamide;
(4R)—N-[4-morpholino-8-(2,3,5-trifluorophenyl)-3-quinolyl]chromane-4-carboxamide;]
3-[1-[[(4R)-chroman-4-yl]amino]-2,2,2-trifluoro-ethyl]-8-(3,5-dichlorophenyl)-N,N-dimethyl-quinolin-4-amine;
3-[1-[[(4S)-chroman-4-yl]amino]-2,2,2-trifluoro-ethyl]-8-(3,5-dichlorophenyl)-N,N-dimethyl-quinolin-4-amine;
N-[(4S)-chroman-4-yl]-8-(3,5-dichlorophenyl)-4-(dimethylamino)quinoline-3-sulfonamide;
N-[8-(3,5-dichlorophenyl)-4-(dimethylamino)-3-quinolyl]-(4R and S)-chromane-4-sulfonamide;
(4S)—N-[8-(3,5-dichlorophenyl)-4-(dimethylamino)-1,7-naphthyridin-3-yl]chromane-4-carboxamide;
(4R)—N-[8-(3,5-dichlorophenyl)-4-(dimethylamino)-1,7-naphthyridin-3-yl]chromane-4-carboxamide;
(4S)—N-[4-morpholino-8-(2,3,5-trifluorophenyl)-1,7-naphthyridin-3-yl]chromane-4-carboxamide;
(4R)—N-[4-morpholino-8-(2,3,5-trifluorophenyl)-1,7-naphthyridin-3-yl]chromane-4-carboxamide;
(4S)—N-[4-(3,5-dichlorophenyl)-8-(dimethylamino)pyrido[3,2-d]pyrimidin-7-yl]chromane-4-carboxamide;
(4R)—N-[4-(3,5-dichlorophenyl)-8-(dimethylamino)pyrido[3,2-d]pyrimidin-7-yl]chromane-4-carboxamide;
(4R)—N-[8-(3,5-dichlorophenyl)-4-(dimethylamino)-1,5-naphthyridin-3-yl]chromane-4-carboxamide;
(4S)—N-[8-(3,5-dichlorophenyl)-4-(dimethylamino)-1,5-naphthyridin-3-yl]chromane-4-carboxamide;
(4S)—N-[8-(3,5-dichlorophenyl)-4-morpholino-1,5-naphthyridin-3-yl]chromane-4-carboxamide;
1-[(4S)-Chroman-4-yl]-3-[8-(3,5-dichlorophenyl)-4-(dimethylamino)-3-quinolyl]urea;
(4R)—N-[5-(3,5-dichlorophenyl)-1-(dimethylamino)-2-naphthyl]chromane-4-carboxamide; and
(4S)—N-[5-(3,5-dichlorophenyl)-1-(dimethylamino)-2-naphthyl]chromane-4-carboxamide; or a salt of each of the above-mentioned compounds.

In one embodiment, E1_7, E2_1″, n and R1 are as defined in WO 2020/131631 A1. In one embodiment, E1_7, E2_1″, n and R1 are as defined in the above-described aspects of WO 2020/131631 A1. In one embodiment, the compound of general formula (I) is a compound of formula I.2 wherein E1_7, E2_1″ and R1 are as defined in the above-described aspects of WO 2020/131631 A1 and n is 0.

### WO 2022/117783 A1

In one embodiment, the compound for use in the long-term prevention and/or treatment of a disease is a compound according to the disclosure of WO 2022/117783 A1. The disclosure of WO 2022/117783 A1 is incorporated herein by reference in its entirety. In one embodiment, the compound for use in the long-term prevention and/or treatment of a disease according to the invention is a compound according to the following aspects of WO 2022/117783 A1:

### Aspect I of WO 2022/117783 A1:

A compound of formula (I′):

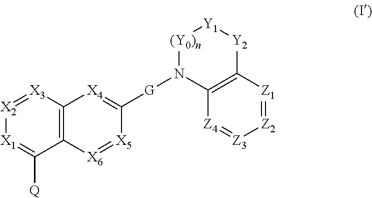

wherein
n is 0 or 1; when n is 1, Y₀ is CH₂ or C═O;
X₁ is selected from the group consisting of N and CR₁;
X₂ is selected from the group consisting of N and CR₂;
X₃ is selected from the group consisting of N and CR₃;
X₄ is selected from the group consisting of N and CR₄;
X₅ is selected from the group consisting of N and CR₅;
X₆ is selected from the group consisting of N and CR₆;
G is selected from the group consisting of

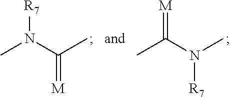

M is selected from the group consisting of N—R₁₃, O, and S;
Y₁ is selected from the group consisting of CR₈R₉, O, S, and NR₁₀;
Y₂ is selected from the group consisting of CR₈R₉, O, S, and NR₁₀; wherein at least one of the groups
Y₁ or Y₂ is CR₈R₉;
Z₁ is selected from the group consisting of N, O, S, and CR₁₁;
Z₂ is selected from the group consisting of nil, N, and CR₁₁;
Z₃ is selected from the group consisting of nil, N and CR₁₁;
Z₄ is selected from the group consisting of N, O, S, and CR₁₁;
wherein no more than 2 of Z₁, Z₂, Z₃, and Z₄ are N and wherein only one of Z₁ and Z₄ is O or S, Z₂ is nil only when Z₁ is O or S, and Z₃ is nil only when Z₄ is O or S;
R₁ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₉ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₂ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₃ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₄ is selected from the group consisting of halogen, cyano, —CHO, hydroxyl, C₁-C₄ alkyl, C₂-C₄ alkenyl, C₂-C₄ alkynyl, C₃-C₆ cycloalkyl, C₁-C₄ halogenoalkyl, C₁-C₄-alkoxy substituted-C₁-C₄ alkyl, benzyl optionally substituted with 1 to 5 halogen atoms, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₁-C₄ alkoxy substituted C₁-C₄ alkyl), —N(C₁-C₄ alkoxy substituted C₁-C₄ alkyl)₂, —N(C(O)C₁-C₄ alkyl)(C₁-C₄ alkyl), —N(C₁-C₄ alkyl)(C₃-C₆-cycloalkyl), —N(C₁-C₄ alkyl)(4- to 7-membered heterocycloalkyl), —NH(4- to 7-membered heterocycloalkyl), —N(C₁-C₄ alkyl)(C₁-C₄ alkoxy), —C(O)NH(C₁-C₄ alkyl), —C(O)N(C₁-C₄ alkyl)₂, —C(O)N(C₁-C₄ alkyl)(4- to 7-membered heterocycloalkyl), —NHSO₂(C₁-C₄ alkyl), —SC₁-C₄ alkyl, —S(O)C₁-C₄ alkyl, —SO₂C₁-C₄ alkyl, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; 6- or 10 membered aryl; a monocyclic heterocycle selected from the group of 4- to 7-membered heterocycloalkyl, 5-membered heteroaryl having at least one nitrogen atom via which the 5-membered heteroaryl ring is connected to the rest of the molecule, and 6-membered heteroaryl having at least one nitrogen atom; each of the aryl, heterocycloalkyl, and heteroaryl rings in R₄ is optionally substituted with 1, 2 or 3 substituents independently selected from the group consisting of halogen, cyano, nitro, hydroxy, oxo, imino, 1-imino-1-oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; wherein the C₃-C₆ cycloalkyl and the heterocycloalkyl rings in R₄ are optionally substituted with a spiro group, wherein said spiro group is a 3- to 6-membered cycloalkyl or 4- to 6-membered heterocycloalkyl containing 1, 2, or 3 heteroatoms independently selected from N, S or O, wherein said spiro group is optionally substituted with 1, 2 or 3 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and wherein each C₁-C₄ alkyl, C₃-C₆ cycloalkyl and C₁-C₄ alkoxy in R₄ may be optionally substituted with 1, 2 or 3 substituents independently selected from the group consisting of halogen, hydroxy, oxo, —NH₂, —NH(C₁-C₄ alkyl), —N(C₁-C₄ alkyl)₂, cyano, carboxy, carbamoyl, C₁-C₄ alkoxycarbonyl, —C(O)NH(C₁-C₄ alkyl), —C(O)N(C₁-C₄ alkyl)₂, C₁-C₄ halogenoalkyl, and C₁-C₄ alkoxy;
R₅ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₆ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₇ is selected from the group consisting of hydrogen, C₁-C₉ alkyl, and C₃-C₆ cycloalkyl optionally substituted with 1 to 5 halogen atoms, —C(H)O, C₂-C₄ alkenyl, C₂-C₄ alkynyl, C₁-C₄ halogenoalkyl, and C₁-C₄-alkoxy;
R₈ is, each time selected, independently selected from the group consisting of hydrogen, fluoro, and C₁-C₄ alkyl;
R₉ is, each time selected, independently selected from the group consisting of hydrogen, fluoro, and C₁-C₄ alkyl;
R₁₀ is selected from the group consisting of hydrogen and C₁-C₄ alkyl;
R₁₁ is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and —N(C₁-C₄ alkyl)₂;
Q is selected from the group consisting of
(i) 6- or 10 membered aryl optionally substituted with 1, 2, 3, 4, or 5 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl, —C(O)NH₂, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl, —SO₂C₁-C₄ halogenoalkyl, and pentafluoro-sulfanyl, wherein the 6- or 10 membered aryl is optionally fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is, valency permitting, substituted with a substituent selected from the group consisting of hydrogen, C₁-C₄ alkyl, and C₃-C₆ cycloalkyl;
(ii) 5- to 10-membered heteroaryl having 1, 2, or 3 heteroatoms independently selected from the group O, S, and N and wherein the carbons of the 5- to 10-membered heteroaryl are optionally substituted with 1, 2, 3, 4, or 5 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, benzyloxy, —C(O)R₁₇, —NH₂, —NH(C₁-C₄ alkyl), —N(C₁-C₄ alkyl)₂, —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, and any N in the heteroaryl, valency permitting, is optionally substituted with a substituent selected from the group consisting of hydrogen, C₁-C₄ alkyl, and C₃-C₆ cycloalkyl;
(iii) 4- to 7-membered heterocycloalkyl having 1, 2, or 3 heteroatoms independently selected from the group O, S, N, wherein the heterocycloalkyl is optionally benzo-fused, wherein the carbons of the 4-to 7-membered heterocycloalkyl or optionally benzo-fused 4- to 7-membered heterocycloalkyl are optionally substituted with 1, 2, 3, or 4 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), and —N(C₁-C₄ alkyl)₂ and any N in the heterocycloalkyl is optionally substituted with a substituent selected from the group consisting of hydrogen, C₁-C₄ alkyl, and C₃-C₆ cycloalkyl;
(iv) 6- or 10 membered aryloxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
(v) 6- or 10 membered arylthio-oxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and
(vi) 5- to 10-membered heteroaryloxy optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
R₁₃ is selected from the group consisting of hydroxy, C₁-C₄ alkoxy, and —NH₂; and
R₁₇ is, each time selected, independently selected from the group consisting of C₁-C₄ alkyl, C₃-C₆ cycloalkyl, C₁-C₄ halogenoalkyl, C₁-C₄ alkoxy, C₁-C₄ halogenoalkoxy, —OH, —NH₂, —NH(C₁-C₄ alkyl), —N(C₁-C₄ alkyl)₂, —NH(C₃-C₆ cycloalkyl), and —N(C₁-C₄ alkyl)(C₃-C₆-cycloalkyl);
or a stereoisomer or salt thereof.

### Aspect II of WO 2022/117783 A1:

The compound of formula (I′) according to Aspect 1 of WO 2022/117783 A1, wherein
n is 0 or 1; when n is 1, Y₀ is CH₂ or C═O;
X₁ is selected from the group consisting of N and CR₁;
X₂ is selected from the group consisting of N and CR₂;
X₃ is selected from the group consisting of N and CR₃;
X₄ is CR₄;
X₅ is CR₅;
X₆ is selected from the group consisting of N and CR₆;
G is selected from the group consisting of

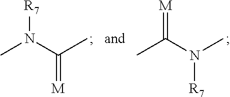

M is selected from the group consisting of O and S;
Y₁ is CR₈R₉;
Y₂ is selected from the group consisting of CR₈R₉, O, and S;
Z₁ is CR₁₁;
Z₂ is CR₁₁;
Z₃ is CR₁₁;
Z₄ is CR₁₁;
R₁ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₉ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₂ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₃ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₄ is selected from the group consisting of halogen, cyano, —CHO, hydroxyl, C₁-C₄ alkyl, C₂-C₄ alkenyl, C₂-C₄ alkynyl, C₃-C₆ cycloalkyl, C₁-C₄ halogenoalkyl, C₁-C₄-alkoxy substituted-C₁-C₄ alkyl, benzyl optionally substituted with 1 to 5 halogen atoms, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₁-C₄ alkoxy substituted C₁-C₄ alkyl), —N(C₁-C₄ alkoxy substituted C₁-C₄ alkyl)₂, —N(C(O)C₁-C₄ alkyl)(C₁-C₄ alkyl),_-N(C₁-C₄ alkyl)(C₃-C₆-cycloalkyl), —N(C₁-C₄ alkyl)(4- to 7-membered heterocycloalkyl), —NH(4- to 7-membered heterocycloalkyl), —N(C₁-C₄ alkyl)(C₁-C₄ alkoxy), —C(O)NH(C₁-C₄ alkyl), —C(O)N(C₁-C₄ alkyl)₂, —C(O)N(C₁-C₄ alkyl)(4- to 7-membered heterocycloalkyl), —NHSO₂(C₁-C₄ alkyl), —SC₁-C₄ alkyl, —S(O)C₁-C₄ alkyl, —SO₂C₁-C₄ alkyl, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; 6- or 10 membered aryl; a monocyclic heterocycle selected from the group of 4- to 7-membered heterocycloalkyl, 5-membered heteroaryl having at least one nitrogen atom via which the 5-membered heteroaryl ring is connected to the rest of the molecule, and 6-membered heteroaryl having at least one nitrogen atom; each of the aryl, heterocycloalkyl, and heteroaryl rings in R₄ is optionally substituted with 1, 2 or 3 substituents independently selected from the group consisting of halogen, cyano, nitro, hydroxy, oxo, imino, 1-imino-1-oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; wherein the C₃-C₆ cycloalkyl and the heterocycloalkyl rings in R₄ are optionally substituted with a spiro group, wherein said spiro group is a 3- to 6-membered cycloalkyl or 4- to 6-membered heterocycloalkyl containing 1, 2, or 3 heteroatoms independently selected from N, S or O, wherein said spiro group is optionally substituted with 1, 2 or 3 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and wherein each C₁-C₄ alkyl, C₃-C₆ cycloalkyl and C₁-C₄ alkoxy in R₄ may be optionally substituted with 1, 2 or 3 substituents independently selected from the group consisting of halogen, hydroxy, oxo, —NH₂, —NH(C₁-C₄ alkyl), —N(C₁-C₄ alkyl)₂, cyano, carboxy, carbamoyl, C₁-C₄ alkoxycarbonyl, —C(O)NH(C₁-C₄ alkyl), —C(O)N(C₁-C₄ alkyl)₂, and C₁-C₄ alkoxy, and C₁-C₄ halogenoalkyl;
R₅ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₆ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₇ is selected from the group consisting of hydrogen, C₁-C₉ alkyl, and C₃-C₆ cycloalkyl optionally substituted with 1 to 5 halogen atoms, —C(H)O, C₂-C₄ alkenyl, C₂-C₄ alkynyl, C₁-C₄ halogenoalkyl, and C₁-C₄-alkoxy;
R₈ is, each time selected, independently selected from the group consisting of hydrogen, fluoro, and C₁-C₄ alkyl;
R₉ is, each time selected, independently selected from the group consisting of hydrogen, fluoro, and C₁-C₄ alkyl;
R₁₁ is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and —N(C₁-C₄ alkyl)₂;
Q is selected from the group consisting of
(i) 6- or 10 membered aryl optionally substituted with 1, 2, 3, 4, or 5 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl, —C(O)NH₂, —C(O)R₂, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl, —SO₂C₁-C₄ halogenoalkyl, and pentafluoro-sulfanyl, wherein the 6- or 10 membered aryl is optionally fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is, valency permitting, substituted with a substituent selected from the group consisting of hydrogen, C₁-C₄ alkyl, and C₃-C₆ cycloalkyl;
(ii) 5- to 10-membered heteroaryl having 1, 2, or 3 heteroatoms independently selected from the group O, S, and N and wherein the carbons of the 5- to 10-membered heteroaryl are optionally substituted with 1, 2, 3, 4, or 5 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, benzyloxy, —C(O)R₁₇, —NH₂, —NH(C₁-C₄ alkyl), —N(C₁-C₄ alkyl)₂, —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, and any N in the heteroaryl, valency permitting, is optionally substituted with a substituent selected from the group consisting of hydrogen, C₁-C₄ alkyl, and C₃-C₆ cycloalkyl;
(iii) 4- to 7-membered heterocycloalkyl having 1, 2, or 3 heteroatoms independently selected from the group O, S, N, wherein the heterocycloalkyl is optionally benzo-fused, wherein the carbons of the 4-to 7-membered heterocycloalkyl or optionally benzo-fused 4- to 7-membered heterocycloalkyl are optionally substituted with 1, 2, 3, or 4 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), and —N(C₁-C₄ alkyl)₂ and any N in the heterocycloalkyl is optionally substituted with a substituent selected from the group consisting of hydrogen, C₁-C₄ alkyl, and C₃-C₆ cycloalkyl;
(iv) 6- or 10 membered aryloxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
(v) 6- or 10 membered arylthio-oxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and
(vi) 5- to 10-membered heteroaryloxy optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —C(O)R₁₂, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and
R₁₇ is, each time selected, independently selected from the group consisting of C₁-C₄ alkyl, C₃-C₆ cycloalkyl, C₁-C₄ halogenoalkyl, C₁-C₄ alkoxy, C₁-C₄ halogenoalkoxy, —OH, —NH₂, —NH(C₁-C₄ alkyl), —N(C₁-C₄ alkyl)₂, —NH(C₃-C₆ cycloalkyl), and —N(C₁-C₄ alkyl)(C₃-C₆-cycloalkyl);
or a stereoisomer or salt thereof

### Aspect III of WO 2022/117783 A1:

The compound of formula (I′) according to Aspect 1 or 2 of WO 2022/117783 A1, wherein

n is 0 or 1; when n is 1, Y₀ is CH₂ or C═O;
X₁ is selected from the group consisting of N and CR₁;
X₂ is selected from the group consisting of N and CR₂;
X₃ is selected from the group consisting of N and CR₃;
X₄ is CR₄;
X₅ is CR₅;
X₆ is selected from the group consisting of N and CR₆;
G is selected from the group consisting of

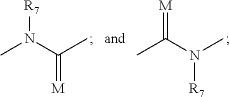

M is O;
Y₁ is CR₈R₉;
Y₂ is selected from the group consisting of CR₈R₉ and O;
Z₁ is CR₁₁;
Z₂ is CR₁₁;
Z₃ is CR₁₁;
Z₄ is CR₁₁;
R₁ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₉ alkyl, C₁-C₄ halogenoalkyl, C₁-C₄-alkoxy;
R₂ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy;
R₃ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy;
R₄ is selected from the group consisting of B(OH)₂, halogen, cyano, —CHO, hydroxyl, C₁-C₄ alkyl, C₂-C₄ alkenyl, C₂-C₄ alkynyl, C₃-C₆ cycloalkyl, C₁-C₄ halogenoalkyl, C₁-C₄-alkoxy substituted-C₁-C₄ alkyl, benzyl optionally substituted with 1 to 5 halogen atoms, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₁-C₄ alkoxy substituted C₁-C₄ alkyl), —N(C₁-C₄ alkoxy substituted C₁-C₄ alkyl)₂, —N(C(O)C₁-C₄ alkyl)(C₁-C₄ alkyl), —N(C₁-C₄ alkyl)(C₃-C₆-cycloalkyl), —N(C₁-C₄ alkyl)(4- to 7-membered heterocycloalkyl), —NH(4- to 7-membered heterocycloalkyl), —N(C₁-C₄ alkyl)(C₁-C₄ alkoxy), —C(O)NH(C₁-C₄ alkyl), —C(O)N(C₁-C₄ alkyl)₂, —C(O)N(C₁-C₄ alkyl)(4- to 7-membered heterocycloalkyl), —NHSO₂(C₁-C₄ alkyl), —SC₁-C₄ alkyl, —S(O)C₁-C₄ alkyl, —SO₂C₁-C₄ alkyl, —B(OR₁₅)(OR₁₆) wherein R₁₅ is, each time taken, selected from the group consisting of hydrogen, C₁-C₄ alkyl, and C₃-C₆ cycloalkyl, R₁₆ is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; 6- or 10 membered aryl; a monocyclic heterocycle selected from the group of 4- to 7-membered heterocycloalkyl, 5-membered heteroaryl having at least one nitrogen atom via which the 5-membered heteroaryl ring is connected to the rest of the molecule, and 6-membered heteroaryl having at least one nitrogen atom; each of the aryl, heterocycloalkyl, and heteroaryl rings in R₄ is optionally substituted with 1, 2 or 3 substituents independently selected from the group consisting of halogen, cyano, nitro, hydroxy, oxo, imino, 1-imino-1-oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; wherein the C₃-C₆ cycloalkyl and the heterocycloalkyl rings in R₄ are optionally substituted with a spiro group, wherein said spiro group is a 3- to 6-membered cycloalkyl or 4- to 6-membered heterocycloalkyl containing 1, 2, or 3 heteroatoms independently selected from N, S or O, wherein said spiro group is optionally substituted with 1, 2 or 3 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and wherein each C₁-C₄ alkyl, C₃-C₆ cycloalkyl and C₁-C₄ alkoxy in R₄ may be optionally substituted with 1, 2 or 3 substituents independently selected from the group consisting of halogen, hydroxy, oxo, —NH₂, —NH(C₁-C₄ alkyl), —N(C₁-C₄ alkyl)₂, cyano, carboxy, carbamoyl, C₁-C₄ alkoxycarbonyl, —C(O)NH(C₁-C₄ alkyl), —C(O)N(C₁-C₄ alkyl)₂, C₁-C₄ alkoxy, and C₁-C₄ halogenoalkyl;
R₅ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy,
R₆ is selected from the group consisting of hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy,
R₇ is selected from the group consisting of hydrogen, C₁-C₉ alkyl, and C₃-C₆ cycloalkyl optionally substituted with 1 to 5 halogen atoms, —C(H)O, C₂-C₄ alkenyl, C₂-C₄ alkynyl, C₁-C₄ halogenoalkyl, and C₁-C₄-alkoxy;
R₈ is, each time selected, independently selected from the group consisting of hydrogen, fluoro, and C₁-C₄ alkyl;
R₉ is, each time selected, independently selected from the group consisting of hydrogen, fluoro, and C₁-C₄ alkyl;
R₁₁ is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and —N(C₁-C₄ alkyl)₂;
Q is selected from the group consisting of
(i) 6- or 10-membered aryl optionally substituted with 1, 2, 3, 4, or 5 substituents independently selected from the group consisting of halogen, cyano, nitro, hydroxyl, —C₁-C₄ alkyl, C₁-C₄ halogenoalkyl, C₁-C₄ alkoxy, C₃-C₆ cycloalkyl, C(O)NH₂, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl, —SO₂C₁-C₄ halogenoalkyl, and pentafluoro-sulfanyl;
(ii) 5- to 10-membered heteroaryl having 1, 2, or 3 heteroatoms independently selected from the group of O, S, and N, an wherein the carbons of the 5- to 10-membered heteroaryl are optionally substituted with 1, 2, 3, 4, or 5 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, benzyloxy, —C(O)R₁₇, —NH₂, —NH(C₁-C₄ alkyl), —N(C₁-C₄ alkyl)₂, —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, and any N in the heteroaryl, valency permitting, is optionally substituted with a substituent selected from the group consisting of hydrogen, C₁-C₄ alkyl, and C₃-C₆ cycloalkyl, and
R₁₇ is, each time selected, independently selected from the group consisting of C₁-C₄ alkyl, C₃-C₆ cycloalkyl, C₁-C₄ halogenoalkyl, C₁-C₄ alkoxy, C₁-C₄ halogenoalkoxy, —OH, —NH₂, —NH(C₁-C₄ alkyl), —N(C₁-C₄ alkyl)₂, —NH(C₃-C₆ cycloalkyl), and —N(C₁-C₄ alkyl)(C₃-C₆-cycloalkyl);
or a stereoisomer or salt thereof.

### Aspect IV of WO 2022/117783 A1:

The compound of formula (I′) according to any one of claims 1 to 3, wherein
n is 0 or 1; when n is 1, Y₀ is CH₂ or C═O;
X₁ is selected from the group consisting of N and CR₁;
X₂ is selected from the group consisting of N and CR₂;
X₃ is selected from the group consisting of N and CR₃;
X₄ is CR₄;
X₅ is CR₅;
X₆ is selected from the group consisting of N and CR₆;
G is selected from the group consisting of

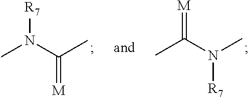

M is O;
Y₁ is CR₈R₉;
Y₂ is selected from the group consisting of CR₈R₉ and O;
Z₁ is CR₁₁;
Z₂ is CR₁₁;
Z₃ is CR₁₁;
Z₄ is CR₁₁;
R₁ is selected from the group consisting of hydrogen, halogen, cyano, and C₁-C₉ alkyl;
R₂ is selected from the group consisting of hydrogen and halogen;
R₃ is hydrogen;
R₄ is selected from the group consisting of B(OH)₂, C₂-C₄ alkenyl, C₃-C₆-cycloalkyl, C₁-C₄ halogenoalkyl, C₁-C₄-alkoxy substituted C₁-C₄ alkyl, —N(C₁-C₄ alkyl)₂, —N(C₁-C₄ alkyl)(C₁-C₄ alkoxy), —N(C₁-C₄ alkyl)(C₁-C₄ alkoxy substituted C₁-C₄ alkyl), —N(C₁-C₄ alkoxy substituted C₁-C₄ alkyl)₂, —N(C(O)C₁-C₄ alkyl)(C₁-C₄ alkyl), —N(C₁-C₄ alkyl)(C₃-C₆-cycloalkyl); a monocyclic heterocycle selected from the group of 4- to 7-membered heterocycloalkyl, each of the heterocycloalkyl in R₄ is optionally substituted with 1, 2 or 3 substituents independently selected from the group consisting of halogen, cyano, nitro, hydroxy, oxo, imino, 1-imino-1-oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl,
wherein the heterocycloalkyl rings in R₄ are optionally substituted with a spiro group, wherein said spiro group is 4- to 6-membered heterocycloalkyl containing 1, 2, or 3 heteroatoms independently selected from N, S or O, wherein said spiro group is optionally substituted with 1, 2 or 3 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, and wherein each C₃-C₆-cycloalkyl in R₄ may be optionally substituted with 1, 2 or 3 substituents independently selected from the group consisting of halogen, hydroxy, oxo, —NH₂, —NH(C₁-C₄ alkyl), —N(C₁-C₄ alkyl)₂, cyano, carboxy, carbamoyl, C₁-C₄ alkoxycarbonyl, —C(O)NH(C₁-C₄ alkyl), —C(O)N(C₁-C₄ alkyl)₂, C₁-C₄ halogenoalkyl, and C₁-C₄ alkoxy;
R₅ is selected from the group consisting of hydrogen, C₁-C₄ alkyl, and C₁-C₄ halogenoalkyl;
R₆ is hydrogen;
R₇ is selected from the group consisting of hydrogen and C₁-C₉ alkyl;
R₈ is, each time selected, independently selected from the group consisting of hydrogen;
R₉ is, each time selected, independently selected from the group consisting of hydrogen and fluoro;
R₁₁ is, each time selected, independently selected from the group consisting of hydrogen and halogen;
Q is selected from the group consisting of
(i) phenyl optionally substituted with 1, 2, 3, 4, or 5 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl, —C(O)NH₂, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl, —SO₂C₁-C₄ halogenoalkyl, and pentafluoro-sulfanyl,
(ii) pyrazole, pyridine, pyrimidine or pyrazine, wherein the carbons of the pyrazole, pyridine, pyrimidine or pyrazine are optionally substituted with 1, 2, 3, 4, or 5 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, benzyloxy, —C(O)R₁₇, —NH₂, —NH(C₁-C₄ alkyl), —N(C₁-C₄ alkyl)₂, —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, and any N in the heteroaryl, valency permitting, is optionally substituted with a substituent selected from the group consisting of hydrogen, C₁-C₄ alkyl, and C₃-C₆ cycloalkyl, and R₁₇ is, each time selected, independently selected from the group consisting of C₁-C₄ alkyl, C₃-C₆ cycloalkyl, C₁-C₄ halogenoalkyl, C₁-C₄ alkoxy, C₁-C₄ halogenoalkoxy, —OH, —NH₂, —NH(C₁-C₄ alkyl), —N(C₁-C₄ alkyl)₂, —NH(C₃-C₆ cycloalkyl), and —N(C₁-C₄ alkyl)(C₃-C₆-cycloalkyl);
or a stereoisomer or salt thereof.

### Aspect V of WO 2022/117783 A1:

The compound of formula (I′) according to any one of Aspects 1 to 4 of WO 2022/117783 A1, wherein
n is 0 or 1; when n is 1, Y₀ is CH₂ or C═O;
X₁ is selected from the group consisting of N and CR₁;
X₂ is selected from the group consisting of N and CR₂;
X₃ is selected from the group consisting of N and CR₃;
X₄ is CR₄;
X₅ is CR₅;
X₆ is selected from the group consisting of N and CR₆;
G is selected from the group consisting of

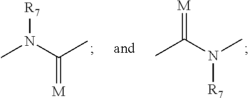

M is O;
Y₁ is CR₈R₉;
Y₂ is selected from the group consisting of CR₈R₉ and O;
Z₁ is CR₁₁;
Z₂ is CR₁₁;
Z₃ is CR₁₁;
Z₄ is CR₁₁;
R₁ is selected from the group consisting of hydrogen, halogen, cyano, and C₁-C₉ alkyl;
R₂ is selected from the group consisting of hydrogen and halogen;
R₃ is hydrogen;
R₄ is selected from the group consisting of B(OH)₂, C₂-C₄ alkenyl, C₃-C₆-cycloalkyl, C₁-C₄ halogenoalkyl, C₁-C₄-alkoxy substituted C₁-C₄ alkyl, —N(C₁-C₄ alkyl)₂, —N(C₁-C₄ alkyl)(C₁-C₄ alkoxy), —N(C₁-C₄ alkyl)(C₁-C₄ alkoxy substituted C₁-C₄ alkyl), —N(C₁-C₄ alkoxy substituted C₁-C₄ alkyl)₂, —N(C(O)C₁-C₄ alkyl)(C₁-C₄ alkyl), —N(C₁-C₄ alkyl)(C₃-C₆-cycloalkyl); a monocyclic heterocycle selected from the group of 4- to 7-membered heterocycloalkyl, each of the heterocycloalkyl in R₄ is optionally substituted with 1, 2 or 3 substituents independently selected from the group consisting of halogen, cyano, hydroxy, oxo, imino, 1-imino-1-oxo, C₁-C₄ halogenoalkyl, C₁-C₄ alkoxy, wherein the heterocycloalkyl rings in R₄ are optionally substituted with a spiro group, wherein said spiro group is 4- to 6-membered heterocycloalkyl containing 1, 2, or 3 heteroatoms independently selected from N or O, and wherein each C₃-C₆-cycloalkyl in R₄ may be optionally substituted with 1, 2 or 3 substituents independently selected from the group of halogen, oxo, C₁-C₄ halogenoalkyl, and C₁-C₄ alkoxy;
R₅ is selected from the group consisting of hydrogen, C₁-C₄ alkyl, and C₁-C₄ halogenoalkyl;
R₆ is hydrogen;
R₇ is selected from the group consisting of hydrogen and C₁-C₉ alkyl;
R₈ is, each time selected, independently selected from the group consisting of hydrogen and fluoro;
R₉ is, each time selected, independently selected from the group consisting of hydrogen and fluoro;
R₁₁ is, each time selected, independently selected from the group consisting of hydrogen and halogen;
Q is selected from the group consisting of
(i) phenyl optionally substituted with 1, 2, 3, 4, or 5 substituents independently selected from the group consisting of halogen, cyano, —C(O)NH₂, C₁-C₄ alkyl, C₁-C₄ halogenoalkyl, —NH₂, and pentafluoro-sulfanyl and
(ii) pyrazole, pyridine, pyrimidine or pyrazine, wherein the carbons of the pyrazole, pyridine, pyrimidine or pyrazine are optionally substituted with 1, 2, 3, 4, or 5 substituents independently selected from the group consisting of halogen, C₁-C₄ halogenoalkyl, C₁-C₄ alkoxy, —SC₁-C₄ alkyl,
or a stereoisomer or salt thereof

### Aspect VI of WO 2022/117783 A1:

The compound of formula (I′) according to any one of Aspects 1 to 5 of WO 2022/117783 A1,
wherein X₁ is CR₁; X₂ is CR₂; X₃ is CR₃; X₄ is CR₄; X₅ is CR₅; and X₆ is N;
or
wherein X₁ is N; X₂ is CR₂; X₃ is CR₃; X₄ is CR₄; X₅ is CR₅; and X₆ is N;
or
wherein X₁ is CR₁; X₂ is CR₂; X₃ is N; X₄ is CR₄; X₅ is CR₅; and X₆ is N;
or
wherein X₁ is CR₁; X₂ is N; X₃ is CR₃; X₄ is CR₄; X₅ is CR₅; and X₆ is N;
or
wherein X₁ is CR₁; X₂ is CR₂; X₃ is CR₃; X₄ is CR₄; X₅ is CR₅; and X₆ is CR₆;
or
wherein X₁ is N; X₂ is CR₂; X₃ is N; X₄ is CR₄; X₅ is CR₅; and X₆ is N;
or a stereoisomer or salt thereof.

### Aspect VII of WO 2022/117783 A1:

The compound of formula (I′) according to any one of Aspects 1 to 6 of WO 2022/117783 A1, wherein
Q is a 6- or 10 membered aryl optionally substituted with 1, 2, 3 or 4 substituents independently selected from the group consisting of halogen, cyano, nitro, hydroxy, C₁-C₄ alkyl, C₁-C₄ halogenoalkyl, C₁-C₄ alkoxy, C₃-C₆ cycloalkyl, —C(O)NH₂, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl, —SO₂C₁-C₄ halogenoalkyl, and pentafluoro-sulfanyl;
or a stereoisomer or salt thereof.

### Aspect VIII of WO 2022/117783 A1:

The compound of formula (I′) according to any one of Aspects 1 to 6 of WO 2022/117783 A1, wherein
Q is a 5- to 10-membered heteroaryl having 1 or 2 heteroatoms selected from the group O, S, and N and wherein the carbons of the heteroaryl are optionally substituted with 1, 2 or 3 substituents independently selected from the group consisting of halogen, cyano, nitro, —OH, C₁-C₄ alkyl, C₃-C₆ cycloalkyl, C₁-C₄ halogenoalkyl, C₁-C₄ alkoxy, —SC₁-C₄, C(O)R₁₇, —NH₂, —NH(C₁-C₄ alkyl), and —N(C₁-C₄ alkyl)₂ and any N in the heteroaryl is optionally substituted with a substituent selected from the group consisting of hydrogen, C₁-C₄ alkyl, and C₃-C₆ cycloalkyl;
or a stereoisomer or salt thereof.

### Aspect IX of WO 2022/117783 A1:

The compound of formula (I′) according to any one of Aspects 1 to 8 of WO 2022/117783 A1, wherein n is 1 and Y₀ is CH₂ or C═O; Y₁ is CR₈R₉, Y₂ is O; Z₁ is CR₁₁, Z₂ is CR₁₁, Z₃ is CR₁₁, Z₄ is CR₁₁,
or a stereoisomer or salt thereof.

### Aspect X of WO 2022/117783 A1:

The compound of formula (I′) according to any one of Aspects 1 to 9 of WO 2022/117783 A1, wherein
G is

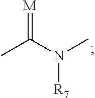

and
M is O;
or a stereoisomer or salt thereof.

### Aspect XI of WO 2022/117783 A1:

The compound of formula (I′) according to any one of Aspects 1 to 10 of WO 2022/117783 A1, wherein
G is

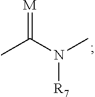

M is O; and
R₇ is hydrogen or C₁-C₉ alkyl, preferably R₇ is hydrogen or nonyl;
or a stereoisomer or salt thereof.

### Aspect XII of WO 2022/117783 A1:

The compound of formula (I′) according to any one of Aspects 1 to 11 of WO 2022/117783 A1, wherein
R₄ is selected from the group consisting of B(OH)₂, C₂-C₄ alkenyl, C₃-C₆-cycloalkyl, C₁-C₄ halogenoalkyl, C₁-C₄-alkoxy substituted C₁-C₄ alkyl, —N(C₁-C₄ alkyl)₂, —N(C₁-C₄ alkyl)(C₁-C₄ alkoxy), —N(C₁-C₄ alkyl)(C₁-C₄ alkoxy substituted C₁-C₄ alkyl), —N(C₁-C₄ alkoxy substituted C₁-C₄ alkyl)₂, —N(C(O)C₁-C₄ alkyl)(C₁-C₄ alkyl), —N(C₁-C₄ alkyl)(C₃-C₆-cycloalkyl); a monocyclic heterocycle selected from the group of 4- to 7-membered heterocycloalkyl, each of the heterocycloalkyl in R₄ is optionally substituted with 1, 2 or 3 substituents independently selected from the group consisting of halogen, cyano, hydroxy, oxo, imino, 1-imino-1-oxo, C₁-C₄ halogenoalkyl, C₁-C₄ alkoxy, wherein the heterocycloalkyl rings in R₄ are optionally substituted with a spiro group, wherein said spiro group is 4- to 6-membered heterocycloalkyl containing 1, 2, or 3 heteroatoms independently selected from N or O, and wherein each C₃-C₆-cycloalkyl in R₄ may be optionally substituted with 1, 2 or 3 substituents independently selected from the group of halogen, oxo, C₁-C₄ halogenoalkyl, and C₁-C₄ alkoxy,
or a stereoisomer or salt thereof.

### Aspect XIII of WO 2022/117783 A1:

The compound of formula (I′) according to any one of Aspects 1 to 12 of WO 2022/117783 A1, or a stereoisomer or salt thereof, having formula (Ia-5″),

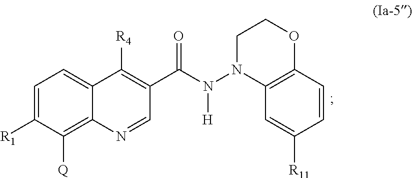

wherein R₁, R₄, R₁₁, and Q are as defined in any one of Aspects 1 to 12.

### Aspect XIV of WO 2022/117783 A1:

The compound of formula (Ia-5″) according to Aspect 13 of WO 2022/117783 A1, or a stereoisomer or salt thereof, wherein R₁ is hydrogen, halogen, cyano or C₁-C₉ alkyl.

### Aspect XV of WO 2022/117783 A1:

The compound of formula (Ia-5″) according to Aspect 13 or 14 of WO 2022/117783 A1, or a stereoisomer or salt thereof, wherein R₄ is selected from:

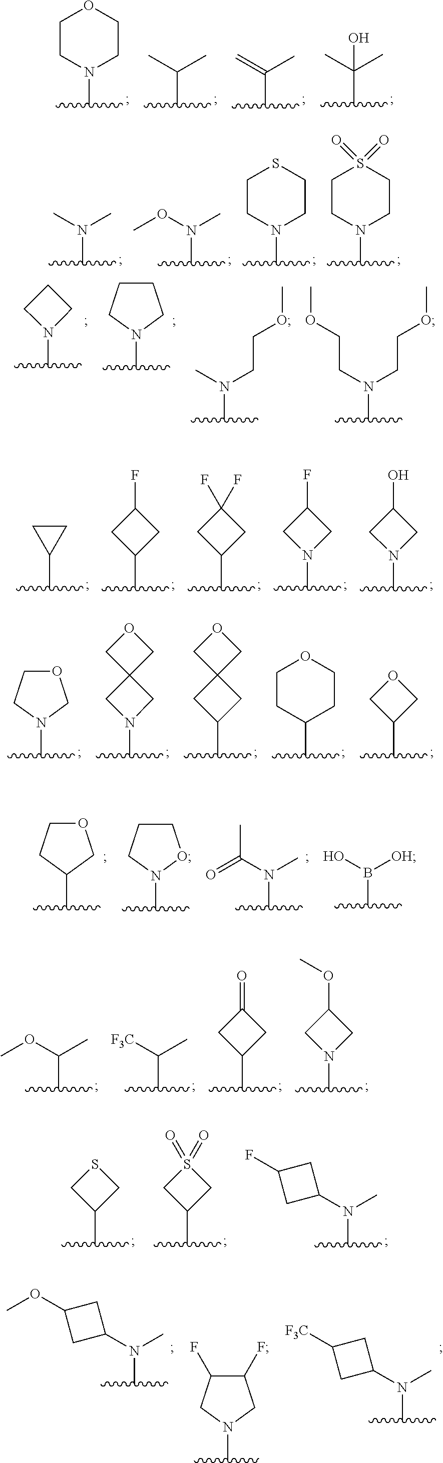

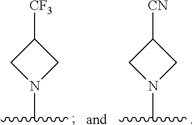 [SMILES: *N1CC(C(F)(F)F)C1 ; ";" ; "and" ; *N1CC(C#N)C1 ; "."]

### Aspect XVI of WO 2022/117783 A1:

The compound of formula (Ia-5″) according to any one of Aspects 13 to 15, or a stereoisomer or salt thereof, wherein R₁₁ is hydrogen or halogen.

### Aspect XVII of WO 2022/117783 A1:

The compound of formula (Ia-5″) according to any one of Aspects 13 to 16, or a stereoisomer or salt thereof, wherein
Q is a 6-membered aryl optionally substituted with 1, 2, 3, 4, or 5 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl, —C(O)NH₂, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl, —SO₂C₁-C₄ halogenoalkyl, and pentafluoro-sulfanyl, wherein the 6- or 10 membered aryl is optionally fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is, valency permitting, substituted with a substituent selected from the group consisting of hydrogen, C₁-C₄ alkyl, and C₃-C₆ cycloalkyl.

### Aspect XVIII of WO 2022/117783 A1:

The compound of formula (Ia-5″) according to any one of Aspects 13 to 17, or a stereoisomer or salt thereof, wherein Q is selected from:

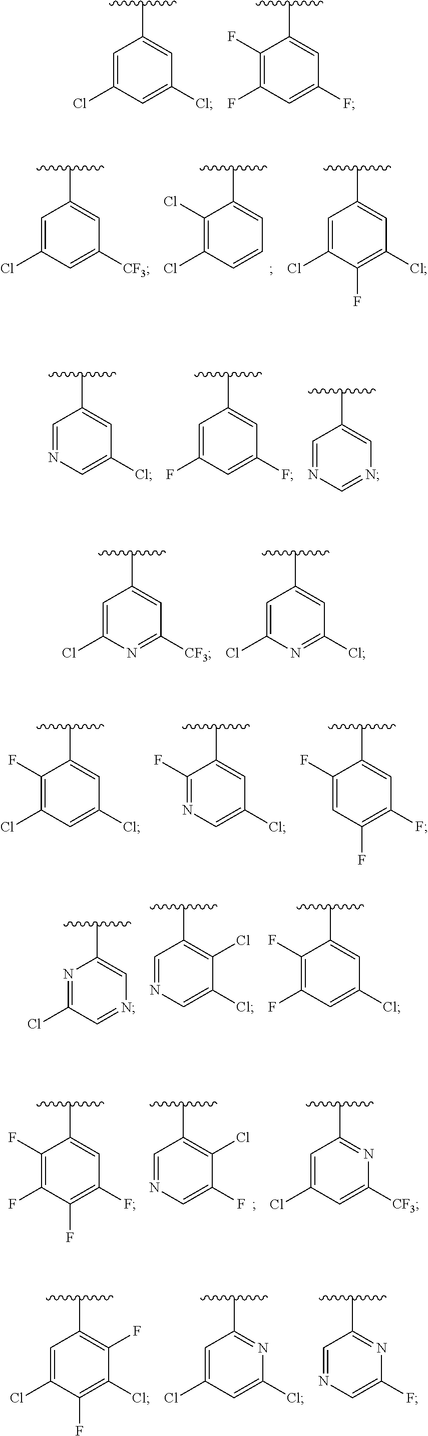

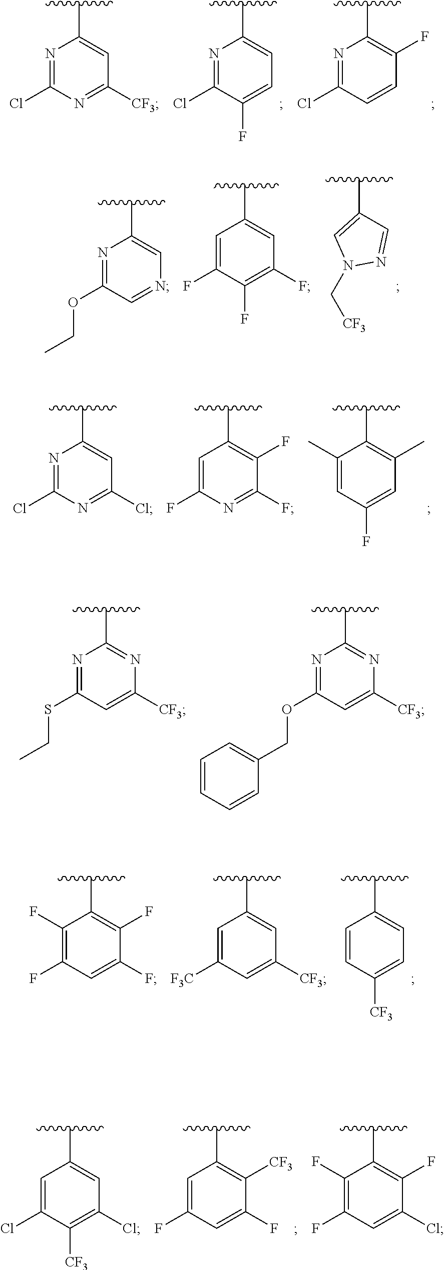

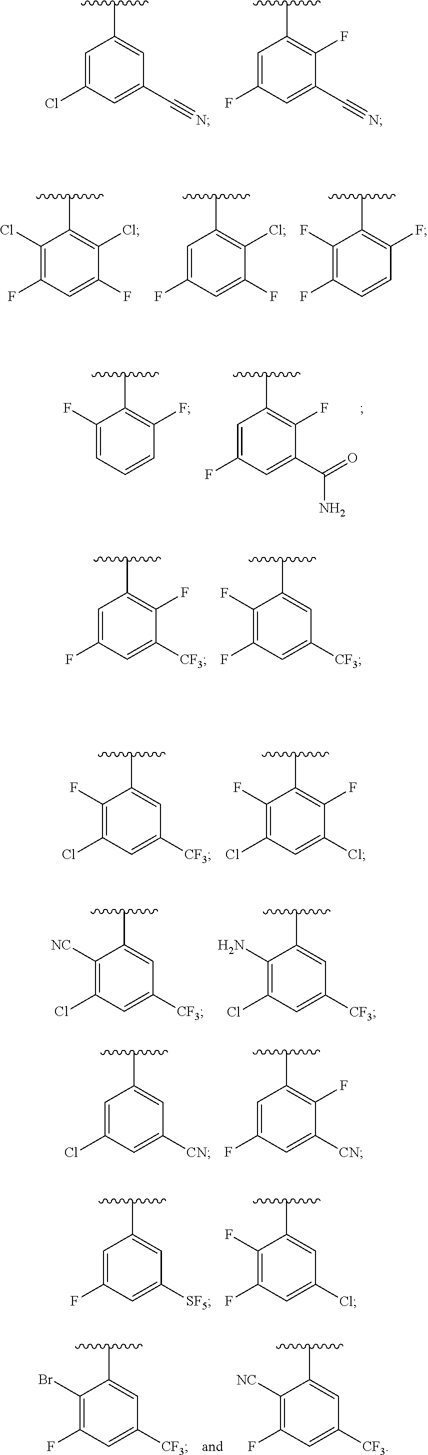

### Aspect XIX of WO 2022/117783 A1:

The compound of formula (I′) according to any one of Aspects 1 to 18 selected from the group consisting of:
N-[8-(3,5-dichlorophenyl)-4-(dimethylamino)-3-quinolyl]-2,3-dihydro-1,4-benzoxazine-4-carboxamide; (Example 1.1)
8-(3,5-dichlorophenyl)-N-(2,3-dihydro-1,4-benzoxazin-4-yl)-4-(dimethylamino)-1,7-naphthyridine-3-carboxamide: (Example 2.1)
8-(3,5-dichlorophenyl)-N-(3,4-dihydro-2H-quinolin-1-yl)-4-(dimethylamino)-1,7-naphthyridine-3-carboxamide; (Example 2.2)
8-(3,5-dichlorophenyl)-N-(2,3-dihydro-1,4-benzoxazin-4-yl)-4-morpholino-1,7-naphthyridine-3-carboxamide; (Example 2.3)
N-(2,3-dihydro-1,4-benzoxazin-4-yl)-4-morpholino-8-(2,3,5-trifluorophenyl)-1,7-naphthyridine-3-carboxamide; (Example 2.4)
N-(2,3-dihydro-1,4-benzoxazin-4-yl)-4-[methoxy(methyl)amino]-8-(2,3,5-trifluorophenyl)-1,7-naphthyridine-3-carboxamide; (Example 2.5)
8-[3-chloro-5-(trifluoromethyl)phenyl]-N-(2,3-dihydro-1,4-benzoxazin-4-yl)-4-morpholino-1,7-naphthyridine-3-carboxamide; (Example 2.6)
N-(2,3-dihydro-1,4-benzoxazin-4-yl)-4-morpholino-8-(2,3-dichlorophenyl)-1,7-naphthyridine-3-carboxamide; (Example 2.7)
8-(3,5-dichloro-4-fluoro-phenyl)-N-(2,3-dihydro-1,4-benzoxazin-4-yl)-4-morpholino-1,7-naphthyridine-3-carboxamide; (Example 2.8)
8-(5-chloro-3-pyridyl)-N-(2,3-dihydro-1,4-benzoxazin-4-yl)-4-morpholino-1,7-naphthyridine-3-carboxamide; (Example 2.9)
N-(2,3-dihydro-1,4-benzoxazin-4-yl)-4-thiomorpholino-8-(2,3,5-trifluorophenyl)-1,7-naphthyridine-3-carboxamide; (Example 2.10)
N-(2,3-dihydro-1,4-benzoxazin-4-yl)-4-(1,1-dioxo-1,4-thiazinan-4-yl)-8-(2,3,5-trifluorophenyl)-1,7-naphthyridine-3-carboxamide; (Example 2.11)
N-(2,3-dihydro-1,4-benzoxazin-4-yl)-4-morpholino-8-(3,4,5-trifluorophenyl)-1,7-naphthyridine-3-carboxamide; (Example 2.12)
8-(3,5-dichlorophenyl)-N-(2,3-dihydro-1,4-benzoxazin-4-yl)-4-(dimethylamino)-1,5-naphthyridine-3-carboxamide; (Example 3.1)
8-(2,3-dichlorophenyl)-N-(2,3-dihydro-1,4-benzoxazin-4-yl)-4-(dimethylamino)-1,5-naphthyridine-3-carboxamide; (Example 3.2)
8-(3,5-dichlorophenyl)-N-(2,3-dihydro-1,4-benzoxazin-4-yl)-4-morpholino-1,5-naphthyridine-3-carboxamide; (Example 3.3)
N-(2,3-dihydro-1,4-benzoxazin-4-yl)-4-morpholino-8-(2,3,5-trifluorophenyl)-1,5-naphthyridine-3-carboxamide; (Example 3.4)
5-(3,5-dichlorophenyl)-N-(2,3-dihydro-1,4-benzoxazin-4-yl)-1-(dimethylamino)naphthalene-2-carboxamide; (Example 4.1)
8-(3,5-dichlorophenyl)-N-(2,3-dihydro-4H-benzo[b][1,4]oxazin-4-yl)-4-(dimethylamino)quinoline-3-carboxamide; (Example 5.1)
8-(2,3-dichlorophenyl)-N-(2,3-dihydro-1,4-benzoxazin-4-yl)-4-morpholino-quinoline-3-carboxamide; (Example 5.2)
8-(3,5-dichlorophenyl)-N-(2,3-dihydro-1,4-benzoxazin-4-yl)-4-morpholino-quinoline-3-carboxamide; (Example 5.3)
N-(2,3-dihydro-1,4-benzoxazin-4-yl)-7-fluoro-4-morpholino-8-(2,3,5-trifluorophenyl)quinoline-3-carboxamide; (Example 5.4)
8-(5-chloro-3-pyridyl)-N-(2,3-dihydro-1,4-benzoxazin-4-yl)-4-morpholino-quinoline-3-carboxamide; (Example 5.5)
N-(2,3-dihydro-1,4-benzoxazin-4-yl)-4-morpholino-8-(2,3,5-trifluorophenyl)quinoline-3-carboxamide; (Example 5.6)
8-(3,5-dichlorophenyl)-N-(2,3-dihydro-1,4-benzoxazin-4-yl)-7-fluoro-4-morpholino-quinoline-3-carboxamide; (Example 5.7)
8-(3,5-difluorophenyl)-N-(2,3-dihydro-1,4-benzoxazin-4-yl)-7-fluoro-4-morpholino-quinoline-3-carboxamide; (Example 5.8)
N-(2,3-dihydro-1,4-benzoxazin-4-yl)-4-morpholino-8-pyrimidin-5-yl-quinoline-3-carboxamide; (Example 5.9)
N-(2,3-dihydro-1,4-benzoxazin-4-yl)-4-thiomorpholino-8-(2,3,5-trifluorophenyl)quinoline-3-carboxamide; (Example 5.10)
8-[2-chloro-6-(trifluoromethyl)-4-pyridyl]-N-(2,3-dihydro-1,4-benzoxazin-4-yl)-4-morpholino-quinoline-3-carboxamide; (Example 5.11)
8-(2,6-dichloro-4-pyridyl)-N-(2,3-dihydro-1,4-benzoxazin-4-yl)-4-morpholino-quinoline-3-carboxamide; (Example 5.12)
8-(3,5-dichloro-2-fluoro-phenyl)-N-(2,3-dihydro-1,4-benzoxazin-4-yl)-4-morpholino-quinoline-3-carboxamide; (Example 5.13)
8-(5-chloro-2-fluoro-3-pyridyl)-N-(2,3-dihydro-1,4-benzoxazin-4-yl)-4-morpholino-quinoline-3-carboxamide; (Example 5.14)
N-(2,3-dihydro-1,4-benzoxazin-4-yl)-4-(1,1-dioxo-1,4-thiazinan-4-yl)-8-(2,3,5-trifluorophenyl)quinoline-3-carboxamide; (Example 5.15)
N-(2,3-dihydro-1,4-benzoxazin-4-yl)-4-morpholino-8-(2,4,5-trifluorophenyl)quinoline-3-carboxamide; (Example 5.16)
8-(6-chloropyrazin-2-yl)-N-(2,3-dihydro-1,4-benzoxazin-4-yl)-4-morpholino-quinoline-3-carboxamide; (Example 5.17)
8-(4,5-dichloro-3-pyridyl)-N-(2,3-dihydro-1,4-benzoxazin-4-yl)-4-morpholino-quinoline-3-carboxamide; (Example 5.18)
8-(5-chloro-2,3-difluoro-phenyl)-N-(2,3-dihydro-1,4-benzoxazin-4-yl)-4-morpholino-quinoline-3-carboxamide; (Example 5.19)
N-(2,3-dihydro-1,4-benzoxazin-4-yl)-4-morpholino-8-(2,3,4,5-tetrafluorophenyl)quinoline-3-carboxamide; (Example 5.20)
8-(4-chloro-5-fluoro-3-pyridyl)-N-(2,3-dihydro-1,4-benzoxazin-4-yl)-4-morpholino-quinoline-3-carboxamide; (Example 5.21)
8-[4-chloro-6-(trifluoromethyl)-2-pyridyl]-N-(2,3-dihydro-1,4-benzoxazin-4-yl)-4-morpholino-quinoline-3-carboxamide; (Example 5.22)
8-(3,5-dichloro-2,4-difluoro-phenyl)-N-(2,3-dihydro-1,4-benzoxazin-4-yl)-4-morpholino-quinoline-3-carboxamide; (Example 5.23)
N-indolin-1-yl-4-morpholino-8-(2,3,5-trifluorophenyl)quinoline-3-carboxamide; (Example 5.24)
8-(4,6-dichloro-2-pyridyl)-N-(2,3-dihydro-1,4-benzoxazin-4-yl)-4-morpholino-quinoline-3-carboxamide; (Example 5.25)
N-(2,3-dihydro-1,4-benzoxazin-4-yl)-8-(6-fluoropyrazin-2-yl)-4-morpholino-quinoline-3-carboxamide; (Example 5.26)
8-[2-chloro-6-(trifluoromethyl)pyrimidin-4-yl]-N-(2,3-dihydro-1,4-benzoxazin-4-yl)-4-morpholino-quinoline-3-carboxamide; (Example 5.27)
8-(6-chloro-5-fluoro-2-pyridyl)-N-(2,3-dihydro-1,4-benzoxazin-4-yl)-4-morpholino-quinoline-3-carboxamide; (Example 5.28)
8-(6-chloro-3-fluoro-2-pyridyl)-N-(2,3-dihydro-1,4-benzoxazin-4-yl)-4-morpholino-quinoline-3-carboxamide; (Example 5.29)
N-(2,3-dihydro-1,4-benzoxazin-4-yl)-8-(6-ethoxypyrazin-2-yl)-4-morpholino-quinoline-3-carboxamide; (Example 5.30)
4-(azetidin-1-yl)-N-(2,3-dihydro-1,4-benzoxazin-4-yl)-8-(2,3,5-trifluorophenyl)quinoline-3-carboxamide; (Example 5.31)
N-(2,3-dihydro-1,4-benzoxazin-4-yl)-4-pyrrolidin-1-yl-8-(2,3,5-trifluorophenyl)quinoline-3-carboxamide; (Example 5.32)
N-(2,3-dihydro-1,4-benzoxazin-4-yl)-4-[2-methoxyethyl(methyl)amino]-8-(2,3,5-trifluorophenyl)quinoline-3-carboxamide; (Example 5.33)
4-[bis(2-methoxyethyl)amino]-N-(2,3-dihydro-1,4-benzoxazin-4-yl)-8-(2,3,5-trifluorophenyl)quinoline-3-carboxamide; (Example 5.34)
7-cyano-8-(3,5-dichlorophenyl)-N-(2,3-dihydro-1,4-benzoxazin-4-yl)-4-morpholino-quinoline-3-carboxamide; (Example 5.35)
4-cyclopropyl-N-(2,3-dihydro-1,4-benzoxazin-4-yl)-8-(2,3,5-trifluorophenyl)quinoline-3-carboxamide; (Example 5.36)
N-(2,3-dihydro-1,4-benzoxazin-4-yl)-4-(3-fluoroazetidin-1-yl)-8-(2,3,5-trifluorophenyl)quinoline-3-carboxamide; (Example 5.37)
N-(2,3-dihydro-1,4-benzoxazin-4-yl)-4-(3-hydroxyazetidin-1-yl)-8-(2,3,5-trifluorophenyl)quinoline-3-carboxamide; (Example 5.38)
N-(2,3-dihydro-1,4-benzoxazin-4-yl)-4-oxazolidin-3-yl-8-(2,3,5-trifluorophenyl)quinoline-3-carboxamide; (Example 5.39)
N-(2,3-dihydro-1,4-benzoxazin-4-yl)-4-(2-oxa-6-azaspiro[3.3]heptan-6-yl)-8-(2,3,5-trifluorophenyl)quinoline-3-carboxamide; (Example 5.40)
N-(2,3-dihydro-1,4-benzoxazin-4-yl)-7-fluoro-4-morpholino-8-(3,4,5-trifluorophenyl)quinoline-3-carboxamide; (Example 5.41)
N-(2,3-dihydro-1,4-benzoxazin-4-yl)-4-isoxazolidin-2-yl-8-(2,3,5-trifluorophenyl)quinoline-3-carboxamide; (Example 5.42)
N-(2,3-dihydro-1,4-benzoxazin-4-yl)-4-morpholino-8-[1-(2,2,2-trifluoroethyl)pyrazol-4-yl]quinoline-3-carboxamide; (Example 5.43)
8-(2,6-dichloropyrimidin-4-yl)-N-(2,3-dihydro-1,4-benzoxazin-4-yl)-4-morpholino-quinoline-3-carboxamide; (Example 5.44)
N-(2,3-dihydro-1,4-benzoxazin-4-yl)-4-tetrahydropyran-4-yl-8-(2,3,5-trifluorophenyl)quinoline-3-carboxamide; (Example 5.45)
4-[acetyl(methyl)amino]-N-(2,3-dihydro-1,4-benzoxazin-4-yl)-8-(2,3,5-trifluorophenyl)quinoline-3-carboxamide; (Example 5.46)
8-(3,5-dichloro-2-fluoro-phenyl)-N-(2,3-dihydro-1,4-benzoxazin-4-yl)-7-fluoro-4-morpholino-quinoline-3-carboxamide; (Example 5.47)
8-(3,5-dichloro-2,4-difluoro-phenyl)-N-(2,3-dihydro-1,4-benzoxazin-4-yl)-7-fluoro-4-morpholino-quinoline-3-carboxamide; (Example 5.48)
N-(2,3-dihydro-1,4-benzoxazin-4-yl)-4-morpholino-8-(2,3,6-trifluoro-4-pyridyl)quinoline-3-carboxamide; (Example 5.49)
N-(2,3-dihydro-1,4-benzoxazin-4-yl)-8-(4-fluoro-2,6-dimethyl-phenyl)-4-morpholino-quinoline-3-carboxamide; (Example 5.50)
N-(2,3-dihydro-1,4-benzoxazin-4-yl)-8-[4-ethylsulfanyl-6-(trifluoromethyl)pyrimidin-2-yl]-4-morpholino-quinoline-3-carboxamide; (Example 5.51)
8-[4-benzyloxy-6-(trifluoromethyl)pyrimidin-2-yl]-N-(2,3-dihydro-1,4-benzoxazin-4-yl)-4-morpholino-quinoline-3-carboxamide; (Example 5.52)
[3-(2,3-dihydro-1,4-benzoxazin-4-ylcarbamoyl)-8-(2,3,5-trifluorophenyl)-4-quinolyl]boronic acid; (Example 5.53)
8-(3,5-dichloro-2,4-difluoro-phenyl)-7-fluoro-N-indolin-1-yl-4-morpholino-quinoline-3-carboxamide; (Example 5.54)
N-(2,3-dihydro-1,4-benzoxazin-4-yl)-4-(1-methoxyethyl)-8-(2,3,5-trifluorophenyl)quinoline-3-carboxamide; (Example 5.55)
8-(3,5-dichloro-2,4-difluoro-phenyl)-N-(2,3-dihydro-1,4-benzoxazin-4-yl)-4-(dimethylamino)-7-fluoro-quinoline-3-carboxamide; (Example 5.56)
N-(2,3-dihydro-1,4-benzoxazin-4-yl)-4-morpholino-8-(2,3,5,6-tetrafluorophenyl)quinoline-3-carboxamide; (Example 5.57)
4-cyclopropyl-8-(3,5-dichloro-2,4-difluoro-phenyl)-N-(2,3-dihydro-1,4-benzoxazin-4-yl)-7-fluoro-quinoline-3-carboxamide; (Example 5.58)
8-[3,5-bis(trifluoromethyl)phenyl]-N-(2,3-dihydro-1,4-benzoxazin-4-yl)-7-fluoro-4-morpholino-quinoline-3-carboxamide; (Example 5.59)
8-(5-chloro-2,3-difluoro-phenyl)-N-(2,3-dihydro-1,4-benzoxazin-4-yl)-7-fluoro-4-morpholino-quinoline-3-carboxamide; (Example 5.60)
8-[3-chloro-5-(trifluoromethyl)phenyl]-N-(2,3-dihydro-1,4-benzoxazin-4-yl)-7-fluoro-4-morpholino-quinoline-3-carboxamide; (Example 5.61)
8-(3,5-dichloro-2,4-difluoro-phenyl)-N-(2,3-dihydro-1,4-benzoxazin-4-yl)-7-fluoro-4-[methoxy(methyl)amino]quinoline-3-carboxamide; (Example 5.62)
N-(2,3-dihydro-1,4-benzoxazin-4-yl)-4-morpholino-8-[4-(trifluoromethyl)phenyl]quinoline-3-carboxamide; (Example 5.63)
8-[3,5-dichloro-4-(trifluoromethyl)phenyl]-N-(2,3-dihydro-1,4-benzoxazin-4-yl)-4-morpholino-quinoline-3-carboxamide; (Example 5.64)
8-(3-chloro-2,5,6-trifluoro-phenyl)-N-(2,3-dihydro-1,4-benzoxazin-4-yl)-7-fluoro-4-morpholino-quinoline-3-carboxamide; (Example 5.65)
8-(3-chloro-5-cyano-phenyl)-N-(2,3-dihydro-1,4-benzoxazin-4-yl)-4-morpholino-quinoline-3-carboxamide; (Example 5.66)
8-(3-cyano-2,5-difluoro-phenyl)-N-(2,3-dihydro-1,4-benzoxazin-4-yl)-4-morpholino-quinoline-3-carboxamide; (Example 5.67)
N-(2,3-dihydro-1,4-benzoxazin-4-yl)-4-(2,2,2-trifluoro-1-methyl-ethyl)-8-(2,3,5-trifluorophenyl)quinoline-3-carboxamide; (Example 5.68)
8-(3-carbamoyl-2,5-difluoro-phenyl)-N-(2,3-dihydro-1,4-benzoxazin-4-yl)-4-morpholino-quinoline-3-carboxamide; (Example 5.69)
8-[2,5-difluoro-3-(trifluoromethyl)phenyl]-N-(2,3-dihydro-1,4-benzoxazin-4-yl)-4-morpholino-quinoline-3-carboxamide; (Example 5.70)
8-(2-chloro-3,5-difluoro-phenyl)-N-(2,3-dihydro-1,4-benzoxazin-4-yl)-7-fluoro-4-morpholino-quinoline-3-carboxamide; (Example 5.71)
8-[2,3-difluoro-5-(trifluoromethyl)phenyl]-N-(2,3-dihydro-1,4-benzoxazin-4-yl)-4-morpholino-quinoline-3-carboxamide; (Example 5.72)
8-[3-chloro-2-fluoro-5-(trifluoromethyl)phenyl]-N-(2,3-dihydro-1,4-benzoxazin-4-yl)-4-morpholino-quinoline-3-carboxamide; (Example 5.73)
8-(3,5-dichloro-2,6-difluoro-phenyl)-N-(2,3-dihydro-1,4-benzoxazin-4-yl)-7-fluoro-4-morpholino-quinoline-3-carboxamide; (Example 5.74)
8-[3-chloro-2-fluoro-5-(trifluoromethyl)phenyl]-N-(2,3-dihydro-1,4-benzoxazin-4-yl)-7-fluoro-4-morpholino-quinoline-3-carboxamide; (Example 5.75)
8-[3-chloro-2-cyano-5-(trifluoromethyl)phenyl]-N-(2,3-dihydro-1,4-benzoxazin-4-yl)-4-morpholino-quinoline-3-carboxamide; (Example 5.76)
8-[2-amino-3-chloro-5-(trifluoromethyl)phenyl]-N-(2,3-dihydro-1,4-benzoxazin-4-yl)-4-morpholino-quinoline-3-carboxamide; (Example 5.77)
N-(2,3-dihydro-1,4-benzoxazin-4-yl)-7-fluoro-4-(3-fluoroazetidin-1-yl)-8-(2,3,5-trifluorophenyl)quinoline-3-carboxamide; (Example 5.78)
8-(5-chloro-2,3-difluoro-phenyl)-N-(2,3-dihydro-1,4-benzoxazin-4-yl)-7-fluoro-4-(3-fluoroazetidin-1-yl)quinoline-3-carboxamide; (Example 5.79)
8-[2-bromo-3-fluoro-5-(trifluoromethyl)phenyl]-N-(2,3-dihydro-1,4-benzoxazin-4-yl)-4-morpholino-quinoline-3-carboxamide; (Example 5.80)
8-[2-cyano-3-fluoro-5-(trifluoromethyl)phenyl]-N-(2,3-dihydro-1,4-benzoxazin-4-yl)-4-morpholino-quinoline-3-carboxamide; (Example 5.81)
N-(2,3-dihydro-1,4-benzoxazin-4-yl)-7-fluoro-4-(3-oxocyclobutyl)-8-(2,3,5-trifluorophenyl)quinoline-3-carboxamide; (Example 5.82)
7-fluoro-N-(6-fluoro-2,3-dihydro-1,4-benzoxazin-4-yl)-4-morpholino-8-(2,3,5-trifluorophenyl)quinoline-3-carboxamide; (Example 5.83)
N-(2,3-dihydro-1,4-benzoxazin-4-yl)-7-fluoro-4-(3-methoxyazetidin-1-yl)-8-(2,3,5-trifluorophenyl)quinoline-3-carboxamide; (Example 5.84)
N-(2,3-dihydro-1,4-benzoxazin-4-yl)-7-fluoro-4-(thietan-3-yl)-8-(2,3,5-trifluorophenyl)quinoline-3-carboxamide; (Example 5.85)
N-(2,3-dihydro-1,4-benzoxazin-4-yl)-4-(1,1-dioxothietan-3-yl)-7-fluoro-8-(2,3,5-trifluorophenyl)quinoline-3-carboxamide; (Example 5.86)
N-(2,3-dihydro-1,4-benzoxazin-4-yl)-7-fluoro-4-[(3-fluorocyclobutyl)-methyl-amino]-8-(2,3,5-trifluorophenyl)quinoline-3-carboxamide; (Example 5.87)
N-(2,3-dihydro-1,4-benzoxazin-4-yl)-7-fluoro-4-[(3-methoxycyclobutyl)-methyl-amino]-8-(2,3,5-trifluorophenyl)quinoline-3-carboxamide; (Example 5.88)
4-(3,4-difluoropyrrolidin-1-yl)-N-(2,3-dihydro-1,4-benzoxazin-4-yl)-7-fluoro-8-(2,3,5-trifluorophenyl)quinoline-3-carboxamide; (Example 5.89)
N-(2,3-dihydro-1,4-benzoxazin-4-yl)-7-fluoro-4-[methyl-[3-(trifluoromethyl)cyclobutyl]amino]-8-(2,3,5-trifluorophenyl)quinoline-3-carboxamide; (Example 5.90)
N-(2,3-dihydro-1,4-benzoxazin-4-yl)-7-fluoro-4-[3-(trifluoromethyl)azetidin-1-yl]-8-(2,3,5-trifluorophenyl)quinoline-3-carboxamide; (Example 5.91)
4-[(3R,4R)-3,4-difluoropyrrolidin-1-yl]-N-(2,3-dihydro-1,4-benzoxazin-4-yl)-7-fluoro-8-(2,3,5-trifluorophenyl)quinoline-3-carboxamide; (Example 5.92)
4-(3-cyanoazetidin-1-yl)-N-(2,3-dihydro-1,4-benzoxazin-4-yl)-7-fluoro-8-(2,3,5-trifluorophenyl)quinoline-3-carboxamide; (Example 5.93)
8-(2,3-difluoro-5-(trifluoromethyl)phenyl)-N-(2,3-dihydro-4H-benzo[b][1,4]oxazin-4-yl)-7-fluoro-4-morpholinoquinoline-3-carboxamide; (Example 5.94)
4-(3,3-difluorocyclobutyl)-N-(2,3-dihydro-4H-benzo[b][1,4]oxazin-4-yl)-7-fluoro-8-(2,3,5-trifluorophenyl)quinoline-3-carboxamide; (Example 5.95)
8-(2,3-difluoro-5-(trifluoromethyl)phenyl)-N-(2,3-dihydro-4H-benzo[b][1,4]oxazin-4-yl)-7-fluoro-4-(3-fluoroazetidin-1-yl)quinoline-3-carboxamide; Example 5.96)
7-fluoro-N-(6-fluoro-2,3-dihydro-4H-benzo[b][1,4]oxazin-4-yl)-4-(tetrahydrofuran-3-yl)-8-(2,3,5-trifluorophenyl)quinoline-3-carboxamide; (Example 5.97)
7-fluoro-N-(6-fluoro-2,3-dihydro-4H-benzo[b][1,4]oxazin-4-yl)-4-(3-fluoroazetidin-1-yl)-8-(2,3,5-trifluorophenyl)quinoline-3-carboxamide; (Example 5.98)
7-fluoro-N-(6-fluoro-2,3-dihydro-4H-benzo[b][1,4]oxazin-4-yl)-4-(prop-1-en-2-yl)-8-(2,3,5-trifluorophenyl)quinoline-3-carboxamide; (Example 5.99)
N-(2,3-dihydro-4H-benzo[b][1,4]oxazin-4-yl)-7-fluoro-4-((1s, 3s)-1-imino-1-oxido-thietan-3-yl)-8-(2,3,5-trifluorophenyl)quinoline-3-carboxamide; (Example 5.100)
N-(2,3-dihydro-4H-benzo[b][1,4]oxazin-4-yl)-7-fluoro-4-(3-fluorocyclobutyl)-8-(2,3,5-trifluorophenyl)quinoline-3-carboxamide; (Example 5.101)
N-(2,3-dihydro-4H-benzo[b][1,4]oxazin-4-yl)-7-fluoro-8-(3-fluoro-5-(pentafluoro-sulfanyl)phenyl)-4-(tetrahydrofuran-3-yl)quinoline-3-carboxamide; (Example 5.102)
7-fluoro-N-(4-fluoro-3,4-dihydroquinolin-1(2H)-yl)-4-morpholino-8-(2,3,5-trifluorophenyl)quinoline-3-carboxamide; (Example 5.103)
N-(2,3-dihydro-4H-benzo[b][1,4]oxazin-4-yl)-4-morpholino-7-nonyl-8-(2,3,5-trifluorophenyl)quinoline-3-carboxamide; (Example 5.104)
N-(2,3-dihydro-4H-benzo[b][1,4]oxazin-4-yl)-6,7-difluoro-4-morpholino-8-(2,3,5-trifluorophenyl)quinoline-3-carboxamide; (Example 5.105)
4-(1,1-dioxidothietan-3-yl)-7-fluoro-N-(6-fluoro-2,3-dihydro-4H-benzo[b][1,4]oxazin-4-yl)-8-(2,3,5-trifluorophenyl)quinoline-3-carboxamide; (Example 5.106)
8-(2,3-difluoro-5-(trifluoromethyl)phenyl)-N-(2,3-dihydro-4H-benzo[b][1,4]oxazin-4-yl)-4-(1,1-dioxidothietan-3-yl)-7-fluoroquinoline-3-carboxamide; (Example 5.107)
8-(3,5-difluoro-2-(trifluoromethyl)phenyl)-7-fluoro-4-morpholino-N-(2,3,4a, 8a-tetrahydro-4H-benzo[b][1,4]oxazin-4-yl)quinoline-3-carboxamide; (Example 5.108)
4-morpholino-N-(3-oxo-2,3-dihydro-4H-benzo[b][1,4]oxazin-4-yl)-8-(2,3,5-trifluorophenyl)quinoline-3-carboxamide; (Example 5.109)
N-(2,3-dihydro-4H-benzo[b][1,4]oxazin-4-yl)-4-morpholino-N-nonyl-8-(2,3,5-trifluorophenyl)quinoline-3-carboxamide; (Example 5.110)
4-(3,5-dichlorophenyl)-N-(2,3-dihydro-1,4-benzoxazin-4-yl)-8-morpholino-pyrido[3,2-d]pyrimidine-7-carboxamide; (Example 6.1)
8-(3,5-dichlorophenyl)-N-(2,3-dihydro-1,4-benzoxazin-4-yl)-4-morpholino-1,6-naphthyridine-3-carboxamide; (Example 7.1)
N-(2,3-dihydro-1,4-benzoxazin-4-yl)-2-methyl-4-morpholino-8-(2,3,5-trifluorophenyl)quinoline-3-carboxamide; (Example 8.1)
N-(2,3-dihydro-1,4-benzoxazin-4-yl)-4-morpholino-2-(trifluoromethyl)-8-(2,3,5-trifluorophenyl)quinoline-3-carboxamide; (Example 8.2)
   or a stereoisomer or salt of any of the foregoing compounds.

In one embodiment, E1_7, E2_2, n and R1 are as defined in WO 2022/117783 A1. In one embodiment, E1_7, E2_2, n and R1 are as defined in the above-described aspects of WO 2022/117783 A1. In one embodiment, the compound of general formula (I) is a compound of formula I.2 wherein E1_7, E2_2 and R1 are as defined in the above-described aspects of WO 2022/117783 A1 and n is 0.

Long-Term Prevention and/or Treatment of a Disease

The claimed invention concerns compounds for use in the long-term prevention and/or treatment of a disease.

In one embodiment, the compound for use in the long-term prevention and/or treatment of a disease has a half-life of at least 20 hours, preferably at least 30 hours, more preferably at least 35 hours, and most preferably at least 40 hours. Compounds for use according to the invention with such a half-life are particularly suitable in the long-term prevention and/or treatment of a disease, in particular in the long-term prevention and/or treatment of an helminthic infection in an animal, preferably in a cat or a dog. Methods to determine the half-life of a given compound are part of the common general knowledge of the skilled person.

In one embodiment, the compound for use in the long-term prevention and/or treatment of a disease has a plasma clearance of less than 2 L/h/kg, preferably less than 1.5 L/h/kg and most preferably less than 1 L/h/kg. Compounds for use according to the invention with such a plasma clearance are particularly suitable in the long-term prevention and/or treatment of a disease, in particular in the long-term prevention and/or treatment of an helminthic infection in an animal, preferably in a cat or a dog. Methods to determine the plasma-clearance of a given compound are part of the common general knowledge of the skilled person.

In one embodiment, the term “long-term” means a period of time of at least one month, preferably at least two months, even more preferably at least three months and most preferably at least six months. It was surprising that the compounds for use according to the claimed invention have this long-term effect of preventing and/or treating a disease, in particular an helminthic infection in an animal, preferably in a cat or a dog.

In one embodiment, the term “long-term” means that the prevention and/or treatment of a disease is held for a period of time of at least one month, preferably at least two months, even more preferably at least three months and most preferably at least six months with an efficacy of at least 40%, preferably at least 50%, more preferably at least 60%, even more preferably at least 70%, still more preferably at least 80% and most preferably at least 90%.

The term “efficacy” as used herein means the power to produce any effect on the prevention and/or treatment a disease. It belongs to the skilled person's common general knowledge to choose and apply a suitable standard technique to determine the efficacy in preventing and/or treating a given disease when using a compound according to the claimed invention. For example, in case the disease is a helminthic infection, the skilled person could determine the prevention and/or treatment efficacies of the used compound as described in Assay 2 and Assay 3 of the Experimental Section below.

In one embodiment, the term “for use in the long-term prevention and/or treatment of a disease” means that the compound prevents and/or treats the onset of a disease occurring or arising at least one month after its administration, preferably at least one and a half months after its administration, more preferably at least two months after its administration, even more preferably at least three months after its administration and most preferably at least six months after its administration. The compounds of the claimed invention are particularly effective to prevent and/or treat the onset of a disease, in particular the onset of an helmintic infection in an animal, preferably in a cat or a dog, when the disease occurs in these time intervals after administration of the compound.

The terms “treating” or “preventing” of a disease or disorder includes preventing or protecting against the disease or disorder (that is, causing the clinical symptoms not to develop), inhibiting the disease or disorder (i.e., arresting or suppressing the development of clinical symptoms), and/or relieving the disease or disorder (i.e., causing the regression of clinical symptoms). It is not always possible to distinguish between “preventing” and “suppressing” a disease or disorder since the ultimate inductive event or events may be unknown or latent. Accordingly, the term “prophylaxis” can also be understood to constitute a type of “treatment” that encompasses both “preventing” and “suppressing”. The term “treatment” can thus include “prophylaxis”.

In one embodiment, the compound for use according to the invention (i.e. the active compound) is formulated in a form that enables a sustained release of the compound when administered to a subject. This has the advantage that the long-term properties of the compounds for use according to the invention can be further modulated and/or enhanced so that a particularly efficient long-term effect regarding the prevention and/or treatment of a disease is achieved, in particular in the long-term prevention and/or treatment of an helminthic infection in an animal, preferably in a cat or a dog.

The term “enables a sustained release” means in particular “ensures a sustained release”.

The term “sustained release” is known by the skilled person and refers to the delivery of a compound at a programmed rate that leads to a compound delivery for a prolonged period of time compared the delivery of a compound formulated in a conventional rapid-release formulation, in particular a conventional rapid-release tablet or suspension administered orally. In one embodiment, the “sustained release” is effected by a subcutaneous injection that has sustained release relative to the PO administration. Such sustained release is derived from the inherent solubility characteristics of the molecule and the route of administration. A conventional rapid-release tablet or suspension is one that is not formulated to release the compound in a modified way. Rapid-release tablets or suspensions are in particular those which, according to the USP release method using apparatus 2 (paddle), have a Q value (30 minutes) of 75%.

The skilled person knows and/or can readily identify by standard techniques suitable formulations that allow a sustained release of the administered compound. For example, micro-encapsulation could be used. Another strategy could be to use compound-polymer conjugates (for example hydrogels) to ensure a sustained-release of the active compound.

### Disease

The claimed invention concerns compounds for use in the long-term prevention and/or treatment of a disease.

In one embodiment, the disease is a helminithic infection. The compounds according to the claimed invention are particularly efficient in the long-term prevention (i.e. prophylaxis) and/or treatment of helminthic infections, preferably gastro-intestinal and extra-intestinal helminthic infections. By using the compounds of the present invention for long-term prevention and/or treatment of a disease, in particular a helminthic infection, a reduction or prevention of illness, cases of deaths and performance reductions are achieved in the subjects in need thereof such as animals (in the case of meat, milk, wool, hides, eggs, honey and the like). This ensures economical and simpler animal keeping and better animal well-being.

Helminths pathogenic for humans or animals include, for example, acanthocephala, nematodes, pentastoma and platyhelmintha (e.g. monogenea, cestodes and trematodes).

Exemplary helminths include, but are not limited to:
Monogenea:
for example Dactylogyrus spp., Gyrodactylus spp., Microbothrium spp., Polystoma spp., Troglocephalus spp.
Cestodes:
from the order of the Pseudophyllidea, for example: Bothridium spp., Diphyllobothrium spp., Diplogonoporus spp., Ichthyobothrium spp., Ligula spp., Schistocephalus spp., Spirometra spp. from the order of the Cyclophyllida, for example: Andyra spp., Anoplocephala spp., Avitellina spp., Bertiella spp., Cittotaenia spp., Davainea spp., Diorchis spp., Diplopylidium spp., Dipylidium spp., Echinococcus spp., Echinocotyle spp., Echinolepis spp., Hydatigera spp., Hymenolepis spp., Joyeuxiella spp., Mesocestoides spp., Moniezia spp., Paranoplocephala spp., Raillietina spp., Stilesia spp., Taenia spp., Thysaniezia spp., Thysanosoma spp.
Trematodes:
from the class of the Digenea, for example: Austrobilharzia spp., Brachylaima spp., Calicophoron spp., Catatropis spp., Clonorchis spp. Collyriclum spp., Cotylophoron spp., Cyclocoelum spp., Dicrocoelium spp., Diplostomum spp., Echinochasmus spp., Echinoparyphium spp., Echinostoma spp., Eurytrema spp., Fasciola spp., Fasciolides spp., Fasciolopsis spp., Fischoederius spp., Gastrothylacus spp., Gigantobilharzia spp., Gigantocotyle spp., Heterophyes spp., Hypoderaeum spp., Leucochloridium spp., Metagonimus spp., Metorchis spp., Nanophyetus spp., Notocotylus spp., Opisthorchis spp., Ornithobilharzia spp., Paragonimus spp., Paramphistomum spp., Plagiorchis spp., Posthodiplostomum spp., Prosthogonimus spp., Schistosoma spp., Trichobilharzia spp., Troglotrema spp., Typhlocoelum spp.
Nematodes:
from the order of the Trichinellida, for example: Capillaria spp., Eucoleus spp., Paracapillaria spp., Trichinella spp., Trichomosoides spp., Trichuris spp. from the order of the Tylenchida, for example: Micronema spp., Parastrongyloides spp., Strongyloides spp.
from the order of the Rhabditina, for example: Aelurostrongylus spp., Amidostomum spp., Ancylostoma spp., Angiostrongylus spp., Bronchonema spp., Bunostomum spp., Chabertia spp., Cooperia spp., Cooperioides spp., Crenosoma spp., Cyathostomum spp., Cyclococercus spp., Cyclodontostomum spp., Cylicocyclus spp., Cylicostephanus spp., Cylindropharynx spp., Cystocaulus spp., Dictyocaulus spp., Elaphostrongylus spp., Filaroides spp., Globocephalus spp., Graphidium spp., Gyalocephalus spp., Haemonchus spp., Heligmosomoides spp., Hyostrongylus spp., Marshallagia spp., Metastrongylus spp., Muellerius spp., Necator spp., Nematodirus spp., Neostrongylus spp., Nippostrongylus spp., Obeliscoides spp., Oesophagodontus spp., Oesophagostomum spp., Ollulanus spp.; Ornithostrongylus spp., Oslerus spp., Ostertagia spp., Paracooperia spp., Paracrenosoma spp., Parafilaroides spp., Parelaphostrongylus spp., Pneumocaulus spp., Pneumostrongylus spp., Poteriostomum spp., Protostrongylus spp., Spicocaulus spp., Stephanurus spp., Strongylus spp., Syngamus spp., Teladorsagia spp., Trichonema spp., Trichostrongylus spp., Triodontophorus spp., Troglostrongylus spp., Uncinaria spp.
from the order of the Spirurida, for example: Acanthocheilonema spp., Anisakis spp., Ascaridia spp.; Ascaris spp., Ascarops spp., Aspiculuris spp., Baylisascaris spp., Brugia spp., Cercopithifilaria spp., Crassicauda spp., Dipetalonema spp., Dirofilaria spp., Dracunculus spp.; Draschia spp., Enterobius spp., Filaria spp., Gnathostoma spp., Gongylonema spp., Habronema spp., Heterakis spp.; Litomosoides spp., Loa spp., Onchocerca spp., Oxyuris spp., Parabronema spp., Parafilaria spp., Parascaris spp., Passalurus spp., Physaloptera spp., Probstmayria spp., Pseudofilaria spp., Setaria spp., Skjrabinema spp., Spirocerca spp., Stephanofilaria spp., Strongyluris spp., Syphacia spp., Thelazia spp., Toxascaris spp., Toxocara spp., Wuchereria spp.
Acantocephala:
from the order of the Oligacanthorhynchida, for example: Macracanthorhynchus spp., Prosthenorchis spp.; from the order of the Moniliformida, for example: Moniliformis spp.
from the order of the Polymorphida, for example: Filicollis spp.; from the order of the Echinorhynchida, for example: Acanthocephalus spp., Echinorhynchus spp., Leptorhynchoides spp.
Pentastoma:
from the order of the Porocephalida, for example: Linguatula spp.

Subjects, in particular animals, in need of treatment or prevention include those already with the disorder or disease condition as well as those in which the disorder or disease condition is to be prevented.

### Pharmaceutical Composition

The claimed invention also concerns a pharmaceutical composition for use in the long-term prevention and/or treatment of a disease comprising at least one compound according the claimed invention and at least one further ingredient.

In one embodiment, the pharmaceutical composition comprises at least one compound of general formula (I) of the present invention and at least one or more further active ingredients, in particular for the treatment and/or prevention of an endo- and/or ectoparasiticidal infection.

The term “active ingredient” as used herein refers to an ingredient that has a pharmaceutical effect, in particular a pharmaceutical effect related to the prevention and/or treatment of an endo- and/or ectoparasiticidal infection.

The term “endoparasite” as used herein is known to the skilled person and refers in particular to helminths.

The term “ectoparasite” as used herein is known to the skilled person and refers in particular to arthropods, particularly insects or acarids.

In one embodiment, the present invention covers a pharmaceutical composition for use in the long-term treatment and/or prevention of a disease comprising:
one or more first active ingredients, in particular compounds of general formula (I) as defined supra, and
ne or more further active ingredients, in particular one or more endo- and/or ectoparasiticides.

Such pharmaceutical compositions have the advantage that they are particularly efficient to prevent and/or treat endo- and ectoparasitic infections simulataneously.

Further active ingredients in form of endo- and/or ectoparasiticides are known by the skilled person and are described, for example, in the Pesticide Manual (“The Pesticide Manual” 16th Ed., British Crop Protection Council 2012) or can be searched in the internet (for example, http://www.alanwood.net/pesticides).

Examples of ectoparasiticides and/or endoparasiticides are insecticides, acaricides and nematicides, and include in particular:
(1) Acetylcholinesterase (AChE) inhibitors, such as, for example, carbamates, for example alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel blockers, such as, for example, cyclodiene-organochlorines, for example chlordane and endosulfan or phenylpyrazoles (fiproles), for example ethiprole and fipronil.
(3) Sodium channel modulators, such as, for example, pyrethroids, e.g. acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomer], deltamethrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans-isomer], prallethrin, pyrethrins (pyrethrum), resmethrin, silafluofen, tefluthrin, tetramethrin, tetramethrin [(1R)-isomer)], tralomethrin and transfluthrin or DDT or methoxychlor.
(4) Nicotinic acetylcholine receptor (nAChR) competitive modulators, such as, for example, neonicotinoids, e.g. acetamiprid, clothianidin, dinotefuran, imidacloprid, nitenpyram, thiacloprid and thiamethoxam or nicotine or sulfoxaflor or flupyradifurone.
(5) Nicotinic acetylcholine receptor (nAChR) allosteric modulators, such as, for example, spinosyns, e.g. spinetoram and spinosad.
(6) Glutamate-gated chloride channel (GluCl) allosteric modulators, such as, for example, avermectins/milbemycins, for example abamectin, emamectin benzoate, lepimectin and milbemectin.
(7) Juvenile hormone mimics, such as, for example, juvenile hormone analogues, e.g. hydroprene, kinoprene and methoprene or fenoxycarb or pyriproxyfen.
(9) Modulators of Chordotonal Organs, such as, for example pymetrozine or flonicamid.
(10) Mite growth inhibitors, such as, for example clofentezine, hexythiazox and diflovidazin or etoxazole.
(12) Inhibitors of mitochondrial ATP synthase, such as, ATP disruptors such as, for example, diafenthiuron or organotin compounds, for example azocyclotin, cyhexatin and fenbutatin oxide or propargite or tetradifon.
(13) Uncouplers of oxidative phosphorylation via disruption of the proton gradient, such as, for example, chlorfenapyr, DNOC and sulfluramid.
(14) Nicotinic acetylcholine receptor channel blockers, such as, for example, bensultap, cartap hydrochloride, thiocylam, and thiosultap-sodium.
(15) Inhibitors of chitin biosynthesis, type 0, such as, for example, bistrifluron, chlorfluazuron, diflubenzuron, flucycloxuron, flufenoxuron, hexaflumuron, lufenuron, novaluron, noviflumuron, teflubenzuron and triflumuron.
(16) Inhibitors of chitin biosynthesis, type 1, for example buprofezin.
(17) Moulting disruptor (in particular for Diptera, i.e. dipterans), such as, for example, cyromazine.
(18) Ecdysone receptor agonists, such as, for example, chromafenozide, halofenozide, methoxyfenozide and tebufenozide.
(19) Octopamine receptor agonists, such as, for example, amitraz.
(20) Mitochondrial complex III electron transport inhibitors, such as, for example, hydramethylnone or acequinocyl or fluacrypyrim.
(21) Mitochondrial complex I electron transport inhibitors, such as, for example from the group of the METI acaricides, e.g. fenazaquin, fenpyroximate, pyrimidifen, pyridaben, tebufenpyrad and tolfenpyrad or rotenone (Derris).
(22) Voltage-dependent sodium channel blockers, such as, for example indoxacarb or metaflumizone.
(23) Inhibitors of acetyl CoA carboxylase, such as, for example, tetronic and tetramic acid derivatives, e.g. spirodiclofen, spiromesifen and spirotetramat.
(25) Mitochondrial complex II electron transport inhibitors, such as, for example, beta-ketonitrile derivatives, e.g. cyenopyrafen and cyflumetofen and carboxanilides, such as, for example, pyflubumide.
(28) Ryanodine receptor modulators, such as, for example, diamides, e.g. chlorantraniliprole, cyantraniliprole and flubendiamide,
further active ingredients such as, for example, Afidopyropen, Afoxolaner, Azadirachtin, Benclothiaz, Benzoximate, Bifenazate, Broflanilide, Bromopropylate, Chinomethionat, Chloroprallethrin, Cryolite, Cyclaniliprole, Cycloxaprid, Cyhalodiamide, Dicloromezotiaz, Dicofol, epsilon-Metofluthrin, epsilon-Momfluthrin, Flometoquin, Fluazaindolizine, Fluensulfone, Flufenerim, Flufenoxystrobin, Flufiprole, Fluhexafon, Fluopyram, Fluralaner, Fluxametamide, Fufenozide, Guadipyr, Heptafluthrin, Imidaclothiz, Iprodione, kappa-Bifenthrin, kappa-Tefluthrin, Lotilaner, Meperfluthrin, Paichongding, Pyridalyl, Pyrifluquinazon, Pyriminostrobin, Spirobudiclofen, Tetramethylfluthrin, Tetraniliprole, Tetrachlorantraniliprole, Tioxazafen, Thiofluoximate, Triflumezopyrim and iodomethane; furthermore preparations based on Bacillus firmus (1-1582, BioNeem, Votivo), and also the following compounds: 1-{2-fluoro-4-methyl-5-[(2,2,2-trifluoroethyl)sulphinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457) (CAS 637360-23-7), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethyl carbonate (known from EP2647626) (CAS 1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoro-propan-2-one (known from WO2013/144213) (CAS 1461743-15-6), N—[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)-benzamide, 4-[5-(3,5-dichlorophenyl)-4,5-dihydro-5-(trifluoromethyl)-3-isoxazolyl]-2-methyl-N-(trans-1-oxido-3-thietanyl)-benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulfinyl]-propanamide, (+)—N-[3-chloro-1-(3-pyridinyl)-1H-pyrazol-4-yl]-N-ethyl-3-[(3,3,3-trifluoropropyl) sulfinyl]-propanamide and (−)—N-[3-chloro-1-(3-pyridinyl)-1H-pyrazol-4-yl]-N-ethyl-3-[(3,3,3-trifluoropropyl)sulfinyl]-propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937 A) (CAS 1105672-77-2), 3-bromo-N-[4-chloro-2-methyl-6-[(methylamino) thioxomethyl]phenyl]-1-(3-chloro-2-pyridinyl)-1H-pyrazole-5-carboxamide, (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-Pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)-pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]-hydrazinecarboxamide (known from CN 101715774 A) (CAS 1232543-85-9); 3-(2,2-dichloroethenyl)-2,2-dimethyl-4-(1H-benzimidazol-2-yl)phenyl-cyclopropanecarboxylic acid ester (known from CN 103524422 A) (CAS 1542271-46-4); (4aS)-7-chloro-2,5-dihydro-2-[[(methoxycarbonyl)[4-[(trifluoromethyl)thio]phenyl]amino]carbonyl]-indeno[1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-, 1-[N-[4-[1-[4-(1,1,2,2,2-pentafluoroethoxy)phenyl]-1H-1,2, 4-triazol-3-yl]phenyl]carbamate]-α-L-mannopyranose (known from US 2014/0275503 A1) (CAS 1181213-14-8); 8-(2-cyclopropylmethoxy-4-trifluoromethyl-phenoxy)-3-(6-trifluoromethyl-pyridazin-3-yl)-3-aza-bicyclo[3.2.1]octane (CAS 1253850-56-4), (8-anti)-8-(2-cyclopropylmethoxy-4-trifluoromethyl-phenoxy)-3-(6-trifluoromethyl-pyridazin-3-yl)-3-aza-bicyclo[3.2.1]octane (CAS 933798-27-7), (8-syn)-8-(2-cyclopropylmethoxy-4-trifluoromethyl-phenoxy)-3-(6-trifluoromethyl-pyridazin-3-yl)-3-aza-bicyclo[3.2.1]octane (known from WO 2007040280 A1, WO 2007040282 A1) (CAS 934001-66-8), N-[3-chloro-1-(3-pyridinyl)-1H-pyrazol-4-yl]-N-ethyl-3-[(3,3,3-trifluoropropyl)thio]-propanamide (known from WO 2015/058021 A1, WO 2015/058028 A1) (CAS 1477919-27-9), N-[4-(aminothioxomethyl)-2-methyl-6-[(methylamino)carbonyl]phenyl]-3-bromo-1-(3-chloro-2-pyridinyl)-1H-pyrazole-5-carboxamide (known from CN 103265527 A) (CAS 1452877-50-7), 5-(1,3-dioxan-2-yl)-4-[[4-(trifluoromethyl)phenyl]methoxy]-pyrimidine (known from WO 2013/115391 A1) (CAS 1449021-97-9), 3-(4-chloro-2,6-dimethylphenyl)-4-hydroxy-8-methoxy-1-methyl-1,8-diazaspiro[4.5]dec-3-en-2-one (known from WO 2010/066780 A1, WO 2011/151146 A1) (CAS 1229023-34-0), 3-(4-chloro-2,6-dimethylphenyl)-8-methoxy-1-methyl-1,8-diazaspiro[4.5]decane-2,4-dione (known from WO 2014/187846 A1) (CAS 1638765-58-8), 3-(4-chloro-2,6-dimethylphenyl)-8-methoxy-1-methyl-2-oxo-1,8-diazaspiro[4.5]dec-3-en-4-yl-carbonic acid ethyl ester (known from WO 2010/066780 A1, WO 2011151146 A1) (CAS 1229023-00-0), N-[1-[(6-chloro-3-pyridinyl)methyl]-2(1H)-pyridinylidene]-2,2,2-trifluoro-acetamide (known from DE 3639877 A1, WO 2012029672 A1) (CAS 1363400-41-2), [N(E)]—N-[1-[(6-chloro-3-pyridinyl)methyl]-2(1H)-pyridinylidene]-2,2,2-trifluoro-acetamide, (known from WO 2016005276 A1) (CAS 1689566-03-7), [N(Z)]—N-[1-[(6-chloro-3-pyridinyl)methyl]-2(1H)-pyridinylidene]-2,2,2-trifluoro-acetamide, (CAS 1702305-40-5), 3-endo-3-[2-propoxy-4-(trifluoromethyl)phenoxy]-9-[[5-(trifluoromethyl)-2-pyridinyl]oxy]-9-azabicyclo[3.3.1]nonane (known from WO 2011/105506 A1, WO 2016/133011 A1) (CAS 1332838-17-1).

Active ingredients with unknown or non-specific mode of action, e.g., fentrifanil, fenoxacrim, cycloprene, chlorobenzilate, chlordimeform, flubenzimine, dicyclanil, amidoflumet, quinomethionate, triarathene, clothiazoben, tetrasul, potassium oleate, petroleum, metoxadiazone, gossyplure, flutenzin, bromopropylate, cryolite;
Active ingredients from other classes, e.g. butacarb, dimetilan, cloethocarb, phosphocarb, pirimiphos (-ethyl), parathion (-ethyl), methacrifos, isopropyl o-salicylate, trichlorfon, sulprofos, propaphos, sebufos, pyridathion, prothoate, dichlofenthion, demeton-S-methylsulphone, isazofos, cyanofenphos, dialifos, carbophenothion, autathiofos, aromfenvinfos (-methyl), azinphos (-ethyl), chlorpyrifos (-ethyl), fosmethilan, iodofenphos, dioxabenzofos, formothion, fonofos, flupyrazofos, fensulfothion, etrimfos;
organochlorines, e.g. camphechlor, lindane, heptachlor; or phenylpyrazoles, e.g. acetoprole, pyrafluprole, pyriprole, vaniliprole, sisapronil; or isoxazolines, e.g. sarolaner, afoxolaner, lotilaner, fluralaner;
pyrethroids, e.g. (cis-, trans-), metofluthrin, profluthrin, flufenprox, flubrocythrinate, fubfenprox, fenfluthrin, protrifenbute, pyresmethrin, RU15525, terallethrin, cis-resmethrin, heptafluthrin, bioethanomethrin, biopermethrin, fenpyrithrin, cis-cypermethrin, cis-permethrin, clocythrin, cyhalothrin (lambda-), chlovaporthrin, or halogenated carbonhydrogen compounds (HCHs);
neonicotinoids, e.g. nithiazine;
dicloromezotiaz, triflumezopyrim;
macrocyclic lactones, e.g. nemadectin, ivermectin, latidectin, moxidectin, selamectin, eprinomectin, doramectin, emamectin benzoate; milbemycin oxime;
triprene, epofenonane, diofenolan;
Biologicals, hormones or pheromones, for example natural products, e.g. thuringiensin,
codlemone or neem components;
dinitrophenols, e.g. dinocap, dinobuton, binapacryl;
benzoylureas, e.g. fluazuron, penfluron;
amidine derivatives, e.g. chlormebuform, cymiazole, demiditraz;
Bee hive varroa acaricides, for example organic acids, e.g. formic acid, oxalic acid.

Non-limiting examples of insecticides and acaricides of particular interest for use in animal health are and include in particular [i.e. Mehlhorn et al Encyclpaedic Reference of Parasitology 4^{th} edition (ISBN 978-3-662-43978-4)]:
Effectors at arthropod ligand gated chloride channels: chlordane, heptachlor, endoculfan. Dieldrin, bromocyclen, toxaphene, lindane, fipronil, pyriprole, sisapronil, afoxolaner, fluralaner, sarolaner, lotilaner, fluxametamide, broflanilide, avermectin, doramectin, eprinomectin, ivermectin, milbemycin, moxidectin, selamectin;
Modulators of arthropod octopaminergic receptors: amitraz, BTS27271, cymiazole, demiditraz;
Effectors at arthropod voltage-gated sodium channels: DDT, methoxychlor, metaflumizone, indoxacarb, cinerin I, cinerin II, jasmolin I, jasmolin II, pyrethrin I, pyrethrin II, allethrin, alphacypermethrin, bioallethrin, betacyfluthrin, cyfluthrin, cyhalothrin, cypermethrin, deltamethrin, etofenprox, fenvalerate, flucythrinate, flumethrin, halfenprox, permethrin, phenothrin, resmethrin, tau-fluvalinate, tetramethrin;
Effectors at arthropod nicotinic cholinergic synapses (acetylcholine esterase, acetylcholine receptors): bromoprypylate, bendiocarb, carbaryl, methomyl, promacyl, propoxur, azamethiphos, chlorfenvinphos, chlorpyrifos, coumaphos, cythioate, diazinon, diclorvos, dicrotophos, dimethoate, ethion, famphur, fenitrothion, fenthion, heptenophos, malathion, naled, phosmet, phoxim, phtalofos, propetamphos, temephos, tetrachlorvinphos, trichlorfon, imidacloprid, nitenpyram, dinotefuran, spinosad, spinetoram;
Effectors on arthropod development processes: cyromazine, dicyclanil, diflubenzuron, fluazuron, lufenuron, triflumuron, fenoxycarb, hydroprene, methoprene, pyriproxyfen, fenoxycarb, hydroprene, S-methoprene, pyriproxyfen.

Exemplary active ingredients from the group of endoparasiticides, as a further or other active ingredient in the present invention, include, without limitation, anthelmintically active compounds and antiprotozoal active compounds.

Anthelmintically active compounds, including, without limitation, the following nematicidally, trematicidally and/or cestocidally active compounds:
from the class of macrocyclic lactones, for example: eprinomectin, abamectin, nemadectin, moxidectin, doramectin, selamectin, lepimectin, latidectin, milbemectin, ivermectin, emamectin, milbemycin;
from the class of benzimidazoles and probenzimidazoles, for example: oxibendazole, mebendazole, triclabendazole, thiophanate, parbendazole, oxfendazole, netobimin, fenbendazole, febantel, thiabendazole, cyclobendazole, cambendazole, albendazole-sulphoxide, albendazole, flubendazole;
from the class of depsipeptides, preferably cyclic depsipetides, in particular 24-membered cyclic depsipeptides, for example: emodepside, PF1022A;
from the class of tetrahydropyrimidines, for example: morantel, pyrantel, oxantel;
from the class of imidazothiazoles, for example: butamisole, levamisole, tetramisole;
from the class of aminophenylamidines, for example: amidantel, deacylated amidantel (dAMD), tribendimidine;
from the class of aminoacetonitriles, for example: monepantel;
from the class of paraherquamides, for example: paraherquamide, derquantel;
from the class of salicylanilides, for example: tribromsalan, bromoxanide, brotianide, clioxanide, closantel, niclosamide, oxyclozanide, rafoxanide;
from the class of substituted phenols, for example: nitroxynil, bithionol, disophenol, hexachlorophene, niclofolan, meniclopholan;
from the class of organophosphates, for example: trichlorfon, naphthalofos, dichlorvos/DDVP, crufomate, coumaphos, haloxon;
from the class of piperazinones/quinolines, for example: praziquantel, epsiprantel; from the class of piperazines, for example: piperazine, hydroxyzine;
from the class of tetracyclines, for example: tetracyclin, chlorotetracycline, doxycyclin, oxytetracyclin, rolitetracyclin;
from diverse other classes, for example: bunamidine, niridazole, resorantel, omphalotin, oltipraz, nitroscanate, nitroxynile, oxamniquine, mirasan, miracil, lucanthone, hycanthone, hetolin, emetine, diethylcarbamazine, dichlorophen, diamfenetide, clonazepam, bephenium, amoscanate, clorsulon.

Antiprotozoal active ingredients in the present invention, including, without limitation, the following active ingredients:
from the class of triazines, for example: diclazuril, ponazuril, letrazuril, toltrazuril;
from the class of polylether ionophore, for example: monensin, salinomycin, maduramicin, narasin;
from the class of macrocyclic lactones, for example: milbemycin, erythromycin;
from the class of quinolones, for example: enrofloxacin, pradofloxacin;
from the class of quinines, for example: chloroquine;
from the class of pyrimidines, for example: pyrimethamine;
from the class of sulfonamides, for example: sulfaquinoxaline, trimethoprim, sulfaclozin;
from the class of thiamines, for example: amprolium;
from the class of lincosamides, for example: clindamycin;
from the class of carbanilides, for example: imidocarb;
from the class of nitrofuranes, for example: nifurtimox;
from the class of quinazolinone alkaloids, for example: halofuginon;
from diverse other classes, for example: oxamniquin, paromomycin;
from the class of vaccines or antigenes from microorganisms, for example: Babesia canis rossi, Eimeria tenella, Eimeria praecox, Eimeria necatrix, Eimeria mitis, Eimeria maxima, Eimeria brunetti, Eimeria acervulina, Babesia canis vogeli, Leishmania infantum, Babesia canis canis, Dictyocaulus viviparus.

All named further active ingredients can, if their functional groups enable this, optionally form salts with suitable bases or acids.

In one embodiment, the pharmaceutical composition is a fixed combination.

The term “fixed combination” is known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical composition wherein a first active ingredient and a further active ingredient are present in one unit without being in admixture.

In one embodiment, the pharmaceutical composition is a non-fixed combination or a kit-of-parts. The terms “non-fixed combination” or “kit-of-parts” are known to persons skilled in the art and are defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

### Subjects

According to the invention, the claimed compounds are used for long-term prevention and/or treatment of a disease in a subject in need thereof.

Subject as used herein in particular means a human and a non-human animal. Unless expressly stated otherwise, the term “animal” as used herein refers to a “non-human animal”. Preferably, the non-human animal is an animal, in particular an animal selected from the group consisting of cattle, poultry, swine and companion animals such as cats and dogs. Preferably, the non-human animal is a mammal. Particularly preferably, the subject is a dog. The compounds for use according to the invention have particularly good long-lasting properties in preventing and/or treating a disease such as a helminthic infection in an animal.

### Use of the Compounds

The claimed invention also concerns the use of a compound according to the invention for long-term prevention and/or treatment of a disease, in particular a helminthic infection in animals.

In one embodiment, the compound or the pharmaceutical composition for use according to the invention, as described supra, are used for the long-term prevention and/or treatment of a disease, in particular of a helminthic infection, particularly of a gastro-intestinal and a extra-intestinal helminth infection, more particularly of a gastro-intestinal and a extra-intestinal infections with nematodes.

In one embodiment, the compound or the pharmaceutical composition for use according to the invention, as described supra, are used for the preparation of a medicament suitable for the long-term prevention and/or treatment of a disease, in particular of a helminthic infection, particularly of a gastro-intestinal and a extra-intestinal helminth infection, more particularly of a gastro-intestinal and a extra-intestinal infections with nematodes.

In one embodiment the compound or the pharmaceutical composition for use according to the invention, as described supra, are used as a long-term anthelmintic agent, in particular as a long-term nematicidal agent, a long-term platyhelminthicidal agent, a long-term acanthocephalicidal agent, or a long-term pentastomicidal agent.

### Method of Treatment

The claimed invention also concerns a method for long-term prevention and/or treatment of a disease comprising the administration of a compound or a pharmaceutical composition according to the invention at an effective dose to a subject in need thereof.

A variety of administration routes are available for administering the compounds and the pharmaceutical composition according to the invention. The particular mode selected will depend upon the particular subject group selected, the age and general health status of the subject, the particular condition being treated and the dosage required for therapeutic and/or prophylactic efficacy. The methods of this invention may be practiced using any mode of administration that produces effective levels of prevention and/or treatment of a disease, in particular a helminthic infection, without causing clinically unacceptable adverse effects.

The method comprises administering an effective amount of the compound or the pharmaceutical composition according to the invention to the subject in need thereof. Such effective amount is any amount that causes a prevention and/or treatment of a disease such as an helminthic infection. The skilled person knows methods to assess the presence and progression of an helminthic infection. A skilled person further knows that the effective amount depends on the subject such as the animal species, age, weight, stage of disease, as well as other factors known in the art.

It is possible for the compounds and the pharmaceutical composition according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

It is possible for the compounds and the pharmaceutical composition for use according to the invention to be administered in suitable administration forms.

In one embodiment, the compound or the pharmaceutical composition for use according to the invention is administered orally. For oral administration, it is possible to formulate the compounds and the pharmaceutical composition according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, chewables (for example soft chewables), powders, emulsions, suspensions, aerosols or solutions. It is possible to incorporate the compounds and the pharmaceutical composition according to the invention in crystalline and/or amorphised and/or dissolved form into said dosage forms.

In one embodiment, the compound or the pharmaceutical composition for use according to the invention is administered parenterally. Parenteral administration can be effected with avoidance of an absorption step (for example intravenously, intraarterially, intracardially, intraspinally or intralumbally) or with inclusion of absorption (for example intramuscularly, subcutaneously, intracutaneously, percutaneously or intraperitoneally). In one embodiment, the compound or the pharmaceutical composition for use according to the invention is administered subcutaneously. Administration forms which are suitable for parenteral administration are, for example, preparations for injection and infusion in the form of solutions, suspensions, emulsions, lyophylisates or sterile powders.

In one embodiment, the compound or the pharmaceutical composition for use according to the invention is administered subcutaneously in a form enabling a sustained release of the compound or the active compound in the pharmaceutical composition. This has the advantage that a particularly efficient long-term effect regarding the prevention and/or treatment of a disease is achieved, in particular in the long-term prevention and/or treatment of an helminthic infection in an animal, preferably in a cat or a dog.

Other suitable administration routes are, for example, pharmaceutical forms for inhalation (inter alia powder inhalers, nebulizers), nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, spot-ons, dusting powders, implants or stents.

The compounds and the pharmaceutical composition according to the invention can be incorporated into suitable administration forms. This can be effected in a manner known by the skilled person, in particular by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

In one embodiment, the total amount of the compound for use according to the invention is administered in ranges from about 0.001 mg/kg to about 200 mg/kg body weight per day, preferably from about 0.005 mg/kg to about 100 mg/kg body weight per day and most preferably from about 0.01 mg/kg to about 50 mg/kg body weight per day. These administration characteristics ensure that the long-term prevention and/or treatment of a disease, in particular the long-term prevention and/or treatment of an helminthic infection in animals, is particularly effective.

In one embodiment, the dosage for administration by injection, including intravenous, intramuscular, subcutaneous and parenteral injections, and use of infusion techniques is from 0.01 to 200 mg/kg of total body weight. In one embodiment, the dosage for rectal administration is from 0.01 to 200 mg/kg of total body weight. In one embodiment, the dosage for vaginal administration is from 0.01 to 200 mg/kg of total body weight.

In one embodiment, the dosage for topical administration is from 0.1 to 200 mg of total body weight. In one embodiment, the dosage for transdermal administration is 0.01 to 200 mg/kg of total body weight. In one embodiment, the dosage for administration by inhalation is from 0.01 to 100 mg/kg of total body weight.

In one embodiment, the compound or the pharmaceutical composition for use according to the invention is administered at most once every month, preferably at most once every one and a half months, more preferable at most once every two months, even more preferably at most once every three months and most preferably at most once every six months. In one embodiment, the compound or the pharmaceutical composition for use according to the invention is administered once every month, preferably once every one and a half months, more preferable once every two months, even more preferably once every three months and most preferably once every six months. This has the advantage that an effective long-term prevention and/or treatment of a disease, in particular of a helminthic infection in animals, is achieved while at the same time a convenient dosage regimen is applied.

In addition, “drug holidays” are possible, in which a subject is not dosed with a drug for a certain period of time. Such drug holidays can be beneficial to the overall balance between pharmacological effect and tolerability.

### EXPERIMENTAL SECTION

The various aspects of the invention described in this application are illustrated by the following examples which are not meant to limit the invention in any way.

### Synthesis of Compounds of the Invention

In the following, the synthesis of compounds of the invention is illustrated for several example compounds.

All other compounds of the invention, for which the synthesis is not described, are either commercially available, or are known compounds or may be formed from known compounds by known methods by a person skilled in the art.

### Analytical and Preparative Liquid Chromatography

Analytical (UP)LC-MS was performed by means of different equipments as described below. The masses (m/z) are reported from the positive mode electrospray ionisation unless the negative mode is indicated (ESI−).

M+1 (or M+H) means the molecular ion peak, plus or minus 1 a.m.u. (atomic mass unit) respectively, as observed in mass spectroscopy by electrospray ionization (ESI + or −).

### LC-MS Method 0:

Measurement of log P values was performed according to EEC directive 79/831 Annex V.A8 by HPLC (High Performance Liquid Chromatography) on reversed phase columns with the following methods, instrument(s): Agilent 1100 LC system, Agilent MSD system, HTS PAL; Waters IClass Acquity UPLC, SQD2 (MS), PDA (UV).
^{[a]}log P value is determined by measurement of LC-UV, in an acidic range, with 0.1% formic acid in water and acetonitrile as eluent (linear gradient from 10% acetonitrile to 95% acetonitrile).
^{[b]}log P value is determined by measurement of LC-UV, in a neutral range, with 0.001 molar ammonium acetate solution in water and acetonitrile as eluent (linear gradient from 10% acetonitrile to 95% acetonitrile).

Calibration was done with straight-chain alkan-2-ones (with 3 to 16 carbon atoms) with known log P values (measurement of log P values using retention times with linear interpolation between successive alkanones). Lambda-max-values were determined using UV-spectra from 200 nm to 400 nm and the peak values of the chromatographic signals.

### Method L1:

Instrument type: Waters ACQUITY SQD UPLC system; column: Waters Acquity UPLC HSS T3 1.8μ 50×1 mm; eluent A: 1 l water+0.25 ml formic acid, eluent B: 1 l acetonitrile+0.25 ml formic acid; gradient: 0.0 min 90% A→1.2 min 5% A→2.0 min 5% A oven: 50° C.; flow: 0.40 ml/min; UV-detection: 208-400 nm.

### Method L2:

MS instrument type: Agilent Technologies 6130 Quadrupole LC-MS; HPLC instrument type: Agilent Technologies 1260 Infinity; column: Waters XSelect (C18, 50×2.1 mm, 3.5μ); flow: 0.8 mL/min; column temp: 35° C.; eluent A: 0.1% formic acid in acetonitrile; eluent B: 0.1% formic acid in water; lin. gradient: t=0 min 5% A, t=3.5 min 98% A, t=6 min 98% A; detection: DAD (220-320 nm); detection: MSD (ESI pos/neg) mass range: 100-800; detection: ELSD (PL-ELS 2100): gas flow 1.2 mL/min, gas temp: 70° C., neb: 50° C.

### Method L1.1:

MS instrument type: Agilent Technologies 6130 Quadrupole LC-MS; HPLC instrument type: Agilent Technologies 1260 Infinity; column: Waters XSelect (C18, 30×2.1 mm, 3.5μ); flow: 1 mL/min; column temp: 35° C.; eluent A: 0.1% formic acid in acetonitrile; eluent B: 0.1% formic acid in water; lin. gradient: t=0 min 5% A, t=1.6 min 98% A, t=3 min 98% A; detection: DAD (220-320 nm); detection: MSD (ESI pos/neg) mass range: 100-800; detection: ELSD (PL-ELS 2100): gas flow 1.2 mL/min, gas temp: 70° C., neb: 50° C.

### Method L2.1:

MS instrument type: Agilent Technologies 6130 Quadrupole LC-MS; HPLC instrument type: Agilent Technologies 1260 Infinity; column: Waters XSelect (C18, 50×2.1 mm, 3.5μ); flow: 0.8 mL/min; column temp: 35° C.; eluent A: 0.1% formic acid in acetonitrile; eluent B: 0.1% formic acid in water; lin. gradient: t=0 min 5% A, t=3.5 min 98% A, t=6 min 98% A; detection: DAD (220-320 nm); detection: MSD (ESI pos/neg) mass range: 100-800; detection: ELSD (PL-ELS 2100): gas flow 1.2 mL/min, gas temp: 70° C., neb: 50° C.

### Method L11:

MS instrument type: Agilent Technologies LC/MSD SL; HPLC instrument type: Agilent Technologies 1100 Series; column: Phenomenex Gemini NX (C18, 50×2.0 mm), 3.0; flow: 0.8 mL/min; column temp: 25° C.; eluent A: 95% acetonitrile+5% 10 mM ammoniumbicarbonate in water in acetonitrile pH=9.0; eluent B: 10 mM ammoniumbicarbonate in water pH=9.0; lin. gradient: t=0 min 5% A, t=3.5 min 98% A, t=6 min 98% A; detection: DAD (220-320 nm); detection: MSD (ESI pos/neg) mass range: 100-800.

### LC-MS, Analytical Method A:

System MS: Thermo Scientific FT-MS; System UHPLC+: Thermo Scientific UltiMate 3000; Column: Waters, HSST3, 2.1×75 mm, C18 1.8 μm; Eluent A: 1 l Water+0.01% Formic acid; Eluent B: 1 1 Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm/Optimum Integration Path 210-300 nm.

### LC-MS, Analytical Method B3:

Instrument: SHIMADZU LCMS-UFLC 20-AD-LCMS 2020 MS detector; Column: Kinetex EVO C18 2.6 μm, 50×3.0 mm; eluent A: water+0.03 vol % ammonium hydroxide, eluent B: acetonitrile; gradient: assigned for each compound; flow 1.5 mL/min; temperature: 40° C.; PDA scan: 190-400 nm.

### Method C:

Analyses were carried out on an Acquity UPLC BEH C18 column of 50 mm length, 2.1 mm internal diameter and 1.7 μm particle size. The mobile phase used was: A1=Water with 0.1% formic acid/B1=CH₃CN with 0.1% formic acid. The injection volume was 0.1 μL. The run was performed at a temperature of 40° C. and a flow rate of 0.6 mL/min, with a gradient elution. Method info (Time (min) and B %): 0-5; 0.3-5; 2.5-95; 3.7-95; 4-5; 4.6-5.

### LC-MS Method 2:

Instrument type: Waters ACQUITY SQD UPLC system; column: Waters Acquity UPLC HSS T3 1.8μ 50×1 mm; eluent A: 1 l water+0.25 ml formic acid, eluent B: 1 l acetonitrile+0.25 ml formic acid; gradient: 0.0 min 90% A→1.2 min 5% A→2.0 min 5% A oven: 50° C.; flow: 0.40 ml/min; UV-detection: 210 nm.

### LC-MS Method 4:

Instrument type: Waters ACQUITY SQD UPLC System; column: Waters Acquity UPLC HSS T3 1.8μ 50×1 mm; eluent A: 1 l water+0.25 ml 99% ige formic acid, Eluent B: 1 l acetonitrile+0.25 ml 99% ige formic acid; gradient: 0.0 min 95% A→6.0 min 5% A→7.5 min 5% A oven: 50° C.; flow: 0.35 ml/min; UV-detection: 210 nm.

### LC-MS LC-MS Method 5:

Instrument type: UPLC with SQD2 and Sample Manager from Waters, column: Zorbax Eclipse Plus C18, 50 mm×2.1 mm, 1.8 μm, eluent A: 1 l acetonitrile+1 ml formic acid, eluent B: 1 l water+0.9 ml formic acid; gradient 0.0 min 90% B→1.7 min 5% B→2.4 min 5% B DAD A: 210±4 nm, reference 360±50 nm, DAD A: 270±2 nm, reference 550±50 nm (only ketones), MSD, 100-1000 Amu, ES-ionisation, positive or negative.

### NMR Peak Lists

¹H-NMR data of selected examples are written in form of ¹H-NMR peak lists. To each signal peak are listed the δ-value in ppm and the signal intensity in round brackets. Between the δ-value—signal intensity pairs are semicolons or commas as delimiters.

The peak list of an example has therefore the form:
δ₁ (intensity₁); δ₂ (intensity₂); . . . ; δᵢ (intensityᵢ); . . . ; δₙ (intensityₙ) or
δ₁ (intensity₁), δ₂ (intensity₂), . . . ; δᵢ (intensityᵢ), . . . , δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for ¹H spectra, we use tetramethylsilane and/or the chemical shift of the solvent used, especially in the case of spectra measured in DMSO. Therefore in NMR peak lists, tetramethylsilane peak can occur but not necessarily.

The ¹H-NMR peak lists are similar to classical ¹H-NMR prints and contains therefore usually all peaks, which are listed at classical NMR-interpretation.

Additionally they can show like classical ¹H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our ¹H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical ¹H-NMR interpretation.

Further details of NMR-data description with peak lists you find in the publication “Citation of NMR Peaklist Data within Patent Applications” of the Research Disclosure Database Number 564025.

### Example 1

### N-[(4S)-3,4-Dihydro-2H-chromen-4-yl]-4-ethyl-7-fluoro-8-(2,3,5-trifluorophenyl)quinoline-3-carboxamide

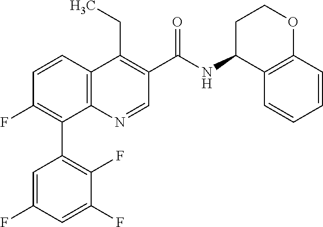 [SMILES: CCc1c(C(=O)N[C@H]2CCOc3ccccc32)cnc2c(-c3cc(F)cc(F)c3F)c(F)ccc12]

Under argon a flask was charged with 8-bromo-N-[(4S)-3,4-dihydro-2H-chromen-4-yl]-4-ethyl-7-fluoroquinoline-3-carboxamide (13.0 g, 30.3 mmol), 2,3,5-trifluorobenzene boronic acid (2.67 g, 15.2 mmol), potassiumcarbonate (8.37 g, 60.6 mmol) and (2′-aminobiphenyl-2-yl)(chloro)palladium-dicyclohexyl(2′,4′,6′-triisopropylbiphenyl-2-yl)phosphine (1:1) (477 mg, 606 gmol). A degassed 5:1 mixture of dioxan/water (140 ml) was added and the mixture stirred at 70° C. for 45 min. Two more portions of 2,3,5-trifluorobenzene boronic acid (2.67 g, 15.2 mmol) were added within 1.5 h and stirring continued for 1.5 h after the last dosage. Then more 2,3,5-trifluorobenzene boronic acid (5.33 g, 30.3 mmol) and (2′-aminobiphenyl-2-yl)(chloro)palladium-dicyclohexyl(2′,4′,6′-triisopropylbiphenyl-2-yl)phosphine (1:1) (238 mg, 303 gmol) were added and stirred at the same temperature for 1 h. Water and ethylacetate were added at RT stirred and the phases separated. The aqueous phase was extracted two times with ethylacetate, the combined organic phases dried and evaporated under diminished pressure. The residue (24 g) was purified by flash chromatography on silica with DCM and methanol (0-2%) and then in a second silica chromatography with cyclohexane-ethylacetate (10-25%) yielding 7.63 g. Remaining mixed fractions were purified by preparative HPLC (RP 18, gradient with 0.1% aqueous formic acid and acetonitrile) to yield more pure material (0.75 g).

Total yield: 8.38 g (58% of theory)

LC-MS (Method 4): Rₜ=3.99 min; MS (ESIpos): m/z=481 [M+H]⁺

¹H-NMR (600 MHz, DMSO-d6) δ[ppm]: 0.005 (0.62), 1.305 (8.10), 1.317 (16.00), 1.318 (15.96), 1.329 (7.70), 1.397 (8.26), 2.020 (0.99), 2.026 (1.42), 2.031 (1.82), 2.037 (1.85), 2.042 (1.90), 2.049 (1.91), 2.054 (2.16), 2.060 (1.62), 2.065 (1.16), 2.181 (0.98), 2.187 (1.48), 2.195 (2.06), 2.203 (1.94), 2.209 (2.04), 2.218 (1.46), 2.223 (1.05), 2.232 (0.71), 2.516 (0.89), 2.520 (0.95), 2.523 (0.96), 3.227 (0.71), 3.236 (1.35), 3.249 (3.27), 3.257 (3.63), 3.261 (4.26), 3.270 (4.39), 3.282 (3.24), 3.294 (1.47), 3.304 (1.17), 4.214 (0.72), 4.219 (1.14), 4.226 (1.21), 4.233 (2.56), 4.239 (3.13), 4.245 (2.80), 4.252 (3.53), 4.255 (3.49), 4.261 (3.70), 4.266 (2.73), 4.272 (3.23), 4.280 (1.06), 4.285 (1.15), 4.291 (0.88), 5.280 (1.26), 5.291 (2.88), 5.301 (2.73), 5.312 (1.11), 6.781 (6.23), 6.783 (6.34), 6.795 (6.65), 6.796 (6.62), 6.907 (3.27), 6.919 (6.32), 6.932 (3.61), 7.153 (2.85), 7.164 (4.85), 7.176 (2.33), 7.295 (2.49), 7.302 (2.41), 7.334 (2.71), 7.345 (4.69), 7.356 (2.50), 7.660 (0.92), 7.665 (1.13), 7.670 (1.32), 7.675 (2.00), 7.678 (1.91), 7.683 (2.01), 7.689 (1.92), 7.692 (1.90), 7.697 (1.20), 7.702 (1.05), 7.707 (0.93), 7.780 (3.82), 7.795 (7.06), 7.811 (3.81), 8.484 (4.02), 8.494 (4.24), 8.500 (4.25), 8.510 (3.87), 8.812 (11.05), 8.818 (10.77), 9.124 (4.07), 9.137 (3.96).

### Example 2

### (S)-3-(3,5-dichloro-4-fluorophenyl)-N-(2,3-dihydro-1H-inden-1-yl)-7-isopropyl-2-methylpyrazolo[1,5-a]pyrimidine-6-carboxamide

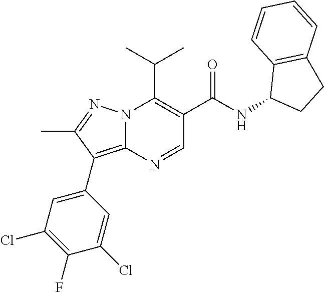 [SMILES: Cc1nn2c(C(C)C)c(C(=O)N[C@H]3CCc4ccccc43)cnc2c1-c1cc(Cl)c(F)c(Cl)c1]

Example 2 was prepared analogously to Example 130 of WO 2017/178416.

¹H-NMR (400.0 MHz, d₆-DMSO): d=9.225(0.9), 9.205(0.9), 8.436(4.0), 7.540(0.7), 7.526(0.8), 7.521(0.9), 7.515(1.1), 7.509(2.1), 7.495(0.3), 7.492(0.3), 7.398(0.3), 7.394(0.6), 7.390(0.3), 7.375(0.7), 7.371(0.7), 7.355(0.3), 7.338(0.8), 7.319(0.9), 7.195(0.4), 7.192(0.4), 7.174(0.9), 7.157(0.6), 7.153(0.5), 6.940(0.6), 6.922(1.1), 6.904(0.5), 6.809(1.2), 6.807(1.1), 6.788(1.1), 5.234(0.5), 5.215(0.6), 4.280(0.5), 4.272(0.4), 4.264(0.5), 4.256(0.4), 4.241(0.4), 4.234(0.5), 4.219(0.4), 4.213(0.6), 3.962(0.5), 3.944(0.7), 3.927(0.5), 3.329(29.8), 2.524(0.5), 2.507(24.6), 2.502(32.1), 2.498(23.8), 2.344(7.9), 2.329(0.3), 2.207(0.3), 2.198(0.3), 2.185(0.4), 2.062(0.4), 2.054(0.4), 1.593(2.9), 1.574(3.0), 1.567(3.3), 1.549(3.0), 1.398(16.0), 0.008(0.5), 0.000(15.7), −0.008(0.7)

### Example 3

### (S)—N-(chroman-4-yl)-3-(3,4-difluorophenyl)-7-isopropyl-2-methylpyrazolo[1,5-a]pyrimidine-6-carboxamide

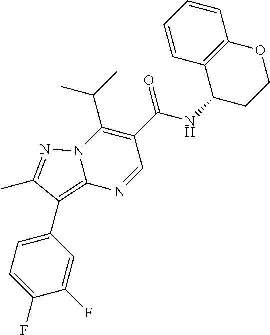 [SMILES: Cc1nn2c(C(C)C)c(C(=O)N[C@H]3CCOc4ccccc43)cnc2c1-c1ccc(F)c(F)c1]

Example 3 was prepared analogously to Example 100 of WO 2017/1784416.

¹H-NMR (400.0 MHz, d₆-DMSO): d=9.221(1.6), 9.201(1.6), 8.529(7.4), 7.826(0.7), 7.822(0.8), 7.806(0.8), 7.802(0.8), 7.795(0.8), 7.790(0.8), 7.775(0.8), 7.770(0.8), 7.603(0.4), 7.598(0.4), 7.590(0.5), 7.581(1.4), 7.576(1.7), 7.571(1.7), 7.564(1.1), 7.552(1.2), 7.547(1.2), 7.530(0.5), 7.525(1.3), 7.504(0.5), 7.346(1.5), 7.328(1.6), 7.203(0.7), 7.200(0.8), 7.182(1.6), 7.165(1.0), 7.161(1.0), 6.953(1.1), 6.950(1.2), 6.932(2.0), 6.916(0.9), 6.913(0.9), 6.817(2.1), 6.815(2.1), 6.796(1.9), 6.794(1.9), 5.260(0.4), 5.245(1.0), 5.226(1.0), 5.211(0.4), 4.307(0.4), 4.299(0.3), 4.287(0.9), 4.279(0.8), 4.271(0.9), 4.262(0.8), 4.256(0.8), 4.248(0.9), 4.235(0.8), 4.227(1.1), 4.207(0.4), 3.980(0.3), 3.962(0.9), 3.944(1.3), 3.927(1.0), 3.909(0.4), 3.349(0.4), 3.329(125.0), 2.676(0.7), 2.671(0.9), 2.667(0.7), 2.619(16.0), 2.524(2.2), 2.511(50.2), 2.507(102.1), 2.502(135.0), 2.498(100.1), 2.493(50.6), 2.333(0.6), 2.329(0.9), 2.324(0.7), 2.320(0.3), 2.226(0.4), 2.213(0.6), 2.205(0.6), 2.192(0.7), 2.180(0.5), 2.078(0.4), 2.069(0.6), 2.062(0.7), 2.051(0.5), 2.046(0.5), 2.035(0.5), 2.027(0.5), 1.572(7.0), 1.554(7.6), 1.548(7.7), 1.530(6.9), 1.398(3.8), 0.008(1.3), 0.000(40.6), −0.008(1.6)

### Example 4

### (S)—N-(chroman-4-yl)-3-(3,4-difluorophenyl)-7-isopropyl-2-methylpyrazolo[1,5-a]pyrimidine-6-carboxamide

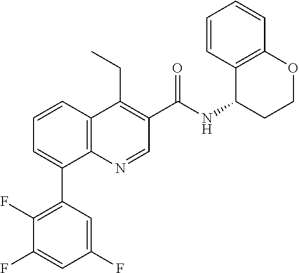 [SMILES: CCc1c(C(=O)N[C@H]2CCOc3ccccc32)cnc2c(-c3cc(F)cc(F)c3F)cccc12]

Example 4 was prepared analogously to Example 655 of WO 2018/087036.

LC-MS (Method L1): Rt=1.18 min; MS (ESIpos): m/z=463 [M+H]⁺

¹H-NMR (500 MHz, DMSO-d6) δ[ppm]: 1.311 (7.80), 1.327 (16.00), 1.341 (7.58), 2.033 (1.52), 2.041 (1.61), 2.047 (1.74), 2.054 (1.78), 2.061 (2.13), 2.067 (1.87), 2.198 (1.93), 2.207 (1.91), 2.215 (1.83), 3.235 (2.53), 3.250 (6.81), 3.265 (6.83), 3.279 (3.01), 4.227 (1.01), 4.244 (3.29), 4.258 (5.03), 4.264 (5.39), 4.276 (3.07), 4.290 (0.97), 5.288 (1.40), 5.300 (2.99), 5.315 (2.94), 5.327 (1.35), 6.781 (5.15), 6.797 (5.61), 6.909 (2.60), 6.924 (5.33), 6.939 (3.03), 7.151 (2.73), 7.165 (4.53), 7.180 (2.23), 7.228 (2.53), 7.236 (2.62), 7.345 (4.77), 7.360 (4.45), 7.601 (2.02), 7.607 (2.10), 7.614 (2.02), 7.782 (2.66), 7.797 (5.11), 7.813 (4.47), 7.845 (6.38), 7.857 (3.89), 8.374 (4.77), 8.389 (4.49), 8.789 (13.74), 9.121 (4.49), 9.137 (4.40).

### Example 5

### (S)—N-(chroman-4-yl)-8-(3,5-dichlorophenyl)-7-fluoro-4-(3-fluoroazetidin-1-yl)quinoline-3-carboxamide

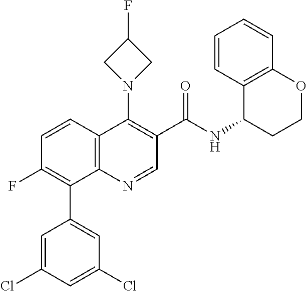 [SMILES: O=C(N[C@H]1CCOc2ccccc21)c1cnc2c(-c3cc(Cl)cc(Cl)c3)c(F)ccc2c1N1CC(F)C1]

Example 5 was prepared analogously to Example 655 of WO 2018/087036.

LC-MS (Method T1): Rt=0.85 min; MS (ESIpos): m/z=522 [M+H]⁺

¹H-NMR (400 MHz, DMSO-d6) δ[ppm]: −0.149 (0.44), −0.008 (3.87), 0.008 (3.75), 0.146 (0.44), 1.235 (0.52), 2.012 (0.57), 2.023 (0.75), 2.033 (0.65), 2.046 (1.16), 2.060 (1.08), 2.073 (1.84), 2.135 (0.41), 2.149 (1.12), 2.163 (1.23), 2.181 (0.72), 2.199 (0.67), 2.327 (0.42), 2.366 (0.41), 2.523 (1.29), 2.670 (0.43), 2.710 (0.44), 4.252 (2.47), 4.264 (4.03), 4.278 (2.39), 4.512 (0.46), 4.544 (1.08), 4.577 (2.07), 4.606 (1.41), 4.637 (1.95), 4.647 (2.10), 4.661 (0.77), 4.676 (0.74), 4.691 (0.69), 4.705 (0.72), 4.718 (0.44), 5.178 (0.62), 5.193 (1.42), 5.212 (1.43), 5.226 (0.63), 5.411 (0.80), 5.557 (0.79), 5.754 (4.87), 6.791 (2.68), 6.812 (2.97), 6.894 (1.29), 6.897 (1.35), 6.915 (2.76), 6.931 (1.62), 6.934 (1.63), 7.150 (1.32), 7.154 (1.43), 7.171 (2.22), 7.189 (1.09), 7.192 (1.09), 7.314 (2.35), 7.331 (2.17), 7.472 (1.95), 7.490 (2.54), 7.493 (2.57), 7.511 (2.33), 7.614 (16.00), 7.737 (3.27), 7.753 (2.63), 7.755 (2.75), 8.065 (2.69), 8.084 (2.39), 8.181 (0.62), 8.488 (9.91), 9.045 (2.49), 9.065 (2.43).

### Example 6

### 8-[2-chloro-6-(trifluoromethyl)-pyrimidin-4-yl]-N-(2,3-dihydro-1,4-benzoxazin-4-yl)-4-morpholino-quinoline-3-carboxamide

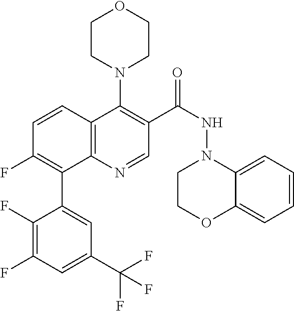 [SMILES: O=C(NN1CCOc2ccccc21)c1cnc2c(-c3cc(C(F)(F)F)cc(F)c3F)c(F)ccc2c1N1CCOCC1]

Example 6 was prepared analogously to Example 5.1 of WO 2022/117783.

Rt=2.23 min, m/z=571.37 [M+H]+/Method C

¹H NMR (400 MHz, DMSO) d [ppm]10.71 (s, 1H), 8.82 (s, 1H), 8.38 (d, J=8.4 Hz, 1H), 8.09 (t, J=7.6 Hz, 1H), 7.92 (d, J=6.8 Hz, 1H), 7.78 (t, J=8 Hz, 2H), 7.72 (d, J=4.4 Hz, 1H), 7.01 (d, J=7.6 Hz, 1H), 6.83 (t, J=6.8 Hz, 1H), 6.72-6.78 (m, 2H), 4.38 (t, J=3.6 Hz, 2H), 3.90 (br s, 4H), 3.69 (br s, 2H), 3.31 (s, 4H).

### Example 7

### N-[(4S)-3,4-Dihydro-2H-chromen-4-yl]-7-fluoro-4-(3-fluoroazetidin-1-yl)-8-(2,3,5-trifluorophenyl)quinoline-3-carboxamide

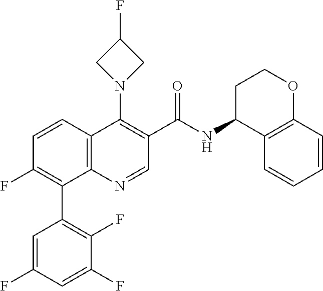 [SMILES: O=C(N[C@H]1CCOc2ccccc21)c1cnc2c(-c3cc(F)cc(F)c3F)c(F)ccc2c1N1CC(F)C1]

A mixture of 8-bromo-N-[(4S)-3,4-dihydro-2H-chromen-4-yl]-7-fluoro-4-(3-fluoroazetidin-1-yl)quinoline-3-carboxamide (Expl. 15A) (1500 mg, 3.163 mmol) and bis(triphenylphosphine)dichloropalladium(II) (255 mg, 0.363 mmol) in 1,4-dioxane (50 mL) was stirred at room temperature for 2 h. After the addition of (2,3,5-trifluorophenyl)boronic acid (2200 mg, 12.51 mmol), sodium carbonate (5000 mg, 47.175 mmol) and water (6.20 mL) stirring was continued for 18 h at 90° C. The mixture was cooled to room temperature, water was added and the aqueous layer was extracted with dichloromethane (3×100 mL). Solvents were dried and removed under reduced pressure. Purification by preparative HPLC (water/acetonitrile 80:20→5:95/afforded 675 mg (40.6% of theory) of the title compound.

LC-MS (Method 5): Rt=0.88 min; m/z=526 (M+H)+

log P (HCOOH) (Method 0)=1.93

¹H-NMR (400 MHz, DMSO-d6) δ 1.99-2.08 (m, 1H), 2.14-2.19 (m, 1H), 4.25 (t, J=3.5 Hz, 2H), 4.57-4.74 (m, 4H), 5.17-5.22 (m, 1H), 5.42-5.60 (m, 1H), 6.78-6.80 (m, 1H), 6.88-6.92 (m, 1H), 7.14-7.23 (m, 2H), 7.30-7.32 (m, 1H), 7.44-7.49 (m, 1H), 7.55-7.65 (m, 1H), 8.20-8.24 (m, 1H), 8.43 (s, 1H), 9.03-9.06 (m, 1H).

### Example 8

### N-[(4S)-3,4-dihydro-2H-chromen-4-yl]-4-(1,1-dioxidothietan-3-yl)-7-fluoro-8-(2,3,5-trifluorophenyl)quinoline-3-carboxamide

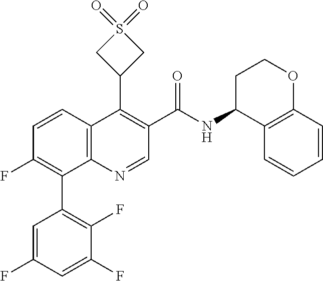 [SMILES: O=C(N[C@H]1CCOc2ccccc21)c1cnc2c(-c3cc(F)cc(F)c3F)c(F)ccc2c1C1CS(=O)(=O)C1]

N-[(4S)-3,4-dihydro-2H-chromen-4-yl]-7-fluoro-4-(thietan-3-yl)-8-(2,3,5-trifluorophenyl)-quinoline-3-carboxamide (400 mg, 0.76 mmol) was suspended in DCM (9 ml). MCPBA (359 mg, 77% purity, 1.60 mmol) was added and the solution formed stirred overnight at RT. The precipitated reaction mixture was diluted with DCM (15 ml) and aq. potassiumcarbonate solution (15 ml, 0.5 M) and vigorously stirred. The remaining precipitate was filtered off. More DCM was added to the filtrate and the phases separated. The aqueous phase was extracted two times with DCM and the combined organic phases were dried and evaporated. The residue was combined with the first precipitate and purified by preparative HPLC (RP 18, gradient with 0.1% aqueous formic acid and acetonitrile).

Yield: 380 mg (90% of theory)

LC-MS (Analytical Method A1): Rₜ=2.08 min; MS (ESIpos): m/z=557 [M+H]⁺

¹H NMR (DMSO-d₆) δ: 9.38 (m, 1H), 8.86 (d, 1H), 8.65 (m, 1H), 7.85 (m, 1H), 7.65-7.71 (m, 1H), 7.43 (d, 1H), 7.27 (m, 1H), 7.18 (m, 1H), 6.92 (m, 1H), 6.80 (d, 1H), 5.23-5.30 (m, 1H), 4.74-4.87 (m, 3H), 4.64-4.72 (m, 2H), 4.20-4.31 (m, 2H), 2.22-2.29 (m, 1H), 2.10-2.16 (m, 1H).

### Example 9

### N—((S)-chroman-4-yl)-7-fluoro-4-((1R,3S)-3-fluorocyclobutyl)-8-(2,3,5-trifluorophenyl)quinoline-3-carboxamide

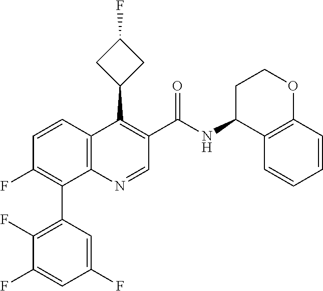 [SMILES: O=C(N[C@H]1CCOc2ccccc21)c1cnc2c(-c3cc(F)cc(F)c3F)c(F)ccc2c1[C@H]1C[C@H](F)C1]

### Step 1:

### Ethyl 7-fluoro-4-hydroxy-8-(2,3,5-trifluorophenyl)quinoline-3-carboxylate

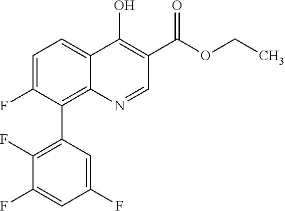 [SMILES: CCOC(=O)c1cnc2c(-c3cc(F)cc(F)c3F)c(F)ccc2c1O]

To a solution of ethyl 8-bromanyl-7-fluoranyl-4-oxidanyl-quinoline-3-carboxylate (10.00 g, 31.84 mmol) in toluene (100 mL) and water (25 mL), was added [2,3,5-tris(fluoranyl)phenyl]boronic acid (6.72 g, 38.20 mmol), Xphos Pd G3 (2.70 g, 3.18 mmol), and CsF (6.31 g, 95.51 mmol). The reaction mixture was stirred for 16 hour 100° C. under nitrogen atmosphere. After cooling to room temperature, the resulting mixture was treated with water and extracted with ethyl acetate. The organic layers were combined, dried over anhydrous sodium sulfate, filtered, and concentrated under reduced pressure. The residue was slurred with petroleum ether. The precipitated solids were collected by filtration and washed with petroleum ether to afford ethyl 7-fluoranyl-4-oxidanyl-8-[2,3,5-tris(fluoranyl)phenyl]quinoline-3-carboxylate (4.00 g, 10.95 mmol, 34.40% yield) as a black solid.

LC-MS (Analytical Method A3, 1.20-1.90 min 95% B): Rt=1.012 min; MS (ESIpos): m/z=366 (M+H)⁺.

### Step 2:

### Ethyl 4-bromo-7-fluoro-8-(2,3,5-trifluorophenyl)quinoline-3-carboxylate

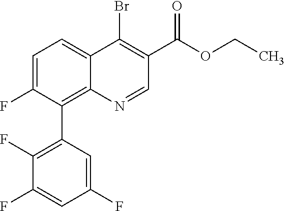 [SMILES: CCOC(=O)c1cnc2c(-c3cc(F)cc(F)c3F)c(F)ccc2c1Br]

To a solution of ethyl 7-fluoranyl-4-oxidanyl-8-[2,3,5-tris(fluoranyl)phenyl]quinoline-3-carboxylate (3.93 g, 10.76 mmol) in DCM (40 mL) and DMF (8 mL) was added POBr₃ (4.63 g, 16.14 mmol) in portions at 0° C. The resulting mixture was stirred for 2 hours at rt. The resulting mixture was treated with water and extracted with ethyl acetate. The organic layers were combined, dried over anhydrous sodium sulfate, filtered, and concentrated under reduced pressure. The residue was purified by silica gel column chromatography (ethyl acetate: petroleum ether=0-50%) to afford ethyl 4-bromanyl-7-fluoranyl-8-[2,3,5-tris(fluoranyl)phenyl]quinoline-3-carboxylate (3.37 g, 7.87 mmol, 73.15% yield).

LC-MS (Analytical Method A3, 1.20-1.90 min 95% B): Rt=1.262 min; MS (ESIpos): m/z=428 (M+H)⁺.

### Step 3:

### Ethyl 4-(3,3-dimethoxy-1-(methoxycarbonyl)cyclobutyl)-7-fluoro-8-(2,3,5-trifluorophenyl)quinoline-3-carboxylate

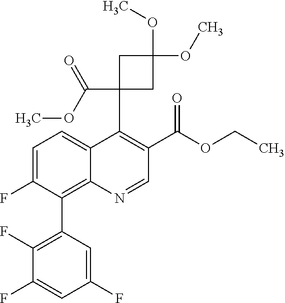 [SMILES: CCOC(=O)c1cnc2c(-c3cc(F)cc(F)c3F)c(F)ccc2c1C1(C(=O)OC)CC(OC)(OC)C1]

To a solution of methyl 3,3-di(methoxy)cyclobutanecarboxylate (2.00 g, 11.46 mmol) in THF (20 mL) were added LiHMDS (15.3 mL, 15.27 mmol, 1M in THF) dropwise at −78° C. under nitrogen atmosphere. The resulting mixture was stirred for 30 min at −78° C. To the above mixture was added ethyl 4-bromanyl-7-fluoranyl-8-[2,3,5-tris(fluoranyl)phenyl]quinoline-3-carboxylate (3.27 g, 7.64 mmol) in THF (20 mL) dropwise at −78° C. The resulting mixture was stirred for additional 1 hour at −78° C. The reaction was quenched with sat. aqueous NH₄Cl solution at 0° C. The resulting mixture was extracted with ethyl acetate. The organic layers were combined, dried over anhydrous sodium sulfate, filtered, and concentrated under reduced pressure. The residue was purified by silica gel column chromatography (ethyl acetate: petroleum ether=0-30%) to afford ethyl 4-[3,3-di(methoxy)-1-methoxycarbonyl-cyclobutyl]-7-fluoranyl-8-[2,3,5 tris(fluoranyl)phenyl]quinoline-3-carboxylate (2.9 g, 5.56 mmol, 72.82% yield).

LC-MS (Analytical Method A3, 1.20-1.90 min 95% B): Rt=1.295 min; MS (ESIpos): m/z=522 (M+H)⁺.

### Step 4:

### 7-fluoro-4-(3-oxocyclobutyl)-8-(2,3,5-trifluorophenyl)quinoline-3-carboxylic acid

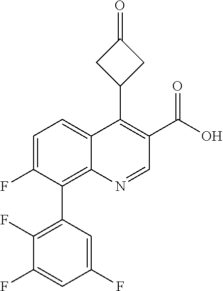 [SMILES: O=C1CC(c2c(C(=O)O)cnc3c(-c4cc(F)cc(F)c4F)c(F)ccc23)C1]

To a solution of ethyl 4-[3,3-di(methoxy)-1-methoxycarbonyl-cyclobutyl]-7-fluoranyl-8-[2,3,5-tris(fluoranyl)phenyl]quinoline-3-carboxylate (2.85 g, 5.47 mmol) in DME (30 mL), was added NaOH (874 mg, 21.86 mmol) in water (2.4 mL). The reaction mixture was stirred for 16 hours at 60° C. Then to the above mixture was added aqueous HCl solution (9 N, 3.6 mL) at 60° C. The resulting mixture was stirred for additional 2 hours at 60° C. After cooling to room temperature, the resulting mixture was extracted with ethyl acetate. The organic layers were combined, dried over anhydrous sodium sulfate, filtered, and concentrated under reduced pressure to afford 7-fluoranyl-4-(3-oxidanylidenecyclobutyl)-8-[2,3,5-tris(fluoranyl)phenyl]quinoline-3-carboxylic acid (2.60 g, 6.68 mmol).

LC-MS (Analytical Method B3, 1.40-1.90 min 95% B): Rt=0.929 min; MS (ESIpos): m/z=390 (M+H)⁺.

### Step 5:

### 7-fluoro-4-((1r, 3r)-3-hydroxycyclobutyl)-8-(2,3,5-trifluorophenyl)quinoline-3-carboxylic acid

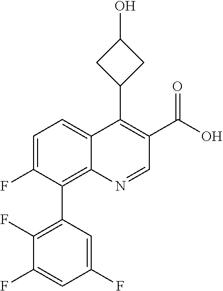 [SMILES: O=C(O)c1cnc2c(-c3cc(F)cc(F)c3F)c(F)ccc2c1C1CC(O)C1]

To a solution of 7-fluoranyl-4-(3-oxidanylidenecyclobutyl)-8-[2,3,5-tris(fluoranyl)phenyl]quinoline-3-carboxylic acid (2.50 g, 6.42 mmol) in MeOH (20 mL) was added sodium borohydride (364 mg, 9.63 mmol) in portions at 0° C. The resulting mixture was stirred for 2 hours at rt. The resulting mixture was treated with water and acidified to pH=5 with HCl (2 N). The resulting mixture was extracted with ethyl acetate. The organic layers were combined, dried over anhydrous sodium sulfate, filtered, and concentrated under reduced pressure to afford 7-fluoranyl-8-(3-fluoranyl-2,5-difluoro-phenyl)-4-(3-hydroxycyclobutyl)quinoline-3-carboxylic acid (1.20 g, 3.07 mmol, 47.75% yield).

LC-MS (Analytical Method C3, 0.01-1.20 min 2-100% B, 1.20-1.75 min 100% B): Rₜ=1.035 min; MS (ESIpos): m/z=392 (M+H)⁺.

### Step 6:

### N—((S)-chroman-4-yl)-7-fluoro-4-((1R,3S)-3-hydroxycyclobutyl)-8-(2,3,5-trifluorophenyl)quinoline-3-carboxamide

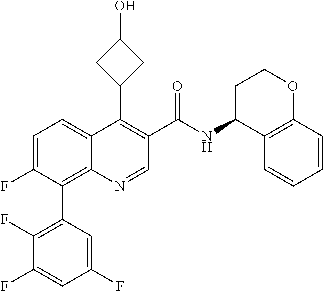 [SMILES: O=C(N[C@H]1CCOc2ccccc21)c1cnc2c(-c3cc(F)cc(F)c3F)c(F)ccc2c1C1CC(O)C1]

To a solution of 7-fluoranyl-8-(3-fluoranyl-2,5-difluoro-phenyl)-4-(3-hydroxycyclobutyl)quinoline-3-carboxylic acid (640 mg, 1.64 mmol) in DMF (6 mL), was added (4S)-chroman-4-amine (488 mg, 3.27 mmol), HATU (932.81 mg, 2.45 mmol) and DIEA (634 mg, 4.91 mmol), The reaction mixture was stirred for 1 hour at rt. The resulting mixture was treated with water and extracted with ethyl acetate. The organic layers were combined, dried over anhydrous sodium sulfate, filtered, and concentrated under reduced pressure. The residue was purified by silica gel column chromatography (ethyl acetate: petroleum ether=0-50%) to afford N-[(4S)-chroman-4-yl]-7-fluoro-4-(3-hydroxycyclobutyl)-8-(2,3,5-trifluorophenyl)quinoline-3-carboxamide (280 mg, 535.90 mol, 32.77% yield) as a white solid.

LC-MS (Analytical Method A3, 1.20-1.90 min 95% B): Rₜ=1.135 min; MS (ESIpos): m/z=523 (M+H)⁺.

### Step 7:

### N—((S)-chroman-4-yl)-7-fluoro-4-((1R,3S)-3-fluorocyclobutyl)-8-(2,3,5-trifluorophenyl)quinoline-3-carboxamide

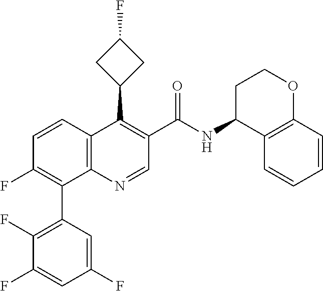 [SMILES: O=C(N[C@H]1CCOc2ccccc21)c1cnc2c(-c3cc(F)cc(F)c3F)c(F)ccc2c1[C@H]1C[C@H](F)C1]

To a stirred solution of N-[(4S)-chroman-4-yl]-7-fluoro-4-(3-hydroxycyclobutyl)-8-(2,3,5-trifluorophenyl)quinoline-3-carboxamide (272 mg, 520.58 mol) in DCM (3 mL) was added BAST (346 mg, 1.56 mmol) dropwise at 0° C. under nitrogen atmosphere. The resulting mixture was stirred for 0.5 hour at 0° C. under nitrogen atmosphere. The resulting mixture was treated with water and extracted with dichloromethane. The organic layers were combined, dried over anhydrous sodium sulfate, filtered, and concentrated under reduced pressure. The residue was purified by Prep-TLC (ethyl acetate: petroleum ether=1:2) and further purified by Prep-HPLC (Mobile Phase A: Water (10 mmol/L NH₄HCO₃), Mobile Phase B: ACN; Gradient: 47% B to 72% B in 9 min) to afford N-[(4S)-chroman-4-yl]-7-fluoranyl-4-(3-fluorocyclobutyl)-8-[2,3,5-tris(fluoranyl)phenyl]quinoline-3-carboxamide (32.3 mg, 60.66 mol, 11.65% yield, 98.5% purity) as a white solid.

¹H-NMR (400 MHz, DMSO-D6): 6 [ppm]=9.17 (d, 1H), 8.77 (d, 1H), 8.28-8.25 (m, 1H), 7.76-7.68 (m, 2H), 7.34-7.29 (m, 2H), 7.18-7.15 (m, 1H), 6.92-6.91 (m, 1H), 6.79 (d, 1H), 5.28-5.14 (m, 2H), 4.76-4.72 (m, 1H), 4.28-4.20 (m, 2H), 2.92-2.62 (m, 4H), 2.25-2.15 (m, 1H), 2.05-2.00 (m, 1H).

LC-MS (Analytical Method B3, 0.01-1.90 min 30-70% B, 1.90-2.00 min 70-95% B, 2.00-2.70 min 95% B): Rₜ, =1.846 min; MS (ESIpos): m/z=525 (M+H)⁺.

### Example 10

### (S)-3-(2-chloro-6-fluorophenyl)-N-(chroman-4-yl)-7-isopropyl-2-methylpyrazolo[1,5-a]pyrimidine-6-carboxamide

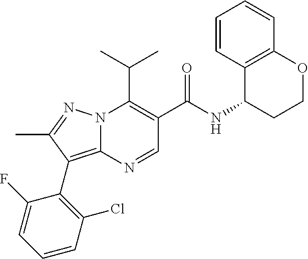 [SMILES: Cc1nn2c(C(C)C)c(C(=O)N[C@H]3CCOc4ccccc43)cnc2c1-c1c(F)cccc1Cl]

Example 10 was prepared analogously to Example 130 of WO 2017/178416.

¹H-NMR (400.0 MHz, d₆-DMSO): δ [ppm]=9.104(0.9), 9.084(0.9), 8.595(4.2), 7.942(4.0), 7.926(4.0), 7.397(0.6), 7.386(0.8), 7.376(0.7), 7.298(0.3), 7.288(0.6), 7.276(1.1), 7.266(0.7), 7.255(2.3), 7.248(1.3), 7.242(1.2), 7.233(1.2), 5.534(0.8), 5.514(0.8), 3.976(0.6), 3.959(0.8), 3.941(0.6), 3.568(1.0), 3.329(30.2), 2.985(0.4), 2.977(0.5), 2.963(0.4), 2.955(0.4), 2.891(0.7), 2.871(0.5), 2.852(0.4), 2.676(0.4), 2.672(0.5), 2.667(0.4), 2.640(9.1), 2.556(0.5), 2.547(0.6), 2.536(0.7), 2.525(1.9), 2.511(30.1), 2.507(60.4), 2.502(79.1), 2.498(58.3), 2.494(29.3), 2.334(0.4), 2.329(0.5), 2.325(0.4), 1.944(0.5), 1.924(0.5), 1.913(0.5), 1.892(0.5), 1.573(4.1), 1.556(7.9), 1.538(4.0), 1.398(16.0), 0.008(0.5), 0.000(14.1), −0.008(0.6)

### Example 11

### (S)—N-(chroman-4-yl)-4-(oxetan-3-yl)-8-(2,3,5-trifluorophenyl)quinoline-3-carboxamide

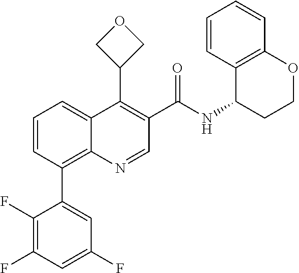 [SMILES: O=C(N[C@H]1CCOc2ccccc21)c1cnc2c(-c3cc(F)cc(F)c3F)cccc2c1C1COC1]

Example 11 was prepared analogously to Example 660 of WO 2018/087036.

LC-MS (Method L2): Rₜ=3.65 min; m/z=491 (M+H)⁺.

¹H-NMR (400 MHz, DMSO-d6) δ 9.26 (d, J=8.1 Hz, 1H), 8.87 (s, 1H), 7.91-7.87 (m, 1H), 7.84-7.73 (m, 2H), 7.70-7.57 (m, 1H), 7.35 (d, J=7.8 Hz, 1H), 7.30-7.23 (m, 1H), 7.19 (t, J=7.8 Hz, 1H), 6.93 (t, J=7.5 Hz, 1H), 6.81 (d, J=8.2 Hz, 1H), 5.31-5.11 (m, 4H), 4.84-4.70 (m, 2H), 4.34-4.21 (m, 2H), 2.25-2.15 (m, 1H), 2.08-2.01 (m, 1H).

### Example 12

### N-[(4S)-3,4-Dihydro-2H-chromen-4-yl]-4-(tetrahydro-2H-pyran-4-yl)-8-(2,3,5-trifluorophenyl)quinoline-3-carboxamide

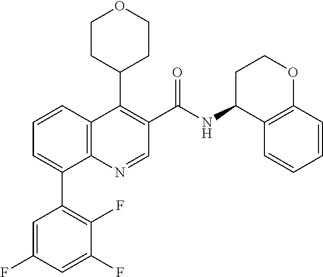 [SMILES: O=C(N[C@H]1CCOc2ccccc21)c1cnc2c(-c3cc(F)cc(F)c3F)cccc2c1C1CCOCC1]

### Procedure 1:

Under argon N-[(4S)-3,4-dihydro-2H-chromen-4-yl]-4-(3,6-dihydro-2H-pyran-4-yl)-8-(2,3,5-trifluorophenyl)quinoline-3-carboxamide (Expl. 1) (500 mg, 0.97 mmol) was dissolved in ethyl acetate/ethanol (2:1, 15 ml). The catalyst, 10% palladium on charcoal (125 mg), was added, argon replaced by hydrogen and the mixture stirred under atmospheric pressure of hydrogen for 18 h. The reaction mixture was filtered over celite, rinsed with ethyl acetate and concentrated in vacuo. The residue (560 mg, a crude mixture of different reduction products) was dissolved in DMSO (4.5 ml) and treated with ammoniumcerium-nitrate (2 M solution in water, 1.9 ml, 3.8 mmol) resulting in a brownish suspension, with was stirred over night at ambient temperature. The mixture was dissolved by addition of more DMSO, acetonitrile and some 5 M formic acid and directly purified via prep. HPLC (C18, gradient: 0.1% aq. formic acid/acetonitrile). Yield: 175 mg (31% of th.) A mixed fraction (75 mg) was repurified by flash chromatography on silica with cyclohexane/ethylacetate (5-50%) yielding a second crop of 37 mg (7% of theory).

LC-MS (Method 4): Rₜ=3.80 min; MS (ESIpos): m/z=519 [M+H]⁺

¹H-NMR (400 MHz, DMSO-d6) δ[ppm]: −0.008 (2.70), 0.008 (2.80), 1.687 (1.87), 1.724 (3.01), 1.763 (2.03), 2.030 (0.72), 2.038 (1.16), 2.045 (1.24), 2.057 (1.32), 2.073 (1.91), 2.080 (1.57), 2.088 (1.14), 2.192 (0.71), 2.201 (1.15), 2.214 (1.69), 2.226 (1.65), 2.235 (1.62), 2.248 (1.14), 2.256 (0.82), 2.269 (0.56), 2.395 (0.93), 2.425 (2.48), 2.454 (2.48), 2.523 (1.11), 3.457 (1.19), 3.486 (2.26), 3.515 (2.39), 3.545 (2.55), 3.572 (1.42), 3.730 (1.41), 4.000 (2.13), 4.010 (3.59), 4.024 (3.66), 4.038 (3.27), 4.204 (0.82), 4.211 (1.03), 4.232 (2.75), 4.239 (2.11), 4.252 (2.47), 4.262 (2.60), 4.273 (2.58), 4.280 (2.20), 4.289 (2.31), 4.301 (0.90), 4.308 (1.00), 4.317 (0.71), 5.286 (1.08), 5.301 (2.49), 5.320 (2.53), 5.335 (1.12), 6.778 (4.25), 6.781 (4.67), 6.799 (4.92), 6.801 (5.15), 6.910 (2.31), 6.913 (2.42), 6.929 (4.82), 6.931 (4.87), 6.947 (2.96), 6.950 (2.90), 7.150 (2.40), 7.154 (2.57), 7.171 (3.98), 7.189 (2.06), 7.193 (2.33), 7.204 (1.61), 7.208 (2.00), 7.220 (1.97), 7.230 (2.00), 7.242 (1.03), 7.398 (4.14), 7.416 (3.86), 7.571 (0.69), 7.578 (0.85), 7.586 (0.98), 7.593 (1.57), 7.598 (1.47), 7.606 (1.60), 7.614 (1.53), 7.620 (1.61), 7.627 (1.01), 7.634 (0.92), 7.642 (0.84), 7.774 (2.56), 7.792 (4.98), 7.813 (5.12), 7.832 (5.89), 7.835 (6.55), 7.850 (3.13), 7.853 (2.79), 8.561 (3.72), 8.564 (3.88), 8.582 (3.67), 8.585 (3.55), 8.738 (16.00), 9.160 (4.40), 9.180 (4.33).

### Procedure 2:

Under argon a flask was charged with 8-bromo-N-[(4S)-3,4-dihydro-2H-chromen-4-yl]-4-(tetrahydro-2H-pyran-4-yl)quinoline-3-carboxamide (Expl. 6A) (850 mg, 1.82 mmol), 2,3,5-trifluorobenzene boronic acid (160 mg, 0.91 mmol), potassiumcarbonate (503 mg, 3.64 mmol), ′, 1′-bis(diphenylphosphino)ferrocene-palladium(II)dichloride dichloromethane complex (44.6 mg, 54.6 gmol) and ′(2′-aminobiphenyl-2-yl)(chloro)palladi-m-dicyclohexy′(2′,4′,6′-triisopropylbiphenyl-2-yl)phosphine (1:1) (42.9 mg, 54.6 gmol) and a degassed 5:1 mixture of dioxane/water (5.1 ml). The mixture was stirred in a preheated bath of 70° C. for 45 min. Then another portion of 2,3,5-trifluorobenzene boronic acid (160 mg, 0.91 mmol) was added and stirred at the same temperature for 45 min. The latter process was repeated once more. After consumption of the starting material, water and ethylacetate were added at RT stirred and the phases separated. The aqueous phase was extracted several times with etylacetate, the combined organic phases dried and evaporated under diminished pressure. The residue (1.15 g) war purified by flash chromatography on silica (100 g) with cyclohexane/ethylacetate (32-40%).

Yield: 833 mg (88% of theory)

LC-MS (Method 2): Rₜ=1.13 min; MS (ESIpos): m/z=519 [M+H]⁺

¹H-NMR (500 MHz, DMSO-d6) δ[ppm]: 1.691 (1.98), 1.716 (2.17), 1.736 (1.98), 1.761 (2.04), 2.038 (0.81), 2.045 (1.25), 2.050 (1.33), 2.060 (1.42), 2.073 (1.82), 2.078 (1.55), 2.085 (1.09), 2.200 (0.78), 2.207 (1.17), 2.217 (1.73), 2.227 (1.71), 2.234 (1.72), 2.245 (1.25), 2.252 (0.84), 2.262 (0.56), 2.405 (1.02), 2.429 (2.67), 2.453 (2.70), 3.464 (1.17), 3.485 (2.16), 3.508 (1.29), 3.523 (1.50), 3.544 (2.52), 3.567 (1.39), 3.730 (1.32), 4.003 (2.20), 4.013 (3.04), 4.025 (4.09), 4.036 (2.77), 4.046 (1.82), 4.212 (0.98), 4.218 (1.16), 4.234 (3.00), 4.240 (2.08), 4.251 (2.37), 4.257 (1.90), 4.268 (1.90), 4.275 (2.44), 4.281 (2.12), 4.288 (2.38), 4.298 (1.00), 4.304 (1.09), 4.310 (0.80), 5.292 (1.22), 5.304 (2.66), 5.320 (2.56), 5.331 (1.16), 5.752 (1.91), 6.783 (5.04), 6.798 (5.49), 6.915 (2.50), 6.930 (5.20), 6.945 (2.93), 7.154 (2.55), 7.157 (2.49), 7.171 (4.18), 7.185 (2.13), 7.188 (1.98), 7.208 (2.12), 7.217 (2.09), 7.225 (2.06), 7.399 (4.31), 7.415 (4.06), 7.574 (0.76), 7.580 (0.94), 7.586 (1.07), 7.592 (1.68), 7.602 (1.71), 7.609 (1.64), 7.613 (1.64), 7.619 (0.99), 7.624 (0.89), 7.631 (0.78), 7.778 (2.81), 7.793 (5.08), 7.810 (4.86), 7.833 (6.27), 7.835 (6.27), 7.847 (3.60), 8.563 (4.14), 8.580 (3.96), 8.738 (16.00), 9.159 (4.57), 9.176 (4.42).

### Example 13

### (S)—N-(2,3-Dihydro-1H-inden-1-yl)-3-(3-fluorophenyl)-7-isopropyl-2-methylpyrazolo[1,5-a]pyrimidine-6-carboxamide

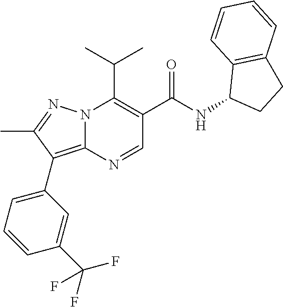 [SMILES: Cc1nn2c(C(C)C)c(C(=O)N[C@H]3CCc4ccccc43)cnc2c1-c1cccc(C(F)(F)F)c1]

### Step 1: Ethyl 2-(ethoxymethylene)-4-methyl-3-oxopentanoate (1B-1)

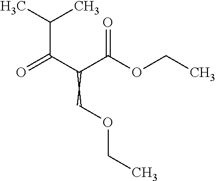

A mixture of ethyl isobutyrylacetate (24.8 g, 157 mmol, 25.3 mL), triethyl orthoformate (46.5 g, 314 mmol, 52.2 mL) and acetic anhydride (32.0 g, 314 mmol, 29.7 mL) was stirred at reflux for 19 h. The volatiles were removed in vacuo (100° C., 0.5 Torr) to afford 28.6 g (133 mmol; 85% of theory) of the title compound. Material was used as such.

### Step 2: Ethyl 7-isopropyl-2-methylpyrazolo[1,5-a]pyrimidine-6-carboxylate (1C-1)

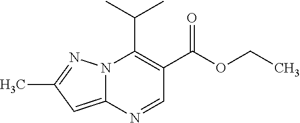 [SMILES: CCOC(=O)c1cnc2cc(C)nn2c1C(C)C]

A solution of 3-amino-5-methylpyrazole (12.95 g, 133 mmol) and ethyl 2-(ethoxymethylene)-4-methyl-3-oxopentanoate (28.56 g, 133 mmol) in absolute ethanol (400 mL) was stirred at reflux for 48 h. The reaction mixture was concentrated in vacuo to afford 32.38 g (128 mmol; 96% of theory) of the title compound.

LC-MS (Method L1): Rₜ=2.19 min; m/z=248 (M+H)+

¹H NMR (300 MHz, Chloroform-d, Method M2) δ 8.71 (s, 1H), 6.46 (s, 1H), 4.55 (dq, J=14.1, 7.0 Hz, 1H), 4.40 (q, J=7.1 Hz, 2H), 2.51 (s, 3H), 1.58 (s, 6H), 1.41 (t, J=7.1 Hz, 3H).

### Step 3: Ethyl 3-bromo-7-isopropyl-2-methylpyrazolo[1,5-a]pyrimidine-6-carboxylate (1D-1)

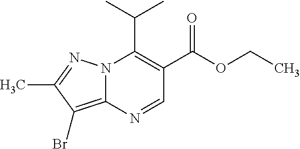 [SMILES: CCOC(=O)c1cnc2c(Br)c(C)nn2c1C(C)C]

To a stirring solution of ethyl 7-isopropyl-2-methylpyrazolo[1,5-a]pyrimidine-6-carboxylate (32.38 g, 131 mmol) in acetonitrile (1.3 L) was added N-bromosuccinimide (23.71 g, 133 mmol). After 20 minutes the reaction mixture was concentrated in vacuo, to afford 71.20 g of a solid, which was triturated in diethyl ether (0.4 L). The solids were filtered off and washed with diethyl ether. The filtrate was concentrated in vacuo to yield 46.50 g of a solid. The material was triturated in diisopropyl ether (1.0 L). The solids were filtered off and the filtrate was treated with active charcoal (6.4 g). The charcoal was filtered off over kieselguhr and the filtrate was concentrated in vacuo to afford 42.06 g (126 mmol; 97% of theory) of the title compound.

LC-MS (Method L1.1): Rₜ=2.32 min; m/z=326/328 (M+H)⁺

¹H NMR (300 MHz, Chloroform-d, Method M2) δ 8.79 (s, 1H), 4.54 (m, 1H), 4.41 (q, J=7.1 Hz, 2H), 2.52 (s, 3H), 1.59 (d, J=7.1 Hz, 6H), 1.42 (t, J=7.1 Hz, 3H).

### Step 4: 3-Bromo-7-isopropyl-2-methylpyrazolo[1,5-a]pyrimidine-6-carboxylic acid (1E-1)

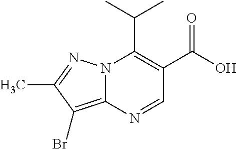 [SMILES: Cc1nn2c(C(C)C)c(C(=O)O)cnc2c1Br]

To a solution of ethyl 3-bromo-7-isopropyl-2-methylpyrazolo[1,5-a]pyrimidine-6-carboxylate (42.0 g, 129 mmol) in tetrahydrofuran (800 mL) was added a solution of lithium hydroxide monohydrate (42.0 g, 1001 mmol) in water (800 mL). The mixture was stirred at room temperature for 5 h. The organic solvent was removed in vacuo. The basic aqueous layer was washed with ethyl acetate (2×400 mL). The organic extracts were set aside. The aqueous layer was acidified with a solution of concentrated hydrochloric acid (50 mL) in water (500 mL) and was extracted with ethyl acetate (2×400 mL). The aqueous layer was further acidified with hydrochloric acid (4N; 200 mL) and was extracted with ethyl acetate (2×400 mL). The combined organic layers were washed with water (400 mL) and brine (400 mL) and were dried with sodium sulfate. Solvents were removed in vacuo and the residue was co-evaporated with toluene and ethyl acetate to afford 26.0 g (84 mmol) of the title compound.

The organic extracts that were obtained from washing the basic aqueous layer were concentrated and partitioned between hydrochloric acid (1N; 500 mL) and ethyl acetate (300 mL). The organic layer was separated and the aqueous layer was extracted with ethyl acetate (2×300 mL). The combined organic layers were washed with water and brine and were dried with sodium sulfate. Solvents were removed in vacuo to afford 8.9 g (27 mmol) of the title compound. In total 34.9 g (111 mmol; 86% of theory) of the title compound were obtained

LC-MS (Method L1.1): Rₜ=2.12 min; m/z=298/300 (M+H)⁺

### Step 5: (S)-3-Bromo-N-(2,3-dihydro-1H-inden-1-yl)-7-isopropyl-2-methylpyrazolo[1,5-a]pyrimidine-6-carboxamide (1F-1)

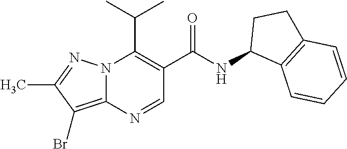 [SMILES: Cc1nn2c(C(C)C)c(C(=O)N[C@H]3CCc4ccccc43)cnc2c1Br]

To a solution of 3-bromo-7-isopropyl-2-methylpyrazolo[1,5-a]pyrimidine-6-carboxylic acid (15.6 g, 52.3 mmol) and (S)-2,3-dihydro-1H-inden-1-amine (6.9 g, 52.3 mmol, 6.7 mL) in dry N,N-dimethylformamide (500 mL) were added N-(3-dimethylaminopropyl)-N′-ethylcarbodiimide hydrochloride (11.0 g, 57.6 mmol) and 1-hydroxy-7-azabenzotriazole (0.7 g, 5.2 mmol) at 0° C. The mixture was stirred at 0° C. for 30 min and at room temperature for 5 h. Water (1.5 L) was added and a precipitate occurred. The suspension was stirred for 30 min after which the solid was filtered off and washed with water. The solid was dried at 40° C. for four days in vacuo to afford 20.2 g (49.0 mmol; 94% of theory) of the title compound.

LC-MS (Method L1.1): Rₜ=2.25 min; m/z=413/415 (M+H)⁺

¹H NMR (300 MHz, DMSO-d6, Method M2) δ 9.06 (d, J=8.2 Hz, 1H), 8.52 (s, 1H), 7.43-7.33 (m, 1H), 7.33-7.19 (m, 3H), 5.51 (q, J=7.8 Hz, 1H), 4.05-3.83 (m, 1H), 3.06-2.78 (m, 2H), 2.61-2.51 (m, 1H), 2.46 (s, 3H), 2.02-1.83 (m, 1H), 1.52 (dd, J=7.0, 5.0 Hz, 6H).

### Step 6: (S)—N-(2,3-Dihydro-1H-inden-1-yl)-3-(3-fluorophenyl)-7-isopropyl-2-methylpyrazolo[1,5-a]pyrimidine-6-carboxamide

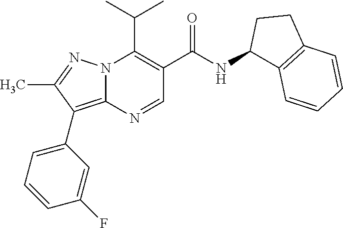 [SMILES: Cc1nn2c(C(C)C)c(C(=O)N[C@H]3CCc4ccccc43)cnc2c1-c1cccc(F)c1]

A solution of (S)-3-bromo-N-(2,3-dihydro-1H-inden-1-yl)-7-isopropyl-2-methylpyrazolo[1,5-a]pyrimidine-6-carboxamide (101 mg, 0.24 mmol), 3-fluorophenylboronic acid (38 mg, 0.27 mmol) and sodium carbonate (78 mg, 0.73 mmol) in a mixture of 1,2-dimethoxyethane (3.0 mL) and water (0.8 mL) was purged with argon for 5 minutes. Bis(triphenylphosphine)-palladium(II) chloride (9 mg, 0.01 mmol) was added and the resulting mixture was stirred at 100° C. for 20 h. The reaction mixture was allowed to cool to room temperature and concentrated in vacuo. Purification by flash column chromatography (Method L7; heptane, 1%-15% ethyl acetate) afforded 76 mg (0.18 mmol; 73% of theory) of the title compound.

LC-MS (Method L2.1): Rₜ=3.78 min; m/z=429 (M+H)⁺

¹H NMR (300 MHz, Chloroform-d, Method M2) δ 8.42 (s, 1H), 7.51-7.20 (m, 7H), 7.00 (m, 1H), 6.09 (d, J=8.4 Hz, 1H), 5.68 (q, J=7.6 Hz, 1H), 4.11 (p, J=7.0 Hz, 1H), 3.12-2.88 (m, 2H), 2.82-2.68 (m, 1H), 2.65 (s, 3H), 2.03-1.88 (m, 1H), 1.65 (dd, J=7.0, 4.1 Hz, 6H).

Example 13: ¹H-NMR (400.0 MHz, d₆-DMSO): δ [ppm]=9.092(1.5), 9.071(1.5), 8.577(7.6), 8.316(0.5), 8.128(2.5), 8.040(1.3), 8.021(1.5), 7.746(0.8), 7.726(1.9), 7.707(1.3), 7.677(1.8), 7.657(0.9), 7.404(0.9), 7.392(1.2), 7.383(1.2), 7.298(0.5), 7.286(1.0), 7.276(1.7), 7.266(1.1), 7.255(3.9), 7.248(2.3), 7.241(2.0), 7.233(2.1), 5.556(0.4), 5.536(1.3), 5.516(1.3), 5.496(0.4), 4.019(0.4), 4.001(0.9), 3.984(1.3), 3.966(1.0), 3.948(0.4), 3.325(128.4), 3.026(0.3), 3.017(0.4), 3.004(0.4), 2.995(0.4), 2.986(0.7), 2.977(0.7), 2.964(0.7), 2.955(0.7), 2.912(0.5), 2.891(1.1), 2.871(0.8), 2.852(0.6), 2.831(0.3), 2.675(0.9), 2.671(1.1), 2.666(0.9), 2.662(0.7), 2.657(1.0), 2.646(16.0), 2.575(0.4), 2.566(0.5), 2.555(0.8), 2.544(1.0), 2.535(1.3), 2.524(3.8), 2.511(61.4), 2.506(124.4), 2.502(163.6), 2.497(116.4), 2.493(55.2), 2.333(0.8), 2.328(1.1), 2.324(0.8), 1.989(0.4), 1.970(0.3), 1.949(0.9), 1.938(0.4), 1.928(0.9), 1.917(0.8), 1.907(0.4), 1.897(0.8), 1.586(7.2), 1.568(13.9), 1.551(7.1), 0.146(0.6), 0.008(5.1), 0.000(142.0), −0.009(5.1), −0.150(0.6)

### Example 14

### (S)—N-(chroman-4-yl)-4-(oxetan-3-yl)-8-(2,3,5-trifluorophenyl)quinoline-3-carboxamide

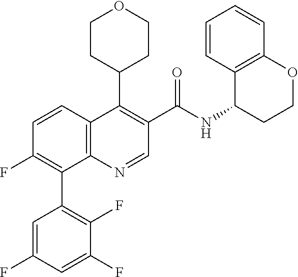 [SMILES: O=C(N[C@H]1CCOc2ccccc21)c1cnc2c(-c3cc(F)cc(F)c3F)c(F)ccc2c1C1CCOCC1]

Example 14 was prepared analogously to Example 3 of WO2019/215182

LC-MS (Method 2): Rt=1.14 min; MS (ESIpos): m/z=537 [M+H]+

¹H-NMR (400 MHz, DMSO-d6) δ[ppm]: 1.679 (4.00), 1.713 (6.39), 1.737 (3.34), 2.033 (2.47), 2.052 (2.85), 2.067 (3.55), 2.211 (2.97), 2.220 (3.09), 2.229 (2.93), 2.243 (2.14), 2.328 (0.95), 2.367 (2.64), 2.388 (4.62), 2.401 (5.48), 2.420 (5.36), 2.432 (5.24), 2.449 (2.76), 2.464 (2.06), 2.670 (0.95), 2.710 (0.91), 3.444 (1.32), 3.471 (3.63), 3.499 (4.04), 3.523 (3.09), 3.543 (4.29), 3.574 (2.23), 3.737 (3.01), 4.016 (7.26), 4.036 (6.06), 4.197 (2.06), 4.219 (4.21), 4.225 (4.91), 4.246 (4.41), 4.270 (4.74), 4.286 (4.16), 5.275 (2.27), 5.288 (5.07), 5.307 (5.11), 5.322 (2.19), 6.778 (9.73), 6.799 (10.85), 6.904 (4.87), 6.923 (10.35), 6.942 (6.10), 7.152 (5.03), 7.170 (8.37), 7.188 (4.00), 7.278 (3.96), 7.286 (4.00), 7.389 (8.87), 7.407 (8.29), 7.642 (1.57), 7.650 (1.90), 7.665 (3.42), 7.678 (3.34), 7.686 (3.34), 7.692 (3.38), 7.698 (2.10), 7.706 (1.86), 7.714 (1.73), 7.761 (5.44), 7.784 (10.27), 7.807 (5.61), 8.664 (5.11), 8.679 (5.65), 8.688 (5.53), 8.703 (4.95), 8.758 (15.75), 8.770 (16.00), 9.155 (6.72), 9.173 (6.47).

### Formulations

The compound of Example 9 was prepared in the following two exemplary formulations:

### Formulation for Intravenous Administration:

### Compound of Example 9 in PEG300 (90%): EtOH (10%)

The compound of Example 9 was weighed out in concentration ranges of 0.5-1 mg/mL and the stock PEG300 (90%): EtOH (10%) solution was added. The resulting suspension was stirred until a full solution was obtained.

### Formulation for Subcutaneous Administration:

### Compound of Example 9 in 0.5% Cellulose (HPMC 5 cP, Mantocel), 1% Lutrol F68 (Poloxamer 188), 0.9% NaCl, Water

The compound of Example 9 was weighed out in concentration ranges of 5-50 mg/mL and the stock 0.5% cellulose (HPMC 5 cP, Mantocel), 1% Lutrol F68 (Poloxamer 188), 0.9% NaCl, water was added. The resulting suspension was stirred. Particle size reduction of the active compound was achieved using a probe sonicator for 30-90 minutes.

### Compound of Example 9 in % Cellulose (HEC), 0.5% Tween 80, 0.15% Paraben, 0.05% Antifoam, Water

The compound of Example 9 was weighed out in concentration ranges of 5-50 mg/mL and the stock 1% cellulose (HEC), 0.5% Tween 80, 0.15% Paraben, 0.05% Antifoam, water was added. The resulting suspension was stirred. Particle size reduction of the active compound was achieved using a probe sonicator for 30-90 minutes.

### Biological Assays

### Assay 1: Determination of Half-Life and Plasma Clearance of Compounds of the Invention Upon Administration to Beagle Dogs

Compounds according to the invention were administered as an intravenous (iv) dose (up to 0.3 mg/kg) to beagle dogs (minimum n=3). Blood samples were collected from these beagle dogs up to 168 h after intravenous administration and up to 3024 h after subcutaneous administration. A portion of each whole blood sample was processed to plasma. Compound concentrations were determined in plasma using LCMS/MS. Phoenix™ Pharmacokinetic and Pharmacodynamic (PK/PD) was used to determine half-life and plasma clearance values. Only for Example 1, an internal tool available to the inventors was used to determine the half-life and plasma clearance values.

Table 1 summarizes the results of Assay 1.

TABLE 1
Half-life and plasma clearance data of example compounds

| | | Half-life | Plasma |
|---|---|---|---|
| | | (h/Form) | Clearance |
| | | iv | (L/h/kg) |
| Example | Structure | geometric mean | geometric mean |
| Example 1 | 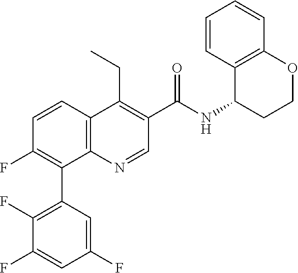 [SMILES: CCc1c(C(=O)N[C@H]2CCOc3ccccc32)cnc2c(-c3cc(F)cc(F)c3F)c(F)ccc12] | 52 | 0.60 |
| Example 2 | 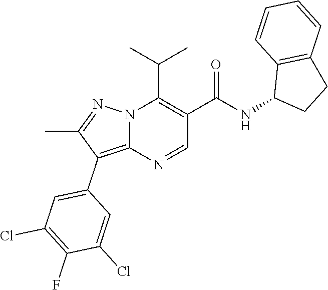 [SMILES: Cc1nn2c(C(C)C)c(C(=O)N[C@H]3CCc4ccccc43)cnc2c1-c1cc(Cl)c(F)c(Cl)c1] | 19.01 | 0.14 |
| Example 3 | 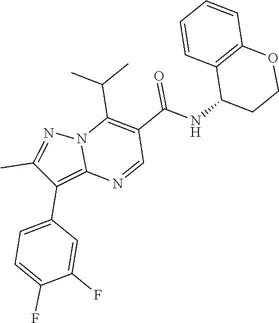 [SMILES: Cc1nn2c(C(C)C)c(C(=O)N[C@H]3CCOc4ccccc43)cnc2c1-c1ccc(F)c(F)c1] | 37.43 | 0.18 |
| Example 4 | 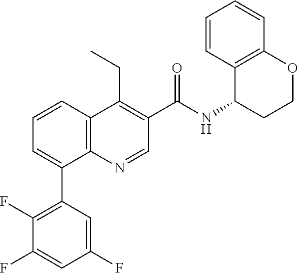 [SMILES: CCc1c(C(=O)N[C@H]2CCOc3ccccc32)cnc2c(-c3cc(F)cc(F)c3F)cccc12] | 34.73 | 1.22 |
| Example 5 | 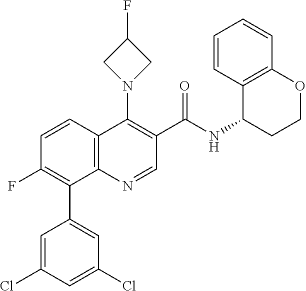 [SMILES: O=C(N[C@H]1CCOc2ccccc21)c1cnc2c(-c3cc(Cl)cc(Cl)c3)c(F)ccc2c1N1CC(F)C1] | 160.34 | 0.07 |
| Example 6 | 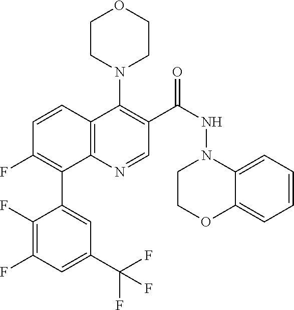 [SMILES: O=C(NN1CCOc2ccccc21)c1cnc2c(-c3cc(C(F)(F)F)cc(F)c3F)c(F)ccc2c1N1CCOCC1] | 125.78 | 0.10 |
| Example 7 | 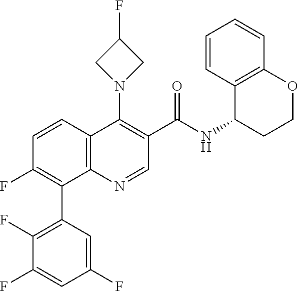 [SMILES: O=C(N[C@H]1CCOc2ccccc21)c1cnc2c(-c3cc(F)cc(F)c3F)c(F)ccc2c1N1CC(F)C1] | 123.7 | 0.05 |
| Example 8 | 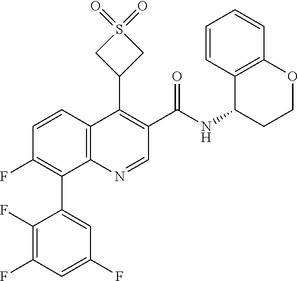 [SMILES: O=C(N[C@H]1CCOc2ccccc21)c1cnc2c(-c3cc(F)cc(F)c3F)c(F)ccc2c1C1CS(=O)(=O)C1] | 43.65 | 0.69 |
| Example 9 | 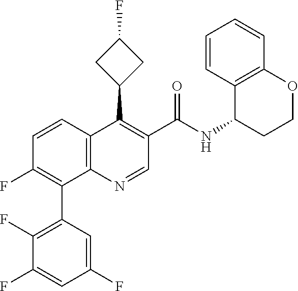 [SMILES: O=C(N[C@H]1CCOc2ccccc21)c1cnc2c(-c3cc(F)cc(F)c3F)c(F)ccc2c1[C@H]1C[C@H](F)C1] | 189.98 | 0.07 |
| Example 10 | 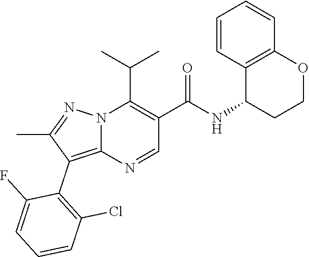 [SMILES: Cc1nn2c(C(C)C)c(C(=O)N[C@H]3CCOc4ccccc43)cnc2c1-c1c(F)cccc1Cl] | 24.66 | 0.37 |
| Example 11 | 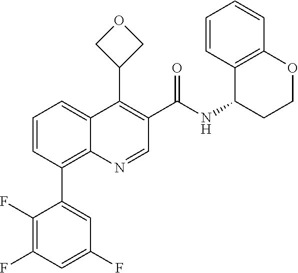 [SMILES: O=C(N[C@H]1CCOc2ccccc21)c1cnc2c(-c3cc(F)cc(F)c3F)cccc2c1C1COC1] | 47.76 | 0.30 |
| Example 12 | 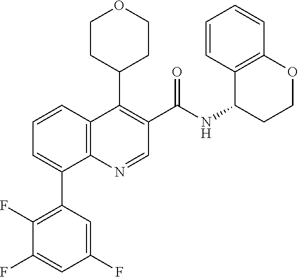 [SMILES: O=C(N[C@H]1CCOc2ccccc21)c1cnc2c(-c3cc(F)cc(F)c3F)cccc2c1C1CCOCC1] | 21.45 | 1.02 |
| Example 13 | 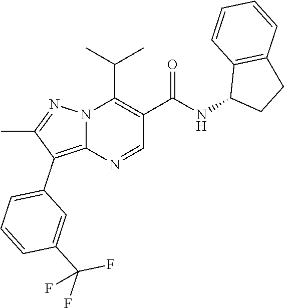 [SMILES: Cc1nn2c(C(C)C)c(C(=O)N[C@H]3CCc4ccccc43)cnc2c1-c1cccc(C(F)(F)F)c1] | 45.43 | 0.56 |
| Example 14 | 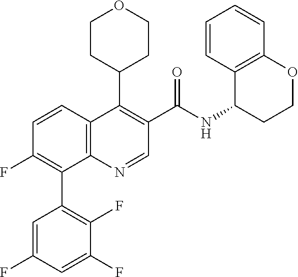 [SMILES: O=C(N[C@H]1CCOc2ccccc21)c1cnc2c(-c3cc(F)cc(F)c3F)c(F)ccc2c1C1CCOCC1] | 43.43 | 0.18 |

### Assay 2: Treatment Efficacy and Residual Protection Efficacy of the Compound of Example 9 Regarding Heartworm Infections

### Study Design:

Eight (8) dogs were allocated to two different groups (n=4 dogs per group):
Group 1: negative control
Group 2: compound of Example 9 at 30 mg/kg body-weight was administered subcutaneously.

All dogs were infected with ~50 3^{rd} stage larvae (L3s) of Dirofilaria immitis via subcutaneous injection in the inguinal region (VICH GL19, Efficacy of Anthelmintics: Specific Recommendations for Canines, June 2001).

90 days later, the compound of Example 9 was administered via subcutaneous injection to dogs in Group 2 at a dose of 30 mg/kg. Dogs in the negative control group (Group 1) received an injection of an equivalent volume of vehicle solution.

### Prevention Test:

To evaluate the effectiveness of the compound of Example 9 at preventing the development of adult heartworms in dogs infected with Dirofilaria immitis 90 days earlier, modified Knott and heartworm antigen tests (DiroCHEKR Canine Heartworm Antigent Test (Zoetis)) were performed on each dog to detect circulating microfilaria and adult female worms, respectively, at intervals of 150, 180 and 210 days after infection.

### Results of the Prevention Test:

The results of the prevention test with the compound of Example 9 are depicted in Table 2 below.

TABLE 2
Results of modified Knott and adult heartworm antigen
testing conducted 150, 180 and 210 days after experimental
Dirofilaria immitis infection in dogs of treatment groups 1 and 2

| Test Type and | | | | | | Compound of |
|---|---|---|---|---|---|---|
| Number of Days | | | | | | Example 9 at |
| after 1^{st} Infection | | Negative Control | | | | 30 mg/kg |
| Modified | 150 | − | | | | − |
| Knott Test | 180 | + | | | | − |
| | 210 | + | | | | − |
| Heartworm | 150 | − | − | + | + | − |
| Antigen Test | 180 | − | + | + | + | − |

| | | |
|---|---|---|
| 210 | + | − |

Circulating microfilaria were detected in all negative control dogs 180 days post-infection. All negative control dogs also had a positive heartworm antigen test 210 days post-infection.

None of the dogs treated with the compound of Example 9 had positive test results in the modified Knott and heartworm antigen tests 150, 180 and 210 days after infection.

This indicates that the compound of Example 9 is 100% effective at preventing the development of patent adult heartworm when administered 90 days after experimental infection.

### Residual Protection Test:

90 days after treatment with the compound of Example 9, all 8 dogs were infected a second time with another ~50 Dirofilaria immitis L3s, using the same infection route as described above in the study design of Assay 2.

Approximately 5 months after infection, a heartworm antigen test was performed.

Moreover, each dog was humanely euthanized and the total numbers of Dirofilaria immitis present in the heart and lungs were determined.

### Results of the Residual Protection Test:

Heartworm antigen test results were positive for all negative control dogs (Group 1) 149 days after the second Dirofilaria immitis infection. In contrast, only 50% of the dogs which had been treated with 30 mg/kg of the compound of Example 9 prior to the second Dirofilaria immitis infection (Group 2) had a positive heartworm antigen test result.

All negative control dogs were heavily infected with Dirofilaria immitis with a geometric group mean count of 60.9 worms (range 50-82 worms per dog) in the heart and lungs. All dogs which had been treated with 30 mg/kg of the compound of Example 9 prior to the second Dirofilaria immitis infection (Group 2) exhibited >80% reduction in worm burden relative to the negative control group in the heart and lungs.

These results indicate that the compound of Example 9 can provide significant residual protection from infection with Dirofilaria immitis.

### Assay 3: Treatment Efficacy and Residual Protection Efficacy of the Compound of Example 9 Regarding Hookworm Infections

### Study Design:

12 dogs were allocated to one of six groups in three cohorts. Each cohort comprised a negative control group (n=2) (‘Control group’) and a group (n=2) treated with the compound of Example 9 (‘Treatment group’).

The compound of Example 9 was administered via subcutaneous injection at a dosage of 30 mg/kg body-weight in the treatment groups. Dogs in negative control groups were administered an equivalent volume of vehicle solution.

All dogs were infected with 100-300 hookworm (Ancylostoma caninum) 3^{rd}-stage larvae (L3s) via oral gavage (W.A.A.V.P. Guidelines, First edition; Jacobs, D. E., A. Arakawa, C. H. Courtney, et al., 1994. Vet. Parasitol. 52: 179-202). The cohorts were treated as follows:
Cohort A: The compound of Example 9 or the vehicle solution was administered 15 days after infection with Ancylostoma caninum
Cohort B: The compound of Example 9 or the vehicle solution was administered 30 days prior to infection with Ancylostoma caninum
Cohort C: The compound of Example 9 or the vehicle solution was administered 60 days prior to infection with Ancylostoma caninum

Fecal egg counts (FECs, eggs per gram of feces) were obtained from each dog 15, 20, 25 and 30 days after hookworm infection.

Following the final FEC, dogs were dewormed with a commercial anthelmintic and feces were collected for 48 hours. Fecal output from each dog was soaked in water for a minimum of 30 minutes and processing through 250 μm, 150 μm and 38 μm sieves to recover expelled adult and immature hookworms. Expelled hookworms were preserved in fixative (5% formalin) and counted.

### Test for Treatment of an Existing Infection:

Cohort A was designed to evaluate effectiveness against patent adult hookworm infections present at the time of treatment with the compound of Example 9.

### Results of the Test for Treatment of an Existing Infection:

All four dogs developed an infection prior to the treatment. Fecal egg count reduction was >90% upon treatment with the compound of Example 9 (see Table 3).

### Residual Efficacy Test:

Cohorts B, and C were designed to evaluate residual efficacy and protection from infections occurring 30 and 60 days after treatment with the compound of Example 9.

### Results of the Residual Efficacy Test:

The combination of FECs and expelled worm counts indicated that the compound of Example 9 provided 100% protection from infections with hookworm larvae for at least 60 days (Table 3) in cohorts B and C.

TABLE 3
Results of fecal egg counts for Cohorts A
(treatment of an existing infection)
as well as B and C (residual efficacy test)

| | | Mean Fecal Egg Count (EPG) at the | | | | Mean |
|---|---|---|---|---|---|---|
| | | Indicated Day After Infection | | | | Number of |
| | | 15 | 20 | 25 | 30 | Recovered |
| Cohort | Group | days | days | days | days | Hookworms |

Therapeutic Treatment/Control of an Existing Infection

| | | | | | | |
|---|---|---|---|---|---|---|
| A | Control | 750 | 225 | 400 | 200 | 6.0 |
| | Treatment: | 0 | 0 | 25 | 0 | 0.0 |
| | Compound | | | | | |
| | of | | | | | |
| | Example 9 | | | | | |

| | | | | | |
|---|---|---|---|---|---|
| % Reduction: | 100 | 100 | 93.75 | 100 | 100 |

30-day Residual Efficacy/Protection from Infection

| | | | | | | |
|---|---|---|---|---|---|---|
| B | Control | 175 | 2150 | 4100 | 14950 | 16.5 |
| | Treatment: | 0 | 0 | 0 | 0 | 0 |
| | Compound | | | | | |
| | of | | | | | |
| | Example 9 | | | | | |

| | | | | | |
|---|---|---|---|---|---|
| % Reduction: | 100 | 100 | 100 | 100 | 100 |

60-day Residual Efficacy/Protection from Infection

| | | | | | | |
|---|---|---|---|---|---|---|
| C | Control | 125 | 3200 | 5625 | 16875 | 30 |
| | Treatment: | 0 | 0 | 0 | 0 | 0 |
| | Compound | | | | | |
| | of | | | | | |
| | Example 9 | | | | | |

| | | | | | |
|---|---|---|---|---|---|
| % Reduction: | 100 | 100 | 100 | 100 | 100 |

1. A method of using a compound of formula I.1 or I.2 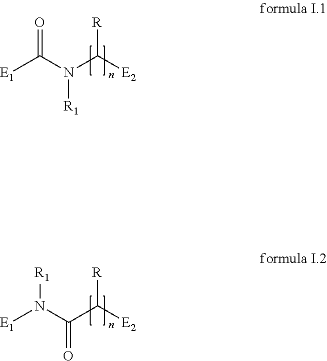
in which:
E1 is an aromatic bicyclic heterocycle substituted with at least one substitutent and wherein the aromatic bicyclic heterocycle is a ring system selected from the group consisting of
a ring system consisting of two 5-membered rings,
a ring system consisting of a 5-membered ring and a 6-membered ring, or
a ring system consisting of two 6-membered rings;
E2 is selected from the group consisting of
an optionally substituted aromatic benzyl,
an optionally substituted 6-membered heterocycle comprising 1, 2, 3 or 4 heteroatoms in form of N, and
an optionally substituted bicyclic residue wherein the bicyclic residue is selected from the group consisting of
a ring system consisting of two 5-membered rings,
a ring system consisting of a 5-membered ring and a 6-membered ring, or
a ring system consisting of two 6-membered rings;
n is 0 or 1;
R1 is selected from the group consisting of hydrogen, cyano, —CHO, —OH, C₁-C₆-alkyl, C₁-C₉-halogenoalkyl having 1 to 5 halogen atoms, C₁-C₄-alkoxy, C₁-C₄-halogenoalkoxy having 1 to 5 halogen atoms, C₃-C₆-cycloalkyl, C₃-C₆-halogenocycloalkyl having 1 to 5 halogen atoms, C₂-C₄-alkenyl, C₂-C₄-alkynyl, C₁-C₄-alkoxy-C₁-C₄-alkyl, C₃-C₆-cycloalkyl-C₁-C₃-alkyl, cyano-C₁-C₄-alkyl, —NH—C₁-C₄-alkyl, —N(C₁-C₄-alkyl)₂, NH₂—C₁-C₄-alkyl-, C₁-C₄-alkyl-NH—C₁-C₄-alkyl-, (C₁-C₄-alkyl)₂N—C₁-C₄-alkyl-, C₁-C₄-alkyl-C(O)—, C₁-C₄-halogenoalkyl-C(O)— having 1 to 5 halogen atoms, C₁-C₄-alkoxy-C(O)—, benzyloxy-C(O)—, C₁-C₄-alkoxy-C₁-C₄-alkyl-C(O)—, —SO₂—C₁-C₄-alkyl, —SO₂—C₁-C₄-halogenoalkyl having 1 to 5 halogen atoms, amino-C₁-C₄-alkyl, C₁-C₄-alkylamino-C₁-C₄-alkyl, di-(C₁-C₄-alkyl)amino-C₁-C₄-alkyl, C₁-C₄-alkylcarbonyl, C₁-C₄-halogenoalkylcarbonyl having 1 to 5 halogen atoms, C₁-C₄-alkoxycarbonyl, benzyloxycarbonyl, C₁-C₄-alkoxy-C₁-C₄-alkylcarbonyl;
and
R is hydrogen, C₁-C₃ alkyl; or
when E2 is E2_5
R and R14 together with the atoms to which they are attached form a 5 or 6-carbon atoms containing unsaturated ring, wherein the unsaturated ring is optionally substituted with one or more C₁-C₃-alkyl, and/or wherein one or more of the ring-forming carbon atoms are optionally replaced by —NH—, —N═, ═N—, —O— or —S— or
R and R14 together with the atoms to which they are attached form a 5 or 6-carbon atoms containing unsaturated ring, wherein the unsaturated ring is optionally substituted with one or more C₁-C₃-alkyl, and/or wherein one or more of the ring-forming carbon atoms are optionally replaced by —NH—, —N═, ═N—, —O— or —S—;
wherein E2_5 is 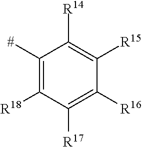
wherein
R₁₅ is independently hydrogen, halogen, C₁₋₃ alkyl, C₁₋₃ alkoxy, or NR^{15′}R^{15″}, wherein R^{15′} and R^{15″} are independently C₁₋₃-alkyl;
R16 is independently hydrogen, halogen, C₁₋₃ alkyl, C₁₋₃ alkoxy, or NR^{16′}R^{16″}, wherein R^{16′} and R^{16″} are independently C₁₋₃-alkyl;
R17 is independently hydrogen, C₁₋₃ alkyl, C₁₋₃ alkoxy, or NR^{17′}R^{17″}, wherein R^{17′} and R^{17″} are independently C₁₋₃-alkyl;
R18 is independently hydrogen, C₁₋₃ alkyl, C₁₋₃ alkoxy, or NR^{18′}R^{18″}, wherein R^{18′} and R¹⁸″ are independently C₁₋₃-alkyl;
or when E2 is E2_6
R and R14 together with the atoms to which they are attached form a 5 or 6-carbon atoms containing non-aromatic ring, wherein the 5 or 6-carbon atoms containing ring is optionally substituted with one or more C1-3-alkyl or ═O, and/or wherein one or more of the ring forming carbon atoms are optionally replaced by —NH—, —N═, ═N—, —O—, —S(O)—, —S(O)2- or —S—, or R and R14 together with the atoms to which they are attached form a 5 or 6-carbon atoms containing aromatic ring, wherein the 5 or 6-carbon atoms containing ring is optionally substituted with one or more C1-3-alkyl, and/or wherein one or more of the ring forming carbon atoms are optionally replaced by —NH—, —N═, ═N—, —O— or —S—;
wherein E2_6 is 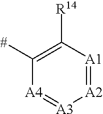
wherein
A1 is N or CR¹⁵, wherein R¹⁵ is independently hydrogen, halogen,
NR^{15′}R^{15″}, wherein R^{15′} and R^{15″} are independently C₁₋₃-alkyl;
A2 is N or CR¹⁶, wherein R¹⁶ is independently hydrogen, halogen,
NR¹⁶R16″, wherein R^{16′} and R^{16″} are independently C₁₋₃-alkyl;
A3 is N or CR¹⁷, wherein R¹⁷ is independently hydrogen, halogen,
NR^{17′}R^{17″}, wherein R^{17′} and R¹⁷ are independently C₁₋₃-alkyl;
A4 is N or CR¹⁸, wherein R¹⁸ is independently hydrogen, halogen
NR^{18′}R^{18″}, wherein R^{18′} and R^{18″} are independently C₁₋₃-alkyl;
or a stereoisomer, a tautomer, an N-oxide, a hydrate, a solvate, or a salt thereof, or a mixture of the same,
in the long-term prevention and/or treatment of a disease.

2. The method of claim 1,
wherein E1 and E2 are selected from the following pairs when the compound of formula I is a compound of formula I.1:
E1_1 and E2_1 wherein n is 0;
E1_2 and E2_1 wherein n is 0;
E1_3 and E2_1 wherein n is 0;
E1_6 and E2_1 wherein n is 0;
E1_7 and E2_2 wherein n is 0;
or
wherein E1 and E2 form the following pair when the compound of formula I is a compound of formula I.2:
E1_7 and E2_2 wherein n is 0;
in which
E1_1 is 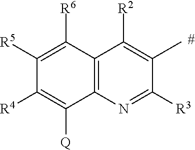
wherein
R2 is selected from the group consisting of hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —NH(C(O)—C₁-C₄-alkyl), —N(C₁-C₄-alkyl)(C(O)—C₁-C₄-alkyl), —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl;
2-oxocyclobutyl, 3-oxocyclobutyl, 2-thiooxocyclobutyl, 3-thiooxocyclobutyl, 3-thietanyl, 2-thietanyl, oxetan-3-yl, oxetan-2-yl, 1-oxidothietan-3-yl, 1-oxidothietan-2-yl, 1-imino-1-oxido-1-thietan-3-yl, 1-imino-1-oxido-1-thietan-2-yl, 1,1-dioxidothietan-3-yl, 1,1-dioxidothietan-2-yl, 1,1-dioxido-1,2-thiazetidin-3-yl, 1,1-dioxido-1,2-thiazetidin-4-yl, 1-oxido-1,2-thiazetidin-3-yl, 1-oxido-1,2-thiazetidin-4-yl, 2-oxido-1,2-oxathietan-3-yl, 2-oxido-1,2-oxathietan-4-yl, 2,2-dioxido-1,2-oxathietan-3-yl, 2,2-dioxido-1,2-oxathietan-4-yl, 4-oxoazetidin-2-yl, 2-oxoazetidin-3-yl, 4-thioxoazetidin-2-yl, 2-thioxoazetidin-3-yl, 2-hydroxycyclobutyl, 3-hydroxycyclobutyl, 2-mercaptocyclobutyl, 3-mercaptocyclobutyl, 2-fluorocyclobutyl, 3-fluorocyclobutyl, 2,2-difluorocyclobutyl, 3,3-difluorocyclobutyl, 2-chlorocyclobutyl, 3-chlorocyclobutyl, 2,2-dichlorocyclobutyl, 3,3-dichlorocyclobutyl, 2-bromocyclobutyl, 3-bromocyclobutyl, 2,2-dibromocyclobutyl, 3,3-dibromocyclobutyl, 2-iodocyclobutyl, 3-iodocyclobutyl, 2,2-diiodocyclobutyl, 3,3-diiodocyclobutyl, 3-methoxyiminocyclobutyl, 2-fluoro-3-(methoxyimino)cyclobutyl, 2,2-difluoro-3-(methoxyimino)cyclobutyl, 2-chloro-3-(methoxyimino)cyclobutyl, 2,2-dichloro-3-(methoxyimino)cyclobutyl, 2-bromo-3-(methoxyimino)cyclobutyl, 2,2-dibromo-3-(methoxyimino)cyclobutyl, 2-iodo-3-(methoxyimino)cyclobutyl, 2,2-diiodo-3-(methoxyimino)cyclobutyl, 3-(hydroxyimino)cyclobutyl, 2-fluoro-3-(hydroxyimino)cyclobutyl, 2,2-difluoro-3-(hydroxyimino)cyclobutyl, 2-chloro-3-(hydroxyimino)cyclobutyl, 2,2-dichloro-3-(hydroxyimino)cyclobutyl, 2-bromo-3-(hydroxyimino)cyclobutyl, 2,2-dibromo-3-(hydroxyimino)cyclobutyl, 2-iodo-3-(hydroxyimino)cyclobutyl, 2,2-diiodo-3-(hydroxyimino)cyclobutyl, tetrahydro-2H-pyran-4-yl, 3,6 dihydro-2H-pyran-4-yl, ethyl and 3-fluoroazetidin-1-yl; and
5- to 10-membered heterocycloalkyl, 5-membered heteroaryl and 6-membered heteroaryl, each of which is optionally substituted by 1, 2 or 3 substituents independently selected from the group consisting of halogen, —OH, -oxo, —NO₂, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R3 is selected from the group consisting of
hydrogen, halogen, —OH, C₁-C₄-alkyl, and C₁-C₄-alkoxy;
R4 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R5 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R6 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R12 and R13 are independently selected from the group consisting of
hydrogen, OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy, C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, OH, cyano, COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms,
—S(O)—C₁-C₄-halogenoalkyl having 1 to 5 halogen atoms —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, OH, cyano, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl,
—S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
hydrogen;
C₁-C₄-alkyl, C₃-C₆-cycloalkyl, phenyl-C₁-C₄-alkyl, each of which is optionally substituted by 1, 2 or 3 substituents independently selected from the group consisting of halogen, OH, cyano, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms,
—S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, OH, oxo, thiono, COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
Q is selected from the group consisting of
(i) 6- to 10-membered aryl and 5- to 10-membered heteroaryl, each of which is optionally substituted by 1, 2, 3, 4 or 5 substituents selected from the group consisting of halogen, SF₅, cyano, —CHO, nitro, oxo, C₁-C₄-alkyl, C₁-C₄-hydroxyalkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms;
(ii) —NH₂, —NH(C₁-C₄-alkyl), —NH(C₃-C₆-cycloalkyl), —NH(phenyl-C₁-C₄-alkyl), —NH(C₁-C₄-alkoxy), —NH(C₁-C₄-alkyl-C(O)—), (—NH(C₁-C₄-alkoxy-C(O)—), —N(C₁-C₄-alkyl)₂, C₁-C₆-alkyl, C₃-C₁₀-cycloalkyl, C₂-C₆-alkenyl, C₃-C₁₀-cycloalkenyl, C₂-C₆-alkynyl, each of which is optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, benzyloxy-C(O)—, C₁-C₄-alkoxy-C₁-C₄-alkyl-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl-C₁-C₄-alkyl-, optionally substituted by 1, 2, 3, 4 or 5 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl-, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-alkyl-C(O)—, C₁-C₄-halogenoalkyl-C(O)— having 1 to 5 halogen atoms, C₁-C₄-alkoxy-C(O)—, C₁-C₄-halogenoalkoxy-C(O)— having 1 to 5 halogen atoms, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
a C- or N-bound monocyclic or bicyclic heterocycle selected from the group consisting of 4- to 10-membered heterocycloalkyl and heterospirocycloalkyl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl; 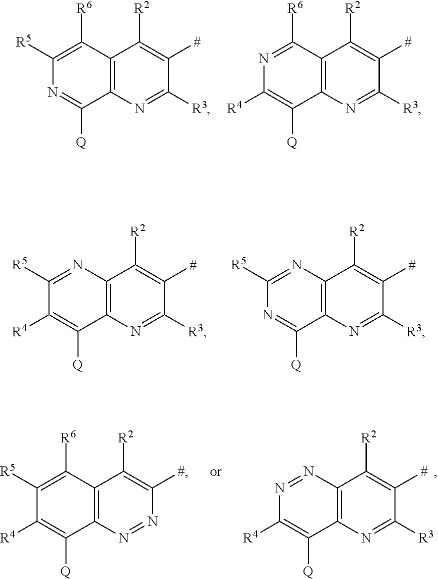
wherein
R2 is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl;
R3 is selected from the group consisting of
hydrogen, halogen and C₁-C₄-alkyl;
R4 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R5 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R6 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R12 and R13 are independently selected from the group consisting of
hydrogen, —OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy,
C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
Q is selected from the group consisting of
6- to 10-membered aryl and 5- to 10-membered heteroaryl, each of which is optionally substituted by 1, 2, 3, 4 or 5 substituents selected from the group consisting of halogen, SF₅, cyano, —CHO, nitro, oxo, C₁-C₄-alkyl, C₁-C₄-hydroxyalkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms;
E1_3 is 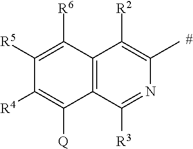
wherein
R2 is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —NH(C(O)—C₁-C₄-alkyl), —N(C₁-C₄-alkyl)(C(O)—C₁-C₄-alkyl), —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl;
R3 is hydrogen or C₁-C₄-alkyl;
R4 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms,
C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
optionally R4 is selected from the group consisting of hydrogen and halogen;
or optionally R4 is selected from the group consisting of fluorine and chlorine;
R5 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R6 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms,
C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R12 and R13 are independently selected from the group consisting of
hydrogen, —OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy,
C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂,
—NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
hydrogen;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂,
—C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl,
—SO₂—C₁-C₄-alkyl, —S—C₁-C₄-halogenoalkyl having 1 to 5 halogen atoms,
—S(O)—C₁-C₄-halogenoalkyl having 1 to 5 halogen atoms and —SO₂—C₁-C₄-halogenoalkyl having 1 to 5 halogen atoms;
Q is selected from the group consisting of 6- or 10-membered aryl and 5- to 10-membered heteroaryl, each of which is optionally substituted by 1, 2, 3, 4 or 5 substituents selected from the group consisting of halogen, SF₅, cyano, —CHO, nitro, oxo, C₁-C₄-alkyl, C₁-C₄-hydroxyalkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl),
—CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms,
wherein when Y is O, S or N—R⁹, none of R⁸, R¹⁰ and R¹¹ is —OH or C₁-C₄-alkoxy, and
wherein when X is O, S or N—R⁹, none of R⁷ and R⁸ is —OH or C₁-C₄-alkoxy;
E1_6 is 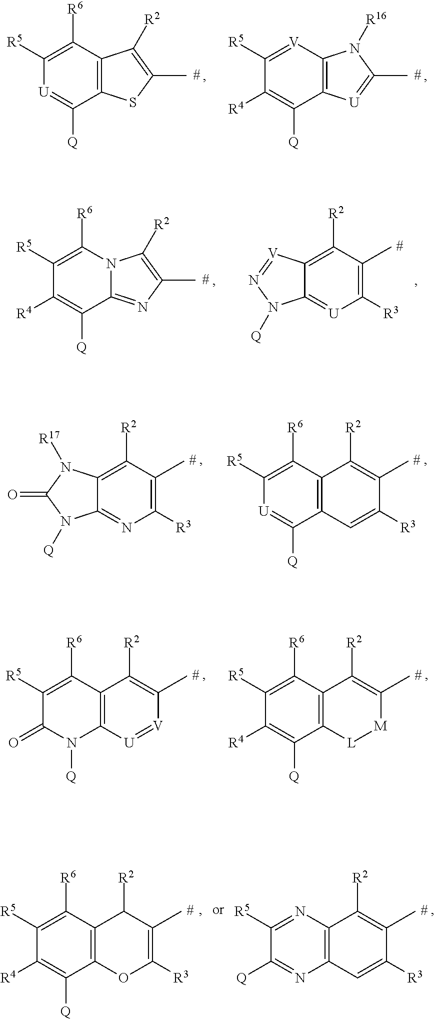
wherein
R2 is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂;
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms,
—NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms,
C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms, and 4- to 10-membered heterocycloalkyl;
R3 is selected from the group consisting of
hydrogen, halogen and C₁-C₄-alkyl;
R4 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms,
C₁-C₄-alkyl-C(O)—, —NH₂, —NH(C₁-C₄-alkyl), —N(C₁-C₄-alkyl)₂, —S—C₁-C₄-alkyl, —S—C₁-C₄-halogenoalkyl having 1 to 5 halogen atoms —S(O)—C₁-C₄-alkyl, —SO₂—C₁-C₄-alkyl;
R5 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-alkyl-C(O)—, —NH₂, —NH(C₁-C₄-alkyl), —N(C₁-C₄-alkyl)₂, —S—C₁-C₄-alkyl, —S(O)—C₁-C₄-alkyl, and —SO₂—C₁-C₄-alkyl;
R6 is selected from the group consisting of
hydrogen, halogen, —OH, cyano, C₁-C₄-alkyl, C₃-C₆-cycloalkyl, C₁-C₄-halogenoalkyl having 1 to 5 halogen atoms, C₁-C₄-alkoxy-C₁-C₄-alkyl, C₁-C₄-alkoxy, C₁-C₄-halogenoalkoxy having 1 to 5 halogen atoms,
C₁-C₄-alkyl-C(O)—, —NH₂, —NH(C₁-C₄-alkyl), —N(C₁-C₄-alkyl)₂, —S—C₁-C₄-alkyl, —S—C₁-C₄-halogenoalkyl having 1 to 5 halogen atoms, —S(O)—C₁-C₄-alkyl, and
—SO₂—C₁-C₄-alkyl;
R7 is selected from the group consisting of
hydrogen, —OH, halogen, C₁-C₄-alkyl and C₁-C₄-alkoxy;
R8 is selected from the group consisting of
hydrogen, —OH, halogen, C₁-C₄-alkyl and C₁-C₄-alkoxy;
or R7 and R8 form, together with the carbon atom to which they are attached, a 3- to 6-membered ring selected from the group consisting of C₃-C₆-cycloalkyl and 3- to 6-membered heterocycloalkyl;
R12 and R13 are independently selected from the group consisting of
hydrogen, —OH, —NH₂, —NH(C₁-C₄-alkyl), —N(C₁-C₄-alkyl)₂, —NH(—C(O)—C₁-C₄-alkyl), —N(C₁-C₄-alkyl)(—C(O)—C₁-C₄-alkyl), C₁-C₄-alkoxy, and
C₁-C₄-alkoxy-C(O)—;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms,
—S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl),
—N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl,
—SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R16 is selected from the group consisting of
hydrogen, C₁-C₄-alkyl, C₁-C₄-halogenoalkyl having 1 to 5 halogen atoms and C₁-C₄-alkoxy;
R17 is selected from the group consisting of
hydrogen, C₁-C₄-alkyl, C₁-C₄-halogenoalkyl having 1 to 5 halogen atoms and C₁-C₄-alkoxy;
U is selected from the group consisting of CR⁷ and N;
V is selected from the group consisting of CR⁷ and N;
M is selected from the group consisting of C═O and CR⁷R⁸;
L is selected from the group consisting of O and NR⁹;
Q is selected from the group consisting of 6- to 10-membered aryl and 5- to 10-membered heteroaryl, each of which is optionally substituted by 1, 2, 3, 4 or 5 substituents selected from the group consisting of halogen, SF₅, cyano, —CHO, nitro, oxo, C₁-C₄-alkyl, C₁-C₄-hydroxyalkyl, C₁-C₄-halogenoalkyl having 1 to 5 halogen atoms, —OH, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms;
E1_7 is 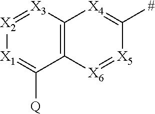
wherein
X1 is selected from the group consisting of N and CR₁;
X2 is selected from the group consisting of N and CR₂;
X3 is selected from the group consisting of N and CR₃;
X4 is selected from the group consisting of N and CR₄;
X5 is selected from the group consisting of N and CR₅;
X6 is selected from the group consisting of N and CR₆;
wherein at least one of X₁, X₂, X₃, and X₅ is N or wherein none of X₁, X₂, X₃, X₄, X₅, and X₆ are N;
R1 is selected from the group consisting of
hydrogen, halogen, hydroxyl, —SH, —SC₁-C₄ alkyl, —S(O)(C₁-C₄ alkyl), —S(O)₂(C₁-C₄ alkyl), cyano, C₁-C₉ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R16 is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R2 is selected from the group consisting of
hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R16 is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R3 is selected from the group consisting of
hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R16 is, each time taken, selected from the group consisting of hydrogen, C₁-C₄ alkyl, and C₃-C₆ cycloalkyl, or R₁₅ and R16 together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R4 is selected from the group consisting of
halogen, cyano, —CHO, hydroxyl, C₁-C₄ alkyl, C₂-C₄ alkenyl, C₂-C₄ alkynyl, C₃-C₆ cycloalkyl, C₁-C₄ halogenoalkyl, C₁-C₄-alkoxy substituted-C₁-C₄ alkyl, benzyl optionally substituted with 1 to 5 halogen atoms, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₁-C₄ alkoxy substituted C₁-C₄ alkyl), —N(C₁-C₄ alkoxy substituted C₁-C₄ alkyl)₂, —N(C(O)C₁-C₄ alkyl)(C₁-C₄ alkyl), —N(C₁-C₄ alkyl)(C₃-C₆-cycloalkyl), —N(C₁-C₄ alkyl)(4- to 7-membered heterocycloalkyl), —NH(4- to 7-membered heterocycloalkyl), —N(C₁-C₄ alkyl)(C₁-C₄ alkoxy), —C(O)NH(C₁-C₄ alkyl), —C(O)N(C₁-C₄ alkyl)₂, —C(O)N(C₁-C₄ alkyl)(4- to 7-membered heterocycloalkyl), —NHSO₂(C₁-C₄ alkyl), —SC₁-C₄ alkyl, —S(O)C₁-C₄ alkyl, —SO₂C₁-C₄ alkyl, —B(OR₁₅)(OR₁₆) wherein R₁₅ is, each time taken, selected from the group consisting of hydrogen, C₁-C₄ alkyl, and C₃-C₆ cycloalkyl, R16 is, each time taken, selected from the group consisting of hydrogen, C₁-C₄ alkyl, and C₃-C₆ cycloalkyl, or R₁₅ and R₁₆ together with the oxygen atoms to which they are attached form a 5- to 7-membered ring which is optionally substituted with 1 to 4 C₁-C₄ alkyl; 6- or 10 membered aryl; a monocyclic heterocycle selected from the group of 4- to 7-membered heterocycloalkyl, 5-membered heteroaryl having at least one nitrogen atom via which the 5-membered heteroaryl ring is connected to the rest of the molecule, and 6-membered heteroaryl having at least one nitrogen atom; each of the aryl, heterocycloalkyl, and heteroaryl rings in R4 is optionally substituted with 1, 2 or 3 substituents independently selected from the group consisting of halogen, cyano, nitro, hydroxy, oxo, imino, 1-imino-1-oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; wherein the C₃-C₆ cycloalkyl and the heterocycloalkyl rings in R4 are optionally substituted with a spiro group, wherein said spiro group is a 3- to 6-membered cycloalkyl or 4- to 6-membered heterocycloalkyl containing 1, 2, or 3 heteroatoms independently selected from N, S or O, wherein said spiro group is optionally substituted with 1, 2 or 3 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and wherein each C₁-C₄ alkyl, C₃-C₆ cycloalkyl and C₁-C₄ alkoxy in R4 may be optionally substituted with 1, 2 or 3 substituents independently selected from the group consisting of halogen, hydroxy, oxo, —NH₂, —NH(C₁-C₄ alkyl), —N(C₁-C₄ alkyl)₂, cyano, carboxy, carbamoyl, C₁-C₄ alkoxycarbonyl, —C(O)NH(C₁-C₄ alkyl), —C(O)N(C₁-C₄ alkyl)₂, C₁-C₄ halogenoalkyl, and C₁-C₄ alkoxy;
R5 is selected from the group consisting of
hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R16 is, each time taken, selected from the group consisting of hydrogen, C₁-C₄ alkyl, and C₃-C₆ cycloalkyl, or R₁₅ and R16 together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R6 is selected from the group consisting of
hydrogen, halogen, hydroxyl, —SH, —SC₁-C₄ alkyl, —S(O)(C₁-C₄ alkyl), —S(O)₂(C₁-C₄ alkyl), cyano, C₁-C₄ alkyl, C₁-C₄ halogenoalkyl, C₁-C₄-alkoxy, —B(OR₁₅)(OR₁₆) wherein R₁₅ is, each time taken, selected from the group consisting of hydrogen, C₁-C₄ alkyl, and C₃-C₆ cycloalkyl, R16 is, each time taken, selected from the group consisting of hydrogen, C₁-C₄ alkyl, and C₃-C₆ cycloalkyl, or R₁₅ and R16 together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
Q is selected from the group consisting of
(i) 6- or 10 membered aryl optionally substituted with 1, 2, 3, 4, or 5 substituents independently selected from the group consisting of halogen, cyano, nitro, hydroxyl, C₁-C₄alkyl, C₁-C₄ halogenoalkyl, C₁-C₄ alkoxy, C₃-C₆ cycloalkyl, —C(O)NH₂, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl, —SO₂C₁-C₄ halogenoalkyl, and pentafluoro-sulfanyl, wherein the 6- or 10 membered aryl is optionally fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is, valency permitting, substituted with a substituent selected from the group consisting of hydrogen, C₁-C₄ alkyl, and C₃-C₆ cycloalkyl;
(ii) 5- to 10-membered heteroaryl having 1, 2, or 3 heteroatoms independently selected from the group O, S, and N and wherein the carbons of the 5- to 10-membered heteroaryl are optionally substituted with 1, 2, 3, 4, or 5 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, benzyloxy, —C(O)R₁₇, —NH₂, —NH(C₁-C₄ alkyl), —N(C₁-C₄ alkyl)₂, —SC_{I}—C₄ alkyl, —S(O)C₁-C₄ alkyl, —SO₂C₁-C₄ alkyl, —S(O)C₁-C₄-halogenoalkyl and —SO₂C₁-C₄ halogenoalkyl, and any N in the heteroaryl, valency permitting, is optionally substituted with a substituent selected from the group consisting of hydrogen, C₁-C₄ alkyl, and C₃-C₆ cycloalkyl;
(iii) 4- to 7-membered heterocycloalkyl having 1, 2, or 3 heteroatoms independently selected from the group O, S, N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1, 2, 3, or 4 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), and —N(C₁-C₄ alkyl)₂ and any N in the heterocycloalkyl is optionally substituted with a substituent selected from the group consisting of hydrogen, C₁-C₄ alkyl, and C₃-C₆ cycloalkyl;
(iv) 6- or 10 membered aryloxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
(v) 6- or 10 membered arylthio-oxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and
(vi) 5- to 10-membered heteroaryloxy optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —C(O)R₁₇, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
E2_1 is 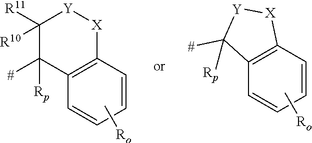
wherein
R10 is selected from the group consisting of hydrogen, —OH, C₁-C₄-alkyl and C₁-C₄-alkoxy;
R11 is selected from the group consisting of hydrogen, C₁-C₄-alkyl and C₁-C₄-alkoxy;
or R10 and R11 form, together with the carbon atom to which they are attached, a 3- to 6-membered ring selected from the group consisting of C₃-C₆-cycloalkyl and 3- to 6-membered heterocycloalkyl;
Rp is hydrogen or C₁-C₄-alkyl;
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms;
o is 0, 1, 2, 3 or 4;
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—;
R7 is selected from the group consisting of hydrogen, —OH, fluorine, C₁-C₄-alkyl and C₁-C₄-alkoxy;
R8 is selected from the group consisting of hydrogen, —OH, halogen, C₁-C₄-alkyl and C₁-C₄-alkoxy, optionally from the group consisting of hydrogen, —OH, fluorine, C₁-C₄-alkyl and C₁-C₄-alkoxy;
or R7 and R8 together form an oxo group (═O);
or R7 and R8 form, together with the carbon atom to which they are attached, a 3- to 6-membered ring selected from the group consisting of C₃-C₆-cycloalkyl and 3- to 6-membered heterocycloalkyl;
R9 is selected from the group consisting of hydrogen, C₁-C₄-alkyl, C₁-C₄-halogenoalkyl having 1 to 5 halogen atoms and C₁-C₄-alkoxy;
wherein when Y is O, S or N—R⁹, none of R⁷, R⁸, R¹⁰ and R¹¹ is —OH, and wherein when X is O, S or N—R⁹, none of R⁸ and R⁵ is —OH;
E2_2 is 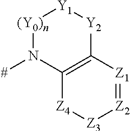
wherein
n is 0 or 1; when n is 1, Y₀ is CH₂ or C═O;
Y₁ is selected from the group consisting of CR₈R₉, O, S, and NR₁₀;
Y₂ is selected from the group consisting of CR₈R₉, O, S, and NR₁₀;
wherein at least one of the groups Y₁ or Y₂ is CR₈R⁹;
Z₁ is selected from the group consisting of N, O, S, and CR₁₁;
Z₂ is selected from the group consisting of Nil, N, and CR₁₁;
Z₃ is selected from the group consisting of Nil, N and CR₁₁;
Z₄ is selected from the group consisting of N, O, S, and CR₁₁;
R8 is each time selected, independently selected from the group consisting of hydrogen, fluoro, and C₁-C₄ alkyl;
R9 is, each time selected, independently selected from the group consisting of hydrogen, fluoro, and C₁-C₄ alkyl;
R10 is selected from the group consisting of hydrogen and C₁-C₄ alkyl;
R11 is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and
—N(C₁-C₄ alkyl)₂;
E2_3 is 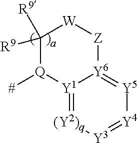
wherein
R9 and R9′ are independently hydrogen, halo, C₁-C₄-alkyl, C₁-C₄-haloalkyl, C₁-C₄-alkoxy, C₁-C₄-haloalkoxy or cycloalkoxy, or R9 together with R9′ form a 2-6-membered chain optionally containing one or two heteroatoms selected from the group consisting of N, O, Si and S to
form carbocyclic or heterocyclic ring together with the carbon atom to which they are attached;
W is CR⁵R⁶, O, SOp, or N—R⁷;
Z is CR⁵R⁶, O, SOp, or N—R⁷;
Q is N or C—R⁸;
Y1 and Y6 are each independently N, C or —CR⁴—;
Y2, Y3, Y4 and Y5 are each independently N, NR′, S, O, —CR⁴— or CR⁴R^{4′};
R4 and R4′ are independently in each occurrence, hydrogen, halogen, cyano, nitro, hydroxyl, optionally substituted alkyl, optionally substituted alkoxy, optionally substituted cycloalkyl, optionally substituted cycloalkoxy, optionally substituted alkylcarbonyl, optionally substituted alkoxycarbonyl, optionally substituted aminocarbonyl, alkylaminocarbonyl, or di(alkyl)aminocarbonyl, optionally substituted alkylcarbonyloxy, optionally substituted alkyl carbonylamino, optionally substituted aryl, optionally substituted heteroaryl, —SF5, —SOp (optionally substituted alkyl or haloalkyl); or R4 together with R4′ together form a 2-6-membered chain optionally containing one or two heteroatoms
selected from the group consisting of N, O, Si and S to form carbocyclic or heterocyclic ring together with the carbon atom to which they are attached; or —NR^{c}R^{d}, wherein R^{C} and R^{d} are independently H or optionally substituted alkyl; or R^{c} and R^{d} may form, with the nitrogen to which they are attached, a 3-, 4-, 5-, 6-, 7-, or 8-membered-heterocyclyl group, which may include one to three additional heteroatoms selected from the group consisting of N, O, Si and S and may be optionally substituted;
R5 and R6 are independently in each occurrence hydrogen, halo, C₁-C₄-alkyl, C₁-C₄-haloalkyl, C₁-C₄-alkoxy, C₁-C₄-haloalkoxy or cycloalkoxy; or R5 together with R6 form a 2-6-membered chain optionally containing one or two heteroatoms selected from the group consisting of N, O, Si and S to form carbocyclic or heterocyclic ring together with the carbon atom to which they are attached;
R7 is hydrogen or C₁-C₄-alkyl;
wherein at most three of Y1, Y2, Y3, Y4, Y5 and Y6 are heteroatoms;
a is 0 or 1;
q is 0 or 1;
p is independently in each occurrence is 0, 1, or 2; 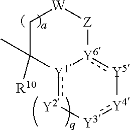
or E2_3 is
wherein
W is CR⁶R⁷, O, S, or N—R⁸
Z is CR⁶R⁷, O, S, or N—R⁸;
Y1′ and Y6′ are each independently N, C, or —CR⁵—;
Y2′, Y3′, Y4′, Y5′ are each independently N, NR², S, O, —CR⁵— or CR⁵R^{5′};
a is 0 or 1;
q is 0 or 1;
R10 hydrogen, halogen, alkyl, haloalkyl, cycloalkyl, alkenyl or alkynyl;
R5 and R5′ are independently in each occurrence, hydrogen, halogen, cyano, nitro, hydroxyl, optionally substituted alkyl, optionally substituted alkoxy, optionally substituted cycloalkyl, optionally substituted cycloalkyloxy, optionally substituted aryl, optionally
substituted heteroaryl, —SF₅, —SOₚ (optionally substituted alkyl or haloalkyl), or —NR^{c}R^{d} wherein R^{C} and R^{d} are independently H or optionally substituted alkyl; or R^{C} and R^{d} may form, with the nitrogen to which they are attached, a 3-, 4-, 5-, 6-, 7-, or 8-membered-heterocyclyl group, which may include one to three additional heteroatoms selected from the group consisting of N, O and S and may be optionally substituted;
R6 and R7 are independently in each occurrence hydrogen, halo, C₁-C₄-alkyl, C₁-C₄-haloalkyl, C₁-C₄-alkoxy, C₁-C₄-haloalkoxy or C₃-C₈-cycloalkoxy;
R8 is hydrogen or C₁-C₄-alkyl; and
wherein at most three of Y1, Y2′, Y3′, Y4′, Y5′ and Y6′ are heteroatoms;
E2_4 is 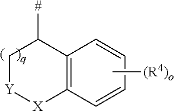
wherein
X and Y are independently CR⁵R⁶, O, S, or N—R⁷, wherein at least one of X and Y is CR⁵R⁶;
R5 and R6 are independently hydrogen, fluorine or C₁-C₄-alkyl;
R7 is hydrogen or C₁-C₄-alkyl;
q is 0 or 1;
o is 0, 1, 2, 3, or 4;
each R4 is independently hydrogen, halogen, cyano, nitro, —OH, optionally substituted alkyl, optionally substituted alkoxy, optionally substituted cycloalkyl, -amino, NH-optionally substituted alkyl, SF₅, or NR^{c}R^{d} wherein R^{c} and R^{d} are independently optionally substituted alkyl;
or R^{c} and R^{d} may form, with the nitrogen to which they are attached, a 3, 4, 5, 6, 7, or 8 membered-heterocyclyl group, which may be optionally substituted, SOₚ (optionally substituted C₁-C₄-alkyl);
p is 0, 1, or 2;
E2_5 is 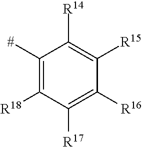
wherein
R and R14 together with the atoms to which they are attached form a 5 or 6-carbon atoms containing non-aromatic ring, wherein the 5 or 6-carbon atoms containing ring is optionally substituted with one or more C₁₋₃-alkyl or ═O, and/or wherein one or more of the ring forming
carbon atoms are optionally replaced by —NH—, —N═, ═N—, —O—, —S(O)—, —S(O)₂— or —S—, or R and R14 together with the atoms to which they are attached form a 5 or 6-carbon atoms containing aromatic ring, wherein the 5 or 6-carbon atoms containing ring is optionally substituted with one or more C₁₋₃-alkyl, and/or wherein one or more of the ring forming carbon atoms are
optionally replaced by —NH—, —N═, ═N—, -0- or —S—;
R15 is independently hydrogen, halogen, C₁₋₃ alkyl, C₁₋₃ alkoxy, or NR^{15′}R^{15″}, wherein R^{15′} and R^{15″} are independently C₁₋₃-alkyl;
R16 is independently hydrogen, halogen, C₁₋₃ alkyl, C₁₋₃ alkoxy, or NR^{16′}R^{16″}, wherein R^{16′} and R^{16″} are independently C₁₋₃-alkyl;
R17 is independently hydrogen, C₁₋₃ alkyl, C₁₋₃ alkoxy, or NR^{17′}R^{17″}, wherein R^{17′} and R^{17″} are independently C₁₋₃-alkyl;
R18 is independently hydrogen, C₁₋₃ alkyl, C₁₋₃ alkoxy, or NR^{18′}R^{18″}, wherein R^{18′} and R^{18″} are independently C₁₋₃-alkyl;
E2_6 is 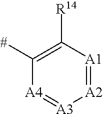
wherein
A1 is N or CR¹⁵, wherein R¹⁵ is independently hydrogen, halogen,
NR^{15′}R^{15″}, wherein R^{15′} and R^{15″} are independently C₁₋₃-alkyl;
A2 is N or CR¹⁶, wherein R¹⁶ is independently hydrogen, halogen,
NR^{16′}R^{16″}, wherein R^{16′} and R^{16″} are independently C₁₋₃-alkyl;
A3 is N or CR¹⁷, wherein R¹⁷ is independently hydrogen, halogen,
NR^{17′}R^{17″}, wherein R^{17′} and R^{17″} are independently C₁₋₃-alkyl;
A4 is N or CR¹⁸, wherein R¹⁸ is independently hydrogen, halogen
NR^{18′}R^{18″}, wherein R^{18′} and R^{18″} are independently C₁₋₃-alkyl;
R14 is hydrogen, C₁₋₃ alkyl, C₁₋₃ alkoxy, NR^{14′}R^{14″}, wherein R^{14′} and R^{14″} are independently C₁₋₃-alkyl;
R and R14 together with the atoms to which they are attached form a 5 or 6-carbon atoms containing non-aromatic ring, wherein the 5 or 6-carbon atoms containing ring is optionally substituted with one or more C1-3-alkyl or ═O, and/or wherein one or more of the ring forming carbon atoms are optionally replaced by —NH—, —N═, ═N—, —O—, —S(O)—, —S(O)2- or —S—, or
R and R14 together with the atoms to which they are attached form a 5 or 6-carbon atoms containing aromatic ring, wherein the 5 or 6-carbon atoms containing ring is optionally substituted with one or more C1-3-alkyl, and/or wherein one or more of the ring forming carbon atoms are optionally replaced by —NH—, —N═, ═N—, —O— or —S—;

3. The method of claim 2, wherein
E1 and E2 are selected from the following pairs when the compound of formula I is a compound of formula I.1:
E1_2 and E2_1 wherein n is 0;
E1_4 and E2_1 wherein n is 0;
E1_7 and E2_2 wherein n is 0.

4. The method of claim 2, wherein
E1 and E2 are selected from the following pairs when the compound of formula I is a compound of formula I.1:
E1_2 and E2_1′ wherein n is 0;
E1_4 and E2_1″ wherein n is 0;
E1_7 and E2_2 wherein n is 0;
in which
E1_2 is 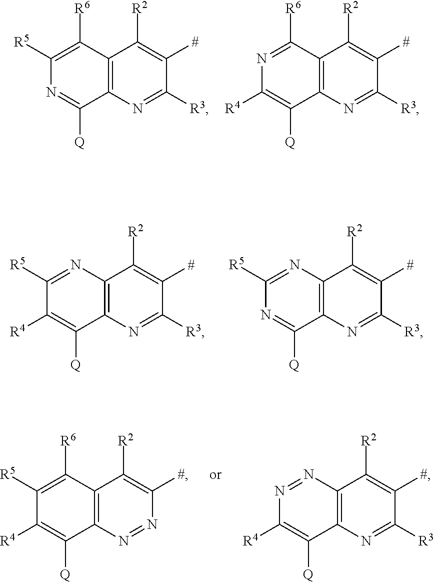
wherein
R2 is selected from the group consisting of
hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂,
—NR¹²R¹³;
—OR¹⁴;
—SR¹⁵, —S(O)R¹⁵, —SO₂R¹⁵;
C₁-C₆-alkyl, C₃-C₆-cycloalkyl, C₂-C₄-alkenyl, C₃-C₆-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, —NO₂, cyano, C₁-C₄-alkyl-C(O)—, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, —OH, —NO₂, cyano, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and 4- to 10-membered heterocycloalkyl;
R3 is selected from the group consisting of hydrogen, halogen or C₁-C₄-alkyl;
R4 is selected from the group consisting of hydrogen, halogen, cyano, C₁-C₄-alkyl, C₁-C₄-halogenoalkyl having 1 to 5 halogen atoms, C₁-C₄-alkoxy;
R5 is selected from the group consisting of hydrogen, halogen, cyano, C₁-C₄-alkyl, C₁-C₄-halogenoalkyl having 1 to 5 halogen atoms, C₁-C₄-alkoxy;
R6 is selected from the group consisting of hydrogen, halogen, cyano, C₁-C₄-alkyl, C₁-C₄-halogenoalkyl having 1 to 5 halogen atoms, C₁-C₄-alkoxy;
R12 and R13 are independently selected from the group consisting of hydrogen, —OH, —NH₂, —NH(C₁-C₄-alkyl), —N(C₁-C₄-alkyl)₂, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)-(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, benzo-C₅-C₆-cycloalkyl, each of which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R14 is selected from the group consisting of
—NH₂, —NH(C₁-C₄-alkyl), —N(C₁-C₄-alkyl)₂;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
R15 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
phenyl, which is optionally substituted by 1, 2 or 3 substituents independently selected from the group consisting of halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, nitro, —OH, oxo, thiono, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, hydroxy-C₁-C₄-alkyl, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms;
Q is a substituted phenyl ring of the formula (Q1) 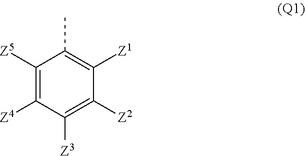
in which:
Z¹, Z², Z³, Z⁴, and Z⁵ are independently selected from the group consisting of hydrogen, halogen, SF₅, cyano, —CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocyclyl, which is optionally substituted with 1 or 2 substituents selected from the group consisting of fluorine, chlorine, bromine, methyl and cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocyclyl which itself is optionally substituted with 1 or 2 substituents selected from the group consisting of fluorine, chlorine, bromine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z¹ and Z² form, together with the carbon atoms that they are connected to, a 5- or 6-membered saturated or partially saturated heterocyclic ring, a 5-membered heteroaryl, or a 6-membered heteroaryl, each of which may be optionally substituted with one or two substitutents selected from the group consisting of methyl, fluorine and oxo, and
Z³, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-alkoxy-C(O)—, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z² and Z³ form, together with the carbon atoms that they are connected to, a 5- or 6-membered saturated or partially saturated heterocyclic ring, a 5-membered heteroaryl, or a 6-membered heteroaryl, each of which may be optionally substituted with one or two substitutents selected from the group consisting of methyl, fluorine and oxo, and
Z¹, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Q is a pyridine ring of the formula (Q2) 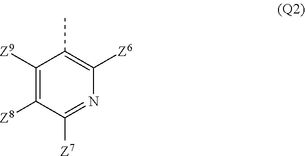
in which:
Z⁶, Z⁷, Z⁸ and Z⁹ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyrimidine ring of the formula (Q3) 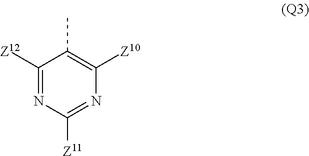
in which:
Z¹⁰, Z¹¹ and Z¹² are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyridine ring of the formula (Q4) 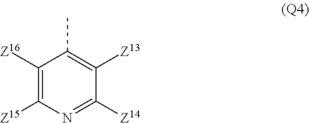
in which:
Z¹³, Z¹⁴, Z¹⁵ and Z¹⁶ are independently selected from the group consisting of hydrogen halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₁-C₄-hydroxyalkyl, NH₂, —NH(C₁-C₄-alkyl), —N(C₁-C₄-alkyl)₂, —NH—CO—C₁-C₄-alkyl, and monocyclic heterocycles selected from the group of 4-to 7-membered heterocycloalkyl or 5-membered heteroaryls having at least one nitrogen atom via which the heteroaryl ring is connected to the pyridine ring, each of which is optionally substituted with 1, 2 or 3 substituents independently selected from the group consisting of halogen, cyano, nitro, —OH, oxo, thiono, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—(C₁-C₄-halogenoalkyl) having 1 to 5 halogen atoms, —S(O)—(C₁-C₄-halogenoalkyl) having 1 to 5 halogen atoms, —SO₂—(C₁-C₄-halogenoalkyl) having 1 to 5 halogen atoms, or
Q is a pyridine ring of the formula (Q5) 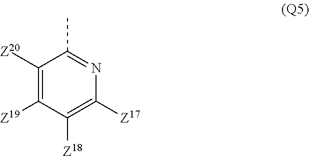
in which:
Z¹⁷, Z¹⁸, Z¹⁹ and Z²⁰ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a 5-membered aromatic heterocycle of the formula (Q6) 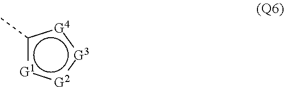
in which:
G¹-G⁴ are independently selected from the group consisting of N, O, S, C—Z²¹ and N—Z²², wherein not more than one of G¹-G⁴ is O, not more than one of G¹-G⁴ is S, not more than one of G¹-G⁴ is N—Z²², and wherein
each Z²¹ is independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, and each Z²² is independently selected from the group consisting of hydrogen, C₁-C₄-alkyl, C₁-C₄-halogenoalkyl having 1 to 5 halogen atoms, C₁-C₄-alkyl-C₃-C₆-cycloalkyl, C₁-C₄-alkoxy-C₁-C₄-alkyl, or
Q is a 5-membered aromatic heterocycle of the formula (Q7) 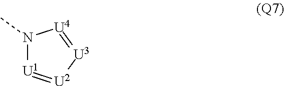
in which:
U¹-U⁴ are independently selected from the group consisting of N and C—Z²³, wherein not more than three of U¹-U⁴ are N, and wherein
each Z²³ is independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
wherein when Y is O, S or N—R⁹, none of R⁷, R⁸, R¹⁰ and R¹¹ is —OH, and wherein when X is O, S or N—R9, none of R and R⁸ is —OH;
E2_1′ is 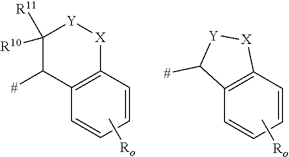
wherein
o is 0, 1, 2, 3 or 4;
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms;
X, Y are independently selected from the group consisting of CR⁷R⁸, O, S, and N—R⁹, wherein at least one of X and Y is CR⁷R⁸, or
X, Y form together a ring member selected from the group consisting of —C(O)—O—, —C(O)—NR⁹—, —S(O)—NR⁹—, —SO₂—NR⁹— and —SO₂—O—;
R7 is selected from the group consisting of hydrogen, —OH, fluorine, C₁-C₄-alkyl and C₁-C₄-alkoxy;
R8 is selected from the group consisting of hydrogen, —OH, fluorine, C₁-C₄-alkyl and C₁-C₄-alkoxy;
R9 is selected from the group consisting of hydrogen, C₁-C₄-alkyl, C₁-C₄-halogenoalkyl having 1 to 5 halogen atoms and C₁-C₄-alkoxy;
R10 is selected from the group consisting of hydrogen, —OH, C₁-C₄-alkyl and C₁-C₄-alkoxy;
R11 is selected from the group consisting of hydrogen, C₁-C₄-alkyl and C₁-C₄-alkoxy;
E1_4 is 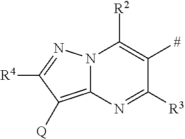
wherein
R2 is selected from the group consisting of hydrogen, halogen, cyano, C₁-C₄-alkyl, C₂-C₄-alkenyl, C₂-C₄-alkinyl, C₃-C₆-cycloalkyl, C₁-C₄-halogenoalkyl having 1 to 5 halogen atoms, C₁-C₄-alkoxy-C₁-C₄-alkyl, benzyl having 1 to 5 halogen atoms, C₁-C₄-alkoxy, —NH₂, —NH(C₁-C₄-alkyl), —N(C₁-C₄-alkyl)₂, —NH(C₃-C₆-cycloalkyl), —N(C₁-C₄-alkyl)(C₃-C₆-cycloalkyl), —NH(4- to 7-membered heterocycloalkyl), —N(C₁-C₄-alkyl)(4- to 7-membered heterocycloalkyl), —NH(C₁-C₄-alkoxy), —N(C₁-C₄-alkyl)(C₁-C₄-alkoxy), —NH—SO₂—(C₁-C₄-alkyl), —N(SO₂—[C₁-C₄-alkyl])(C₁-C₄-alkyl), (C₁-C₄-alkyl)-NH—C₁-C₄-alkyl-, (C₁-C₄-alkyl)₂-N—C₁-C₄-alkyl-, —S—C₁-C₄-alkyl, —S(O)—C₁-C₄-alkyl, —SO₂—C₁-C₄-alkyl, (C₁-C₄-alkoxyimino)-C₁-C₄-alkyl, and a monocyclic heterocycle selected from the group of 4- to 7-membered heterocycloalkyl, 5-membered heteroaryl having at least one nitrogen atom via which the heteroaryl ring is connected to the rest of the molecule, and 6-membered heteroaryl having at least one nitrogen atom, each of which in R² is optionally substituted with 1, 2 or 3 substituents independently selected from the group consisting of hydrogen, halogen, cyano, nitro, —OH, oxo, thiono, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms, and wherein each C₁-C₄-alkyl, C₃-C₆-cycloalkyl and C₁-C₄-alkoxy in R² may be optionally substituted with halogen, OH, NH₂, —NH(C₁-C₄-alkyl), —N(C₁-C₄-alkyl)₂, cyano, carboxy, carbamoyl, alkoxycarbonyl, —C(O)—NH(C₁-C₄-alkyl), —C(O)—N(C₁-C₄-alkyl)₂, C₁-C₄-alkoxy, —S—C₁-C₄-alkyl, —S(O)—C₁-C₄-alkyl, —SO₂—C₁-C₄-alkyl, or optionally substituted by a monocyclic heterocycle selected from the group of azetidines, pyrrolidines, morpholines, piperidines, piperazines, pyrrolidinones, morpholinones, piperidinones, piperazinones, pyrazoles, triazoles, imidazoles and pyrroles, wherein a heteroaryl ring is connected to the C₁-C₄-alkyl or C₃-C₆-cycloalkyl via one of its nitrogen atoms, each of which as a substituent of C₁-C₄-alkyl, C₃-C₆-cycloalkyl and C₁-C₄-alkoxy in R² is optionally substituted with 1, 2 or 3 substituents independently selected from the group consisting of hydrogen, halogen, cyano, oxo, C₁-C₄-alkyl, C₁-C₄-halogenoalkyl having 1 to 5 halogen atoms;
R3 is selected from the group consisting of hydrogen, halogen, C₁-C₄-alkyl, C₁-C₄-halogenoalkyl having 1 to 5 halogen atoms, C₃-C₆-cycloalkyl;
R4 is selected from the group consisting of hydrogen, halogen, C₁-C₄-alkyl, C₁-C₄-halogenoalkyl having 1 to 5 halogen atoms, C₃-C₆-cycloalkyl, —S—C₁-C₄-alkyl, —S(O)—C₁-C₄-alkyl, —SO₂—C₁-C₄-alkyl;
Q is selected from the group consisting of 6- or 10-membered aryl and 5- to 10-membered heteroaryl, each of which may be optionally substituted with 1, 2, 3, 4 or 5 substituents;
E2_1″ is 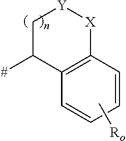
wherein
o is 0, 1, 2, 3 or 4;
R is selected from the group consisting of hydrogen, halogen, C₁-C₄-alkyl and C₁-C₄-halogenoalkyl having 1 to 5 halogen atoms;
n is 0 or 1;
X, Y are independently selected from the group consisting of CR⁵R⁶, O, S, and N—R, wherein at least one of X and Y is CR⁵R6;
R5 is selected from the group consisting of hydrogen, fluorine or C₁-C₄-alkyl;
R6 is selected from the group consisting of hydrogen, fluorine or C₁-C₄-alkyl;
R7 is selected from the group consisting of hydrogen or C₁-C₄-alkyl;
E1_7 is 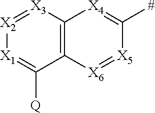
X₁ is CR₁; X₂ is CR₂; X₃ is CR₃; X₄ is CR₄; X₅ is CR₅; and X₆ is CR₆;
E2_2 is 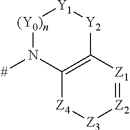
as defined in claim 2.

5. The method of claim 2, wherein
E1 and E2 are selected from the following pairs when the compound of formula I is a compound of formula I.1:
E1_2 and E2_1′ wherein n is 0;
E1_4 and E2_1″ wherein n is 0;
E1_7 and E2_2 wherein n is 0;
in which
E1_2 is 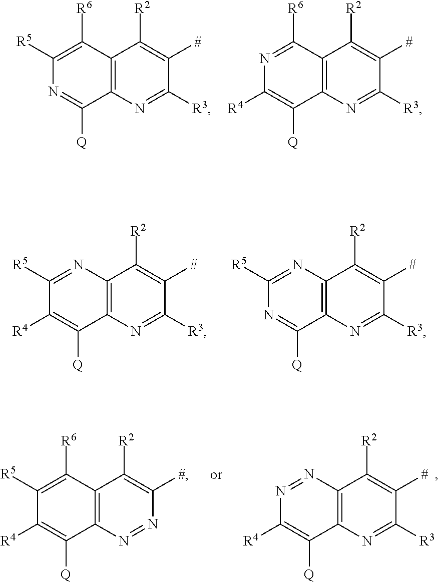
wherein
R2 is selected from the group consisting of hydrogen, halogen, cyano, —COOH, C₁-C₄-alkoxy-C(O)—, —C(O)—NH₂, —C(O)—NH(C₁-C₄-alkyl), —C(O)—N(C₁-C₄-alkyl)₂,
—NR¹²R¹³;
—OR¹⁴;
—SR¹⁵, —S(O)R¹⁵, —SO₂R¹⁵;
C₁-C₄-alkyl, C₃-C₆-cycloalkyl, C₂-C₄-alkenyl, C₃—C-cycloalkenyl, C₂-C₄-alkynyl or phenyl-C₁-C₄-alkyl, each of which is optionally substituted by 1, 2, 3, 4 or 5 substituents independently selected from the group consisting of halogen, —OH, cyano, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-halogenoalkyl having 1 to 5 halogen atoms, C₁-C₄-alkoxy, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms and —SO₂—C₁-C₄-halogenoalkyl having 1 to 5 halogen atoms; and
a monocyclic or a bicyclic heterocycle selected from the group consisting of 4- to 10-membered heterocycloalkyl, heterospirocycloalkyl, 5-membered heteroaryl and 6-membered heteroaryl, each of which is optionally substituted by 1, 2, 3 or 4 substituents independently selected from the group consisting of halogen, cyano, —OH, oxo, —COOH, C₁-C₄-alkoxy-C(O)—, —C(O)—NH₂, —C(O)—NH(C₁-C₄-alkyl), —C(O)—N(C₁-C₄-alkyl)₂, C₁-C₄-alkyl, C₁-C₄-alkyl-C(O)—, C₁-C₄-halogenoalkyl having 1 to 5 halogen atoms, C₁-C₄-alkoxy, hydroxy-C₁-C₄-alkyl, C₁-C₄-alkoxy-C₁-C₄-alkyl-, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, and 4- to 10-membered heterocycloalkyl;
R3 is selected from the group consisting of hydrogen, halogen or C₁-C₄-alkyl;
R4 is selected from the group consisting of hydrogen, halogen, cyano, C₁-C₄-alkyl, C₁-C₄-halogenoalkyl having 1 to 5 halogen atoms, C₁-C₄-alkoxy;
R5 is selected from the group consisting of hydrogen, halogen, cyano, C₁-C₄-alkyl, C₁-C₄-halogenoalkyl having 1 to 5 halogen atoms, C₁-C₄-alkoxy;
R6 is selected from the group consisting of hydrogen, halogen, cyano, C₁-C₄-alkyl, C₁-C₄-halogenoalkyl having 1 to 5 halogen atoms, C₁-C₄-alkoxy;
R12 and R13 are independently selected from the group consisting of hydrogen, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, —COOH, C₁-C₄-alkoxy-C(O)—, —C(O)—NH₂, —C(O)—NH(C₁-C₄-alkyl), —C(O)—N(C₁-C₄-alkyl)₂, —NH—C(O)—C₁-C₄-alkyl, —N(C₁-C₄-alkyl)-(—C(O)—C₁-C₄-alkyl), C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl having 1 to 5 halogen atoms, —S(O)—C₁-C₄-halogenoalkyl having 1 to 5 halogen atoms, —SO₂—C₁-C₄-halogenoalkyl having 1 to 5 halogen atoms and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
phenyl, benzo-C₅-C₆-cycloalkyl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms; and
a monocyclic or a bicyclic heterocycle selected from the group of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
R14 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl; and
heterocyclyl-C₁-C₄-alkyl, wherein the heterocyclyl substitutent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
R15 is selected from the group consisting of
C₁-C₄-alkyl, phenyl-C₁-C₄-alkyl, each of which is optionally substituted by 1, 2 or 3 substituents independently selected from the group consisting of halogen, —OH, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
Q is a substituted phenyl ring of the formula (Q1) 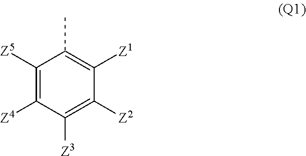
in which:
Z¹, Z², Z³, Z⁴, and Z⁵ are independently selected from the group consisting of hydrogen, halogen, cyano, C₁-C₄-alkyl, C₁-C₄-halogenoalkyl having 1 to 5 halogen atoms, hydroxy, C₁-C₄-alkoxy, C₁-C₄-halogenoalkoxy having 1 to 5 halogen atoms, —NH(C₁-C₄-alkyl), —N(C₁-C₄-alkyl)₂, 4- to 6-membered heterocyclyl, which is optionally substituted with 1 or 2 substituents selected from the group consisting of fluorine, chlorine, bromine, methyl and cyano, —S—(C₁-C₄-alkyl), —S(O)—(C₁-C₄-alkyl), —SO₂—(C₁-C₄-alkyl), or
Z¹ and Z² form, together with the carbon atoms that they are connected to, a 5- or 6-membered heterocycloalkyl, a 5-membered heteroaryl, or a 6-membered heteroaryl, each of which may be optionally substituted with one or two substitutents selected from the group consisting of methyl, fluorine and oxo, and Z³, Z⁴, and Z⁵ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-alkoxy-C(O)—, C₁-C₄-halogenoalkoxy having 1 to 5 halogen atoms, or
Z² and Z³ form, together with the carbon atoms that they are connected to, a 5- or 6-membered saturated or partially saturated heterocyclic ring, a 5-membered heteroaryl, or a 6-membered heteroaryl, each of which may be optionally substituted with one or two substitutents selected from the group consisting of methyl, fluorine and oxo, and
Z¹, Z⁴, and Z⁵ are independently selected from the group consisting of hydrogen, halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms;
E2_1′ is 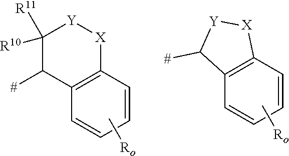
o is 0, 1 or 2;
R is selected from the group consisting of hydrogen, halogen, C₁-C₄-alkyl and C₁-C₄-alkoxy, cyano, C₁-C₄-halogenoalkyl having 1 to 5 halogen atoms;
X, Y are independently selected from the group consisting of CR⁷R⁸, O, S, and N—R9, wherein at least one of X and Y is CRR;
R7 is selected from the group consisting of hydrogen and C₁-C₄-alkyl;
R8 is selected from the group consisting of hydrogen and C₁-C₄-alkyl;
R9 is C₁-C₄-alkyl;
R10 is selected from the group consisting of hydrogen, —OH, C₁-C₄-alkyl and C₁-C₄-alkoxy;
R11 is hydrogen;
E1_4 is 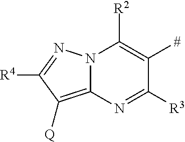
wherein
R2 is selected from the group consisting of hydrogen, C₁-C₄-alkyl, C₃-C₆-cycloalkyl, C₁-C₄-halogenoalkyl having 1 to 5 halogen atoms, C₁-C₄-alkoxy-C₁-C₄-alkyl, benzyl having 1 to 5 halogen atoms, C₁-C₄-alkoxy, —NH(C₁-C₄-alkyl), —N(C₁-C₄-alkyl)₂, —NH(C₃-C₆-cycloalkyl), —N(C₁-C₄-alkyl)(C₃-C₆-cycloalkyl), —N(C₁-C₄-alkyl)(6-membered heterocycloalkyl), —N(C₁-C₄-alkyl)(C₁-C₄-alkoxy), (C₁-C₄-alkyl)₂-N—C₁-C₄-alkyl-, and 4- to 6-membered heterocycloalkyl having at least one nitrogen atom via which the heterocycloalkyl ring is connected to the rest of the molecule, wherein a heterocycloalkyl group in R² may be optionally substituted with 1 to 4 substituents selected from the group consisting of fluorine, chlorine, cyano, oxo, C₁-C₄-alkyl, C₁-C₄-alkoxy, —N(C₁-C₄-alkyl)₂, and wherein each C₁-C₄-alkyl, C₃-C₆-cycloalkyl and C₁-C₄-alkoxy in R² may be optionally substituted with halogen, OH, NH₂, —NH(C₁-C₄-alkyl), —N(C₁-C₄-alkyl)₂, cyano, carboxy, carbamoyl, alkoxycarbonyl, —C(O)—NH(C₁-C₄-alkyl), —C(O)—N(C₁-C₄-alkyl)₂, C₁-C₄-alkoxy, —S—C₁-C₄-alkyl, —S(O)—C₁-C₄-alkyl, —SO₂—C₁-C₄-alkyl, or optionally substituted by a monocyclic heterocycle selected from the group of azetidines, pyrrolidines, morpholines, piperidines, and piperazines, each of which as a substituent of C₁-C₄-alkyl, C₃-C₆-cycloalkyl and C₁-C₄-alkoxy in R² is optionally substituted with 1, 2 or 3 substituents independently selected from the group consisting of hydrogen, halogen, cyano, oxo, C₁-C₄-alkyl, C₁-C₄-halogenoalkyl having 1 to 5 halogen atoms;
R3 is selected from the group consisting of hydrogen and C₁-C₄-alkyl;
R4 is selected from the group consisting of hydrogen, halogen, C₁-C₄-alkyl, C₁-C₄-halogenoalkyl having 1 to 5 halogen atoms, C₃-C₆-cycloalkyl, —S—C₁-C₄-alkyl, —S(O)—C₁-C₄-alkyl, —SO₂—C₁-C₄-alkyl;
Q is a substituted phenyl ring of the formula (Q1) 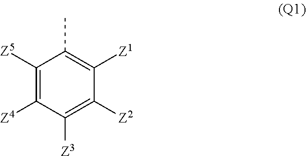
in which:
Z¹, Z², Z³, Z⁴, and Z⁵ are independently selected from the group consisting of hydrogen, halogen, SF₅, cyano, —CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, —O—(C₃-C₆-cycloalkyl), cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl and cyano, or 5-membered heteroaryl having at least one nitrogen atom via which the heteroaryl ring is connected to the rest of the molecule, —CH₂—O—(C₁-C₄-alkyl), —CH₂—NH(C₁-C₄-alkyl), —CH₂—N(C₁-C₄-alkyl)₂, methyl substituted with C₁-C₄-halogenoalkoxy having 1 to 5 halogen atoms, methyl substituted with C₃-C₆-cycloalkyl-C₁-C₄-alkoxy or methyl substituted with a 4- to 6-membered heterocycloalkyl which itself is optionally substituted with 1 or 2 substituents selected from the group consisting of fluorine, methyl and cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl), —S(O)—(C₁-C₄-halogenoalkyl), —SO₂—(C₁-C₄-halogenoalkyl), —S—(C₁-C₄-cycloalkyl), —S(O)—(C₁-C₄-cycloalkyl), —SO₂—(C₁-C₄-cycloalkyl), —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z¹ and Z² form, together with the carbon atoms that they are connected to, a 5- or 6-membered heterocycloalkyl, a 5-membered heteroaryl, or a 6-membered heteroaryl, each of which may be optionally substituted with one or two substituents selected from the group consisting of methyl, fluoro and oxo, and
Z³, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl), —S(O)—(C₁-C₄-halogenoalkyl), —SO₂—(C₁-C₄-halogenoalkyl), —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Z² and Z³ form, together with the carbon atoms that they are connected to, a 5- or 6-membered cycloalkyl or heterocycloalkyl, a 5-membered heteroaryl, or a 6-membered heteroaryl, each of which may be optionally substituted with one or two substituents selected from the group consisting of methyl, fluoro and oxo, and
Z¹, Z⁴, and Z⁵ are independently selected from the group consisting of hydrogen, halogen, SF₅, cyano, CHO, nitro, C₁-C₄-alkyl, C₁-C₄-halogenoalkyl having 1 to 5 halogen atoms, hydroxy, C₁-C₄-alkoxy, C₃-C₆-cycloalkyl-C₁-C₄-alkoxy, cyano-C₁-C₄-alkoxy, C₁-C₄-halogenoalkoxy having 1 to 5 halogen atoms, —NH(C₁-C₄-alkyl), —N(C₁-C₄-alkyl)₂, —NH—SO₂—(C₁-C₄-alkyl), —N(SO₂—[C₁-C₄-alkyl])(C₁-C₄-alkyl), (C₁-C₄-alkoxyimino)-C₁-C₄-alkyl, 4- to 6-membered heterocycloalkyl which is optionally substituted with 1 or 2 substituents selected from the group consisting of fluorine, methyl or cyano, —CH₂—O—(C₁-C₄-alkyl), —CH₂—NH(C₁-C₄-alkyl), —CH₂—N(C₁-C₄-alkyl)₂, methyl substituted with a 4- to 6-membered heterocycloalkyl which itself is optionally substituted with 1 or 2 substituents selected from the group consisting of fluorine, methyl or cyano, —CH₂—S—(C₁-C₄-alkyl), —CH₂—S(O)—(C₁-C₄-alkyl), —CH₂—SO₂—(C₁-C₄-alkyl), —S—(C₁-C₄-alkyl), —S(O)—(C₁-C₄-alkyl), —SO₂—(C₁-C₄-alkyl), —S—(C₁-C₄-halogenoalkyl), —S(O)—(C₁-C₄-halogenoalkyl), —SO₂—(C₁-C₄-halogenoalkyl), —CONH(C₁-C₄-alkyl), —CONH(C₃-C₆-cycloalkyl), —NHCO(C₁-C₄-alkyl), —NHCO(C₃-C₆-cycloalkyl), —NHCO(C₁-C₄-halogenoalkyl) having 1 to 5 halogen atoms, or
Q is a pyridine ring of the formula (Q2) 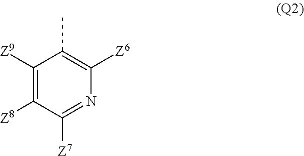
in which:
Z⁶, Z⁷, Z⁸ and Z⁹ are independently selected from the group consisting of hydrogen fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyrimidine ring of the formula (Q3) 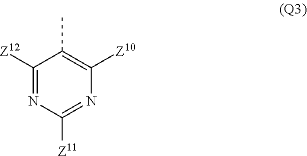
in which:
Z¹⁰, Z¹¹ and Z¹² are independently selected from the group consisting of hydrogen, fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q is a pyridine ring of the formula (Q4) 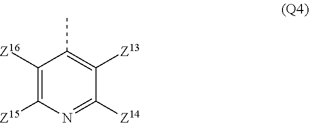
in which:
Z¹³, Z¹⁴, Z¹⁵ and Z¹⁶ are independently selected from the group consisting of hydrogen, fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH₂, —NH(C₁-C₄-alkyl), —N(C₁-C₄-alkyl)₂, —NH—CO—C₁-C₄-alkyl, and monocyclic heterocycles selected from the group of 4- to 7-membered heterocycloalkyl or 5-membered heteroaryls having at least one nitrogen atom via which the heteroaryl ring is connected to the pyridine ring, each of which is optionally substituted with 1, 2 or 3 substituents independently selected from the group consisting of hydrogen, halogen, cyano, nitro, —OH, oxo, thiono, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms, or
Q is a pyridine ring of the formula (Q5) 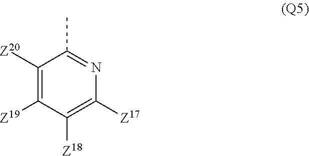
in which:
Z¹⁷, Z¹⁸, Z¹⁹ and Z²⁰ are independently selected from the group consisting of hydrogen, fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, —NH(C₁-C₄-alkyl), —N(C₁-C₄-alkyl)₂, or
Q a pyrazole ring of the formula (Q6) 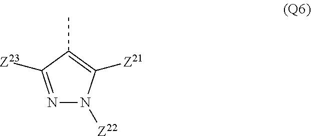
in which:
Z²¹ and Z²³ are independently selected from the group consisting of hydrogen, C₁-C₄-alkyl, C₁-C₄-halogenoalkyl having 1 to 5 halogen atoms, and
Z²² is selected from the group consisting of hydrogen, C₁-C₄-alkyl, C₁-C₄-halogenoalkyl having 1 to 5 halogen atoms, C₁-C₄-alkyl-C₃—C-cycloalkyl, C₁-C₄-alkoxy-C₁-C₄-alkyl, (C₁-C₄-alkyl)₂-N—C₁-C₄-alkyl-, morpholino-C₁-C₄-alkyl, (C₁-C₄-alkyl)-NH—C₁-C₄-alkyl-, or
Q is a pyrazole ring of the formula (Q7) 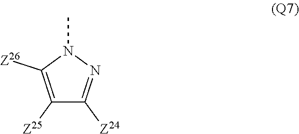
in which:
Z²⁴, Z²⁵ and Z²⁶ are independently selected from the group consisting of hydrogen, fluorine, chlorine, bromine, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
E2_1″ is 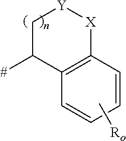
wherein
o is 0, 1, 2, 3 or 4;
R is selected from the group consisting of hydrogen, fluorine, chlorine, C₁-C₄-alkyl;
n is 0 or 1;
X, Y are independently selected from the group consisting of CR⁵R⁶, O, S, and N—R, wherein at least one of X and Y is CR⁵R6;
R5 is hydrogen or methyl;
R6 is hydrogen or methyl;
R7 is hydrogen or methyl;
E1_7 is 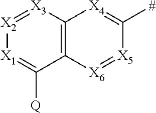
X₁ is N; X₂ is CR₂; X₃ is CR₃; X₄ is CR₄; X₅ is CR₅; and X₆ is N;
E2-2 is 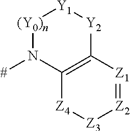
as defined in claim 2.

6. The method of claim 2, wherein E1 and E2 are selected from the following pairs when the compound of formula I is a compound of formula I.1:
E1_2 and E2_1′ wherein n is 0;
E1_4 and E2_1″ wherein n is 0;
E1_7 and E2_2 wherein n is 0;
in which
E1_2 is 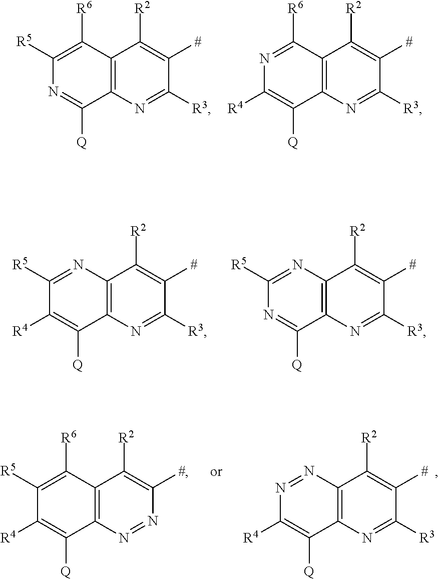
wherein
R2 is selected from the group consisting of
hydrogen, halogen,
—NR¹²R¹³;
—OR¹⁴;
—SR¹⁵, —S(O)R¹⁵, —SO₂R¹⁵;
C₁-C₄-alkyl, C₃-C₆-cycloalkyl, C₂-C₄-alkenyl or C₃-C₆-cycloalkenyl, each of which is optionally substituted by 1, 2, 3, 4 or 5 substituents independently selected from the group consisting of halogen, cyano, C₁-C₄-alkoxy-C(O)— and —C(O)—NH₂; and
a monocyclic or a bicyclic heterocycle selected from the group consisting of 4- to 10-membered heterocycloalkyl, heterospirocycloalkyl, 5-membered heteroaryl, and 6-membered heteroaryl, each of which is optionally substituted by 1, 2, 3 or 4 substituents independently selected from the group consisting of halogen, —OH, oxo, —COOH, C₁-C₄-alkoxy-C(O)—, —C(O)—NH₂, C₁-C₄-alkyl, C₁-C₄-alkyl-C(O)—, C₁-C₄-halogenoalkyl having 1 to 5 halogen atoms, hydroxy-C₁-C₄-alkyl-, C₁-C₄-alkoxy-C₁-C₄-alkyl-, —NH₂, —N(C₁-C₄-alkyl)₂, and 4- to 10-membered heterocycloalkyl,
R3 is selected from the group consisting of hydrogen, halogen or C₁-C₄-alkyl;
R4 is selected from the group consisting of hydrogen, halogen, cyano, C₁-C₄-alkyl, C₁-C₄-halogenoalkyl having 1 to 5 halogen atoms, C₁-C₄-alkoxy;
R5 is selected from the group consisting of hydrogen, halogen, cyano, C₁-C₄-alkyl, C₁-C₄-halogenoalkyl having 1 to 5 halogen atoms, C₁-C₄-alkoxy;
R6 is selected from the group consisting of hydrogen, halogen, cyano, C₁-C₄-alkyl, C₁-C₄-halogenoalkyl having 1 to 5 halogen atoms, C₁-C₄-alkoxy;
R12 and R13 are independently selected from the group consisting of
hydrogen, —NH(—C(O)—C₁-C₄-alkyl), C₁-C₄-alkoxy;
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, —COOH, C₁-C₄-alkoxy-C(O)—, —C(O)—NH₂, —C(O)—N(C₁-C₄-alkyl)₂, —NH—C(O)—C₁-C₄-alkyl, C₁-C₄-alkyl, C₁-C₄-alkoxy, C₃-C₆-cycloalkyl, —NH₂, —N(C₁-C₄-alkyl)₂, —S—C₁-C₄-alkyl, —S(O)—C₁-C₄-alkyl, —SO₂—C₁-C₄-alkyl, and (C₁-C₄-alkoxy)₂P(═O)—;
heterocyclyl-C₁-C₄-alkyl, wherein the heterocyclyl substituent is selected from the group consisting of 4- to 10-membered heterocycloalkyl, 5-membered heteroaryl and 6-membered heteroaryl, each of which is optionally substituted by 1, 2 or 3 substituents independently selected from the group consisting of halogen, cyano, —OH, oxo, C₁-C₄-alkyl, C₁-C₄-halogenoalkyl having 1 to 5 halogen atoms and C₁-C₄-alkoxy;
phenyl and benzo-C₅-C₆-cycloalkyl, each of which is optionally substituted by 1, 2 or 3 substituents independently selected from the group consisting of halogen, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms; and
a monocyclic or a bicyclic heterocycle selected from the group of 4- to 10-membered heterocycloalkyl, 5-membered heteroaryl and 6-membered heteroaryl each of which is optionally substituted by 1, 2 or 3 substituents independently selected from the group consisting of halogen, —OH, oxo, cyano, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms,
R14 is selected from the group consisting of
C₁-C₄-alkyl, C₃-C₆-cycloalkyl, phenyl-C₁-C₄-alkyl, each of which is optionally substituted by 1, 2 or 3 substituents independently selected from the group consisting of halogen, —OH, C₁-C₄-alkyl, C₁-C₄-alkoxy and C₃-C₆-cycloalkyl; and
4- to 10-membered heterocycloalkyl,
R15 is selected from the group consisting of
C₁-C₄-alkyl, which is optionally substituted by 1, 2 or 3 substituents independently selected from the group consisting of —OH and —COOH; and a 6-membered heteroaryl;
Q is a substituted phenyl ring of the formula (Q1) 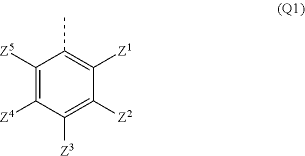
in which:
Z¹ is selected from the group consisting of hydrogen, halogen, C₁-C₄-alkyl and C₁-C₄-alkoxy,
Z² is selected from the group consisting of hydrogen, halogen, —OH, C₁-C₄-alkyl, C₁-C₄-alkoxy, —NH(C₁-C₄-alkyl), —N(C₁-C₄-alkyl)₂, —NH(C₃-C₆-cycloalkyl), —N(C₁-C₄-alkyl)(C₃-C₆-cycloalkyl), C₁-C₄-halogenoalkyl having 1 to 5 halogen atoms, C₁-C₄-halogenoalkoxy having 1 to 5 halogen atoms, —S—(C₁-C₄-alkyl) and a 4- to 6-membered heterocycloalkyl, and
Z³ is selected from the group consisting of hydrogen, halogen, C₁-C₄-alkyl, C₁-C₄-alkoxy, —NH(C₁-C₄-alkyl) and —N(C₁-C₄-alkyl)₂,
Z⁴ is selected from the group consisting of hydrogen, halogen, —OH, C₁-C₄-alkyl, C₁-C₄-alkoxy, —NH(C₁-C₄-alkyl), —N(C₁-C₄-alkyl)₂, —NH(C₃-C₆-cycloalkyl), —N(C₁-C₄-alkyl)(C₃-C₆-cycloalkyl), C₁-C₄-halogenoalkyl having 1 to 5 halogen atoms, C₁-C₄-halogenoalkoxy having 1 to 5 halogen atoms, —S—(C₁-C₄-alkyl) and a 4- to 6-membered heterocycloalkyl,
Z⁵ is selected from the group consisting of hydrogen, halogen, C₁-C₄-alkyl, and C₁-C₄-alkoxy;
E2_1′ is 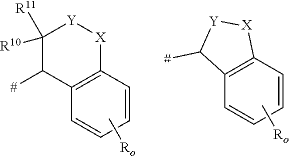
o is 0 or 1;
R is selected from the group consisting of hydrogen, halogen, C₁-C₄-alkyl and C₁-C₄-alkoxy;
X is selected from the group consisting of CR⁷R⁸, O, S, and
N—R⁹;
Y is CR⁷R⁸;
R7 is selected from the group consisting of hydrogen and C₁-C₄-alkyl;
R⁸ is selected from the group consisting of hydrogen and C₁-C₄-alkyl;
R⁹ is C₁-C₄-alkyl;
R¹⁰ is selected from the group consisting of hydrogen, —OH and C₁-C₄-alkyl;
R¹¹ is hydrogen;
E1_4 is 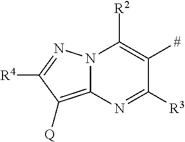
wherein
R2 is selected from the group consisting of hydrogen, C₁-C₄-alkyl, C₃-C₆-cycloalkyl, C₁-C₄-fluoroalkyl having 1 to 5 fluorine atoms, C₁-C₄-alkoxy-C₁-C₄-alkyl, benzyl having 1 to 5 halogen atoms, C₁-C₄-alkoxy, NH(C₁-C₄-alkyl), N(C₁-C₄-alkyl)₂, —NH(C₃-C₆-cycloalkyl), —N(C₁-C₄-alkyl)(C₃-C₆-cycloalkyl), —N(C₁-C₄-alkyl)(6-membered heterocycloalkyl), —N(C₁-C₄-alkyl)(C₁-C₄-alkoxy), morpholino optionally substituted with 1 to 2 C₁-C₄-alkyl groups, C₁-C₄-alkyl-N(C₁-C₄-alkyl)₂, wherein each C₁-C₄-alkyl in R² may be optionally substituted with halogen, —N(C₁-C₄-alkyl)₂, C₁-C₄-alkoxy which itself may be substituted with C₁-C₂-alkoxy-substituted C₁-C₂-alkoxy, —S—C₁-C₄-alkyl, —S(O)—C₁-C₄-alkyl, —SO₂—C₁-C₄-alkyl, or optionally substituted by a monocyclic heterocycle selected from the group of 4- to 7-membered heterocycloalkyl, which itself may be substituted with methyl or oxo;
R3 is selected from the group consisting of hydrogen and C₁-C₄-alkyl;
R4 is selected from the group consisting of hydrogen, halogen, C₁-C₄-alkyl, C₁-C₄-halogenoalkyl having 1 to 5 halogen atoms, C₃-C₆-cycloalkyl, —S—C₁-C₄-alkyl, —S(O)—C₁-C₄-alkyl, —SO₂—C₁-C₄-alkyl; and
Q is a substituted phenyl ring of the formula (Q1) 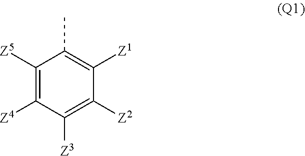
in which:
Z¹, Z², Z³, Z⁴, and Z⁵ are independently selected from the group consisting of hydrogen, fluorine, chlorine, bromine, cyano, methyl, propyl, difluoromethyl, trifluoromethyl, hydroxy, methoxy, ethoxy, isopropoxy, —O-cyclopropyl, —OCH₂-cyclopropyl, —OCH₂CN, trifluoromethoxy, difluoromethoxy, trifluoroethoxy, methylamino, dimethylamino, methylethylamino, diethylamino, acetylamino, methylsulfonamide, trifluoroacetylamino, —SO₂Me, —SO₂— cyclopropyl, —CH₂—O-methyl, —CH₂—O-ethyl, —CH₂—O—CH₂-cyclopropyl, —CH₂—O-isopropyl, —CH₂—N(CH₃)₂, —CH₂—N(CH₂CH₃)₂, —CH₂—N(CH₃)(CH₂CH₃), —CH₂—SCH₃, —CH₂—S(O)CH₃, —CH₂—SO₂—CH₃, —C(O)NH-cyclopropyl, and 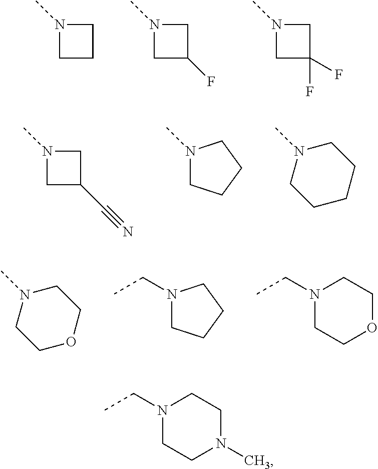
or
Z¹ and Z² form, together with the carbon atoms that they are connected to, a 5-membered heterocycloalkyl or a 5-membered heteroaryl, each of which may be optionally substituted with one or two substituents selected from the group consisting of methyl, fluorine and oxo, and
Z³, Z⁴, and Z⁵ are independently selected from the group consisting of hydrogen, fluorine, chlorine, bromine, cyano, methyl, propyl, difluoromethyl, trifluoromethyl, hydroxy, methoxy, ethoxy, isopropoxy, —O-cyclopropyl, —OCH₂-cyclopropyl, —OCH₂CN, trifluoromethoxy, difluoromethoxy, trifluoroethoxy, methylamino, dimethylamino, methylethylamino, diethylamino, acetylamino, methylsulfonamide, trifluoroacetylamino, —SO₂Me, —SO₂— cyclopropyl, —CH₂—O-methyl, —CH₂—O-ethyl, —CH₂—O—CH₂-cyclopropyl, —CH₂—O-isopropyl, —CH₂—N(CH₃)₂, —CH₂—N(CH₂CH₃)₂, —CH₂—N(CH₃)(CH₂CH₃), —CH₂—SCH₃, —CH₂—S(O)CH₃, —CH₂—SO₂—CH₃, —C(O)NH-cyclopropyl, and 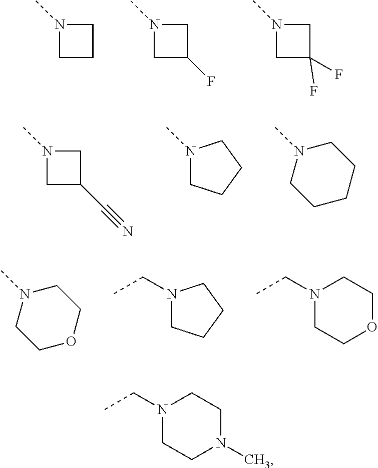
or
Z² and Z³ form, together with the carbon atoms that they are connected to, a 5-membered cycloalkyl or heterocycloalkyl or a 5-membered heteroaryl, each of which may be optionally substituted with one or two substituents selected from the group consisting of methyl, fluorine and oxo, and
Z¹, Z⁴, and Z⁵ are independently selected from the group consisting of hydrogen, fluorine, chlorine, bromine, cyano, methyl, propyl, difluoromethyl, trifluoromethyl, hydroxy, methoxy, ethoxy, isopropoxy, —O-cyclopropyl, —OCH₂-cyclopropyl, —OCH₂CN, trifluoromethoxy, difluoromethoxy, trifluoroethoxy, methylamino, dimethylamino, methylethylamino, diethylamino, acetylamino, methylsulfonamide, trifluoroacetylamino, —SO₂Me, —SO₂— cyclopropyl, —CH₂—O-methyl, —CH₂—O-ethyl, —CH₂—O—CH₂-cyclopropyl, —CH₂—O-isopropyl, —CH₂—N(CH₃)₂, —CH₂—N(CH₂CH₃)₂, —CH₂—N(CH₃)(CH₂CH₃), —CH₂—SCH₃, —CH₂—S(O)CH₃, —CH₂—SO₂—CH₃, —C(O)NH-cyclopropyl, and 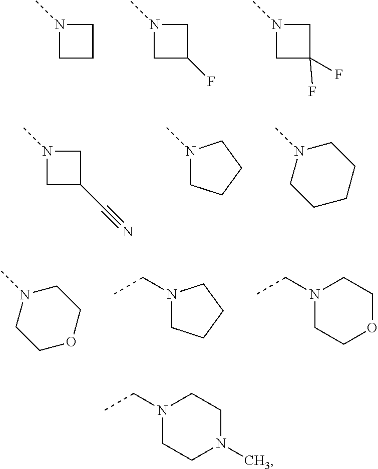
or
Q is a pyridine ring of the formula (Q2) 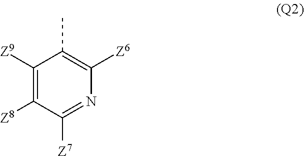
in which:
Z⁶, Z⁷, Z⁸ and Z⁹ are independently selected from the group consisting of hydrogen, fluorine or chlorine, or
Q is a pyrimidine ring of the formula (Q3) 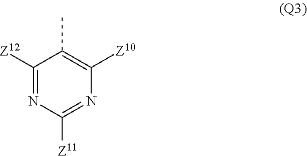
in which:
Z¹⁰, Z¹¹ and Z¹² are independently selected from the group consisting of hydrogen, fluorine, chlorine, C₁-C₄-alkyl, or
Q is a pyridine ring of the formula (Q4) 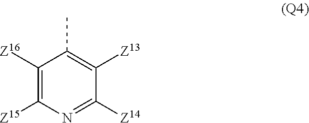
in which:
Z¹³, Z¹⁴, Z¹⁵ and Z¹⁶ are independently selected from the group consisting of hydrogen, fluorine, chlorine, C₁-C₄-alkyl, NH₂, —NH(C₁-C₄-alkyl), —N(C₁-C₄-alkyl)₂, —NH—CO—C₁-C₄-alkyl, and morpholino, pyrazoles, triazoles, imidazoles and pyrroles, wherein a heteroaryl ring is connected to the pyridine ring via one of its nitrogen atoms, each of which is optionally substituted with 1, 2 or 3 substituents independently selected from the group consisting of hydrogen, halogen, cyano, C₁-C₄-alkyl, C₁-C₄-halogenoalkyl having 1 to 5 halogen atoms, or
Q is a pyridine ring of the formula (Q5) 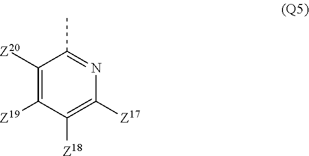
in which:
Z¹⁷, Z¹⁸, Z¹⁹ and Z²⁰ are independently selected from the group consisting of hydrogen, fluorine, chlorine, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, or
Q a pyrazole ring of the formula (Q6) 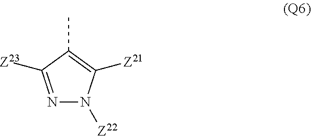
in which:
Z²¹ and Z²³ are hydrogen, and
Z²² is selected from the group consisting of hydrogen, C₁-C₄-alkyl, C₁-C₄-halogenoalkyl having 1 to 5 halogen atoms, C₁-C₄-alkyl-C₃-C₆-cycloalkyl, C₁-C₄-alkoxy-C₁-C₄-alkyl, C₁-C₄-alkyl-N(C₁-C₄-alkyl)₂, morpholino-C₁-C₄-alkyl, or
Q is a pyrazole ring of the formula (Q7) 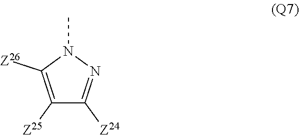
in which:
Z²⁴, Z²⁵ and Z²⁶ are independently selected from the group consisting of hydrogen, fluorine, chlorine, cyano, methyl, trifluoromethyl;
E2_1″ is 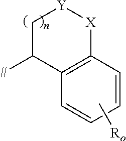
wherein
o is 0 or 1;
R is selected from the group consisting of hydrogen, fluorine, chlorine, C₁-C₄-alkyl;
n is 0 or 1;
X, Y are independently selected from the group consisting of CH₂ and O, wherein at least one of X and Y is CH₂;
E1_7 is 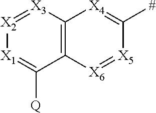
wherein
X₁ is CR₁; X₂ is CR₂; X₃ is CR₃; X₄ is CR₄; X₅ is N; and X₆ is N;
E2_2 is 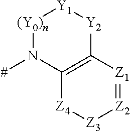
as defined in claim 2.

7. The method of claim 2, wherein
E1 and E2 are selected from the following pairs when the compound of formula I is a compound of formula I.1:
E1_2 and E2_1′ wherein n is 0;
E1_4 and E2_1″ wherein n is 0;
E1_7 and E2_2 wherein n is 0;
in which
E1_2 is 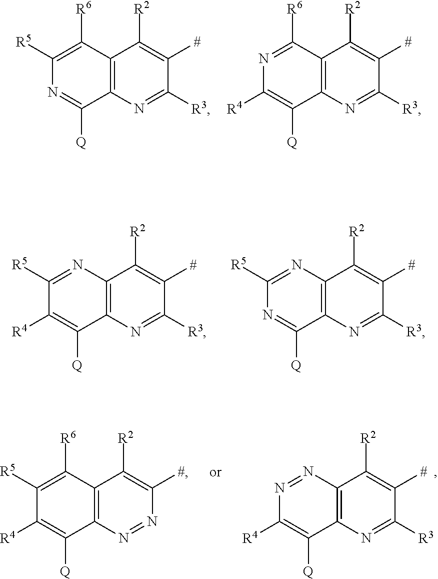
wherein
R2 is selected from the group consisting of hydrogen, chlorine, fluorine, bromine;
—NR¹²R¹³;
—OR¹⁴;
—SR¹⁵, —S(O)R¹⁵, —SO₂R¹⁵;
methyl, ethyl, propyl, isopropyl, cyclopropyl, cyclohexyl, propenyl, cyclopentenyl, cyclohexenyl, each of which is optionally substituted by 1 or 2 substituents independently selected from the group consisting of cyano, ethoxy-C(O)—, and —C(O)—NH₂; and
a monocyclic or a bicyclic heterocycle selected from the group consisting of azetidine, pyrrolidine, pyrazolidine, imidazolidine, 1,2,4-triazolidine, piperidine, piperazine, tetrahydropyridine, dihydro-2H-pyrane, tetrahydropyrane, 1,2-oxazolidine, 1,2-oxazine, morpholine, thiomorpholine, 3,4-dihydroisoquinoline, 2,3-dihydro-indole, 1,3-dihydro-isoindole, 3,9-dioxa-7-azabicyclo[3.3.1]nonane, 6-oxa-3-azabicyclo[3.1.1]heptane, 8-oxa-3-azabicyclo[3.2.1]octane, imidazole, pyrazole, 1,2,4-triazole, 1,2,3-triazole, 4-oxa-7-azaspiro[2.5]octane, each of which is optionally substituted by 1, 2, 3 or 4 substituents independently selected from the group consisting of fluorine, chlorine, cyano, —OH, oxo, —COOH, methoxy-C(O)—, ethoxy-C(O)—, tert-butoxy-C(O)—, —C(O)—NH₂, methyl, methyl-C(O)—, trifluoromethyl, hydroxymethyl-, methoxymethyl-, —NH₂, —NMe₂, pyrrolidine;
R3 is hydrogen, chlorine or methyl;
R4 is selected from the group consisting of hydrogen, halogen, cyano, C₁-C₄-alkyl, C₁-C₄-halogenoalkyl having 1 to 5 halogen atoms, C₁-C₄-alkoxy;
R5 is selected from the group consisting of hydrogen, fluorine, chlorine and methyl;
R6 is selected from the group consisting of hydrogen, fluorine, chlorine, methyl and methoxy;
R12 and R13 are independently selected from the group consisting of hydrogen, —NH(—C(O)-methyl), methoxy;
methyl, ethyl, propyl, isopropyl, butyl, isobutyl, cyclopropyl, cyclobutyl, benzyl, 1-phenylethyl, each of which is optionally substituted by 1, 2 or 3 substituents independently selected from the group consisting of fluorine, —OH, —COOH, methoxy-C(O)—, ethoxy-C(O)—, tert-butoxy-C(O)—, —C(O)—NH₂, —C(O)—NMe₂, —NH—C(O)-methyl, methyl, methoxy, cyclopropyl, —NH₂, NMe₂, S-methyl, S(O)-methyl, SO₂-methyl, and (EtO)₂P(═O)—;
heterocyclyl-methyl, heterocyclyl-ethyl, wherein the heterocyclyl substituent is selected from the group consisting of pyrrolidine, morpholine, pyrazole, 1, 2, 4-oxadiazole, pyridine, each of which is optionally substituted by 1 substituent independently selected from the group consisting of fluorine, chlorine, —OH, oxo and methyl;
phenyl; and
a monocyclic or a bicyclic heterocycle selected from the group of oxetane, thietane, pyrrolidine, morpholine, tetrahydropyrane, pyridine and pyrazole, each of which is optionally substituted by 1 or 2 substituents independently selected from the group consisting of fluorine, chlorine, —OH, oxo, methyl;
R14 is selected from the group consisting of
methyl, ethyl, isopropyl, butyl, cyclopentyl, benzyl, each of which is optionally substituted by 1 or 2 substituents independently selected from the group consisting of fluorine, —OH, methyl, methoxy and cyclopentyl; and
a monocyclic or a bicyclic heterocycle selected from the group consisting of pyrrolidine and tetrahydropyrane,
R15 is selected from the group consisting of
methyl and ethyl, each of which is optionally substituted by 1 substituent independently selected from the group consisting of —OH and —COOH; and
pyridine,
Q is a substituted phenyl ring of the formula (Q1) 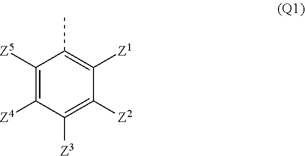
in which:
Z¹ and Z⁵ are independently selected from the group consisting of hydrogen, fluorine, chlorine, methyl and methoxy,
Z² and Z⁴ are independently selected from the group consisting of hydrogen, fluorine, chlorine, —OH, methyl, ethyl, —NHMe, —NMe₂, trifluoromethyl, methoxy, trifluoromethoxy, —SMe and morpholinyl, and
Z³ is independently selected from the group consisting of hydrogen, fluorine, chlorine, methyl, methoxy and —NMe₂;
wherein E2_1′ is 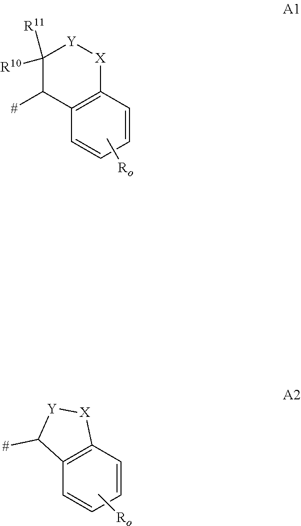
wherein
o is 0 or 1;
R is selected from the group consisting of hydrogen, halogen, C₁-C₄-alkyl and C₁-C₄-alkoxy;
X is selected from the group consisting of CR⁷R⁸, O, S, and N—R⁹;
Y is CR⁷R⁸;
R7 is selected from the group consisting of hydrogen and C₁-C₄-alkyl;
R8 is selected from the group consisting of hydrogen and C₁-C₄-alkyl;
R9 is C₁-C₄-alkyl;
R10 is selected from the group consisting of hydrogen, —OH and C₁-C₄-alkyl;
R11 is hydrogen;
E1_4 is 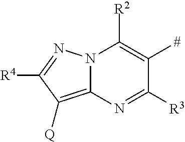
wherein
R2 is selected from the group consisting of hydrogen, methyl, ethyl, isopropyl, isobutyl, sec-butyl, cyclopropyl, methoxymethyl, difluoromethyl, trifluoromethyl, 4-fluorobenzyl, methoxy, methylamino, dimethylamino, cyclopropylamino,
—N(CH₃)(cyclopropyl), —N(CH₃)(CH₂—N(CH₃)₂), —N(CH₃)(CH₂—CHF₂), —N(CH₃)((CH₂)₂O(CH₂)₂)O(CH₂)₂)OCH₃), —N(CH₃)((CH₂)₂—S—CH₃), —N(CH₃)((CH₂)₂—S(O)—CH₃), —N(CH₃)((CH₂)₂—SO₂—CH₃), —N(CH₃)(1-methyl-piperidin-4-yl), —N(CH₃)((CH₂)₂-(oxopyrrolidin-1-yl)), morpholino, CH₂—N(CH₃)₂;
R3 is selected from the group consisting of hydrogen and methyl;
R4 is selected from the group consisting of hydrogen, chlorine, methyl, cyclopropyl, difluoromethyl, trifluoromethyl, —S-methyl, —S-ethyl, —S-isopropyl, —S(O)₂-methyl, —S(O)₂-ethyl, —S(O)₂-isopropyl; and
Q is a substituted phenyl ring of the formula (Q1) 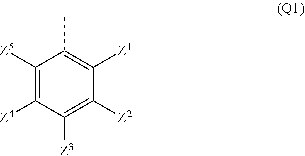
in which:
Z¹, Z², Z³, Z⁴, and Z⁵ are independently selected from the group consisting of hydrogen, fluorine, chlorine, bromine, cyano, methyl, propyl, difluoromethyl, trifluoromethyl, hydroxy, methoxy, ethoxy, isopropoxy, —O-cyclopropyl, —OCH₂-cyclopropyl, —OCH₂CN, trifluoromethoxy, difluoromethoxy, trifluoroethoxy, methylamino, dimethylamino, methylethylamino, diethylamino, acetylamino, methylsulfonamide, trifluoroacetylamino, —SO₂Me, —SO₂— cyclopropyl, —CH₂—O-methyl, —CH₂—O-ethyl, —CH₂—O—CH₂-cyclopropyl, —CH₂—O-isopropyl, —CH₂—N(CH₃)₂, —CH₂—N(CH₂CH₃)₂, —CH₂—N(CH₃)(CH₂CH₃), —CH₂—SCH₃, —CH₂—S(O)CH₃, —CH₂—SO₂—CH₃, —C(O)NH-cyclopropyl, and 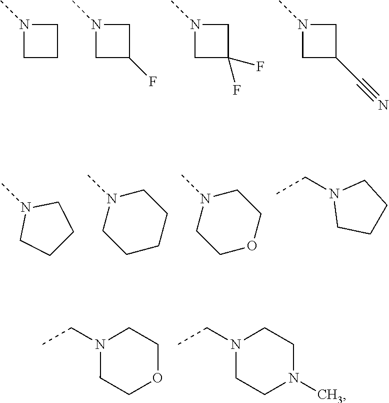
wherein at least two of Z¹, Z², Z³, Z⁴, and Z⁵ are hydrogen, or
Q is a pyridine ring of the formula (Q2) 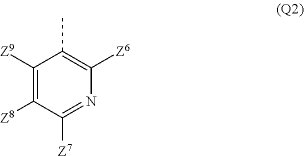
in which:
Z⁶ is hydrogen,
Z⁷, Z⁸ are independently selected from the group consisting of hydrogen, fluorine, chlorine, and
Z⁹ is selected from the group consisting of hydrogen and chlorine, or
Q is a pyrimidine ring of the formula (Q3) 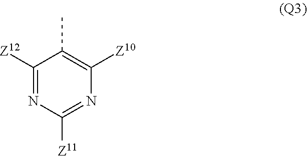
in which:
Z¹⁰ and Z¹² are hydrogen, and
Z¹¹ is selected from the group consisting of hydrogen and chlorine, or
Q is a pyridine ring of the formula (Q4) 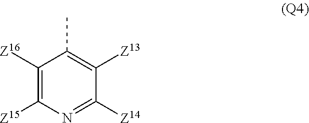
in which:
Z¹³, Z¹⁵, and Z¹⁶ are hydrogen, and
Z¹⁴ is selected from the group consisting of hydrogen and chlorine, NH₂, —NH—CO—C₁-C₄-alkyl, —NH(C₁-C₄-alkyl), —N(C₁-C₄-alkyl)₂, morpholino, or
Q is a pyridine ring of the formula (Q5) 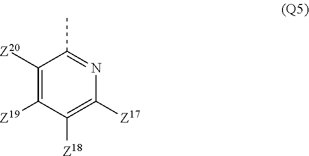
in which:
Z¹⁷ is selected from the group consisting of fluorine, chlorine, methoxy, trifluoromethyl,
Z¹⁸ and Z²⁰ are selected from the group consisting of hydrogen and chlorine,
Z¹⁹ is hydrogen, or
Q a pyrazole ring of the formula (Q6) 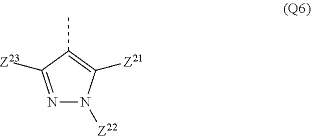
in which:
Z²¹ and Z²³ are hydrogen, and
Z²² is selected from the group consisting of hydrogen, methyl, ethyl, isopropyl, methoxyethyl, —CH₂-cyclopropyl, —CH₂CF₃, —CH₂CHF₂, —CH₂-morpholino, —CH₂—CH₂—N(CH₃)₂, and/or —CH₂—CH₂-morpholino, or
Q is a pyrazole ring of the formula (Q7) 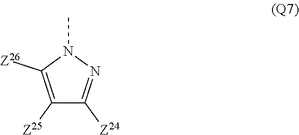
in which:
Z²⁴ and Z²⁶ are hydrogen, and
Z²⁵ is selected from the group consisting of hydrogen and chlorine, or
Q is selected from the group consisting of 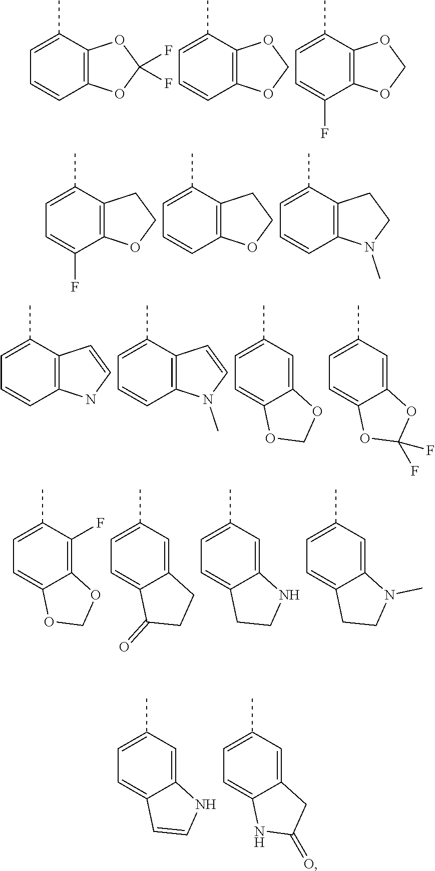
E2_1″ is 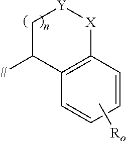
wherein
o is 0 or 1;
R is selected from the group consisting of hydrogen, fluorine, chlorine, methyl;
n is 0 or 1;
X is selected from the group consisting of CH₂ and O; and
Y is CH₂;
E1_7 is 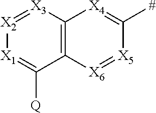
wherein
X₁ is CR₁; X₂ is CR₂; X₃ is CR₃; X₄ is CR₄; X₅ is N; and X₆ is CR₆;
E2_2 is 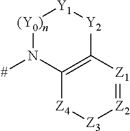
as defined in claim 2.

8. The method of claim 1, wherein
E1 and E2 are selected from the following pairs when the compound of formula I is a compound of formula I.1:
E1_7 and E2_1″ wherein n is 0;
E1_4 and E2_1″ wherein n is 0;
in which
E1_4 is 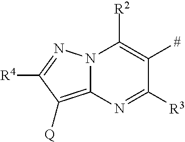
wherein
R4 is methyl;
Q is optionally substituted phenyl, wherein the optional substituent of phenyll is selected from the group consisting of halogen, alkyl optionally substituted with halogen, alkoxy optionally substituted with halogen, and —SF5;
R2 is selected from the group consisting of alkyl, cycloalkyl optionally substituted with halogen, CN, SO₂, 5- or 6-membered lactone, and 5- or 6-membered lactame;
R3 is H;
E1_7 is 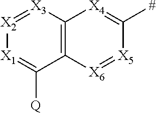
wherein
X1 is N or CR₁;
X2 is N or CR₂;
X3 is N or CR₃;
R1, R2 and R3 are selected from the group consisting of halogen, alkyl optionally substituted with halogen, alkoxy optionally substituted with halogen, —SF₅;
X4 is CR₄;
R4 is selected from the group consisting of alkyl, cycloalkyl optionally substituted with halogen, CN, SO₂, 5- or 6-membered lactone, and 5- or 6-membered lactame optionally substituted with halogen, 4- to 8-membered heterocycloalkyl optionally substituted with halogen;
X5 is CH;
X6 is N;
wherein at least one of X₁, X₂, X₃, is N;
E2_1″ is 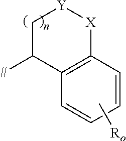
wherein
X is —CH₂— or 0;
Y is —CH₂—;
n is 0 or 1;
R is selected from the group consisting of hydrogen, halogen, cyano, nitro, —OH, C₁-C₄-alkyl, C₁-C₄-halogenoalkyl having 1 to 5 halogen atoms, C₁-C₄-alkoxy, C₁-C₄-halogenoalkoxy having 1 to 5 halogen atoms, C₃-C₆-cycloalkyl, —NH₂, —NH(C₁-C₄-alkyl), —N(C₁-C₄-alkyl)₂, —S—C₁-C₄-alkyl, —S(O)—C₁-C₄-alkyl, —SO₂—C₁-C₄-alkyl, —S—C₁-C₄-halogenoalkyl, —S(O)—C₁-C₄-halogenoalkyl and —SO₂—C₁-C₄-halogenoalkyl having 1 to 5 halogen atoms;
o is 0, 1,2,3 or 4.

9. The method of claim 1, wherein the compound has
a half-life of at least 20 hours, optionally at least 30 hours, or optionally at least 35 hours, or optionally at least 40 hours; and/or
a plasma clearance of less than 2 L/h/kg, optionally less than 1.5 L/h/kg, or optionally less than 1 L/h/kg.

10. The method of claim 1, wherein the compound prevents and/or treats the onset of a disease occurring at least one month after its administration, optionally at least one and a half months after its administration, or optionally at least two months after its administration, or optionally at least three months after its administration, or optionally at least six months after its administration.

11. The method of claim 1, wherein the disease is a helminthic infection.

12. The method of claim 1, wherein the compound is used for long-term prevention and/or treatment of a disease in an animal, optionally in a companion animal, or optionally in a cat or a dog, or optionally in a dog.

13. A method of using a pharmaceutical formulation in the long-term prevention and/or treatment of a disease, the formulation comprising at least one compound of claim 1 and at least one further ingredient, and the method comprising administration of the formulation to a subject in need thereof.

14. A product comprising the compound of claim 1 for long-term prevention and/or treatment of a disease.

15. A method for long-term prevention and/or treatment of a disease comprising the administration of the compound of claim 1 at an effective dose to a subject in need thereof.
